(19) United States
(12) Patent Application Publication
(10) Pub. No.: US 2025/0157616 A1
(43) Pub. Date: May 15, 2025

(54) COMBINATION MEDICAL TREATMENTS WITH ENERGY SYSTEMS

(71) Applicant: SANUWAVE, INC., Alpharetta, GA (US)

(72) Inventors: Iulian Cioanta, Alpharetta, GA (US); John Jackson, Alpharetta, GA (US); Kevin Richardson, Alpharetta, GA (US); Cary McGhin, Alpharetta, GA (US)

(21) Appl. No.: 18/934,218

(22) Filed: Oct. 31, 2024

Related U.S. Application Data
(63) Continuation of application No. PCT/US24/45837, filed on Sep. 9, 2024.
(60) Provisional application No. 63/581,503, filed on Sep. 8, 2023.

(51) Int. Cl. G16H 20/30

(52) U.S. Cl. CPC G16H 20/30

(57) ABSTRACT

A single medical treatment unit incorporates and coordinates operation of a plurality of different energy-producing technologies for synergistically treating a condition of a target region of a living thing with different types of energy.

### CROSS-REFERENCE TO RELATED APPLICATIONS

This application is a continuation of PCT Application No. PCT/US24/45837, filed Sep. 9, 2024, which claims the benefit of priority to U.S. Provisional Application No. 63/581,503 filed Sep. 8, 2023, each of which is incorporated herein by reference in its entirety.

### BACKGROUND OF THE INVENTION

There are various types of energy-based medical technologies that can successfully be utilized to enhance wound/lesions closure and treat or healing of different medical afflictions. In the case of wounds, all energy-based therapies or their combinations should be used in conjunction with appropriate moist wound healing practices according to the advanced therapy algorithms. The application of the exogenous energies has been shown to augment cellular, tissue, and vascular responses in both acute and chronic wounds/lesions.

Also, various types of energy-based medical technologies can be used for the treatment of different cells, tissues, and organs situated inside the human or animal bodies, or plants, which is facilitated by the possibility of the energy-based medical technologies to penetrate at controllable and precise depths inside the human or animal bodies or plants. This ultimately constitutes a significant advantage when compared with other existing medical technologies.

Furthermore, the combination treatment regimens with energy-based medical technologies or combination energy devices/systems that can deliver simultaneously multiple energy-based technologies can be utilized to heal different afflictions for human or animals that affect different tissues or organs situated superficially or internally relatively to the skin of the respective organisms. The combination energy-based medical devices or treatment regimens are able to tap in fundamental healing mechanisms of a leaving body, can restore equilibrium and also get rid of harmful bacteria, fungi, viruses, or other micro-organisms that are producing various illnesses for living beings, as humans and animals and also for plants. Currently in many medical situations, different medications/drugs are given systemically or locally to help with symptoms or target different mechanism of action for specific disease or to harm precise invading microbes. However, the chemicals/medications used to kill these microbes are effective only for specific ones and if the microbes can be reached. The fact is, over twenty percent of these microbes are under the surface of the skin, hiding in the pores, glands, and along the hair follicles that permeate the skin. The surgical preparations available today do not get to these areas and, therefore, cannot kill these bacteria/fungi. Additionally, the bacteria, fungi, viruses, or other micro-organisms hide inside the human or animal bodies in places difficult to reach by the blood supply, hibernate and reactivate when conditions are favorable (as it happens in shingles), and in the end the body cannot get rid of them. The combination treatment regimens with energy-based medical technologies or combination energy devices/systems that can deliver simultaneously multiple energy-based technologies have the advantages of being applied mostly from outside the body (extracorporeally), although can be also applied from inside the body (intracorporeally), and can reach any depth inside the human or animal body. Also, another advantage is giving by the fact that the combination treatment regimens with energy-based medical technologies or combination energy devices/systems that can deliver simultaneously multiple energy-based technologies are not specific for a certain type of microbes and thus do not generate any type of adaptations or mutations, which avoids the drug-resistance of the microbes that is seen more and more in the medical field. These characteristics of the combination treatment regimens with energy-based medical technologies or combination energy devices/systems that can deliver simultaneously multiple energy-based technologies makes them easily to be applied for many types of diseases and eliminate low efficiency therapies or treatments that can generate significant side-effects.

Another important action of the combination treatment regimens with energy-based medical technologies or of the combination energy devices/systems that can deliver simultaneously multiple energy-based technologies is the possibility to easily facilitate the efficient delivery of cellular material, as genes, DNA, RNA, mRNA, etc., inside cells, tissues or organs, which can reverse different genetic diseases or produce an efficient vaccination against infection with different microbes or as a treatment against cancer.

Furthermore, the combination treatment regimens with energy-based medical technologies or combination energy devices/systems that can deliver simultaneously multiple energy-based technologies can be used for the transfer of stem cells inside the body, and in helping with their survival and differentiation inside the body, which can reverse the damage of tissues or organs and restore their functionality.

The combination treatment regimens with energy-based medical technologies or combination energy devices/systems that can deliver simultaneously multiple energy-based technologies can also be used for the transfer of cellular and biological materials in the skin or inside internal tissues or organs. The cellular or biological material can comprise of a placental extracellular matrix composition or a placental connective tissue matrix composition. The placental extracellular matrix composition or placental connective tissue matrix composition can be prepared from whole placenta, placental decidua, placental amniotic membrane, or placental chorionic membrane. The cellular or biological material can comprise a population of adherent cells. The cellular or biological material can comprise a mixture of adherent and non-adherent cells. The cellular or biological material can comprise platelet rich plasma or placental perfusate. The cellular or biological material can be micronized by grinding, milling, freeze drying, or heat drying. The efficient delivery of the cellular or biological material via the combination treatment regimens with energy-based medical technologies or combination energy devices/systems that can deliver simultaneously multiple energy-based technologies will help with a faster integration and healing of the targeted tissues or organs.

Currently there are energy-based noninvasive, wearable medical devices to help women with stress urinary incontinence that is the inability to control the urge to urinate, which is a condition impacting about one in three women. Such device delivers an electrical stimulation to tone and strengthen pelvic floor muscles and treat stress incontinence.

Shockwaves, non-contact ultrasound or contact ultrasound, electromagnetic waves, light therapies, cold plasma, radio-frequency, vacuum, electrical currents, and other energy-based technologies are used now to treat a range of acute and chronic wounds or skin conditions with various grade of success.

Fluorescence biomodulation) can be used for specific skin/soft tissue conditions and diseases. The technology impacts cellular signaling pathways, which stimulate the skin's own biological processes and repair mechanisms.

Other technologies are specifically targeting chronic and acute complex wounds by providing fluorescence at a cellular level to impact the inflammation, proliferation and remodeling, which are the three critical phases of wound healing. Upon LED (light-emitting diode) activation, the Light Absorbing Molecule (LAM) topical generates fluorescence, which penetrates the skin at various depths to deliver high response rates and ignite the fast onset of wound regression, while providing an excellent safety profile. Such technology is an active wound care solution that delivers high response rates and ignites the onset of wound regression, while providing an excellent safety profile.

Another new approach for medical treatments is the use of the 4D hydrogel-based materials that can undergo multiple shape changes in response to environmental cues or to energy stimuli, which can be produced by the combination treatment regimens with energy-based medical technologies or combination energy devices/systems that can deliver simultaneously multiple energy-based technologies. These 4D hydrogels can morph multiple times in a pre-programmed or on-demand manner in response to external triggers and may lead to the development of human tissues, and even organs, that more closely resemble their natural counterparts, according to researchers.

The photobiomodulation is well-known adjunctive treatment for pain management, wound healing, lymphedema, cellulitis, pneumonia, chronic obstructive pulmonary disease (COPD), asthma and even COVID-19. The near-infrared laser light treatment that can be used to produce photobiomodulation, which can increase oxygenation, micro-organism deactivation, and overall tissue regeneration.

Furthermore, the energy devices are currently used to treat and heal musculoskeletal afflictions with different rates of success. In general, the energy is promoting the healing of muscles, tendons, ligaments, organs, glands, joints, blood and lymphatic vessels, nerves, skin, gonads, and bones and were successfully administered as extracorporeal systems to avoid surgeries and more invasive medical approaches. Based on the clinical literature, it seems that the energy medical systems tap and coopt body's multiple healing mechanisms, by reminding the body to restart the repair of certain areas/regions and to promote growth factors, revascularization, modulate inflammation, reduce or eliminate pain and ultimately promote tissue regeneration. The effect is the same regardless if the action goes on soft, semi-soft, or hard tissue for both humans and animals or even for plants.

The energy systems also show promises in regulating the functionality of different organs by normalizing their bio-currents, through reduction of inflammation, elimination of harmful micro-organisms, bacterial or viral infections, by helping with cellular mechanotransduction and substances exchange at the cellular level, DNA or RNA intake to modify and improve cell functionality, by coopting circulating stem cells in diseased areas and promoting implanted stem cells intake and differentiation, by starting or restarting the cellular communication to promote the repair process, through producing revascularization of ischemic areas, to name a few. The combination energy systems or different combination treatment regimens with energy-based medical technologies can be also used for localized drug delivery, which can eliminate the side effects produced by the systemic delivery of such drugs.

Additionally, the energy systems show a great rate of success in destroying planktonic bacteria, viruses, funguses, and other micro-organisms and disrupting the bacterial biofilms, which all can generate significant chronicity and infections inside the human or animal body or for plants. In general, for humans and animals the application of energy is done from outside the body (extracorporeal), but lately miniaturized energy systems are developed that can act intracorporeally by using normal cavities or blood vessels to reach the problematic areas.

Lately, such energy devices are also used to treat cancer through ablation or by turning the cancerous cells visible to the body's own immune system. Also, the energy systems can be used to reverse important side effects of different cancer treatments that produce irritation inside the mouth, or avascular bone necrosis, inflammation, lymph edema, as few examples of such delirious effects that affect the quality of life for the cancer patients.

The energy systems can be used together with complementary and alternative medicine or can even replace traditional approaches as acupuncture to stimulate the body's own healing mechanism and reduce pain by rebalancing the flow of energy inside the body via its meridian energy pathways. The complementary and alternative medicine is an umbrella term used to describe healthcare-related therapies, practices, and products that are not considered part of conventional medicine. Complementary or integrative medicine describes treatments used in conjunction with conventional medicine. Alternative medicine refers to therapies that are used in place of conventional medicine. The common belief within all complementary and alternative methods is that a body in balance is able to heal itself. When a body is out of balance, signs and symptoms of disease develop. At the risk of oversimplifying the philosophies of various traditional medicines, chronic disease states are signs that the body is out of balance within itself, and its local environment, and the surrounding energies from the environment. Restoring balance is the main focus of all-natural healing methods for traditional Chinese medicine, homeopathy, naturopathy, Ayurveda (traditional Indian medicine), and local traditional medicines. Based on their already proven mechanism of action, the energy systems can reestablish the normal bio-currents and the substance balance inside the organs and body in general, which in the end can promote healing and energetic equilibrium. The static or pulsed magnetic energy therapy can be applied to activate the body's natural electromagnetic impulses to assist healing. This is based on premise the human body is composed of electromagnetic frequencies that promote at certain equilibrium and health. Massage therapy can be produced by the energy systems at superficial level or deep inside the body as a therapeutic healing technique that involves the manipulation of superficial and deeper layers of muscle and connective tissue to enhance their function, promote relaxation, and ultimately healing.

### Electrical Stimulation

The first energy systems that were introduced in the medical applications are based on the electrical stimulation. Endogenous (internal originating from within the body) bioelectrical potentials measured across the skin of many animals and humans appear to be important in the healing process, as a strong correlation exists between their presence and rapid progression of the healing process. Electrical stimulation uses an electrical current to transfer energy to the tissue. Human or animal tissues are able to react to the application of electricity fields from the outside the body, sensing them and reacting according to a complex paradigm in relation to the intensity, polarity, time, and point of application. The same stands for plants. The electric energy produces a number of cellular processes and physiological responses that are important for cellular, tissue and organ healing. Also, the electrical stimulation can facilitate “restarting” the stalled healing sequences of events and it is therapeutically efficacious in cellular, tissue or organ repair and healing.

For humans, the electrical stimulation is presented in the form of low intensity direct current (LIDC), high voltage pulsed current (HVPC), asymmetrical biphasic pulsed current (ABPC), alternative current (AC), transcutaneous electrical nerve stimulation (TENS) for pain, and interferential current therapy (IFC). IFC is a deeper form of the common TENS therapy option, designed to relieve pain and to accelerate the self-healing process. The high frequency signals are capable of penetrating through the skin into deeper lying muscle tissues without painful stimulation to the superficial ones. For sure in the future new forms of electrical stimulation with very low currents mimicking the bio-currents will be used to equilibrate the body's energy balance and restore health to the cells, tissues, and organs. In general, these electrical stimulation devices are commonly battery powered, which increases their portability and the ease-of-use.

Electrical stimulation using local cathodal high-voltage pulsed current, neuromuscular stimulation, and transcutaneous electrical nerve stimulation has been shown to improve tissue perfusion and reduce edema formation in persons with peripheral vascular disease. These circulatory changes will indirectly stimulate the healing process by facilitating oxygen delivery. The bactericidal effect of enhanced blood flow together with the direct antibacterial effects of cathodal stimulation will act to prevent delayed healing due to the presence of infection. Similar mechanism of action, should also help with destruction of fungal particles that generate fungal infections or against any other harmful micro-organisms. Also, the electrical stimulation has been shown to be clinically useful in orthopedics for enhancing bone repair in non-unions following fracture, incorporation of bone graft, osteotomy, and arthrodesis. Additionally, the electrical current has been shown to induce cellular actions in virtually all phases of the wound healing cascade. Exogenous (externally applied) electrical current used for chronic wounds reproduces the endogenous potential and stimulates a number of cellular processes that are known to be important for wound healing. These include stimulation of several activities of fibroblasts including enhanced collagen and DNA synthesis, increased number of receptor sites for certain growth factors, and alteration of the direction of fibroblast migration along the direction of electrical currents. In-vitro studies on neutrophils, macrophages, epithelial cells, and fibroblasts collectively suggest that electrical stimulation contributes to accelerated wound repair by stimulating the migration and activation of key cells in the wound site. In vivo studies have revealed that electrical stimulation of these important cellular processes results in more collagen deposition and angiogenesis, greater wound tensile strength, and faster wound contraction rate.

Numerous different stimulus parameters have been shown to be effective in accelerating healing. More recently, electrical stimulation portable devices can be programmed to turn off and on and thus administer several treatment sessions throughout the day.

When applied as much as 30-minute treatments, 3 to 7 times per week for at least 4 weeks, electrical stimulation can produce the following:
a. Increases blood flow, especially through microcirculation due to angiogenesis and neovascularization
b. Generates peripheral nerve stimulation
c. Stimulates the production of cytokines and other proteins involved in the inflammatory process modulation
d. Produces stimulation of DNA and protein synthesis
e. Increases tissue oxygenation/perfusion
f. Reduces neuropathic pain
g. Stimulates growth factors, such as epidermal growth factor (EGF) and vascular endothelial growth factor (VEGF)
h. Increases the number of receptor sites for growth factors
i. Produces edema reduction/prevention
j. Stimulates acupuncture points
k. Produces antibacterial effect
l. Generates antifungal effects and destroys harmful micro-organisms
m. Increases tissue tensile strength
n. Guides the homing of bone-marrow-derived mesenchymal cells
o. Regenerates nerves
p. Stimulates collagen production
q. Increases fibroblast and endothelial cells proliferation and movement towards the injured tissue

### Electromagnetic Fields

In general, human and animal bodies are able to produce electricity and use it for a range of functions, including the nerve conduction of information, muscle contraction, polarizing cells, inducing biochemical reactions, separating body fluids, along with a number of other functions. The main source of self-produced electricity is the ion exchange through the cellular membrane, which is a natural dielectric structure, normally charged with negative potentials on the outside and positive potentials on the inside. This potential can be measured and the changes are related to cells and tissues functions as well as their dysfunctions. For example, the reduction or nullification of this electric potential is a sign of sufferance and death of cells. Unlike electrical fields, the magnetic fields are not produced by the cells and living tissue, but can be generated by the electric fields when they change rhythmically. In the body, these changes are generally very short-lived. The generated magnetic fields are therefore called pulsatile electromagnetic fields.

Our tissues are able to react to the application of magnetic fields from the outside the body, sensing them and reacting in relation to the intensity, polarity, time and point of application. Interesting to note that external magnetic fields can penetrate the cells and tissue while electric fields are stopped by the cell membranes. Electromagnetic fields (EMFs) and pulsed electromagnetic fields (PEMF) interact with electrically charged molecules present in tissue resulting in the production of induced electrical currents.

Among the many aspects of human physiology that are influenced or regulated by external electromagnetic fields are tissue repair and wound healing, which are probably the healthcare areas that have accumulated the most evidence. Additionally, the diagnostic and therapeutic applications of electromagnetic fields have strong roots in these areas. In general, any tissue lesion produces an interruption in the normal polarization of tissues, and a disturbance in the electromagnetic fields. Electromagnetic fields not only can be used in chronic ulceration, but also in a variety of chronic inflammatory conditions of both skin and the osteo-muscular apparatus, such as tenosynovitis, arthrosis, and traumas. Also, in other conditions characterized by poor regenerative activity, such as osteoporosis, the electromagnetic fields have benefic effects. Thus, the pulsed electromagnetic fields can play an important role in the local control of inflammation and, in particular, the prevention of articular cartilage degeneration and in the repair activity that has been observed in osteonecrosis of the femoral head through stimulating neovascularization and new bone formation.

A new generation of devices using the electromagnetic fields have been implemented and tested in chronic wounds with positive results. Their way of interaction with the biologic systems is different from the traditional versions as they do not act directly due to their extremely low intensity. Instead, they ‘communicate’ with the electromagnetic fields present inside the cells by frequency modulation sequences that characterize these electromagnetic fields. This is phenomenon is known as bio-resonance. It modifies the frequencies of the electromagnetic fields inside the cells. For this reason, they have been identified with the generic term ‘therapeutic magnetic resonance’.

Electromagnetic fields that are artificially produced by technological means (any electric current generates such electromagnetic fields) have a much lower intensity, but are more intense than the Earth's background magnetic field. They interact with our organisms, exerting a variety of effects of tissue and organ physiology. This happens either by directly interfering with the magnetic-sensitive molecules (all of the ones that contains charged ions or metallic components) or by directing and orienting the movements of molecules and organelles.

Any external electromagnetic field applied to a proteinaceous structure (consisting of protein) is able to determine a movement and a change in its structure, and a typical example of this is the alignment that collagen fibers show when an electromagnetic field is applied to them. This is one of the bases of the therapeutic application of external electromagnetic fields for tissue repair and wound healing. The first and more documented applications of electromagnetic fields was in the treatment of bone non-unions. In this therapeutic application, the electromagnetic fields are associated with faster and more stable, stabilization of the fractures with an increase in the speed and amount of callus formed and with a better alignment of the matrix fibers and an improved calcification. More recent evidence, including in vitro and in vivo studies, focused on the many positive effects that electromagnetic field can exert on virtually all of the phases of tissue repair.

The electromagnetic fields interact with electrically charged molecules present in tissue resulting in the production of induced electrical currents. The usual treatment needs between 6 to 8 hours per day for a period up to 6 months. An external electromagnetic field has the following effects:
a. Increases perfusion
b. Generates anti-inflammatory action, mediated by the shift in the production of cytokines from a pro-inflammatory pattern to an anti-inflammatory pattern.
c. Produces growth factors
d. Generates fibroblasts proliferation and activation
e. Produces collagen fibers orientation
f. Increases angiogenesis and neovascularization alongside the orientating effect on collagen fibers
g. Stimulates cellular communication and multiplication
h. Regenerates nerves
i. Reduces tissue edema
j. Some electromagnetic fields can produce tissue heating and others not

### Ultrasound

Therapeutic ultrasound delivers mechanical energy through a conducting media in the form of sound waves at a frequency above detection by the human ear. This modality is a commonly used therapeutic modality across the world to treat numerous musculoskeletal and integumentary disorders, including chronic ulcers. Ultrasound stimulates release of chemoattractant and mitogenic factors from inflammatory cells such as platelets, macrophages, and mast cells, enhances cell proliferation, and upregulates the synthesis and secretion of collagen.

Sound waves produced by ultrasound equipment is used for musculoskeletal disorders and to treat chronic wounds using both direct and indirect application techniques. Reports of accelerated wound closure have been achieved by delivering low frequency sound waves (30 kHz) from a large stationary sound head immersed in a water bath. Lower frequency sound waves have been also delivered using a novel mist technology (see U.S. Pat. Nos. 11,331,520 and 11,331,520). The delivery of non-contact, low frequency, and non-thermal ultrasound via a liquid mist to wounds has been shown to effectively debride necrotic tissue, eradicate some strains of bacteria from the wound, stimulate tissue regeneration, and facilitate the wound healing process. Recent studies have suggested that non-contact low frequency ultrasound as beneficial in helping to resolve or mitigate the severity of suspected deep tissue injury, when applied early in the sequelae of tissue destruction from these wounds. The ultrasound (contact and non-contact) treatments can be applied for 15-45 min per session, 3 times per week and for max 3 weeks.

Low frequency ultrasound therapy is believed to promote tissue healing via two mechanisms. The first mechanism is the ultrasound cavitation, which is the production and vibration of micron-sized bubbles within the coupling medium and fluids within the tissues. The second mechanism is the generation of acoustic streaming, which is the movement of fluids along acoustic boundaries. The combination of cavitation and acoustic streaming, both of which occur more frequently with kilohertz than megahertz ultrasound, provides a mechanical energy capable of altering cell membrane activity and, therefore, cellular activity.

Cavitation is the vibrational effect of ultrasound on micron-sized gas bubbles that form in the blood and lymph and tissue fluids and in the ultrasound coupling media solutions. Periods of high and low pressure in treated tissue can cause the bubbles to increase and decrease in size. Cavitation can be both stable and unstable. The stable cavitation acts to enhance the acoustic streaming phenomena and the unstable/transient cavitation occurs when the microbubbles significantly increase in size and violently implode during the low-pressure part of the ultrasound wave cycle. On implosion, the bubbles release a large amount of energy, which is destructive to bacteria while being essentially harmless to healthy viable tissue and cells, if the treatment is well controlled timewise. Similar mechanism of action, should also help with destruction of fungal particles that generate fungal infections or against any other harmful micro-organisms. Also, the tiny shockwaves produced by the cavitation bubble implosions cause preferential and rapid liquefaction and fragmentation of adherent necrotic fibrin (fibrinolysis) and the destruction of microorganisms.

Acoustic streaming has been shown to alter cell membrane permeability and messenger activity, which in turn may result to increased production of growth factors as well as macrophage activity, which results in fibroblast proliferation and migration creating a collagen-rich connective tissue matrix that ultimately facilitate the tissue repair. The mechanical energy from an ultrasound wave is absorbed by an individual protein molecule, inducing a conformational change. Signal transduction pathways can also be stimulated from ultrasound-generated mechanical energy. This may result in a broad range of cellular effects that impact tissue healing, including leukocyte adhesion, growth factor and collagen production, increased angiogenesis, increased macrophage responsiveness, increased fibrinolysis, and increased nitric oxide levels.

In contrast to the noncontact nonthermal energy transfer devices, the contact thermal ultrasound devices can accomplish sharp selective and excisional debridement and are often referred to as ultrasound-guided debridement. Such device provides selective, precise, and gentle fragmentation of soft and hard tissues, with preservation of healthy tissue through ultrasonic separation of damaged tissue. All ultrasound-guided debridement devices allow for deep tissue penetration of the ultrasonic energy and cause bacterial cell destruction within the wound bed through combined vibrational and cavitational effects. Continuous irrigation with the ultrasound coupling solution provides a medium for cavitation and flushes the wound of necrotic tissue fragments and fibrin deposits mobilized during the debridement, while preserving the granulation tissue.

The high-frequency ultrasound is currently used in the range of 1 to 3 MHz range to promote soft tissue injury healing and occasionally reported to facilitate wound healing. High-frequency ultrasound devices create their effect by running electricity through a crystal in the sound head, causing the crystal to “vibrate.” The vibrations are then passed through the sound head via an ultrasound coupling medium into the tissues, causing them to vibrate and creating a local thermal effect.

The contact or non-contact ultrasound treatment can produce the following effects inside the human body:
a. Produces modulation of inflammation
b. Accelerates mast cells and macrophages release to clean necrotic tissue
c. Augments local tissue oxygenation/perfusion through vasodilation, with possible thermal effects for some types of ultrasounds
d. Reduces bacteria and bacterial biofilm, which cuts infection and chronicity of the affliction
e. Generates antifungal effects and destroys harmful micro-organisms
f. Elevates capillary permeability that increases calcium uptake
g. Increases nitric oxide (NO) levels to produce blood vessels dilation and consequently blood flow rise into affected area
h. Reduces pro-inflammatory cytokine and matrix metallopeptidase 9 (MMP-9) levels, which produces inflammation modulation
i. Increases cellular communication to get the affected area from chronic state to acute state
j. Produces angiogenesis and neovascularization that increases capillary density, and promotes faster wound contraction
k. Stimulates cells proliferation, including the fibroblastic activity
l. Produces nerve regeneration and peripheral nerves stimulation
m. Generates collagen stimulation and fiber orientation

### Phototherapy

The use of light for medical purposes dates back to ancient times when phototherapy was empirically prescribed for a number of clinical conditions ranging from skin pathologies to asthma, behavioral disorders and chronic wounds. The light has an important role in the synthesis of vitamin D, the photo-modulation of biorhythms and the anti-depressive effects. The non-thermal phototherapy implies an interaction of light with endogenous photo-receptors.

The definition of phototherapy or low-level light therapy is a form of light therapy that utilizes non-ionizing forms of light sources in the visible and infrared spectrum. It is a non-thermal process that involves endogenous light absorbing molecules (chromophores) that elicit photophysical and photochemical events at various biological scales. The light absorbing molecules are implicated in the generation of reactive oxygen species (ROS) after an interaction with photons. The secondary reactions include the effects induced within the metabolism of the cells and the tissues by transduction and amplification of the original signal, leading to a photo response. These processes result in beneficial therapeutic outcomes including, but not limited to, the alleviation of pain or inflammation, immunomodulation, and promotion of wound healing and tissue regeneration. Furthermore, phototherapy can be used to reduce the dosage of medication, which would have a corresponding reduction in the drug's side effects. Finally, the combination of phototherapy with specific medication produces a beneficial effect that surpasses the effect of either being used alone to treat the respective medical affliction.

The antimicrobial effect of phototherapy has been confirmed for use on biofilm-producing bacteria that include the majority of the cases of chronic wounds colonization, and which are particularly resistant to systemic antibiotic therapy. The light application can eradicate biofilm and infected wounds efficiently and safely.

Light comes in different colors, spreading across the rainbow of hues known as the visible spectrum. Each color in this spectrum corresponds to a different wavelength. The visible light spectrum ranges from short wavelengths of the violet or purple color to long wavelengths, which are red. Photons of light from the violet end of the spectrum have the highest energies and the highest frequencies, while red photons have lower energies and lower frequencies. Currently, there is a relatively narrow range of light wavelengths that can actually interact with the photo-receptors to exert photo-biomodulation, and they are comprised between 380 and 2500 nm. The sources of light for medical purposes may be generated either by LED (light emitting diode) or OLED (organic light emitting diode) or by LASER (light amplification by stimulated emission of radiation). In all the cases, the emitted light is monochromatic. However, in the case of LED/OLED generated light, the emission is not unidirectional, while LASER generated light is unidirectional and coherent, reaching much higher intensity with the same amount of energy, concentrating the area of application.

Instead of LED technology, the OLED (organic LED) technology can be used for the phototherapy or laser applications. In this technology organic photonics may be used that offer a thin, flexible and easy-to-manufacture substances. They require less space, can be divided into several segments and controlled independently, which offer dynamic sequences with different levels of brightness and color-changing capability with broad spectral tunability and ultrafast responses. All of these characteristics can be a significant advantage in the medical field over the classic LED technology.

### A. LED/OLED Phototherapy

The phototherapy based on the light emitting diode (LED) or organic light emitting diode (OLED) technologies is based on using the majority of the spectrum available 380 and 2500 nm, based on the specific requirements of the treatment and the targeted tissue.

White-light therapy (380 to 760 nm) produced with LED/OLED technologies penetrates the deepest and works to produce tissue tightening and reduces inflammation.

Blue light therapy (400 to 525 nm) produced with LED/OLED technologies is most often used to treat acne. It may do this by reducing activity in the sebaceous glands, so they produce less of the oil that can plug the hair follicles, leading to acne. Blue light may also kill acne-causing bacteria known as Cutibacterium acnes. That is accomplished by the fact that the blue light activates chemicals inside Cutibacterium acnes bacteria, which ruptures their outer wall and thus destroying the cell and their ability to form acne and other imperfections. Rosacea, psoriasis, wrinkles and sun damage are all ideal candidates for blue light therapy treatments.

Green-light therapy (495 to 570 nm) produced with LED/OLED technologies is used to treat pigmentation and bulky lesions of the skin. It helps to lighten hyper-pigmentation spots revealing a brighter complexion. The calming effect also has anti-inflammatory properties that soothe the surface of the skin. Green LED/OLED therapy is used to treat dilated capillaries, sagging skin around the eyes, under eye circles, hyperpigmentation and sun spots.

Yellow-light therapy (565 to 590 nm) produced with LED/OLED technologies is used to increase wound healing, collagen induction, skin hydration and the overall health of the skin. The LED/OLED yellow-light therapies are the perfect skin wavelength light that will leave the skin looking healthier and radiant, due to stimulation of the rejuvenation process within the cells to improve the density of the skin. It also works to reduce redness by increasing dermal blood flow.

Amber-light therapy (600 to 615 nm) produced with LED/OLED technologies is effective in the treatment of skin issues involving redness such as rosacea. It is ideal for sensitive skin because of its calming and soothing. Amber light therapy helps to flush waste from the skin, boost lymphatic flow, and increase cellular growth.

Red light therapy (620 to 750 nm) produced with LED/OLED technologies, as a stand-alone, is generally considered safe, although too much light may damage skin tissue, but too little might not work as well. Red light therapy is a new breakthrough technology that uses a specific kind of therapeutic light that penetrates, opens up fat cells and liquefies the fat in them. The fat then easily leaves the cells to be burned as energy. This results in the fat cells shrinking and your body visibly becoming smaller. Also, the red light is most commonly used to promote circulation.

Research is showing that near-infrared (NIR with wavelength of 800 to 2,500 nm) and red light applied through the skull to the brain has numerous positive effects. The effects include increased cerebral blood flow, increased adenosine triphosphate (ATP) energy production, modulation of immune response, increased neuroprotection and brain repair, and reduced inflammation and pain.

The LED/OLED technologies can be used to directly irradiate the targeted tissue or can be used to irradiate special gels containing chromophores activated by LED/OLED generated visible light. The light absorbing molecules from the gel release large spectra of photons at different wavelengths in the visible range. The gel is applied on the targeted surface and is not absorbed by the tissue, but it is activated by the light, which is applied for a duration of 5 minutes twice per week.

Another interesting and new LED/OLED phototherapy is called Photodynamic Therapy (PDT). This king of light therapy is a treatment that involves light-sensitive medicine or gels and a light source to destroy abnormal cells. It can be used to treat some skin and eye conditions, as well as certain types of cancer. Also. the photodynamic therapy is a well-established, non-invasive treatment for a variety of dermatologic disorders, including actinic keratosis. Treatment is particularly effective for improvement of fine wrinkles, skin roughness, actinic elastosis and mottled hyperpigmentation.

In general, the LED/OLED phototherapy can produce the following:
a. Increases production of nitric oxide (NO) levels to produce blood vessels dilation and consequently blood flow rise into affected area
b. Produces bacteria killing and bacterial biofilms destruction
c. Reduces matrix metallopeptidase 9 (MMP-9) levels
d. Produces anti-inflammatory increased response
e. Removes exudates from wounds
f. Activates lymphocytes, macrophages and mast cells to clean the dead cells/tissues and control infection
g. Produces intracellular increase of reactive oxygen species (ROS)
h. Increases of RNA and DNA synthesis
i. Augments permeability of cell membrane
j. Increases synthesis of interleukins and growth factors
k. Produces cellular metabolism amplification
l. Generates nerve regeneration
m. Increases intracellular calcium levels
n. Produces fibroblast proliferation

### B. Laser Phototherapy

LASER is an acronym for light amplification by stimulating emission of radiation. Lasers in the red visible wavelength and infrared lasers, are class III lasers, which do not result in any significant elevation in tissue temperature. The clinical practice suggest that laser is often the modality of choice for therapists treating chronic wounds in some countries. In general, for wound care the lasers are used for 5 treatments per week for 3 weeks. Similarly, the laser phototherapy is used for the treatment of musculoskeletal disorders, acute soft tissue injuries, shingles, regeneration for both nerve injuries and diabetic peripheral neuropathy, and reducing postoperative pain. Lately, the lasers are used for the successful treatment of hair loss with the treatment time of 5-10 min every other day for 6 months.

Lasers are also used for different acute and chronic skin conditions or for oral mucositis, a debilitating condition caused by radio-therapy or chemotherapy. Chemotherapy-induced oral mucositis causes the mucosal lining of the mouth to atrophy and break down, and thus forming ulcers inside the mouth, with debilitating consequences related to pain and the inability to proper eat food or drink liquids.

When discussing laser phototherapy in regeneration medical field, one is usually referring to a low-level or cold laser, which is used for tissue regeneration, as opposed to coherent laser beams of high-intensity light, which is used for surgical incisions. The net effect of a laser may depend on many factors as light amplitude that dictates laser's color and penetration, duration of the pulse, the presence of a continuous or intermittent current. The laser application technique employs a transparent film barrier in conjunction with a contact point for the lasers. A noncontact, sweeping technique can also be used; which involves less light energy delivered to the targeted tissue.

The laser phototherapy can produce the following benefic effects related to tissue regeneration:
a. Accelerates biochemical reactions
b. Produces mast cell degranulation
c. Augmented immune cell activity
d. Stimulates growth factors
e. Increases leucocyte proliferation
f. Produces anti-inflammatory effects
g. Stimulates neovascularization and angiogenesis
h. Upregulates intracellular processes, such as single oxygen molecules buildup that influences the formation of adenosine triphosphate (AP), which in turn leads to replication of DNA
i. Increases DNA synthesis leads to increased neurotransmission
j. Promotes lymphocyte activation and immune system activation
k. Has analgesic effects
l. Augments cellular membrane permeability
m. Increases intracellular calcium levels
n. Photons from a laser probe are absorbed into the mitochondria and cell membranes of the cells, which activates cellular communication and proliferation
o. Accelerates fibroblasts activity
p. Augments procollagen mRNA expression that enhances collagen metabolism and extracellular matrix synthesis
q. Amplifies keratinocyte migration and secretion
r. Produces nerve regeneration
s. Induces cascade of metabolic effects that result in various physiological changes

### Ultraviolet Light

Ultraviolet light (UV) has been used for centuries to treat a myriad of health and skin problems in the form of natural sunlight and more recently by artificial UV-generated sources. It involves exposing the affected areas to UV light that has a 100 to 400 nm wavelength range. There are four forms of UV light called UV light A (UVA), UV light B (UVB), UV light C (UVC) and vacuum UV. For skin conditions, the UV light reduces inflammation and slows the production of skin cells. The treatment is usually given three times a week in a dermatology practice or hospital. UV light therapy is a good treatment for a variety of other skin conditions as psoriasis, eczema, vitiligo, mycosis fungoides, and other skin problems. Most patients require 20-30 treatments to see results, depending on their condition. The amount of light energy delivered is known to be dependent on the duration of the treatment and the distance and angle of the light source.

Ultraviolet light A (UVA with 315-400 nm wavelength) and ultraviolet light B (UVB with 280-315 nm wavelength) are responsible for the pigmentation and erythema (and sometimes blistering) often seen in light-skinned individuals after significant sun exposure. UVB rays have a short wavelength that reaches the outer layer of the skin (the epidermis). UVA rays have a longer wavelength that can penetrate the middle layer of the skin (the dermis). UVB light assists in wound healing by inducing an inflammatory reaction, stimulating the growth of granulation tissue, and promoting breakdown and elimination of dead tissue from the wound. UVA and UVB are also used in the treatment of psoriasis. The exposure time should be short less than 90 seconds to prevent adverse events as carcinogenic or burn effects.

Ultraviolet light C (UVC with 200 to 280 nm wavelength) is the form of UV light most often used in the treatment of chronic wounds specifically for its bactericidal effects. UVC is capable of killing strains of bacteria in laboratory cultures, in animal tissue, and in patients with chronic ulcers infected with methicillin-resistant Staphylococcus aureus (MRSA) and Pseudomonas aeruginosa infections. For medical purposes, the UVC is the most used in chronic wounds treatments, where the lamp is maintained perpendicular to the wound and at a distance of 2.5 cm or 1 inch. UVC radiation is the highest energy portion of the UV radiation spectrum, but has also the smallest penetration depth. The antibacterial effect of UVC, which peaks at a wavelength of 254 nm, is believed to speed healing via removal of a bioburden to the natural debridement system and thereby allowing more rapid progress through the inflammation phase of wound healing. By using filters specific to UVC, potentially carcinogenic effects of UVA and UVB wavelengths can be reduced, and the risks of skin burns can be virtually eliminated.

The fourth form of UV is the vacuum UV that has a wavelength of 100-200 nm, and as its name suggests is characteristic for a vacuum environment. The medical systems that use vacuum UV are trying to reduce the strongly absorption of the UV light by the air, with the idea to optimize the actual medical treatment.

In nature, the vacuum UV together with UVC are completely blocked by the ionosphere, while UVB and UVA are in contact with our bodies with different grades of penetration, with UVA being capable to penetrate deeper than UVB.

Ultraviolet Light therapy can produce different effects on the targeted tissue treatment, as follows:
a. Produces epithelial migration and proliferation
b. Releases chemical mediators to produce erythema
c. Increases local cutaneous blood flow
d. Has antibacterial effects

### Topical Negative Pressure

Negative pressure wound therapy (NPWT) applies sub-atmospheric pressure, or suction, to the wound bed by way of a device that is attached to a wound contact layer (interface dressing) through a plastic tube. Several types of interfaces are used with these devices, including foams, moistened cotton gauzes, and nonwoven polyester layers joined by a silicone elastomer. The interface dressings are covered with a transparent film or thin hydrocolloid, depending on the device used, that seals the wound and dressing to maintain the vacuum effect. Interface dressings containing silver or other antibiotics are available from some manufacturers. Initially, the changing of the dressing is recommended 48 hours after beginning of the treatment, then two to three times per week. The negative pressure created by the pump is in the range of 0 to 200 mm Hg depending on the system used.

NPWT enhances wound closure based on externally applied stress, via delivery of energy in the form of negative pressure that creates macro-strains and micro-strains in the wound bed and in individual cells. Macro-strains are the physical response that can be seen immediately as the negative pressure contracts the wound. In addition, this mechanical stress creates changes at the cells' surface (also known as micro-deformations or micro-strains), causing growth factors and cytokines to “upregulate” fibroblastic activity, increasing production of the extracellular matrix and cell proliferation within the wound.

Lately, the NPWT is combined with the instillation of infusion solutions into the wound interface. A variety of solutions have been reported and include normal saline, hypochlorous acid, and other antimicrobials, which serve to enhance wound irrigation and cleansing as well as a decrease bacterial colonization across the surface of the wound.

NPWT reportedly facilitates wound closure and healing through several mechanisms of action, as follows:
a. Augments blood circulation and oxygenation in the treatment targeted area
b. Eliminates edema, which improves nutrient and oxygen delivery
c. Removes wound exudate to reduce bacterial colonization
d. Enhances anti-microbial activity of leukocytes
e. Promotes angiogenesis
f. Stimulates growth factors
g. Reduces bacterial growth
h. Decreases harmful levels of proinflammatory agents, such as matrix metalloproteases (MMPs) that produces a modulation of inflammation
i. Upregulates fibroblastic activity
j. Increases production of the extracellular matrix and cell proliferation
a. Facilitates wound contraction/retraction

### Shockwaves

In general, the focused acoustic pressure shockwaves or specially-designed pressure waves produced by the proposed embodiments from this invention will have a compressive phase and a tensile phase during one cycle of the acoustic pressure shockwaves/pressure waves. In the compressive phase, positive compressive pressures are produced and in tensile phase significant negative pressures are generated that produce cavitation bubbles, which when reaching their full dimensions implode/collapse with high-speed jets in excess of 100 m/s. On their implosion, the cavitation bubbles release a large amount of energy, which if enough energy is incorporated in them can be destructive to bacterial or viral or fungal or harmful micro-organisms outer shells, while being essentially harmless to healthy viable and surrounding tissue and cells, if the treatment is well controlled timewise. In conclusion, the two synergetic effects produced by the compressive and tensile phases generated by shockwaves, work in tandem to stimulate cells and tissues via different mechanisms to open the cell membranes, increase RNA production, call-in of different regeneration and growth factors, etc., and simultaneously destroy bacterial or viral or fungal or harmful micro-organisms outer shells, rendering them unharmful.

The high mechanical tension and pressures, found at the front of the focused acoustic pressure shockwave or of specially-designed pressure waves, distinguish the shockwaves from other kinds of sound waves, such as ultrasonic waves. The focused acoustic pressure shockwaves or specially-designed pressure waves consist of a dominant compressive pressure pulse, which climbs steeply up to maximum one hundred Mega-Pascals (MPa; 1 MPa=10 bar) within a few nanoseconds and then falls back to zero within a few microseconds. The final portion of the pressure profile is characterized by negative pressures of minus five to fifteen Mega-Pascals (tensile region of the acoustic pressure shockwave/pressure waves), with potential to generate cavitation bubbles in any kind of fluids. The bubble's diameter grows as the energy is delivered to the bubble. This energy is released from the cavitation bubble during its collapse (implosion) in the form of high-speed pressure micro-jets and also produces rapid transient high temperatures. However, the short duration of the shockwave pulse results in a temperature rise of <1° C., producing negligible thermal effects. For acoustic pressure shockwaves or specially-designed pressure waves to be effective, they must be focused or concentrated (semi-focused) or completely unfocused when sent towards the point or region at which their effect is needed. In general, in the targeted region, there are two basic effects, with the first being characterized as direct generation of mechanical forces (primary effect from the positive, compressive high-pressure rise), and the second being the indirect generation of mechanical forces (high velocity pressure micro jets) produced by implosion of cavitation bubbles (secondary effect from the negative, tensile pressure region).

A focused acoustic pressure shockwave or a specially-designed pressure wave can travel large distances easily (based on the amount of energy put in them at the point of origination), as long as the acoustic impedance of the medium remains the same. At the point where the acoustic impedance changes, energy is released and the acoustic pressure shockwave or pressure waves are reflected or transmitted with attenuation. Thus, the greater the change in acoustic impedance in between different substances, the greater the release of energy is generated at the interface of substances of different acoustic impedance.

The acoustic pressure focused shockwaves or specially-designed pressure waves (planar, radial, or unfocused waves) are highly controlled to generate an energy output that will not produce any undesired damage to the tissue or cell cultures or instrumentation where they are used. The means of controlling the energy and the penetrating depth of the extracorporeal acoustic pressure shockwaves used to treat living tissue or organs is done via the amount of energy generated from the acoustic pressure shockwave generators or energy settings (energy input into acoustic pressure shockwaves or specially-designed pressure waves (planar, pseudo-planar, radial, or unfocused waves)), the total number of the acoustic pressure shockwaves/pulses delivered in one session to the targeted region, the repetition frequency of the acoustic pressure shockwaves and the special construction of the shockwave reflectors and/or membranes used in the acoustic pressure shockwave applicators or delivery devices. Based on the type of reflector, the shockwaves can be focused, planar, pseudo-planar, radial, or unfocused pressure waves, and the height of the shockwave applicator membrane (sits on top of the reflector) dictates shockwave penetration under the skin of an animal or human, or from the surface of a plant subjected to shockwaves or pressure waves.

There are different methods of generating focused, unfocused, planar, planar, pseudo-planar or radial extracorporeal acoustic pressure shockwaves using an acoustic pressure shockwave generator or generators, as follows:
a. electrohydraulic generators using spark gap high voltage discharges
b. electrohydraulic generators using one or multiple laser sources
c. piezoelectric generators using piezo crystals/piezo ceramics
d. piezoelectric generators using piezo fibers
e. electromagnetic generators using a flat coil and an acoustic lens
f. electromagnetic generators using a cylindrical coil

Shockwaves have been demonstrated to increase vascular endothelial growth factor (VEGF) and nitric oxide (NO) concentrations, which promote angiogenesis and neovascularization. Other observations are related to the reduction in the production of pro-inflammatory cytokines, specific gene upregulation, bacterial cells destruction, and bacterial films cracking and dislodging. In general, shockwaves are also able to induce cells proliferation for osteoclasts, fibroblast, keratinocytes, endothelial and stem cells, to name a few, both in vitro and in vivo.

Shockwave treatments for different afflictions can be applied in various regimens. However, for significant better patient compliance was determined that one (1) treatment per week for up to 10 weeks, works pretty well for wound care. For orthopedic treatments, the number of treatment sessions can be significantly reduced, since the energy deposited by shockwaves in one treatment session is significantly higher when compared to wound care. In other words, tissue regeneration using shockwave therapy requires specific regimens based on each type of tissue and explicit aim of the treatment.

Shockwave therapy is capable of producing the following effects on the targeted tissues or organs that receive their energy:
a. Increases cellular permeability by opening pores on their membrane via mechanotransduction that facilitate the transport across membrane and intake of drugs, antibiotics, vaccines, genetic material, etc.
b. Elevates nitric oxide (NO) levels to produce blood vessels dilation and consequently augmentation of local tissue oxygenation/perfusion in the treatment targeted area
c. Increases cellular communication to get the affected area from chronic state to acute state
d. Eliminates edema, which improves nutrient and oxygen delivery
e. Accelerates mast cells and macrophages release to clean necrotic tissue
f. Enhances anti-microbial activity of leukocytes
g. Increases capillary permeability that intensifies calcium uptake
h. Promotes angiogenesis and neovascularization that increases capillary density, and overall generates faster wound contraction and healing through tissue regrowth
i. Stimulates growth factors
j. Reduces bacterial growth and produces bacterial biofilms fragmentation, which reduces infection and chronicity of the affliction
k. Generates antifungal effects and destroys harmful micro-organisms
l. Stimulates acupuncture points
m. Reduces pro-inflammatory cytokine and matrix metallopeptidase 9 (MMP-9) levels, which produce inflammation modulation
n. Produces cells proliferation including the fibroblastic activity
o. Stimulates peripheral nerves and regenerates nerves in general
p. Reduces neuropathic pain
q. Stimulates collagen
r. Increases production of the extracellular matrix and cell proliferation

### Nanotechnologies (NT)

The term nanotechnology (NT) refers to the research and application fields in the nanoscale dimension, which ranges between one and 100 nanometers (1 nm=1 billionth of a meter). Within medicine, many promising applications related to NT have been accomplished ranging from oncology to diagnostics and pharmacology and many others, including wound healing. The interest that nanotechnology raised within wound healing due to the physical characteristics of nanoparticles as well as their versatility and tunability, which make them suitable for use in the different phases of tissue repair independently or in conjunction with energy devices.

Nanoparticles can be classified into different types according to their size, morphology, physical, and chemical properties. Nanoparticles can be carbon-based nanoparticles, ceramic nanoparticles, metallic nanoparticles, semiconductor nanoparticles, polymeric nanoparticles, and lipid-based nanoparticles. Due to their small size and large surface area, drug nanoparticles show increase solubility and thus enhanced bioavailability, ability to cross the blood brain barrier, possibility to enter the pulmonary system, and also to be absorbed through the tight junctions of endothelial cells of the skin. Nanoparticles are taken up by cells more efficiently than larger micro-molecules and therefore, could be used as effective transport and delivery systems. For therapeutic applications, drugs and other active substances can either be integrated in the matrix of the nanoparticles or attached to the nanoparticle surface.

The high surface area/volume ratio makes it possible for nanoparticles to have a high probability of interaction with the cellular elements and an enhanced penetration deep into the tissues. Nanoparticles also allow a higher bioavailability at lower concentration with a lower toxicity as a result. Metallic or non-metallic nanoparticles or nanocrystals could be used as antibacterial agents due to their ability to kill bacteria and to disrupt biofilms, or can be modulatory elements on inflammation and tissue repair and also vehicles of bioactive agents that can be released via energy systems. For antibacterial effects metallic nanoparticles as silver, gold, or zinc oxide as well as non-metallic are used to support the general efforts to reduce the risk of antibiotic resistance in chronically infected tissues. Similarly, the nanoparticles should also help with destruction of fungal particles that generate fungal infections or against any other harmful micro-organisms. Carbon fullerenes in form of a hollow sphere, ellipsoid or tube have been demonstrated to significantly reduce inflammation and reduce the oxidative stress level in models of chronical inflammation.

Nanoparticles also can be used as vehicles and carriers for bio-active molecules, such as nitric oxide (NO), antibiotic compounds, and antioxidants. These substances are incorporated in nanoparticles made of Chitosan, or polylactic-CO-glycolic acid (PLGA), or polyethyleneimine (PEI), or alginate, or Xanthan gum, or cellulose, or liposomes, or polymeric micelles, or dendrimers, or inorganic nanoparticles, to name few options. By controlling their nano-dimensions certain types tissues can be targeted, based on their density and intercellular spaces, which allows the absorption of only nanoparticles of a certain dimension. Once the targeted tissue is reached and nanoparticles are absorbed inside the tissue, the nanoparticles can slowly deliver the active substances (due to their biodegradable nature) or special extracorporeal energy systems can be used to break the nanoparticles and locally deliver the active substances in sufficient high dosages for superior therapy results, without creating any systemic adverse reaction. That opens the door for the locally delivery of certain drug therapies that initially failed due to their adverse effects generated by the systemic delivery, although the compound effects could be beneficial for treating a certain disease, tissue, organ, etc. This creates the opportunity to reconsider the use in the medical field of some drugs and therapies that initially were rejected, due their systemic toxicity. The same approach could be used for delivering antibiotics, antioxidants, traditional natural medical substances, vaccines, proliferation factors, angiogenesis factors, growth factors, cytokines, exosomes, enzymes, stem cells antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., in a time/dose efficient way.

Nanoparticles therapy can produce the following actions, when administered for medical treatments in conjunction with energy devices:
a. Enhances antibacterial and antifungal effects and bacterial biofilms destruction
b. Augments oxygenation and perfusion
c. Produces anti-inflammatory effects
d. Delivers for different afflictions active substances or particles as drugs, antibiotics, antioxidants, traditional natural medical substances, vaccines, proliferation factors, angiogenesis factors, growth factors, cytokines, exosomes, enzymes, stem cells antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc.

### Pneumatic Compression Therapy

Administration of external pressure with an intermittent or sequential protocol can substantially promote venous return, prevent deep vein thrombosis, and eliminate limb edema. This is why the pneumatic compression therapy is considered essential standard therapy for the treatment of chronic leg wounds due to venous insufficiency. This compression therapy is believed to aid wound healing through reducing venous congestion, improving tissue perfusion, and promoting systemic fibrinolytic activity. The same type of pressure devices can be used to stop bleeding, to vibrated chest cavity, prevent inflammation, etc.

Pneumatic compression therapy can produce the following actions, when administered for medical purposes:
a. Prevents blood, fibrin, and protein leakage
b. Forces blood into the deep venous system and enhances blood flow
c. Increases oxygenated blood flow and the removal of potentially harmful toxic waste
d. Stimulates the detachment of lung mucus and its elimination
e. Aides with weight loss and burn fat
f. Reduces muscle soreness after exercise
g. Decreases stress hormone cortisol

### Hydrotherapy

Hydrotherapy is involving the delivery of water or other fluids to the wound bed and it is one of the oldest therapeutic modalities used by physical therapists in the treatment of chronic wounds. Hydrotherapy is most commonly applied using whirlpools, but more recently, other irrigation systems such as pulsed lavage with concurrent suction (PLWCS) have been designed for wound care. Wound cleansing using hydrotherapy removes necrotic and devitalized tissue, debrides loosely adherent yellow fibrinous or gelatinous exudate, and removes any unwanted dirt, foreign contaminants, or harmful residues of topical agents. In the end, the hydrotherapy produces a mechanical debridement and superficial tissue massage, which will aid wound healing indirectly due to reduced bacterial burden within the wound and cellular stimulation. Based on this mechanism of action of hydrotherapy, this therapeutic modality is indicated for open nonhealing wounds that have substantial amounts of necrotic tissue. Hydrotherapy should be discontinued when wounds are clean.

Most published protocols for the use of whirlpool on chronic wounds suggest that the limb should be immersed in water of a neutral temperature (92-96° F.) for 10 to 20 minutes. If agitation is added to the whirlpool water, care must be taken to avoid excessive pressures that can cause mechanical damage to fragile new tissue deposited in the wound bed. Antimicrobial agents are often added to the whirlpool water to prevent wound infection from waterborne contaminants during hydrotherapy treatments.

Hydrotherapy ca also be applied as pulsed lavage with concurrent suction (PLWCS) that applies a stream or spray under controlled pressure to remove loosely adherent necrotic tissue or foreign material, in the form of portable units with disposable attachments. Care must be taken to avoid irrigation with excessive pressures or with water of high temperatures for prolonged periods of time. Safe irrigation pressures are between 4-15 psi and water temperature from 80-104° F. (26.6-40° C.).

The hydrotherapy treatment that transfers energy in the form of heat and pressure on the targeted tissue have the following effects:
a. Cleans the local debris, slough, and necrotic tissue
b. Reduces inflammation
c. Controls infection through the removal of debris and exudate
d. Stimulates local blood perfusion of tissues
e. Enhances cellular and tissue activation
f. Increases analgesia through its neuronal influence
g. Produces tissue regrowth due to thermal and mechanical stimulation

### Radio Frequency

Human, animal or plant cells exchange information and regulate body functions through the sending and receiving of specific frequencies. This cell signaling precedes and regulates all biochemical actions. When these natural frequencies are affected, it can reduce the body ability to self-regulate and maintain healthy function or on the contrary by applying external specific radio frequencies, the normal functioning of cells can be restored.

Radio frequency medical equipment can be used to produce non-thermal or thermal effects, depending on the need of each medical treatment. The non-thermal radio frequency in the form of pulsed radio frequency energy is a therapy modality that can be used in the treatment of chronic lower limb ulcers and for soft and semi-soft tissues. Also, such treatment has a role in reducing the inflammation and pain associated with different medical conditions. The low radio frequency waves are linked to “delta” and “theta” states of the brain, which can boost relaxation and improve sleep. Higher radio frequencies can boost the brain waves into a “gamma” state which may make a person more alert, focused, or better able to recall memories.

The thermal radio frequency or diathermy equipment typically operates in the short-wave radio frequency (range 1-100 MHz). An application for the thermal radio frequency is for skin or cosmetic treatments. In such cases, the effects of radio frequency treatments will continue to improve over the next few months, as the skin produces new collagen. Results typically last for a minimum of six months, although with ongoing treatments, results are often long-lasting. Radio frequency is a safe and relatively effective method for improving skin appearance by stimulating the production of collagen. Since fat also responds to a lower temperature when compared with other cells from the body, heat can be used to destroy the fat cells and decrease subcutaneous fat, especially in the abdomen and thighs, without damaging surrounding skin and tissues. In addition, safety and relatively short time for the radio frequency treatments, represent important advantages of such energy treatment modality.

Another important effect of the radio frequency is the antibacterial effect, due to the shaking apart bacterial cells like an opera singer shattering wine glasses and thus destroying the bacteria. Also based on similar mode of action, with sufficient radio frequency energy, the viral protective shell undergoes alternating compression and rarefaction and resonates, which induces mechanical stress severe enough to shatter the virus protective shell, rendering the virus inactive. Similar mechanism of action, should also help with destruction of fungal particles that generate fungal infections or against any other harmful micro-organisms. In conclusion, besides the antibacterial effect the radio frequency can be an effective antiviral or anti-fungal energy treatment modality.

Pulsed radio frequency energy therapy can produce the following actions, when administered for medical purposes:
a. Produces the expression of genes involved in angiogenesis
b. Generates immunomodulation
c. Enhances antibacterial, antiviral, and antifungal effects
d. Forms new collagen fibers
e. Produces the destruction of fat cells
f. Upregulates growth factors

### Cold Atmospheric Plasma

Biological effects of cold atmospheric plasma are largely dependent on plasma-generated reactive species in the gas phase, which diffuse or react with proteins and lipids of cells or tissues. The treatment with cold atmospheric plasma generates charged particles; free electrons and ions, thermal, visible, and ultraviolet radiation; electrical fields, and highly active species at the site of interest. This may directly affect the function of cellular signaling or redox-reactive molecules. Reactive oxygen species produced by cold atmospheric plasma therapy, such as ozone, hydroxyl radicals, superoxide, and singlet oxygen, and reactive nitrogen species, such as nitric oxide or peroxynitrite, are expected to act as active compounds in the targeted tissue, which ultimately starts or restarts the healing cascade and tissue regeneration. Cold atmospheric plasma has been proposed as a tool for various biological and medical applications, due its capacity to reduce bacterial load in a wound and to initiate wound healing. This is why, the cold atmospheric plasma has been associated with benefits in wound infection and healing.

Cold plasma energy therapy can produce the following actions, when administered for medical purposes:
a. Enhances production of reactive oxygen species reactive nitrogen species
b. Upregulates growth factors
c. Augments bacterial killing
d. Accelerates body healing

### SUMMARY OF THE INVENTION

There is sufficient evidence available that the energy medical treatments are applying different amounts of stress to the cytoskeleton of the cells (mechano-biological, chemical, electric, magnetic, photonic, or thermal interactions), which generates a number of effects, including the repression/depression of genes and changes in protein synthesis of a number of cells, including keratinocytes, fibroblasts, endothelial cells, and bone marrow stromal cells. Besides that, the energy medical treatments are capable of killing bacteria, viruses, funguses, micro-organisms, etc. and have a destruction action on biofilms, which has a significant importance in elimination of infections and chronicity of many diseases. Furthermore, the majority of the energy medical treatments through different mechanisms are capable of enhancing blood circulation and tissue oxygenation. Even more, they are capable of stimulation of angiogenesis and neovascularization with significant effects in increasing the microcirculation in the treatment targeted areas. Ultimately, the energy medical treatments are capable to modulate inflammation in a benefic way, which is conducive to the restart of healing, but in the same time limiting the inflammation time with benefic results in reduction of fibrotic tissue and scars formation. Since the inflammation is at the base of many human and animal afflictions, the modulation of inflammation is critical in the healing and regeneration of cells, tissues, and organs for humans and animals.

The success of different energy medical treatments is depending on each type of technology used, the treatment regimen, and the type of disease. The important thing is that the energy medical treatments tap in different natural mechanisms of action/healing that are based on mechanical, chemical, photonic, electrical, magnetic, and thermal interactions, to name a few. To make these individual/distinctive energy treatments more effective, this invention is proposing the use of different combination of these energy technologies in treatment combination energy protocols or in the construction of energy combination medical devices/systems incorporating multiple energy technologies in one device/system.

Thus, the first aim of this invention is to propose medical systems that can apply multiple synergetic energy technologies incorporated in one medical device/system, which can apply these modalities in the same time and thus combining different mechanisms of action inside the targeted cells, tissues, or organs. The combination of different mechanisms of action will enhance the probability of cell, tissue or organ to react beneficially to enhance and accelerate the healing and regeneration.

The second aim of this invention is to show different treatment regimens where individual and specific energy medical technologies can be used in combination, but as separate and subsequent events, in order to keep the benefic upregulation of different healing factors or downregulation/inhibition of factors that impede healing. Usually, for the energy medical systems as part of a normal therapy regimen there is a pause or time period in between different treatment sessions. It will be beneficially if during these inter-treatments pause or time period, another energy medical system can be used with synergetic and benefic results to keep or enhance the effects produced by the primary energy treatment. The art is to combine the right and specific energy medical systems, which can be additive in their effects for a certain disease and based on the detailed patient health status that takes into account different comorbidities, physical characteristic, and life style.

The present invention generally includes methods and devices that produce specially modulated energy outcome for treating different cells, tissues, or organs (for both humans and animals), for elimination of infection and pain by using a personalized approach that might influence the successful outcome of the treatment. The main idea is to combine different technologies and treatment options that involve the use of energy that can come in many forms as heat, pressure, mechanical vibration, electrical, electromagnetic, radio frequency, photonic, plasma, etc. The combination of these energy technologies can be synergetic in their effects and in the end accelerate the healing, repair or regeneration of different cells, tissues, or organs for both humans and animals and in some cases for plants.

Practically, the energy treatments can be combined in novel devices and medical systems that incorporate multiple technologies or can be applied subsequently with specific energy devices in a complementary manner. Thus, the combination energy treatments can be applied in one therapy session using multiple energy delivery systems incorporated in one device/system or by combining and applying different energy modalities with separate devices in distinct and subsequent treatment sessions. In both situations, this combination of energy treatments approach will tap into different mechanism of actions and overlap them or apply them subsequently to keep the benefic effects at the highest level for a faster healing, repair or regeneration of cells, tissues, or organs for humans, animals, or plants.

Also, the descriptions of the invention will mention the medical afflictions that can benefit from such combination of different energy medical treatments, either as part of a single device employing in the same time multiple energy technologies/approaches or by applying distinct and specific energy treatments in subsequent manner with synergetic effects.

Currently, for cellular, tissue, or organ conditions (both chronic and acute), the treatment regimens that employ combination energy medical treatments are specific for a certain medical disorder and they are not tuned based on patient's comorbidities, medical history, biometrics or personal habits (smoking, drinking, etc.). For example, patients with significant cardio-vascular problems would respond much slower to any type of treatment related to a chronic tissue or organ condition. Poor blood circulation and ischemic conditions will impair the healing process, due to the lack of nutrients in the affected area. The same can be said about the patients that have diabetes mellitus. To address these shortcomings, the present invention teaches the use of different combination energy medical treatments for treating acute or chronic cellular, tissue, or organ conditions (for both humans and animals) in a personalized/tailored manner based on the general health status, personal habits, and biometrics of the patient and the situation of damaged tissue or organ.

The tailored/personalized combination energy medical treatments to a certain patient (as presented in the exemplary embodiments of this invention) is based on customized algorithms that can be created to change the actual energy delivered to the cells, tissues, or organs and also related to the total number of treatments. The customization can be realized with the aid of artificial intelligence (AI) depicted regimens, based on the known historical clinical data and its relation to different patient specific conditions, race, sex, environmental, geographical, or socio-economic influences.

Furthermore, closed-loop systems can be used that are assessing and monitoring biological conditions in real time and adjust or change the combination of different energy medical treatments accordingly. For example, in the case of tissue damage treatments, the closed-loop systems can monitor the blood circulation, tissue oxygenations, etc.

The novel combination energy medical systems will be described to understand their components, the driving mechanisms or energy sources, and how they are constructed to proper control their outputs. Also, the interface with the patient and the user will also be discussed, based on the specificity of each embodiment or as a general approach.

### BRIEF DESCRIPTION OF THE DRAWINGS

FIG. 1A is a schematic representation of an energy combination applicator that simultaneously delivers shockwaves and a pressurized active substance to the targeted region, according to one embodiment of the present invention.

FIG. 1B is a schematic structural representation of an energy combination system, which includes the energy combination applicator presented in FIG. 1A and its driving control console, system that simultaneously delivers shockwaves and a pressurized active substance to the targeted region, according to one embodiment of the present invention.

FIG. 2A is a schematic representation of an energy combination applicator that simultaneously delivers shockwaves and electrical currents to the targeted region, according to one embodiment of the present invention.

FIG. 2B is a schematic structural representation of an energy combination system, which includes the energy combination applicator presented in FIG. 2A and its driving control console, system that simultaneously delivers shockwaves and electrical currents to the targeted region, according to one embodiment of the present invention.

FIG. 3A is a schematic representation of an energy combination applicator that simultaneously delivers shockwaves and electromagnetic fields to the targeted region, according to one embodiment of the present invention.

FIG. 3B is a schematic structural representation of an energy combination system, which includes the energy combination applicator presented in FIG. 3A and its driving control console, system that simultaneously delivers shockwaves and electromagnetic fields to the targeted region, according to one embodiment of the present invention.

FIG. 4A is a schematic representation of an energy combination applicator that simultaneously delivers shockwaves and LED/OLED phototherapy to the targeted region, according to one embodiment of the present invention.

FIG. 4B is a schematic structural representation of an energy combination system, which includes the energy combination applicator presented in FIG. 4A and its driving control console, system that simultaneously delivers shockwaves and LED/OLED phototherapy to the targeted region, according to one embodiment of the present invention.

FIG. 5A is a schematic representation of an energy combination applicator that simultaneously delivers shockwaves and laser phototherapy to the targeted region, according to one embodiment of the present invention.

FIG. 5B is a schematic structural representation of an energy combination system, which includes the energy combination applicator presented in FIG. 5A and its driving control console, system that simultaneously delivers shockwaves and laser phototherapy to the targeted region, according to one embodiment of the present invention.

FIG. 6A is a three-dimensional view of an energy combination system (applicator and control console) that simultaneously delivers shockwaves and laser phototherapy to the targeted region with four laser heads, according to one embodiment of the present invention.

FIG. 6B is a frontal view of a combination energy applicator from FIG. 6A that simultaneously delivers shockwaves and laser phototherapy to the targeted region from four laser heads, according to one embodiment of the present invention.

FIG. 7A is a schematic representation of an energy combination applicator that simultaneously delivers shockwaves and ultraviolet light to the targeted region, according to one embodiment of the present invention.

FIG. 7B is a schematic structural representation of an energy combination system, which includes the energy combination applicator presented in FIG. 7A and its driving control console, system that simultaneously delivers shockwaves and ultraviolet light to the targeted region, according to one embodiment of the present invention.

FIG. 8A is a schematic representation of an energy combination applicator that simultaneously delivers shockwaves and non-contact ultrasound transmitted via a mist solution to the targeted region, according to one embodiment of the present invention.

FIG. 8B is a schematic structural representation of an energy combination system, which includes the energy combination applicator presented in FIG. 8A and its driving control console, system that simultaneously delivers shockwaves and non-contact ultrasound transmitted via a mist solution to the targeted region, according to one embodiment of the present invention.

FIG. 9A is a schematic representation of an energy combination applicator that simultaneously delivers shockwaves and contact ultrasound to the targeted region, according to one embodiment of the present invention.

FIG. 9B is a schematic structural representation of an energy combination system, which includes the energy combination applicator presented in FIG. 9A and its driving control console, system that simultaneously delivers shockwaves and contact ultrasound to the targeted region, according to one embodiment of the present invention.

FIG. 10A is a schematic representation of an energy combination applicator that simultaneously delivers shockwaves and radio frequency fields to the targeted region, according to one embodiment of the present invention.

FIG. 10B is a schematic structural representation of an energy combination system, which includes the energy combination applicator presented in FIG. 10A and its driving control console, system that simultaneously delivers shockwaves and radio frequency fields to the targeted region, according to one embodiment of the present invention.

FIG. 11 is a schematic representation of an energy combination applicator that simultaneously delivers shockwaves and with overlapping angled radio frequency fields to the targeted region, according to one embodiment of the present invention.

FIG. 12 is a schematic representation of an energy combination system (applicator and control console) that simultaneously delivers to the targeted region shockwaves and ultrasound with the piezoelectric crystal integrated in the membrane, according to one embodiment of the present invention.

FIG. 13A is a schematic representation of a three-dimensional energy combination applicator that simultaneously delivers to the targeted region shockwaves, coaxial electromagnetic field, and active substances from two peripheral locations, according to one embodiment of the present invention.

FIG. 13B is a partial sectional representation of an energy combination system that includes the energy combination applicator from FIG. 13A and its control console, system that simultaneously delivers to the targeted region shockwaves (produced via electromagnetic principle), coaxial electromagnetic fields, and active substances from two peripheral locations, according to one embodiment of the present invention.

FIG. 13C is a schematic cross-sectional representation of an energy combination system that includes the energy combination applicator from FIG. 13A and its control console, system that simultaneously delivers to the targeted region shockwaves (produced via electromagnetic principle), coaxial electromagnetic fields, and peripheral LED/OLED phototherapy or ultraviolet light therapy from two locations, according to one embodiment of the present invention.

FIG. 13D is a schematic cross-sectional representation of an energy combination system that includes the energy combination applicator from FIG. 13A and its control console, system that simultaneously delivers to the targeted region shockwaves (produced via piezoelectric principle), coaxial electromagnetic fields, and peripheral LED/OLED phototherapy or ultraviolet light therapy from two locations, according to one embodiment of the present invention.

FIG. 13E is a schematic cross-sectional representation of an energy combination system that includes the energy combination applicator from FIG. 13A and its control console, system that simultaneously delivers to the targeted region shockwaves (produced via electromagnetic principle), coaxial electromagnetic fields, and peripheral non-contact ultrasound via mist solution from two locations, according to one embodiment of the present invention.

FIG. 14A is a schematic three-dimensional representation of an energy combination system (applicator and control console) that can simultaneously deliver to the targeted region three types of medical energy modalities in the form of shockwaves (produced via electromagnetic principle), peripheral LED/OLED phototherapy or ultraviolet light, and active substances from a ring, according to one embodiment of the present invention.

FIG. 14B is a schematic three-dimensional representation of the energy combination applicator that is a part of the energy combination system from FIG. 14A, which can simultaneously deliver to the targeted region three types of medical energy modalities in the form of shockwaves (produced via electromagnetic principle), peripheral LED/OLED phototherapy or ultraviolet light, and active substances from a ring, according to one embodiment of the present invention.

FIG. 14C is a schematic frontal representation of the energy combination applicator from FIG. 14B that is a part of the energy combination system from FIG. 14A, which can simultaneously deliver to the targeted region three types of medical energy modalities in the form of shockwaves, peripheral LED/OLED phototherapy or ultraviolet light, and active substances from a ring, according to one embodiment of the present invention.

FIG. 14D is a schematic three-dimensional representation of the output action produces by an energy combination system from FIG. 14A and the energy combination applicator from FIG. 14B and FIG. 14C, system that simultaneously delivers to the targeted region four types of medical energy modalities in the form of shockwaves, coaxial electromagnetic fields, peripheral LED/OLED phototherapy or ultraviolet light, and active substances from a ring, according to one embodiment of the present invention.

FIG. 14E is a schematic cross-sectional representation of the energy combination system from FIG. 14A and FIG. 14D that is using the energy combination applicator presented in FIG. 14A, FIG. 14B, FIG. 14C, and FIG. 14D, system that simultaneously delivers to the targeted region four types of medical energy modalities in the form of shockwaves, coaxial electromagnetic fields, peripheral LED/OLED phototherapy or ultraviolet light, and active substances from a ring, according to one embodiment of the present invention.

FIG. 15 is a schematic representation of an energy combination system that simultaneously delivers non-contact ultrasound via a mist solution and electric currents to the targeted region, according to one embodiment of the present invention.

FIG. 16 is a schematic representation of an energy combination system that simultaneously delivers non-contact ultrasound via a mist solution and electromagnetic fields to the targeted region, according to one embodiment of the present invention.

FIG. 17 is a schematic representation of an energy combination system that simultaneously delivers non-contact ultrasound via a mist solution and LED/OLED phototherapy to the targeted region, according to one embodiment of the present invention.

FIG. 18 is a schematic representation of an energy combination system that simultaneously delivers non-contact ultrasound via a mist solution and laser phototherapy to the targeted region, according to one embodiment of the present invention.

FIG. 19 is a schematic representation of an energy combination system that simultaneously delivers non-contact ultrasound via a mist solution and ultraviolet light to the targeted region, according to one embodiment of the present invention.

FIG. 20 is a schematic representation of an energy combination system that simultaneously delivers non-contact ultrasound via a mist solution and radio frequency fields to the targeted region, according to one embodiment of the present invention.

FIG. 21A is a three-dimensional schematic representation of a revolving energy combination applicator that subsequently can deliver to the targeted region either non-contact ultrasound via a mist solution or shockwaves, according to one embodiment of the present invention.

FIG. 21B is another schematic representation of a revolving energy combination applicator from FIG. 21A that subsequently can deliver to the targeted region either non-contact ultrasound via a mist solution or shockwaves, according to one embodiment of the present invention.

FIG. 21C is a schematic representation of the revolving energy combination applicator from FIG. 21A and FIG. 21B that subsequently can deliver to the targeted region either non-contact ultrasound via a mist solution or shockwaves, where this figure is showing only the revolving mechanism that incorporates an ultrasound head and a separate shockwave head, according to one embodiment of the present invention.

FIG. 21D is a schematic representation of a revolving energy combination applicator from FIG. 21A, FIG. 21B, and FIG. 21C that subsequently can deliver to the targeted region either non-contact ultrasound via a mist solution or shockwaves, where this figure is showing the ultrasound head active and performing the treatment, according to one embodiment of the present invention.

FIG. 21E is a schematic representation of a revolving energy combination system from FIG. 21A, FIG. 21B, FIG. 21C, and FIG. 21D that subsequently can deliver to the targeted region either non-contact ultrasound via a mist solution or shockwaves, where this figure is showing the shockwave head active and performing the treatment, according to one embodiment of the present invention.

FIG. 22 is a schematic representation of a smart bandage capable of producing electrical currents that can be used in combination energy protocols for medical treatments, according to one embodiment of the present invention.

FIG. 23 is a schematic representation of a smart bandage capable of using energy to produce oxygen and oxygen species that can be in used combination energy protocols for medical treatments, according to one embodiment of the present invention.

FIG. 24 is a schematic representation of a smart bandage capable of using energy to produce drug delivery via energy that can be in used combination energy protocols for medical treatments, according to one embodiment of the present invention.

FIG. 25 is a schematic representation of a smart bandage capable of producing electromagnetic fields that can be used in combination energy protocols for medical treatments, according to one embodiment of the present invention.

FIG. 26 is a schematic representation of a smart bandage capable of producing ultrasound fields that can be used in combination energy protocols for medical treatments, according to one embodiment of the present invention.

FIG. 27 is a schematic representation of a smart bandage capable of producing LED/OLED phototherapy that can be used in combination energy protocols for medical treatments, according to one embodiment of the present invention.

FIG. 28A is a three-dimensional view of an à la carte/menu-based modular control console with three energy combination applicator stations that employs artificial intelligence-generated algorithms for selection of appropriate combination energy medical treatment protocols, based on disease type and stage, specificity of patient medical history, comorbidities, biometrics, and associated symptoms, according to one embodiment of the present invention.

FIG. 28B is a frontal view of an à la carte/menu-based modular control console with three energy combination applicator stations, as presented in FIG. 28A, that employs artificial intelligence-generated algorithms for selection of appropriate combination energy medical treatment protocols, based on disease type and stage, specificity of patient medical history, comorbidities, biometrics, and associated symptoms, according to one embodiment of the present invention.

FIG. 29A is a three-dimensional view of another design for an à la carte/menu-based modular control console with detachable tablet and three energy combination applicator stations that employs artificial intelligence-generated algorithms for selection of appropriate combination energy medical treatment protocols, based on disease type and stage, specificity of patient medical history, comorbidities, biometrics, and associated symptoms, according to one embodiment of the present invention.

FIG. 29B is a three-dimensional view of the orthopedic option for detachable/portable display or tablet for the à la carte/menu-based modular control console with three energy combination applicator stations from FIG. 29A, according to one embodiment of the present invention.

FIG. 29C is a three-dimensional view of a general menu for detachable/portable display or tablet for the à la carte/menu-based modular control console with three energy combination applicator stations from FIG. 29A, according to one embodiment of the present invention.

FIG. 30A is a three-dimensional view of the à la carte/menu-based modular control console with three energy combination applicator stations presented in FIG. 29A, this time with three different types of energy combination medical treatment applicators connected to the console, according to one embodiment of the present invention.

FIG. 30B is a frontal view of the à la carte/menu-based modular control console with three energy combination applicator stations presented in FIG. 29A and FIG. 30A, this time with three different types of energy combination medical treatment applicators connected to the console, according to one embodiment of the present invention.

FIG. 31 is a first diagram of an artificial intelligence-based algorithm that can be applied to identify the combination energy medical treatment protocols performed via independent energy-based devices (using only one energy technology) in subsequent and different treatment sessions or to identify energy combination medical devices/systems (using multiple energy technologies simultaneously) that should be used for optimal medical treatment outcomes, according to one embodiment of the present invention.

FIG. 32 is a second continuing diagram of the artificial intelligence-based algorithm of FIG. 31 that can be applied to identify the combination energy medical treatment protocols performed via independent energy-based devices (using only one energy technology) in subsequent and different treatment sessions or to identify energy combination medical devices/systems (using multiple energy technologies simultaneously) that should be used for optimal medical treatment outcomes, according to one embodiment of the present invention.

FIG. 33 is a third continuing diagram of the artificial intelligence-based algorithm of FIG. 31 that can be applied to identify the combination energy medical treatment protocols performed via independent energy-based devices (using only one energy technology) in subsequent and different treatment sessions or to identify energy combination medical devices/systems (using multiple energy technologies simultaneously) that should be used for optimal medical treatment outcomes, according to one embodiment of the present invention.

FIG. 34 is a fourth continuing diagram of the artificial intelligence-based algorithm of FIG. 31 that can be applied to identify the combination energy medical treatment protocols performed via independent energy-based devices (using only one energy technology) in subsequent and different treatment sessions or to identify energy combination medical devices/systems (using multiple energy technologies simultaneously) that should be used for optimal medical treatment outcomes, according to one embodiment of the present invention.

FIG. 35 is a fifth continuing diagram of the artificial intelligence-based algorithm of FIG. 31 that can be applied to identify the combination energy medical treatment protocols performed via independent energy-based devices (using only one energy technology) in subsequent and different treatment sessions or to identify energy combination medical devices/systems (using multiple energy technologies simultaneously) that should be used for optimal medical treatment outcomes, according to one embodiment of the present invention.

FIG. 36A is a legend table that defines numeric or alpha codes for different medical conditions, energy medical technologies, smart bandages that incorporate energy technologies, and treatment protocols, which are used to describe different combination energy treatment protocols or energy combination systems used for various medical treatments, based on the artificial intelligence algorithm shown in FIGS. 31-35.

FIG. 36B is a legend table that includes the alpha code related to different energy combination systems and associated energy combination applicators together with the corresponding figures for their actual embodiments as presented in FIGS. 1A-21E of an embodiment of the invention.

FIG. 37A is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols A 10016 and B 10017, as determined per the artificial intelligence algorithm presented in FIGS. 31-35.

FIG. 37B is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols C 10018 and D 10019, as determined per the artificial intelligence algorithm presented in FIGS. 31-35.

FIG. 37C is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols E 10020 and F 10021, as determined per the artificial intelligence algorithm presented in FIGS. 31-35.

FIG. 37D is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols G 10022, H 10023, and I 10024, as determined per the artificial intelligence algorithm presented in FIGS. 31-35.

FIG. 37E is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols J 10025, K 10026, and L 10027, as determined per the artificial intelligence algorithm presented in FIGS. 31-35.

FIG. 37F is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols M 10028, N 10029, and O 10030, as determined per the artificial intelligence algorithm presented in FIGS. 31-35.

FIG. 37G is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols P 10031, Q 10032, and R 10033, as determined per the artificial intelligence algorithm presented in FIGS. 31-35.

FIG. 37H is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols S 10034, T 10035, U 10036, and V 10037, as determined per the artificial intelligence algorithm presented in FIGS. 31-35.

FIG. 37I is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols W 10038, X 10039, and Y 10040, as determined per the artificial intelligence algorithm presented in FIGS. 31-35.

FIG. 37J is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols Z 10041, AA 10042, and BB 10043, as determined per the artificial intelligence algorithm presented in FIGS. 31-35.

FIG. 37K is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols CC 10044, DD 10045, and EE 10046, as determined per the artificial intelligence algorithm presented in FIGS. 31-35.

FIG. 37L is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols FF 10047, GG 10048, and HH 10049, as determined per the artificial intelligence algorithm presented in FIGS. 31-35.

FIG. 37M is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols II 10050, JJ 10051, and KK 10052, as determined per the artificial intelligence algorithm presented in FIGS. 31-35.

FIG. 37N is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols LL 10053 and MM 10054, as determined per the artificial intelligence algorithm presented in FIGS. 31-35.

FIG. 37O is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols NN 10055, OO 10056, and PP 10057, as determined per the artificial intelligence algorithm presented in FIGS. 31-35.

FIG. 37P is a table identifying the medical conditions and the corresponding choices for different energy combination medical devices/systems (using multiple energy technologies simultaneously) and their associated treatment protocols or combination energy medical treatment protocols performed via independent energy-based devices (using only one energy technology) that can be used for the treatment of different tissue lesions and for specific medical conditions, which are corresponding to the protocols QQ 10058, RR 10059, and SS 10060, as determined per the artificial intelligence algorithm presented in FIGS. 31-35.

### DETAILED DESCRIPTION OF THE INVENTION

Embodiments of the invention will be described with reference to the accompanying figures, wherein like numbers represent like elements throughout. Further, it is to be understood that the phraseology and terminology used herein is for the purpose of description and should not be regarded as limiting. The use of “including”, “comprising”, or “having” and variations thereof herein is meant to encompass the items listed thereafter and equivalents thereof, as well as additional items. The terms “connected”, and “coupled” are used broadly and encompass both direct and indirect mounting, connecting, and coupling. Further, “connected” and “coupled” are not restricted to physical or mechanical connections or couplings.

It is an objective of the present inventions to provide combination energy generating devices that are modular, do not need high maintenance and can, if needed, be applied/used in conjunction with other devices, active substances (drugs, medications, etc.), methods and existing treatments for cellular, tissue or organ conditions (for both therapy and prophylactic use).

It is a further objective of the present inventions to provide a means of controlling the energy via the amount of energy generated from specific generators (energy setting) for each type of energy technology incorporated in energy combination medical systems or treatment regimes, total number of the treatment sessions and if they are performed concomitantly or subsequently, and special construction of the energy application devices (applicators, wands, etc.) and their associated electromechanical control consoles, which incorporate specially designed software to ensure the functionality of the medical system and the interface with the user.

It is a further objective of the present inventions to provide a variety of novel constructions of energy combination medical systems to be used in manufacturing processes and for treating living tissue, determined by the number of energy technologies housed in one applicator or wand or device/system, the specific treatment sequence, treatment parameters and the possibility to apply the treatment on a specific direction and for a predetermined penetration, to reach the appropriate human or animal cells, tissue, or organs that are the subject of the medical treatment.

The inventions summarized in this patent are defined by the enumerated claims and are better understood by referring to the following detailed description, which is preferably read in conjunction with the accompanying drawing/figure. The detailed description of a particular embodiment, is set out to enable one to practice the invention, it is not intended to limit the enumerated claims, but to serve as a particular example thereof.

Also, the list of embodiments presented in this invention is not an exhaustive one and for those skilled in the art, new applications and optimization methods can be found within the scope of the invention.

When multiple technologies are applied simultaneously by one multi-purpose device, the treatment volumes/areas/regions produced by different technologies used by the multi-purpose device/system can be adjacent or overlapping. If the targeted volumes/areas/regions are overlapping, then the amount of energy delivered in one pass is higher inside the targeted treatment zone, with benefic results for treatments that require more energy that triggers multiple mechanisms of action. If the targeted volumes/areas/regions are not overlapping and are more likely adjacent, then the total volume of tissue treated in one position of the applicator is increased, which means that for treatments that need to cover large areas (as in burns, cosmetic treatments, etc.) the efficiency of the treatment is augmented by covering in one pass larger targeted treatment zones.

The use of multiple energy technologies simultaneously or sequential effectively allow a wound to move through the inflammatory phase quickly and into the proliferation (cell growth) phase of healing. The shortened inflammatory phase noticed after acoustic pressure combination energy medical treatment may be beneficial to long term healing. During inflammation, undifferentiated collagen tissue is produced as the body attempts to simply cover and constrict an open wound. By shortening this inflammatory phase, the scar tissue is less apparent, more subtle and does not constrict mobility.

The introduction of the genetic material inside the cells is done via a process called electroporation. Electroporation, or electro-permeabilization, is a microbiology technique in which an electrical field is applied to cells in order to increase the permeability of the cell membrane, allowing chemicals, drugs, or DNA/other genetic material to be introduced into the cell (also called electro-transfer). Electroporation has proven efficient for use on tissues in vivo, for in utero applications. However, one downside to electroporation is that after the treatment the gene expression of over 7,000 genes can be affected. This can cause problems in studies where gene expression has to be controlled to ensure accurate and precise results. Although bulk electroporation has many benefits over physical delivery methods such as microinjections and gene guns, it still has limitations including low cell viability. Furthermore, the devices associated with the electroporation are complicated and have moving parts that makes them less reliable.

This is why in certain embodiments of this invention, different energy delivery treatments/technologies (combination of electric currents, electromagnetic fields, ultrasound, lasers, shockwaves, nanotechnologies, or radio frequency treatment modalities, etc.) may also be provided to facilitate genetic modifications, such as in conjunction with gene therapies, or to facilitate stem cell therapies, such as promotion and acceleration of differentiation of stem cells. Such energy delivery treatments/technologies can also facilitate a combination of gene and stem cells therapies—including application of different energy treatment modalities simultaneously with gene therapy and stem cell therapy.

The present invention generally includes methods and devices that produce and use different combinations of energy technologies applied for medical purpose, by using a personalized approach that might influence the successful outcome of the treatment. The main idea is to combine different medical technologies and treatment options that involve the use of energy that can come in many forms as heat, pressure, mechanical vibration, electrical, electromagnetic, radio frequency, etc. These energy technologies in combination can be synergetic in their effect and in the end accelerate the healing, repair or regeneration of different tissues or organs for both humans and animals.

New medical devices/systems are presented in this invention that are capable to simultaneously deliver multiple forms of energy to enhance the treatment and tap into different mechanism of actions to enhance and expedite healing. For example, these new medical devices/systems incorporate combination of shockwaves with drug delivery mechanism in a form of a mist, where the mist is delivered simultaneously with the shockwaves.

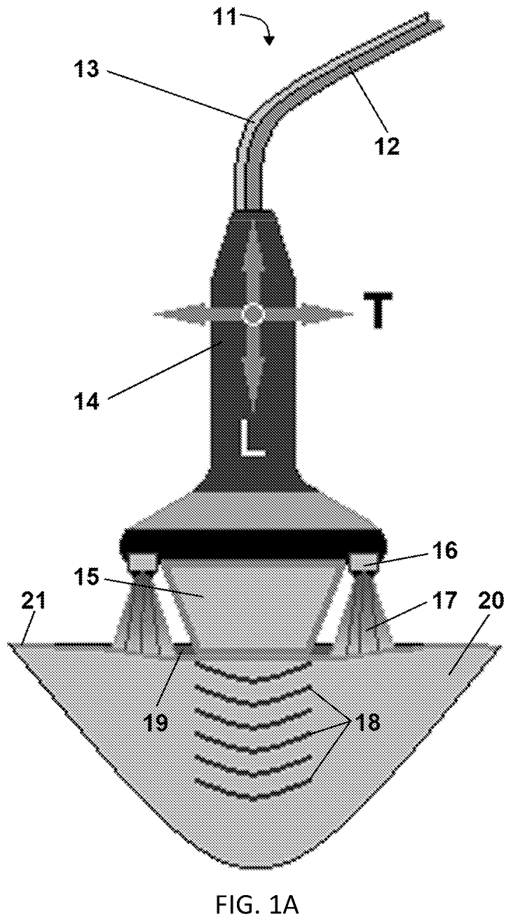

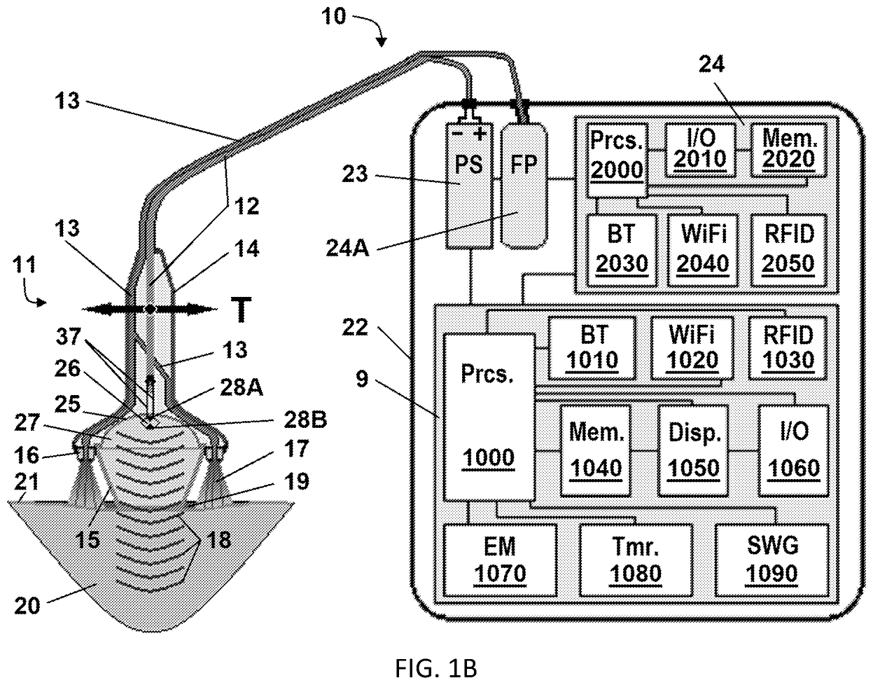

As presented in U.S. Pat. No. 8,556,813 Cioanta et al., when a holding fixture is used for the shockwave applicators, special pressurized reservoirs can be part of the holding fixture. These pressurized reservoirs could contain different liquids that can be sprayed during treatment over the skin or targeted area, as presented in U.S. Pat. No. 8,556,813 Cioanta et al. The same liquid substances can be pulverized via nozzles on the treatment area using air and thus creating aerosols. The medication combined with air movement can have besides the treatment advantages also an analgesic effect. During one treatment, different substances can be sprayed, via multiple spray jets on the treatment area based on necessities and physician's indications. Also, the front spray (placed in the front of the applicator) could contain a different substance from the back spray (placed in the back of the applicator). Substances that might be used with this design can be analgesics, antibiotics, saline solutions, liquid gels, antibacterial substances, etc. However, the design presented in U.S. Pat. No. 8,556,813 can be simplified if the holding fixture with the pressurized reservoirs is eliminated and the shockwave applicator has integrated specialized nozzles into its body to be able to spray different active substances on the treatment zone/area in parallel with the delivery of shockwaves. Such design is presented in FIGS. 1A and 1B that show applicators used in the combination treatment regimens with energy-based medical technologies or combination devices/systems, which can deliver simultaneously multiple energy-based technologies. In this case, the combination applicators 11 will have a special design that can facilitate a medical treatment using the shockwaves/pressure waves 18 in combination with active medical substances (sprayed on the wound/lesion/tissue condition 19) from the outside/exterior of the human or animal body (extracorporeally) and towards the targeted treatment zone.

Thus, the combination shockwaves/pressure waves and pressurized active substance medical system 10 from FIG. 1B is capable in the same device to produce shockwaves/pressure waves in combination with positive pressure delivery of medicine/active substance as mist solution. These specially designed applicators and medical systems that use combination treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to generate the mist solution of liquid medicine/solution, which is delivered simultaneously with the shockwaves/pressure waves 18 (see FIGS. 1A and 1B) for stimulation, healing, and regeneration of cells, tissues, or organs, for both humans and animals. The liquid medicine/active substance can be in the form of drugs, antibiotics, antioxidants, traditional natural medical substances, growth factors, cytokines, exosomes, enzymes, stem cells, antibodies, bio-nanoparticles, genetic material, vaccines, scaffolds or matrices to mimic extracellular matrices, nano-robots, specific proteins, etc., to name a few, which can be used for healing of different afflictions or regeneration of cells, tissues, or organs for both human and animal subjects.

As seen from FIGS. 1A and 1B, the combination shockwaves/pressure waves and pressurized active substance medical system 10 includes a combination applicator 11 that uses a high voltage cable 12 and an active substance tubing 13 (as independent elements or welded together in a multi-capability cable) to connect to the control console/unit 22 shown in FIG. 1B. The applicator body 14 is used by the user to move during treatment the combination applicator 11 along the longitudinal movement L and transversal movement T, to allow the covering of the entire targeted treatment area or region of the human or animal body. The applicator/coupling membrane 15 sits on top of the reflector 25 (see FIG. 1B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles as electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. The shockwaves/pressure waves 18 are passing through the wound/lesion/tissue condition 19 that can be positioned superficially, or immediately under the skin 21, or deep inside the human/animal body 20. On the peripheral part of the bulbous region of the applicator body 14 and next to the applicator/coupling membrane 15, at least two or three or four or more spraying nozzles 16 are present, which are capable of spraying an active substance that has medical effects on the wound/lesion/tissue condition 19. The active substance can be a fluid, as purified water, saline, isotonic solutions, hypotonic solutions, hypertonic solutions, etc., loaded with active liquid medicine that can be in the form of drugs, antibiotics, antioxidants, traditional natural medical substances, growth factors, proliferation factors, angiogenesis factors, cytokines, exosomes, enzymes, stem cells, bio-nanoparticles, antibodies, genetic material, vaccines, scaffolds or matrices to mimic extracellular matrices, nano-robots, specific proteins, etc. for healing of different afflictions or regeneration of cells, tissues, or organs for both human and animal subjects. The active substance can be sprayed for maximum efficiency against the wound/lesion/tissue condition 19 using a predetermined active substance spraying pattern 17 by each of the spraying nozzles 16. The combined effects of the shockwaves/pressure waves 18 and of the active substance, which is sprayed through the active substance spraying patterns 17 via the spraying nozzles 16, will enhance and expedite the healing of the wound/lesion/tissue condition 19. Such energy combination medical system is beneficial for a successful medical treatment and thus becomes more attractive from financial point of view and less time consuming for both the patients and the medical personnel.

In FIG. 1B, the focus is on the configuration of the control console/unit 22 and the internal structure of the combination applicator 11. The combination applicator 11 internal structure contains different elements as the high voltage cable 12, shockwave/pressure wave electrodes wiring 37 for the first electrode 28A and the second electrode 28B of the spark-gap 26, the reflector 25, the applicator/coupling membrane 15, the active substance tubing 13, and the spraying nozzles 16, which are placed inside, or sit on, or are attached to the applicator body 14.

The high voltage cable 12 provides the connection in between the combination applicator 11 and the control console/unit 22 and it goes inside the applicator body 14 and via the shockwave/pressure wave electrodes wiring 37 it reaches the first electrode 28A and the second electrode 28B of the spark-gap 26, where the shockwaves/pressure waves 18 are generated inside the fluid-filled reflector and membrane cavity 27. The high voltage that travels through high voltage cable 12 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, the fluid field generator 24 and its associated fluid pump (FP) 24A are sharing a common power supply (PS) 23, which can be incorporated inside the control console/unit 22 (as presented in FIG. 1B). In some other embodiments, two separate internal power supplies can be used or an external power supply can be used that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the fluid field generator 24, power supplies that can be internal to the control console/unit 22 or in other embodiment can be external to the control console/unit 22 as part of an independent module/ancillary system.

The shockwaves/pressure waves field generator 9 includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090.

Also, the active substance tubing 13, brings and distributes evenly the active substance to all the spraying nozzles 16. In the exemplification from FIG. 1B there are two (2) spraying nozzles 16 each of them with a dedicated active substance tubing 13. The active substance delivery to the wound/lesion/tissue condition 19 is controlled by the fluid field generator 24 that is directly connected to the fluid pump (FP) 24A. The fluid field generator 24 includes the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050. The fluid pump processor 2000 has a slave-master relationship with the control console/unit processor 1000, which means that the control console/unit processor 1000 controls the activity of the fluid pump processor 2000 via a continuous interaction process of sharing information and commands. Thus, the control console/unit processor 1000 dictates the functionality of the entire fluid field generator 24 and its associated fluid pump (FP) 24A. Through this process the control console/unit processor 1000 together with the fluid pump processor 2000 control the active substance dosage, pressures, time of activation, total time for spraying, and the active substance spraying pattern 17. The active substance or substances can be extracted for the medical treatment by the fluid pump (FP) 24A from dedicated reservoirs that are part of the control console/unit 22 or from independent reservoirs, which are not part of the control console/unit 22. These reservoirs are not shown in FIGS. 1A and 1B for the simplicity of these figures and also for the fact that they are well known in the art.

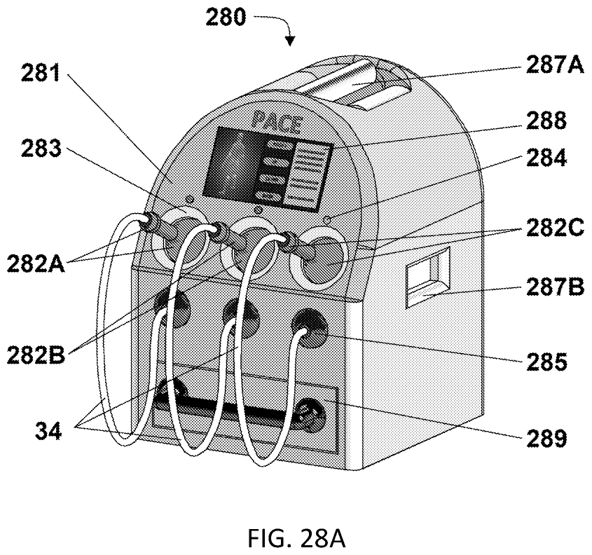

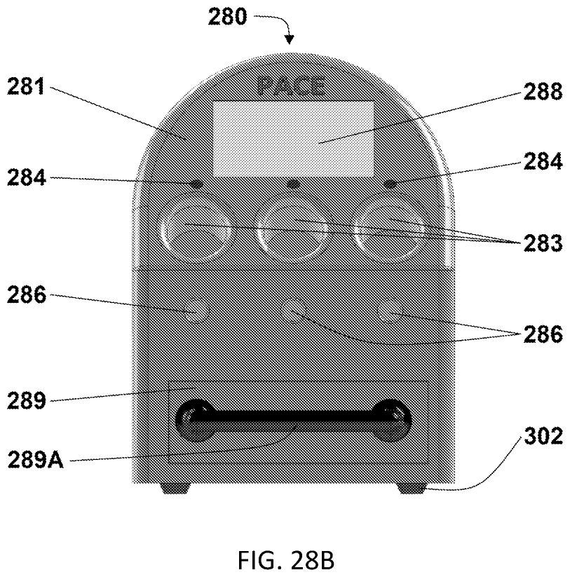

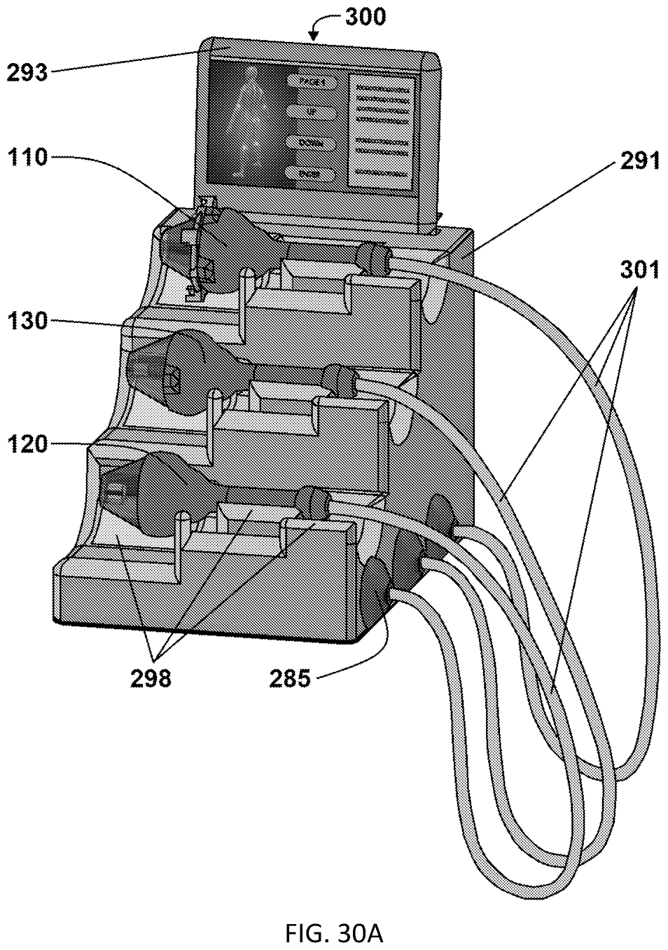

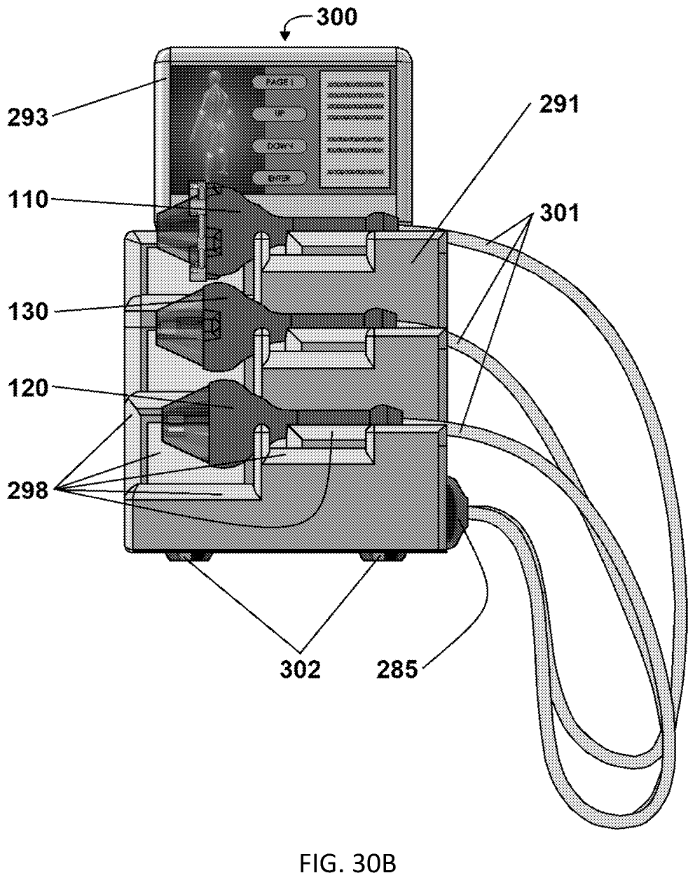

The control console/unit processor 1000 practically controls the whole activity of any combination energy medical systems, including the combination shockwaves/pressure waves and pressurized active substance medical system 10 presented in FIGS. 1A and 1B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the active substance dosage, pressures, time of activation, total time for spraying, and the active substance spraying pattern 17. The data transfer protocol in between the control console/unit 22 and combination applicator 11 is secured via an authentication process that includes processor generated passcodes and passwords, which is performed when the high voltage cable 12 and the active substance tubing 13 are connected to the control console/unit 22 and the power is “On” for the control console/unit 22. For all embodiments presented in this invention, the connection between the combination applicator 11 and the control console/unit 22 is done via a combination applicator power connector 285, as presented in FIGS. 28A, 30A, and 30B. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, active substance quantity/dosage, pressures, time of activation, total time for spraying, active substance spraying pattern 17, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet-based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030.

In one embodiment, the control console/unit memory 1040 may be any type of memory configured to store downloaded information regarding one or more energy combination treatment settings, optimization algorithm, treatment outcomes, errors, warnings, parameters used during the delivered therapy/treatment, patient information, follow-up data, monitoring data, tutorials, troubleshooting, and in general device functionality. In some embodiments, the control console/unit memory 1040 is any type of memory configured to store control information indicative of the one or more energy combination treatment settings or control signals for controlling the combination applicator 11. The control console/unit memory 1040 may store downloaded information regarding the actual settings of one or more performed treatments, errors, warnings, and in general the functionality of any combination energy medical systems, including the combination shockwaves/pressure waves and pressurized active substance medical system 10. This data from the control console/unit memory 1040 can be downloaded for further analysis directly via the control console/unit I/O (input/output) element 1060. Also, this treatment data and device functionality can be shared via the secured data transfer protocol in between the combination applicator 11 and the control console/unit 22, data that can also be shared with an external artificial intelligence (A/I) device, as described before.

In one embodiment, the control console/unit I/O (input/output) element 1060 receives information indicative of one or more energy combination treatment settings, processes the received information using the control console/unit processor 1000 to generate a control signal for controlling combination energy medical treatments. Also, the control console/unit I/O (input/output) element 1060 may be configured to receive information, such as updated information indicative of new treatment settings resultant from a previously completed medical treatment. In another embodiment, the control console/unit I/O (input/output) element 1060 may receive information indicating that a treatment has been administered and transmit the information to the control console/unit processor 1000, which may decrement by one a treatment setting indicative of the number of treatments/treatment sessions allowed to a patient that was calculated, optimized, and personalized for patient comorbidities and life style using algorithms, as the ones presented in U.S. Pat. No. 10,888,715. The updated treatment setting may then be written on control console/unit memory 1040 for subsequent accessing and use during a next energy combination medical treatment or to an external the artificial intelligence (A/I) device for subsequent metadata analysis.

The I/O (input/output) element 1060 can be a fixed display incorporated into the control console/unit 22 or a desktop computer, or a smart phone, or a tablet, or a laptop, or an artificial intelligence (A/I) device, devices that also serve as the input/output (I/O) device in this case. If the control console/unit 22 is remotely connected via the control console/unit Bluetooth (BT) module 1010 with a desktop computer, or a smart phone, or a tablet, or a laptop, or an artificial intelligence (A/I) device, in one embodiment those devices can also dictate remotely (via a master-slave electronic relationship) the whole functionality of the control console/unit 22 related to the treatment parameters, security, display of treatment parameters and data, coordination of different subsystems or subcomponents, etc. The control console/unit 22 can dictate the input and output information that may be displayed on the desktop computer, or a smart phone, or a tablet, or a laptop, or on an artificial intelligence (A/I) device. For example, in one embodiment, the control console/unit 22 is a device that can be connected to a desktop computer, or a smart phone, or a tablet, or a laptop and be used by a patient or by an in-home caregiver. The I/O (input/output) element 1060 of the control console/unit 22 may include an on/off mechanism that can be used by user for turning the desktop computer, or a smart phone, or a tablet, or a laptop on and off, respectively.

In another separate embodiment, the control console/unit 22 can function autonomously and at the end of the treatment only transfer the treatment parameters and data via the control console/unit Bluetooth (BT) module 1010 to the remotely connected devices as a desktop computer, or a smart phone, or a tablet, or a laptop, or an artificial intelligence (A/I) device. The transmitted data can be used for monitoring healing progression over multiple treatments/treatment sessions, or providing input data for treatment parameters adjustment from one treatment session to a subsequent one based on healing progress, or delivering data for reimbursement or financial payments or insurance assessment, or logging patient personal and medical data, comorbidities, medication, and nutritional facts, in a Health Insurance Portability and Accountability Act (HIPPA) compliant fashion, or recording system functional monitoring data, input data, treatment settings and parameters, output data, and treatment duration, or tracking adverse events, or logging the devices or equipment and other ancillary technologies or devices or treatment kits or components, which were used during actual treatment, or recording date and timing of the treatment session, etc.

The above information/data can be also transferred via control console/unit Wi-Fi module 1020 or any other type of internet connection from the control console/unit 22, or the desktop computer, or the smart phone, or the tablet, or the laptop, or the artificial intelligence (A/I) device towards internet-based data storage/cloud database. The data stored into the internet based data storage/cloud database can be further transferred via Wi-Fi connection or any other type of internet connection to an artificial intelligence (AI) server for processing via artificial intelligence (AI) algorithms to extract deep information within the medical treatment data to solve highly complex medical challenges and treatment shortcomings, predict synergetic or non-synergetic interaction in between different devices and treatment modalities, to advance scientific research, and better predict medical events and patient (human or animal) behavior during treatment, in between subsequent treatment sessions, or after the entire treatment regimen is finished. Ultimately, by using the artificial intelligence (AI) data analysis algorithms of the artificial intelligence (AI) server, adjustments can be done to determine or alter or tune the treatment settings in accordance with patient's medical situation, comorbidities, daily medication regimen, nutritional facts, daily habits (drinking, smoking, etc.), ethnicity, gender, age, genetic make-up, etc., which will make the medical treatment more efficacious and efficient, or to assess the treatment targeted tissue location and condition (vascularization, ischemia, perfusion, bacterial load, etc.) that will dictate the treatment selection, or to offer treatment options and decision trees based on the healing progress or non-healing progress, or to adjust device settings based on the correlation in between treatment settings and treatment targeted region characteristics (type of tissue, area, volume, depth inside the body, etc.). Furthermore, the artificial intelligence (AI) data analysis algorithms can be used to propose changes to the device to improve its functionality, to increase its user interface capabilities, and to expand its inter-connectivity with other devices/technologies, or to produce spreadsheets for tracking treatments and send information for payment, insurance coding, security, inventory tracking, or medical treatment setting provisioning, or any combination of these functions, or to produce customized reports to meet clinical and compliance requirements, etc.

The control console/unit display module 1050 may provide a visual display of graphics and/or text indicative of the one or more energy combination medical treatment settings and selections, patient identity information, patient comorbidities and medication, patient insurance information, ancillary treatment apparatus choice, photos of treatment area, control console/unit I/O (input/output) element 1060 to introduce or extract data, training materials, troubleshooting, treatment apparatus activation or the like. Additionally, in some embodiments, control console/unit display module 1050 may provide a visual display of navigational treatment setting choices and current treatment parameters. In this case, the control console/unit display module 1050 may provide an indicator of the amount of treatment remaining before, during or after a medical treatment, or provide a display of one or more body regions targeted by the treatment.

The control console/unit 22 and its control console/unit processor 1000 control the shockwaves/pressure waves 18 setting (energy input, frequency, activation sequence, treatment time, etc.) and then output optimum combination energy medical treatments. For that, the control console/unit processor 1000 is using the control console/unit emission mechanism (EM) module 1070, the control console/unit timer module 1080, and the control console/unit shockwave or pressure wave generator (SWG) module 1090. The control console/unit shockwave or pressure wave generator (SWG) module 1090 may include hardware or components for providing, via the combination applicator 11, one or more focused or unfocused acoustic pressure shockwaves, or radial acoustic pressure waves, or planar acoustic pressure waves, or cylindrical acoustic pressure waves, by electromechanical, electromagnetic, electrohydraulic, or piezoelectric methods (e.g., crystal, thin films or fibers), which are well-known by those skilled in the art. The control console/unit timer module 1080 provides timing sequence for emitting the one or more generated acoustic pressure shockwaves, or radial acoustic pressure waves, or planar acoustic pressure waves, or cylindrical acoustic pressure waves at a selected frequency, as dictated by the one or more energy combination treatment settings. The one or more focused or unfocused acoustic pressure shock waves, or radial acoustic pressure waves, or planar acoustic pressure waves, or cylindrical acoustic pressure waves are emitted in a manner controlled by the control console/unit emission mechanism (EM) module 1070 towards the combination applicator 11, via a secured data transfer protocol, as mentioned before.

Depending on the capabilities of the control console/unit 22, it may also include the control console/unit Bluetooth (BT) module 1010, the control console/unit Wi-Fi module 1020, and the control console/unit RFID module 1030 to ensure a proper and rapid communication, via the data transfer protocol in between the combination applicator 11 and the control console/unit 22. In one embodiment, only one of these communication modules may be present, in another embodiment two of them, and in yet another embodiment all three of these communication modules may be present.

The control console/unit Bluetooth (BT) module 1010 or the control console/unit Wi-Fi module 1020 may be used to transmit control info wirelessly or receive info to make software updates, turn “On” and “Off” internal functions or components incorporated into the control console/unit 22.

Another way to communicate data to the control console/unit 22 and the combination applicator 11 from an outside radio frequency identification device (RFID) is by using a proximity RFID protocol employing the control console/unit RFID module 1030 and the fluid pump RFID module 2050. An external information storage device (not shown in any of the figures) can be in the form of an RFID system/device that may be used to communicate with the control console/unit RFID module 1030. In various embodiments, the external RFID treatment information storage device may be a tag, label, or chip, and may include passive, active, or semi-passive technology. In some embodiments, the RFID device may include RFID chip-less technology or electronic product code technology. Chipless RFID devices may allow for discrete identification of RFID tags without an integrated circuit, thereby allowing tags to be printed directly onto the surface of a treatment kit at lower costs than traditional tags. In one embodiment, treatment information RFID storage device may be a passive tag that requires no electrical supply for powering the tag. The tag may be inside of a medical treatment kit or it can be a label placed on the outside of the kit. In another embodiment, the treatment information storage device may be a passive RFID tag incorporating electronic product code technology. In various embodiments, the treatment information storage device may be a polymer tag such as that manufactured by PolyIC, which is located in Germany, or that is manufactured by Phillips, which is located in the Netherlands. The protected RFID communication protocol can facilitate the exchange of any data from such external device or from combination applicator 11 (applicator that should have an RFID chip incorporated into its connector/connection of the high voltage cable 12 to the control console/unit 22), which can be transferred and stored in control console/unit memory 1040 or fluid pump memory 2020 via the control console/unit processor 1000.

In various embodiments, an RFID device (in the form of control console/unit RFID module 1030 or RFID external treatment information storage device) may communicate according to the International Standards Organization (“ISO”) 14443 and/or the International Electrotechnical Commission (“IEC”) 18000-6 standards. The RFID external treatment information storage device may communicate up to a distance of 10 cm (i.e., 4 inches) from the control console/unit RFID module 1030, in accordance with ISO 14443. The RFID device may be included in a smart label governed by ISO 15693. In one embodiment, the RFID device is a 13.567 MHz device.

Furthermore, by way of example, but not limitation, external treatment information storage device may be besides an RFID tag, in the form of a label or chip, memory stick, smart card, credit card, barcode, floppy disk, CD-ROM, digital versatile disk (“DVD”) or any device configured to store information and from which information may be read.

The fluid field generator 24 directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050. As mentioned before, the fluid pump processor 2000 has a slave-master relationship with the control console/unit processor 1000, which means that the control console/unit processor 1000 controls the activity of the fluid pump processor 2000 via a continuous interaction process of sharing information and commands. As part of the fluid field generator 24, the fluid pump processor 2000 controls the activities of the fluid pump I/O (input/output) element 2010, the fluid pump memory 2020, the fluid pump Bluetooth module 2030, the fluid pump Wi-Fi module 2040, and the fluid pump RFID module 2050. In general, the functions of fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050 are similar to the ones presented above for the control console/unit processor 1000, the control console/unit Bluetooth (BT) module 1010, the control console/unit Wi-Fi module 1020, the control console/unit RFID module 1030, the control console/unit memory 1040, and the control console/unit I/O (input/output) element 1060. The fluid pump processor 2000 together with the control console/unit processor 1000 mainly can control the active substance dosage, pressures, time of activation, total time for spraying, and the active substance spraying pattern 17.

In other embodiment, the fluid field generator 24 contains only the fluid pump processor 2000. Additionally, in some embodiments depending on the capabilities of the fluid field generator 24 and control console/unit 22, the fluid field generator 24 may also include the fluid pump Bluetooth module 2030, the fluid pump Wi-Fi module 2040, and the fluid pump RFID module 2050 to ensure a proper and rapid communication with the control console/unit 22. In one embodiment, only one of these communication modules may be present, in another embodiment two of them, and in yet another embodiment all three of these communication modules may be present. Furthermore, in other embodiment, either the fluid pump I/O (input/output) element 2010 or the fluid pump memory 2020 can be present or none of them are present in the construction of the fluid field generator 24. The advantage of having the fluid pump I/O (input/output) element 2010 is that it offers the possibility to independently input or output data for the functionality of the fluid field generator 24 and the output of the fluid pump (FP) 24A. Regarding the fluid pump memory 2020, this module allows to store the functionality of the fluid field generator 24 and the output of the fluid pump (FP) 24A separately from the control console/unit memory 1040 of the shockwaves/pressure waves field generator 9.

The FIGS. 1A and 1B also show that the combination applicator 11 can be moved in the longitudinal movement L and the translational movement T, movements that can be done manually or automatically. This allows the complete coverage of the treatment region by the combination applicator 11. When the L and T movements are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11.

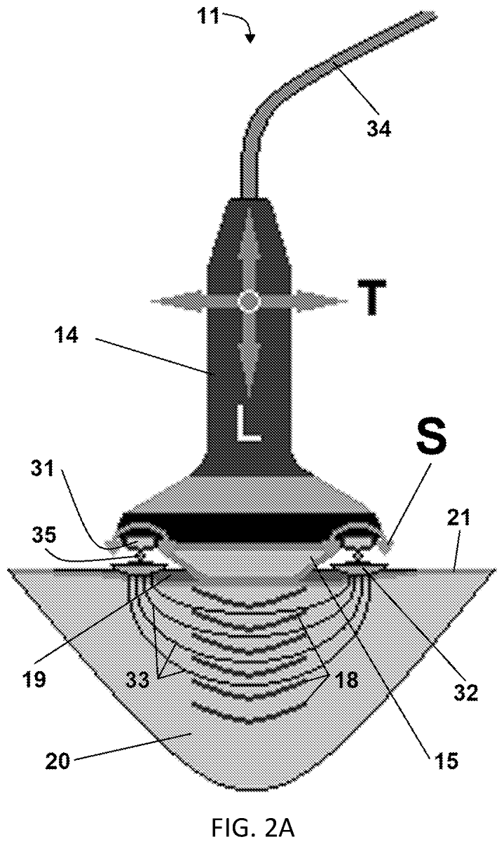

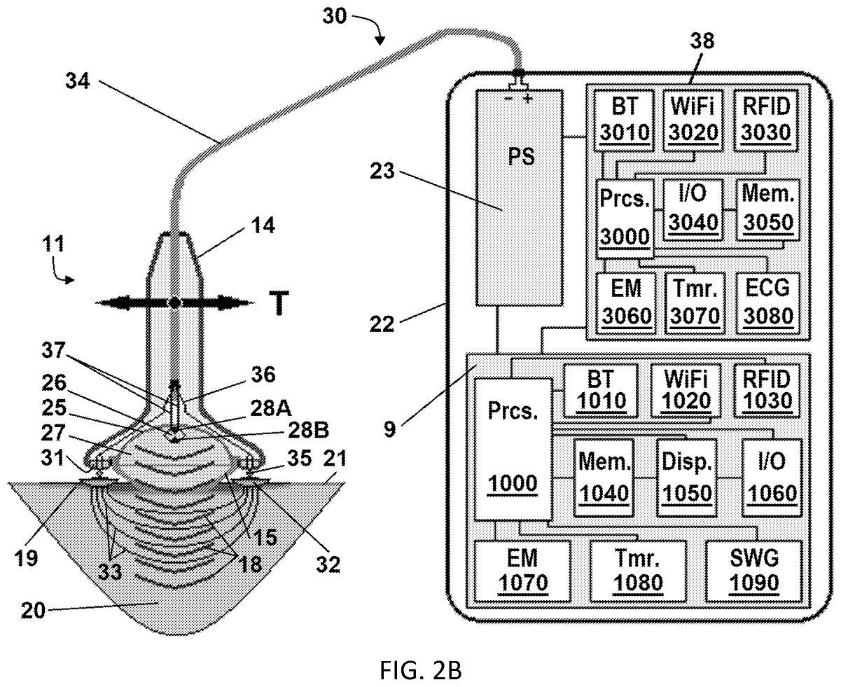

FIGS. 2A and 2B show the combination of shockwaves/pressure waves and electric current fields medical system 30 that is capable in the same device to produce shockwaves/pressure waves in combination with mobile electric fields. These specially designed applicators and medical systems that use combination treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous electric fields that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with electric fields from the outside/exterior of the human or animal body (extracorporeally) and towards the targeted treatment zone.

As seen from FIGS. 2A and 2B, the combination of shockwaves/pressure waves and electric current fields medical system 30 includes a combination applicator 11 connected to the control console/unit 22 shown in FIG. 2B via a combination power cable 34, which contains multiple power cables at different voltages in its construction, as dictated by the needs of the energy technologies that are activated inside the combination applicator 11. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 2B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid for correct propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The specially designed coupling bladder or spacer containing fluids can allow the formation of cavitation (produced by the shockwaves/pressure waves 18) on top of a superficial wound/lesion/tissue condition 19, with benefic effects for healing. The shockwaves/pressure waves 18 are passing through the wound/lesion/tissue condition 19 that can be positioned superficial, immediately under the skin 21 or deep inside the human/animal body 20. On the peripheral part of the bulbous region of the applicator body 14 and next to the applicator/coupling membrane 15, at least two or three or four or more electric current emission elements 31 are present that are connected via the swiveling hinges 35 to the swiveling electric current electrodes 32, which are capable to create electric current field patterns 33 inside the wound/lesion/tissue condition 19. In FIGS. 2A and 2B there are two (2) electric current emission elements 31, each of them with a dedicated electric current emission element wiring 36. The swiveling movement S (see FIG. 2A) allows the correct position of the swiveling electric current electrodes 32 and constant contact with the wound/lesion/tissue condition 19. The combined effects of the shockwaves/pressure waves 18 and of the electric current field patterns 33 will enhance and expedite the healing of the wound/lesion/tissue condition 19, which is beneficial for a successful medical treatment and thus becomes more financially viable and less time consuming for both the patients and the medical personnel.

In FIG. 2B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the reflector 25, the applicator/coupling membrane 15, the electric current emission element wiring 36, and the electric current emission elements 31, which are placed inside or sit on or are attached to the applicator body 14.

The combination power cable 34 provides the connection in between the combination applicator 11 and the control console/unit 22 and it goes inside the applicator body 14, via the shockwave/pressure wave electrodes wiring 37, until it reaches the first electrode 28A and the second electrode 28B of the spark-gap 26, where the shockwaves/pressure waves 18 are generated inside the fluid-filled reflector and membrane cavity 27. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, and the electric current field generator (ECG) 38 are sharing a common power supply (PS) 23, which can be incorporated inside the control console/unit 22 (as presented in FIG. 2B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the electric current field generator (ECG) 38, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 2B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The electric current generator processor 3000 has a slave-master relationship with the control console/unit processor 1000, which means that the control console/unit processor 1000 controls the activity of the electric current generator processor 3000 via a continuous interaction process of sharing information and commands. Thus, the control console/unit processor 1000 dictates the functionality of the entire electric current field generator (ECG) 38. Through this process the control console/unit processor 1000 together with the electric current generator processor 3000 control the electric current form (low intensity direct current (LIDC), high voltage pulsed current (HVPC), asymmetrical biphasic pulsed current (ABPC), alternative current (AC), etc.), time of activation, intensity, polarity, points of application, and the electric current field patterns 33.

The control console/unit processor 1000 practically controls the whole activity of any combination energy medical systems, including the combination shockwaves/pressure waves and electric current fields medical system 30 presented in FIGS. 2A and 2B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the electric current field patterns 33. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, electric current form (low intensity direct current (LIDC), high voltage pulsed current (HVPC), asymmetrical biphasic pulsed current (ABPC), alternative current (AC), etc.), time of activation, intensity, polarity, points of application, electric current field patterns 33, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the electric current field generator (ECG) 38 includes the electric current generator processor 3000, the electric current generator Bluetooth (BT) module 3010, the electric current generator Wi-Fi module 3020, the electric current generator RFID module 3030, the electric current generator I/O (input/output) element 3040, the electric current generator memory 3050, the electric current generator emission mechanism (EM) module 3060, the electric current generator timer module 3070, and the electric current field generator (ECG) module 3080.

In another embodiment, the electric current field generator (ECG) 38 contains only the electric current generator processor 3000. Additionally, in some embodiments depending on the capabilities of the electric current field generator (ECG) 38 and control console/unit 22, the electric current field generator (ECG) 38 may also include the electric current generator Bluetooth (BT) module 3010, the electric current generator Wi-Fi module 3020, and the electric current generator RFID module 3030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the electric current generator I/O (input/output) element 3040 or the electric current generator memory 3050 can be present or none of them are present in the construction of the electric current field generator (ECG) 38. The advantage of having the electric current generator I/O (input/output) element 3040 is that it offers the possibility to independently input or output data for the functionality of the electric current field generator (ECG) 38. Regarding the electric current generator memory 3050, this module allows to store the functionality of the electric current field generator (ECG) 38 separately from the control console/unit memory 1040 of the shockwaves/pressure waves field generator 9. The electric current field generator (ECG) 38 and its electric current generator processor 3000 control the electric current form (low intensity direct current (LIDC), high voltage pulsed current (HVPC), asymmetrical biphasic pulsed current (ABPC), alternative current (AC), etc.), time of activation, intensity, polarity, points of application, and the electric current field patterns 33. For that, the electric current generator processor 3000 is using the electric current generator emission mechanism (EM) module 3060, the electric current generator timer module 3070, and the electric current field generator (ECG) module 3080. The electric current field generator (ECG) module 3080 may include hardware or components, which are well-known to those skilled in the art, for providing via the combination applicator 11 the proper electric currents and the associated electric filed patterns 33. The electric current generator timer module 3070 provides timing sequence for emitting the one or more generated electric current field patterns 33 into the wound/lesion/tissue condition 19, as dictated by the one or more energy combination treatment settings. The one or more electric current field patterns 33 are emitted in a manner controlled by the electric current generator emission mechanism (EM) module 3060 and transmitted from the control console/unit 22 through the combination power cable 34 towards the combination applicator 11, via a secured data transfer protocol, as mentioned before.

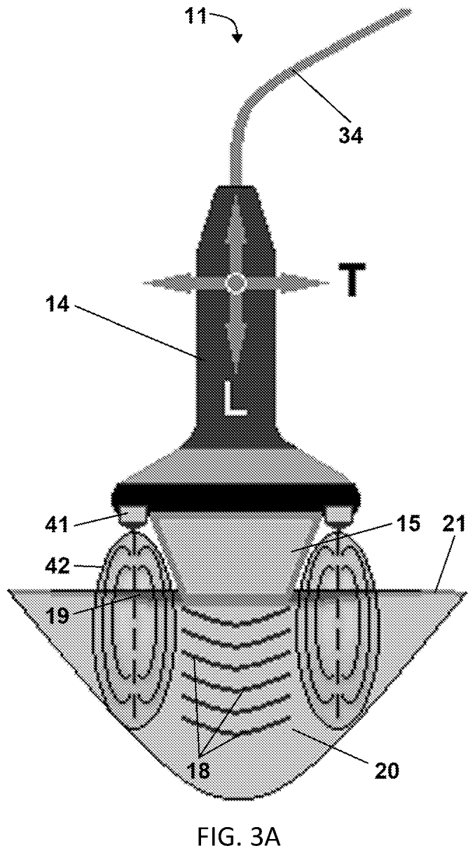

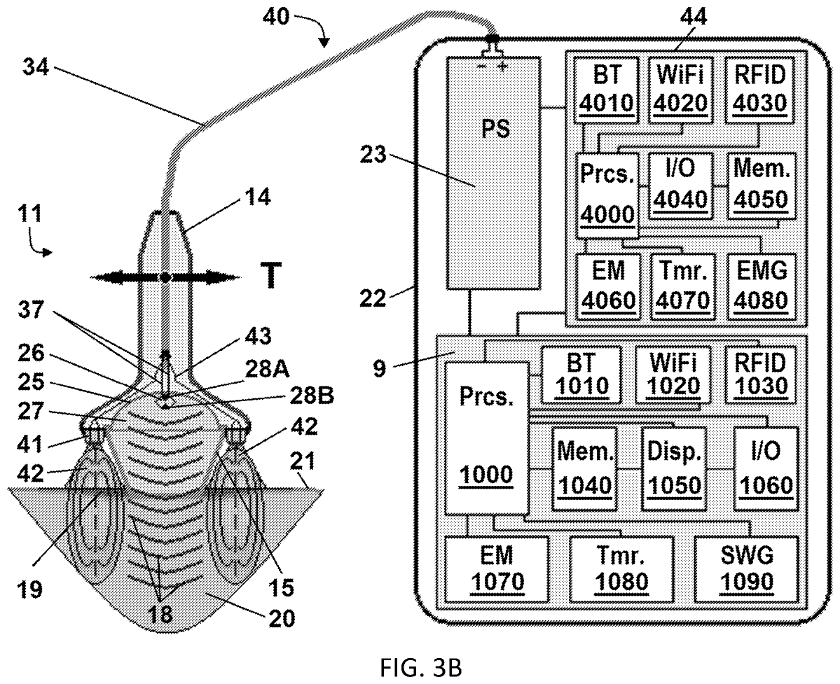

FIGS. 3A and 3B show the combination shockwaves/pressure waves and electromagnetic fields medical system 40 that is capable in the same device to produce shockwaves/pressure waves together with electromagnetic fields. These specially designed applicators and medical systems that use combination treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to generate pulsed or continuous electromagnetic fields that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with electromagnetic fields from the outside/exterior of the human or animal body (extracorporeally) and towards the targeted treatment zone.

As seen from FIGS. 3A and 3B, the combination shockwaves/pressure waves and electromagnetic fields medical system 40 includes a combination applicator 11 that uses a combination power cable 34 to connect to the control console/unit 22 shown in FIG. 3B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 3B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel for correct propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, immediately under the skin 21 or deep inside the human/animal body 20. On the peripheral part of the bulbous region of the applicator body 14 and next to the applicator/coupling membrane 15, at least two or three or four or more electromagnetic emission elements 41 are present, which are capable to create electromagnetic field patterns 42 inside the wound/lesion/tissue condition 19. In FIGS. 3A and 3B there are two (2) electromagnetic emission elements 41, each of them with a dedicated electromagnetic emission element wiring 43. The electromagnetic emission elements 41 can be at a certain distance from the skin 21 or wound/lesion/tissue condition 19 (as seen in FIGS. 3A and 3B) or from a specially designed coupling bladder or spacer (mentioned before in this paragraph) or in another embodiment the electromagnetic emission elements 41 can be modified/designed to be positioned in direct contact with the skin 21 or wound/lesion/tissue condition 19 or with a specially designed coupling bladder or spacer. The combined effects of the shockwaves/pressure waves 18 and of the electromagnetic field patterns 42 will enhance and expedite the healing of the wound/lesion/tissue condition 19, which is beneficial for a successful medical treatment and thus becomes more financially viable and less time consuming for both the patients and the medical personnel.

In FIG. 3B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the reflector 25, the applicator/coupling membrane 15, the electromagnetic emission element wiring 43, and the electromagnetic emission elements 41, which are placed inside or sit on or are attached to the applicator body 14.

The combination power cable 34 provides the connection in between the combination applicator 11 and the control console/unit 22 and it goes inside the applicator body 14, via the shockwave/pressure wave electrodes wiring 37 until it reaches the first electrode 28A and the second electrode 28B of the spark-gap 26, where the shockwaves/pressure waves 18 are generated inside the fluid-filled reflector and membrane cavity 27. The high voltage that travels through combination power cable 34 is produced inside the control console/unit 22 by the power supply (PS) 23, which is controlled and directly connected to the shockwaves/pressure waves field generator 9. It is also important to note that the combination applicator 11, the control console/unit 22, the shockwaves/pressure waves field generator 9, and the electromagnetic generator 44 are sharing a common power supply (PS) 23, which can be incorporated inside the control console/unit 22 (as presented in FIG. 3B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the electromagnetic generator 44, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 3B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The electromagnetic generator processor 4000 has a slave-master relationship with the control console/unit processor 1000, which means that the control console/unit processor 1000 controls the activity of the electromagnetic generator processor 4000 via a continuous interaction process of sharing information and commands. Thus, the control console/unit processor 1000 dictates the functionality of the entire electromagnetic generator 44. Through this process the control console/unit processor 1000 together with the electromagnetic generator processor 4000 control the type of electromagnetic fields (continuous electromagnetic fields (EMFs) or pulsed electromagnetic fields (PEMF)), electric current form intensity used to create electromagnetic fields, time of activation, intensity, polarity, points of application, and the electromagnetic field pattern 42.

The control console/unit processor 1000 practically controls the whole activity of any combination energy medical systems, including the combination shockwaves/pressure waves and electromagnetic fields medical system 40 presented in FIGS. 3A and 3B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the electromagnetic field patterns 42. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of combination energy medical treatments, shockwaves/pressure waves input energy, frequency of shockwaves/pressure waves per second, type of electromagnetic fields (continuous electromagnetic fields (EMFs) or pulsed electromagnetic fields (PEMF)), electric current form intensity used to create electromagnetic fields, time of activation, intensity, polarity, points of application, electromagnetic field pattern 42, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

The electromagnetic generator 44 includes the electromagnetic generator processor 4000, the electromagnetic generator Bluetooth (BT) module 4010, the electromagnetic generator Wi-Fi module 4020, the electromagnetic generator RFID module 4030, the electromagnetic generator I/O (input/output) element 4040, the electromagnetic generator memory 4050, the electromagnetic generator emission mechanism (EM) module 4060, the electromagnetic generator timer module 4070, and the electromagnetic field generator (EMG) module 4080.

In another embodiment, the electromagnetic generator 44 contains only the electromagnetic generator processor 4000. Additionally, in some embodiments depending on the capabilities of the electromagnetic generator 44 and control console/unit 22, the electromagnetic generator 44 may also include the electromagnetic generator Bluetooth (BT) module 4010, the electromagnetic generator Wi-Fi module 4020, and the electromagnetic generator RFID module 4030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the electromagnetic generator I/O (input/output) element 4040 or the electromagnetic generator memory 4050 can be present or none of them are present in the construction of the electromagnetic generator 44. The advantage of having the electromagnetic generator I/O (input/output) element 4040 is that it offers the possibility to independently input or output data for the functionality of the electromagnetic generator 44. Regarding the electromagnetic generator memory 4050, this module allows to store the functionality of the electromagnetic generator 44 separately from the control console/unit memory 1040 of the shockwaves/pressure waves field generator 9. The electromagnetic generator 44 and its electromagnetic generator processor 4000 control the type of electromagnetic fields (continuous electromagnetic fields (EMFs) or pulsed electromagnetic fields (PEMF)), electric current form intensity used to create electromagnetic fields, time of activation, intensity, polarity, points of application, and the electromagnetic field pattern 42. For that, the electromagnetic generator processor 4000 is using the electromagnetic generator emission mechanism (EM) module 4060, the electromagnetic generator timer module 4070, and the electromagnetic field generator (EMG) module 4080. The electromagnetic field generator (EMG) module 4080 may include hardware or components, which are well-known to those skilled in the art, for providing via the combination applicator 11 the proper electromagnetic fields and the associated electromagnetic field patterns 42. The electromagnetic generator timer module 4070 provides timing sequence for emitting the one or more generated electromagnetic field patterns 42 into the wound/lesion/tissue condition 19, as dictated by the one or more energy combination treatment settings. The one or more electromagnetic field patterns 42 are emitted in a manner controlled by the electromagnetic generator emission mechanism (EM) module 4060 and transmitted from the control console/unit 22 through the combination power cable 34 towards the combination applicator 11, via a secured data transfer protocol, as mentioned before.

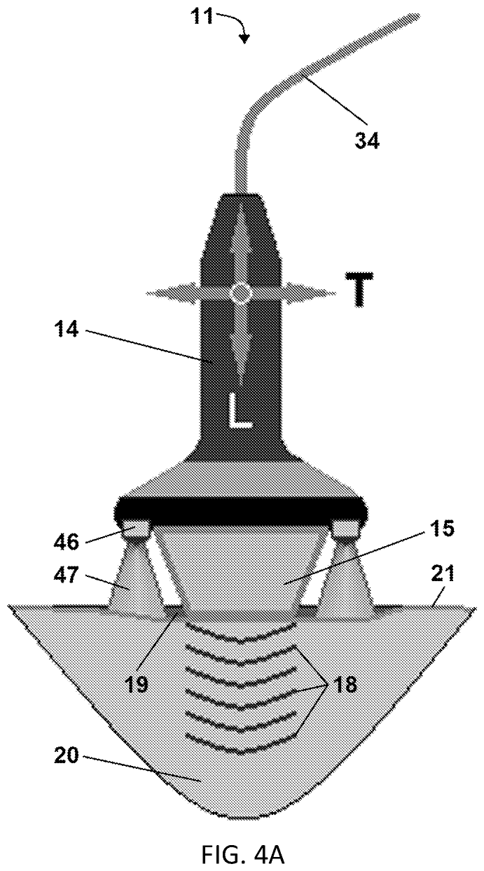

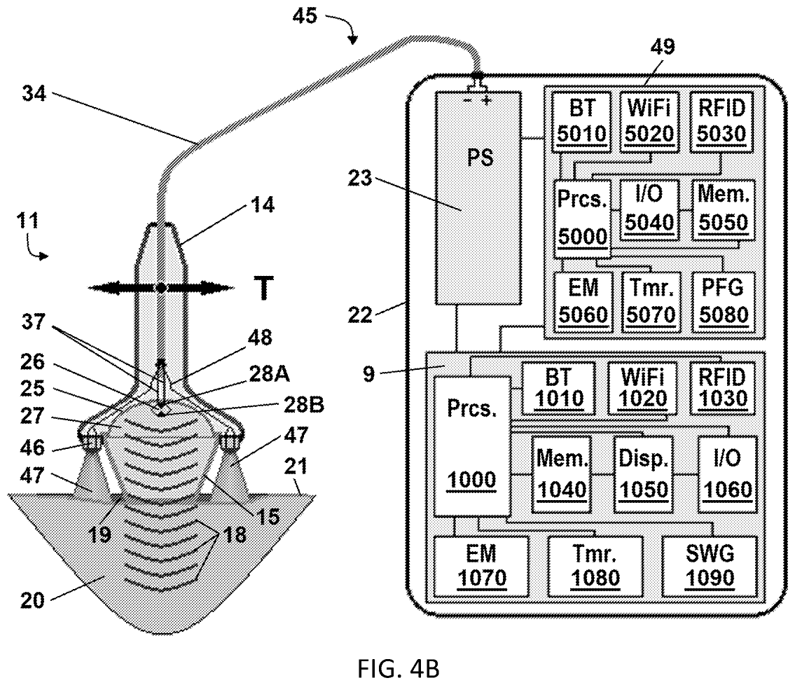

FIGS. 4A and 4B show the combination shockwaves/pressure waves and LED/OLED phototherapy medical system 45 that is capable in the same device to produce shockwaves/pressure waves in combination with LED/OLED (light emitting diode/organic light emitting diode) phototherapy in the same device. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce phototherapy fields that are delivered simultaneously with the shockwaves/pressure waves 18 for stimulation, healing, and regeneration of cells, tissues, or organs. In some designs LED/OLED photodynamic therapy can be used, which also require special light-sensitive medicine or gels and a light source to stimulate or in some cases to destroy abnormal cells. In this case, the combination applicators 11 will have a special design that can facilitate a medical treatment using the shockwaves/pressure waves 18 in combination with pulsed or continuous LED/OLED phototherapy from the outside/exterior of the human or animal body (extracorporeally) and towards the targeted treatment zone.

As seen from FIGS. 4A and 4B, the combination shockwaves/pressure waves and LED/OLED phototherapy medical system 45 includes a combination applicator 11 that uses a combination power cable 34 to connect to the control console/unit 22 shown in FIG. 4B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 4B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with the skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more LEDs/OLEDs 46 are present, which are capable to create LED/OLED phototherapy field patterns 47 on the surface or inside the wound/lesion/tissue condition 19. In FIGS. 4A and 4B there are two (2) LEDs/OLEDs 46, each of them with a dedicated LED/OLED wiring 48. The combined effects of the shockwaves/pressure waves 18 and of the LED/OLED phototherapy field patterns 47 will enhance and expedite the healing of the wound/lesion/tissue condition 19, which is beneficial for a successful medical treatment and thus becomes more financially viable and less time consuming for both the patients and the medical personnel.

In FIG. 4B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the reflector 25, the applicator/coupling membrane 15, the LED/OLED wiring 48, and the LEDs/OLEDs 46, which are placed inside or sit on or are attached to the applicator body 14.

The combination power cable 34 provides the connection in between the combination applicator 11 and the control console/unit 22 and it goes inside the applicator body 14, via the shockwave/pressure wave electrodes wiring 37, until it reaches the first electrode 28A and the second electrode 28B of the spark-gap 26, where the shockwaves/pressure waves 18 are generated inside the fluid-filled reflector and membrane cavity 27. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, and photolight generator 49 are sharing a common power supply (PS) 23, which can be incorporated inside the control console/unit 22 (as presented in FIG. 4B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the photolight generator 49, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 4B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The photolight generator processor 5000 has a slave-master relationship with the control console/unit processor 1000, which means that the control console/unit processor 1000 controls the activity of the photolight generator processor 5000 via a continuous interaction process of sharing information and commands. Thus, the control console/unit processor 1000 dictates the functionality of the entire photolight generator 49. Through this process the control console/unit processor 1000 together with the photolight generator processor 5000 control the type of light (white, blue, green, yellow, amber, red, or near-infrared), time of activation, intensity, points of application, levels of brightness, color-changing capability with broad spectral tunability and ultrafast responses, and the LED/OLED phototherapy field patterns 47.

The control console/unit processor 1000 practically controls the whole activity of any combination energy medical systems, including the combination shockwaves/pressure waves and LED/OLED phototherapy medical system 45 presented in FIGS. 4A and 4B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the LED/OLED phototherapy field patterns 47. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, type of light (white, blue, green, yellow, amber, red, or near-infrared), time of activation, intensity, points of application, levels of brightness, color-changing capability with broad spectral tunability and ultrafast responses, LED/OLED phototherapy field patterns 47, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the photolight generator 49 includes the photolight generator processor 5000, the photolight generator Bluetooth (BT) module 5010, the photolight generator Wi-Fi module 5020, the photolight generator RFID module 5030, the photolight generator I/O (input/output) element 5040, the photolight generator memory 5050, the photolight generator emission mechanism (EM) module 5060, the photolight generator timer module 5070, and photolight field generator (PFG) module 5080.

In another embodiment, the photolight generator 49 contains only the photolight generator processor 5000. Additionally, in some embodiments depending on the capabilities of the photolight generator 49 and control console/unit 22, the photolight generator 49 may also include the photolight generator Bluetooth (BT) module 5010, the photolight generator Wi-Fi module 5020, and the photolight generator RFID module 5030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the photolight generator I/O (input/output) element 5040 or the photolight generator memory 5050 can be present or none of them are present in the construction of the photolight generator 49. The advantage of having the photolight generator I/O (input/output) element 5040 is that it offers the possibility to independently input or output data for the functionality of the photolight generator 49. Regarding the photolight generator memory 5050, this module allows to store the functionality of the photolight generator 49 separately from the control console/unit memory 1040 of the shockwaves/pressure waves field generator 9. The photolight generator 49 and its photolight generator processor 5000 control the type of light (white, blue, green, yellow, amber, red, or near-infrared), time of activation, intensity, points of application, levels of brightness, color-changing capability with broad spectral tunability and ultrafast responses, and the LED/OLED phototherapy field patterns 47. For that, the photolight generator processor 5000 is using the photolight generator emission mechanism (EM) module 5060, the photolight generator timer module 5070, and photolight field generator (PFG) module 5080. The photolight field generator (PFG) module 5080 may include hardware or components, which are well-known to those skilled in the art, for providing via the combination applicator 11 the proper LED/OLED phototherapy field patterns 47. The photolight generator timer module 5070 provides timing sequence for emitting the one or more generated LED/OLED phototherapy field patterns 47 into the wound/lesion/tissue condition 19, as dictated by the one or more energy combination treatment settings. The one or more electric LED/OLED phototherapy field patterns 47 are emitted in a manner controlled by the photolight generator emission mechanism (EM) module 5060 and transmitted from the control console/unit 22 through the combination power cable 34 towards the combination applicator 11, via a secured data transfer protocol, as mentioned before.

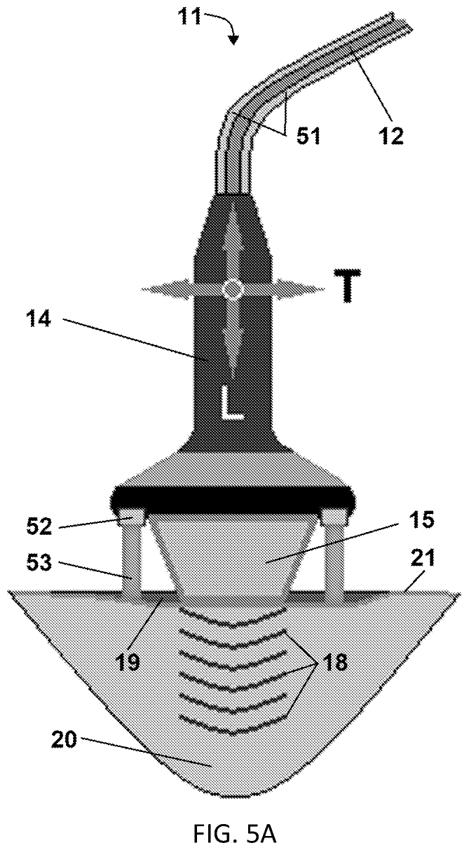

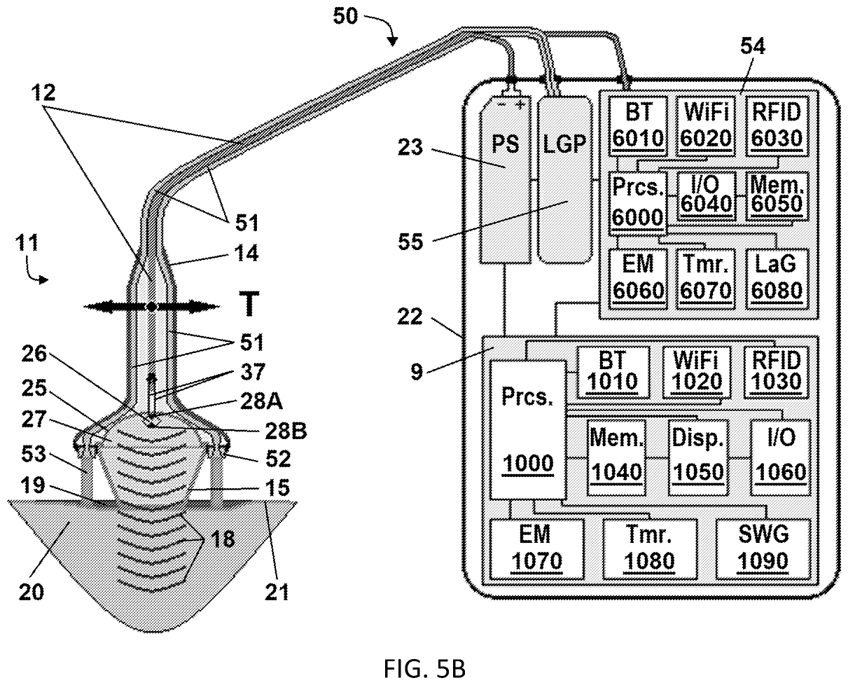

FIGS. 5A and 5B show the combination shockwaves/pressure waves and laser phototherapy medical system 50 that is capable in the same device to produce shockwaves/pressure waves in combination with laser phototherapy fields. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous laser phototherapy fields that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with laser phototherapy from the outside/exterior of the human or animal body (extracorporeally) and towards the targeted treatment zone.

As seen from FIGS. 5A and 5B, the combination shockwaves/pressure waves and laser phototherapy medical system 50 includes a combination applicator 11 that uses a high voltage cable 12 and laser fiber optic cables 51 to connect to the control console/unit 22 shown in FIG. 5B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 5B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more laser fiber optic cable exit lenses 52 are present, which are capable to create laser phototherapy field patterns 53 on the surface or inside the wound/lesion/tissue condition 19. In FIGS. 5A and 5B there are two (2) laser fiber optic cable exit lenses 52, each of them with a dedicated laser fiber optic cable 51. The combined effects of the shockwaves/pressure waves 18 and of the laser phototherapy field patterns 53 will enhance and expedite the healing of the wound/lesion/tissue condition 19, which is beneficial for a successful medical treatment and thus becomes more financially viable and less time consuming for both the patients and the medical personnel.

In FIG. 5B, the focus is on the configuration of the control console/unit 22 and the internal structure of the combination applicator 11. The combination applicator 11 internal structure contains different elements as the high voltage cable 12, the shockwave/pressure wave electrodes wiring 37 for the first electrode 28A and the second electrode 28B of the spark-gap 26, the reflector 25, the applicator/coupling membrane 15, the laser fiber optic cables 51, and the laser fiber optic cable exit lenses 52, which are placed inside or sit on or are attached to the applicator body 14.

The high voltage cable 12 and laser fiber optic cables 51 provide the connection in between the combination applicator 11 and the control console/unit 22 and they go inside the applicator body 14, via the shockwave/pressure wave electrodes wiring 37, until it reaches the first electrode 28A and the second electrode 28B of the spark-gap 26, where the shockwaves/pressure waves 18 are generated inside the fluid-filled reflector and membrane cavity 27. The high voltage that travels through high voltage cable 12 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, the laser generator 54, and its associated laser generator pump (LGP) 55 are sharing a common power supply (PS) 23, which can be incorporated inside the control console/unit 22 (as presented in FIG. 5B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the laser generator 54, and its associated laser generator pump (LGP) 55, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 5B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The laser generator processor 6000 has a slave-master relationship with the control console/unit processor 1000, which means that the control console/unit processor 1000 controls the activity of the laser generator processor 6000 via a continuous interaction process of sharing information and commands. Thus, the control console/unit processor 1000 dictates the functionality of the entire laser generator 54 and its associated laser generator pump (LGP) 55. The role of the laser generator pump (LGP) 55 is to absorb energy from the power supply (PS) 23 and under the control of the laser generator 54, to produce an excited state of the atoms present in the medium from inside the pump. This is the way the laser light is produced inside the laser generator pump (LGP) 55. Through this process the control console/unit processor 1000 together with the laser generator processor 6000 control the type of laser (red, infrared, etc.), laser amplitude that dictates laser's color and penetration, duration of the pulse, time of activation, intensity, points of application, levels of brightness, continuous or intermittent application, and the laser phototherapy field patterns 53.

The control console/unit processor 1000 practically controls the whole activity of any combination energy medical systems, including the combination shockwaves/pressure waves and laser phototherapy medical system 50 presented in FIGS. 5A and 5B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the laser phototherapy field patterns 53. The data transfer protocol in between the control console/unit 22 and combination applicator 11 is secured via an authentication process that includes processor generated passcodes and passwords, authentication process that is performed when the high voltage cable 12 and laser fiber optic cables 51 are connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, type of laser (red, infrared, etc.), laser amplitude that dictates laser's color and penetration, duration of the pulse, time of activation, intensity, points of application, levels of brightness, continuous or intermittent application, laser phototherapy field patterns 53, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the laser generator 54 includes the laser generator processor 6000, the laser generator Bluetooth (BT) module 6010, the laser generator Wi-Fi module 6020, the laser generator RFID module 6030, the laser generator I/O (input/output) element 6040, the laser generator memory 6050, the laser generator emission mechanism (EM) module 6060, the laser generator timer module 6070, and the laser field generator (LaG) module 6080.

In another embodiment, the laser generator 54 contains only the laser generator processor 6000. Additionally, in some embodiments depending on the capabilities of the laser generator 54 and control console/unit 22, the laser generator 54 may also include the laser generator Bluetooth (BT) module 6010, the laser generator Wi-Fi module 6020, and the laser generator RFID module 6030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the laser generator I/O (input/output) element 6040 or the laser generator memory 6050 can be present or none of them are present in the construction of the laser generator 54. The advantage of having the laser generator I/O (input/output) element 6040 is that it offers the possibility to independently input or output data for the functionality of the laser generator 54 and its associated laser generator pump (LGP) 55. Regarding the laser generator memory 6050, this module allows to store the functionality of the laser generator 54 and its associated laser generator pump (LGP) 55 separately from the control console/unit memory 1040 of the shockwaves/pressure waves field generator 9. The laser generator 54 and its laser generator processor 6000 control the type of laser (red, infrared, etc.), laser amplitude that dictates laser's color and penetration, duration of the pulse, time of activation, intensity, points of application, levels of brightness, continuous or intermittent application, and the laser phototherapy field patterns 53. For that, the laser generator processor 6000 is using the laser generator emission mechanism (EM) module 6060, the laser generator timer module 6070, and the laser field generator (LaG) module 6080. The laser field generator (LaG) module 6080 may include hardware or components, which are well-known to those skilled in the art, for providing via the combination applicator 11 the proper laser phototherapy field patterns 53. The laser generator timer module 6070 provides timing sequence for emitting the one or more generated laser phototherapy field patterns 53 into the wound/lesion/tissue condition 19, as dictated by the one or more energy combination treatment settings. The one or more laser phototherapy field patterns 53 are emitted in a manner controlled by the laser generator emission mechanism (EM) module 6060 and transmitted from the control console/unit 22 through the laser fiber optic cables 51 towards the combination applicator 11, via a secured data transfer protocol, as mentioned before.

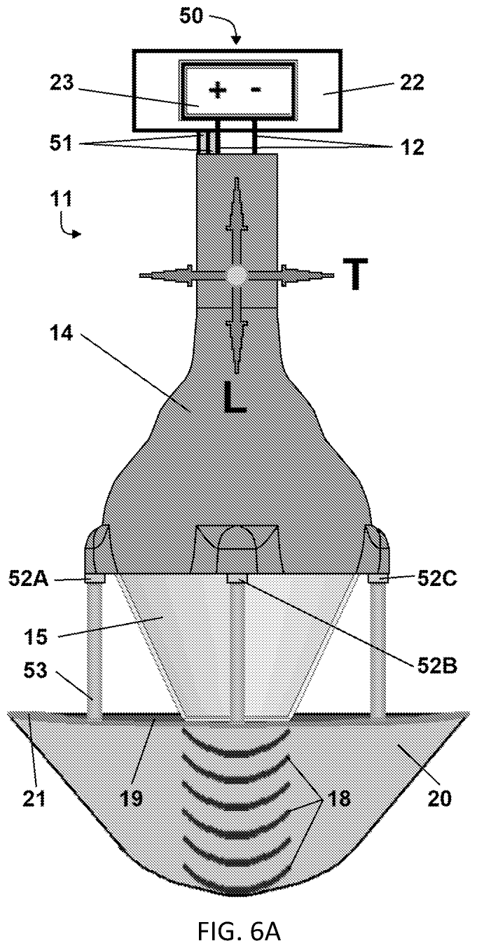

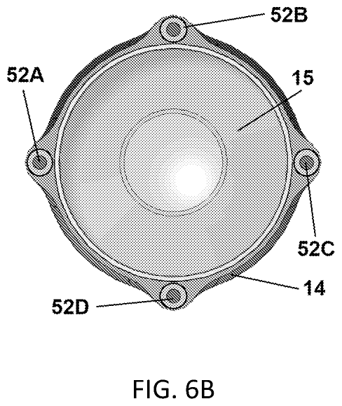

As mentioned for FIGS. 5A and 5B, the combination shockwaves/pressure waves and laser phototherapy medical system 50 can use more than two (2) lasers. Thus, in FIGS. 6A and 6B is presented a combination shockwaves/pressure waves and laser phototherapy medical system 50 that simultaneously delivers shockwaves and laser phototherapy with four (4) lasers. As seen in FIG. 6B, four laser fiber optic cable exit lenses are disposed equally at 45 degrees on the circumference of bulbous part of the applicator body 14. As seen from FIGS. 5A and 5B, the combination shockwaves/pressure waves and laser phototherapy medical system 50 includes a combination applicator 11 that uses a high voltage cable 12 and laser fiber optic cables 51 to connect to the control console/unit 22 and its associated power supply (PS) 23. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen for FIGS. 5A and 5B) that actuates an external motorized ancillary system (not specifically presented in any of the figures of this patent for simplicity) that also contains sensors, motors, cameras, and clamping mechanism for the combination applicator 11. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, the first laser fiber optic cable exit lens 52A, the second laser fiber optic cable exit lens 52B, the third laser fiber optic cable exit lens 52C, and the fourth laser fiber optic cable exit lens 52D can be seen, which are capable to create four laser phototherapy field patterns 53 on the surface or inside the wound/lesion/tissue condition 19. This allows a more efficient coverage of the wound/lesion/tissue condition 19 during treatment, which enhance the efficiency of the energy combination treatment using the combination shockwaves/pressure waves and laser phototherapy medical system 50 from FIGS. 6A and 6B, when compared to the combination shockwaves/pressure waves and laser phototherapy medical system 50 from FIGS. 5A and 5B. Of course, more than four lasers can be used for any of the construction of the combination shockwaves/pressure waves and laser phototherapy medical system 50. The combined effects of the shockwaves/pressure waves 18 and of the four laser phototherapy field patterns 53 will enhance and expedite the healing of the wound/lesion/tissue condition 19, which is beneficial for a successful medical treatment and thus becomes more financially viable and less time consuming for both the patients and the medical personnel. Finally, the purpose, structure, construction, usability and functionality of all elements of the combination shockwaves/pressure waves and laser phototherapy medical system 50 from FIGS. 6A and 6B are similar to those presented before for FIGS. 5A and 5B.

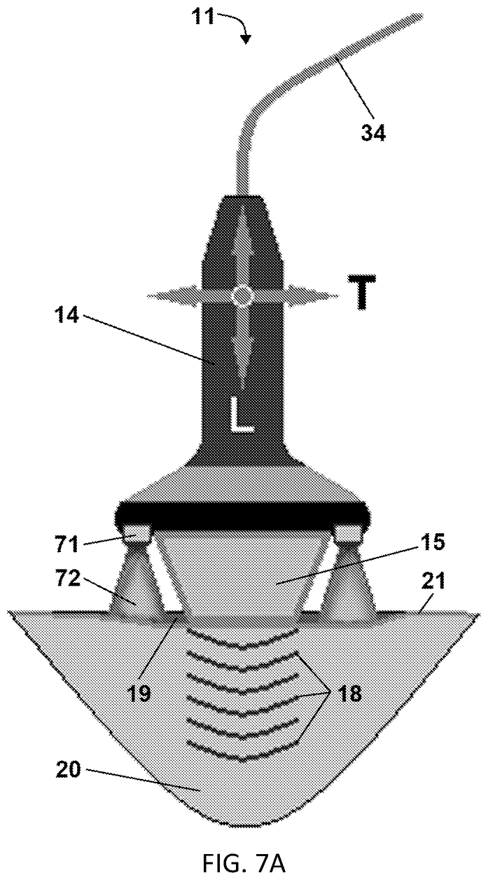

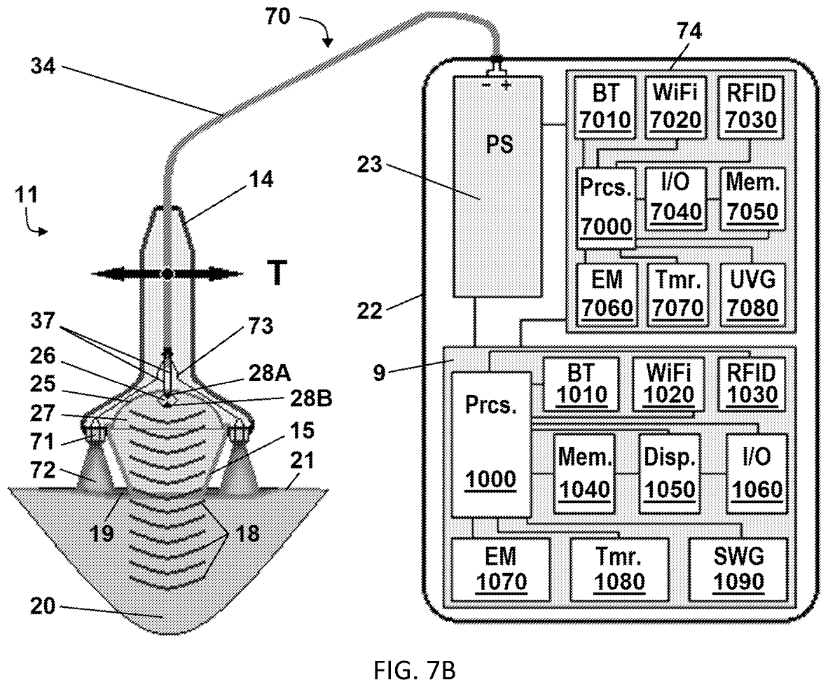

FIGS. 7A and 7B show the combination shockwaves/pressure waves and ultraviolet light medical system 70 that is capable in the same device to produce shockwaves/pressure waves in combination with ultraviolet (UV) light fields. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous UV light fields that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with UV light fields 72 from the outside/exterior of the human or animal body (extracorporeally) and towards the targeted treatment zone.

As seen from FIGS. 7A and 7B, the combination shockwaves/pressure waves and ultraviolet light medical system 70 includes a combination applicator 11 that uses a combination power cable 34 to connect to the control console/unit 22 shown in FIG. 7B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 7B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more ultraviolet light lamps 71 are present, which are capable to create ultraviolet light field patterns 72 inside the wound/lesion/tissue condition 19. In FIGS. 7A and 7B there are two (2) ultraviolet light lamps 71, each of them with a dedicated ultraviolet light lamp wiring 73. The combined effects of the shockwaves/pressure waves 18 and of the ultraviolet light field patterns 72 will enhance and expedite the healing of the wound/lesion/tissue condition 19, which is beneficial for a successful medical treatment and thus becomes more financially viable and less time consuming for both the patients and the medical personnel.

In FIG. 7B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the reflector 25, the applicator/coupling membrane 15, the ultraviolet light lamp wiring 73, and the ultraviolet light lamps 71, which are placed inside or sit on or are attached to the applicator body 14.

The combination power cable 34 provides the connection in between the combination applicator 11 and the control console/unit 22 and it goes inside the applicator body 14, via the shockwave/pressure wave electrodes wiring 37, until it reaches the first electrode 28A and the second electrode 28B of the spark-gap 26, where the shockwaves/pressure waves 18 are generated inside the fluid-filled reflector and membrane cavity 27. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, and the ultraviolet light generator (UVG) 74 are sharing a common power supply (PS) 23, which can be incorporated inside the control console/unit 22 (as presented in FIG. 7B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the ultraviolet light generator (UVG) 74, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 7B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The ultraviolet light generator processor 7000 has a slave-master relationship with the control console/unit processor 1000, which means that the control console/unit processor 1000 controls the activity of the ultraviolet light generator processor 7000 via a continuous interaction process of sharing information and commands. Thus, the control console/unit processor 1000 dictates the functionality of the entire ultraviolet light generator (UVG) 74. Through this process the control console/unit processor 1000 together with the ultraviolet light generator processor 7000 control the UV light type (UV light A (UVA), UV light B (UVB), UV light C (UVC), or vacuum UV), time of activation, intensity, distance and angle of the light source, and the ultraviolet light field pattern 72.

The control console/unit processor 1000 practically controls the whole activity of any combination energy medical systems, including the combination shockwaves/pressure waves and ultraviolet light medical system 70 presented in FIGS. 7A and 7B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the ultraviolet light field patterns 72. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, UV light type (UV light A (UVA), UV light B (UVB), UV light C (UVC), or vacuum UV), time of activation, intensity, distance and angle of the light source, ultraviolet light field pattern 72, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the ultraviolet light generator (UVG) 74 includes the ultraviolet light generator processor 7000, the ultraviolet light generator Bluetooth (BT) module 7010, the ultraviolet light generator Wi-Fi module 7020, the ultraviolet light generator RFID module 7030, the ultraviolet light generator I/O (input/output) element 7040, the ultraviolet light generator memory 7050, the ultraviolet light generator emission mechanism (EM) module 7060, the ultraviolet light generator timer module 7070, and the ultraviolet light field generator (UVG) module 7080.

In another embodiment, the ultraviolet light generator (UVG) 74 contains only the ultraviolet light generator processor 7000. Additionally, in some embodiments depending on the capabilities of the ultraviolet light generator (UVG) 74 and control console/unit 22, the ultraviolet light generator (UVG) 74 may also include the ultraviolet light generator Bluetooth (BT) module 7010, the ultraviolet light generator Wi-Fi module 7020, and the ultraviolet light generator RFID module 7030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the ultraviolet light generator I/O (input/output) element 7040 or the ultraviolet light generator memory 7050 can be present or none of them are present in the construction of the ultraviolet light generator (UVG) 74. The advantage of having the ultraviolet light generator I/O (input/output) element 7040 is that it offers the possibility to independently input or output data for the functionality of the ultraviolet light generator (UVG) 74. Regarding the ultraviolet light generator memory 7050, this module allows to store the functionality of the ultraviolet light generator (UVG) 74 separately from the control console/unit memory 1040 of the shockwaves/pressure waves field generator 9. The ultraviolet light generator (UVG) 74 and its ultraviolet light generator processor 7000 control the UV light type (UV light A (UVA), UV light B (UVB), UV light C (UVC), or vacuum UV), time of activation, intensity, distance and angle of the light source, and the ultraviolet light field patterns 72. For that, the ultraviolet light generator processor 7000 is using the ultraviolet light generator emission mechanism (EM) module 7060, the ultraviolet light generator timer module 7070, and the ultraviolet light field generator (UVG) module 7080. The ultraviolet light field generator (UVG) module 7080 may include hardware or components, which are well-known to those skilled in the art, for providing via the combination applicator 11 the proper associated ultraviolet light field patterns 72. The ultraviolet light generator timer module 7070 provides timing sequence for emitting the one or more generated ultraviolet light field patterns 72 into the wound/lesion/tissue condition 19, as dictated by the one or more energy combination treatment settings. The one or more ultraviolet light field patterns 72 are emitted in a manner controlled by the ultraviolet light field generator (UVG) module 7080 and transmitted from the control console/unit 22 through the combination power cable 34 towards the combination applicator 11, via a secured data transfer protocol, as mentioned before.

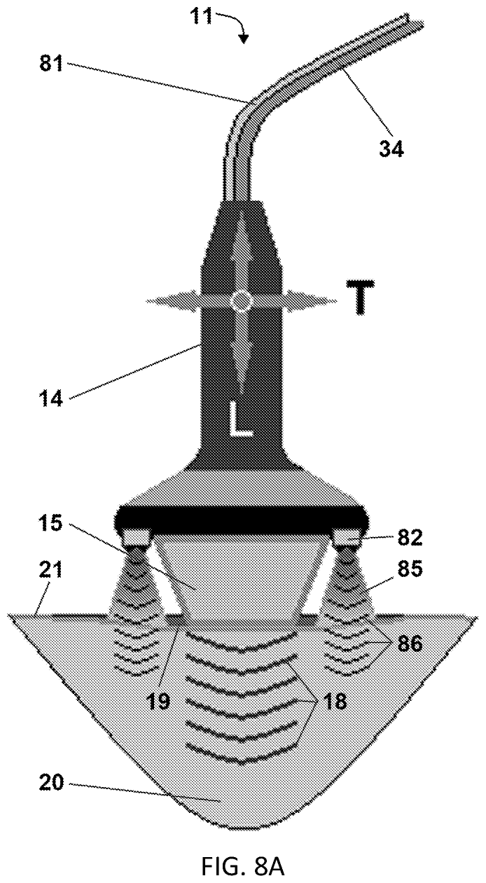

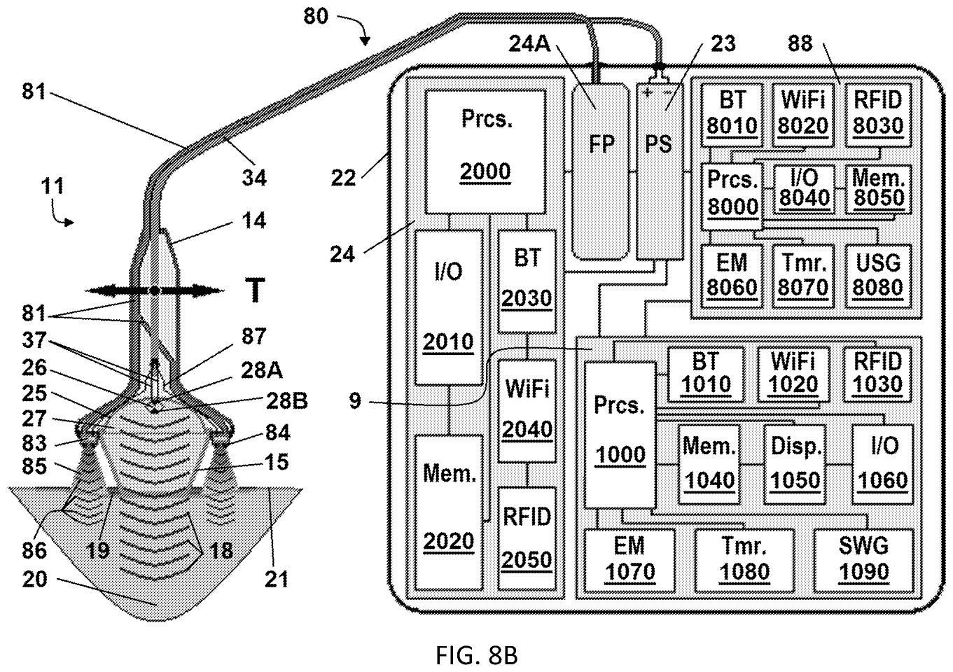

FIGS. 8A and 8B show the combination shockwaves/pressure waves and non-contact ultrasound medical system 80 that is capable in the same device to produce shockwaves/pressure waves in combination with non-contact ultrasound fields, which use a mist solution to transmit the ultrasound towards intended targeted treatment zone/region. The ultrasound waves 86 can be low frequency (20 kHz to 500 kHz), medium (501 kHz to 2 MHz), or high frequency ultrasound (2.1 MHz to 20 MHz). The higher the frequency of the ultrasound the higher is the probability to produce heating effects inside the wound/lesion/tissue condition 19. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous non-contact ultrasound fields and ultrasound waves 86 that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with application of non-contact ultrasound waves 86 from the outside/exterior of the human or animal body (extracorporeally) and towards the targeted treatment zone.

As seen from FIGS. 8A and 8B, the combination shockwaves/pressure waves and non-contact ultrasound medical system 80 includes a combination applicator 11 that uses a combination power cable 34 and the mist solution tubing 81 to connect to the control console/unit 22 shown in FIG. 8B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 8B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more ultrasound piezo ceramic/piezo crystal and mist generator subassemblies 82 are present (see FIG. 8A), which are capable to create ultrasound transmission mist solution field patterns 85 to transmit the ultrasound waves 86 towards the wound/lesion/tissue condition 19. Each of the ultrasound piezo ceramic/piezo crystal and mist generator subassemblies 82 contain an ultrasound piezo ceramic/piezo crystal emission element 83 to produce ultrasound waves 86 and a mist solution nozzle/generator 84 that is used to generate the spraying pattern or ultrasound transmission mist solution field pattern 85. The ultrasound transmission mist solution field pattern 85 is used to conduct the ultrasound waves 86 towards the wound/lesion/tissue condition 19, as seen in FIG. 8B. In FIGS. 8A and 8B there are two (2) ultrasound piezo ceramic/piezo crystal and mist generator subassemblies 82, each of them with a dedicated mist solution tubing 81 and ultrasound piezo ceramic/piezo crystal wiring 87. The combined effects of the shockwaves/pressure waves 18 and of the non-contact ultrasound waves 86 that are transmitted via ultrasound transmission mist solution field pattern 85 will enhance and expedite the healing of the wound/lesion/tissue condition 19, which is beneficial for a successful medical treatment and thus becomes more financially viable and less time consuming for both the patients and the medical personnel. The mist solution that conducts the ultrasound towards the treatment targeted zone can be a saline solution or can incorporate different active fluid substances or specialized fluid medicine to help with the healing of the targeted area.

In FIG. 8B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the ultrasound piezo ceramic/piezo crystal wiring 87 for the ultrasound piezo ceramic/piezo crystal and mist generator subassemblies 82 (see FIG. 8A) that are placed inside or sit on or are attached to the applicator body 14, the ultrasound piezo ceramic/piezo crystal emission element 83 (see FIG. 8B), the mist solution tubing 81, the mist solution nozzle/generator 84, the reflector 25, and the applicator/coupling membrane 15.

The combination power cable 34 provides the connection in between the combination applicator 11 and the control console/unit 22 and it goes inside the applicator body 14, via the shockwave/pressure wave electrodes wiring 37 (reaching the first electrode 28A and the second electrode 28B of the spark-gap 26, where the shockwaves/pressure waves 18 are generated inside the fluid-filled reflector and membrane cavity 27) and also via ultrasound piezo ceramic/piezo crystal wiring 87 until it reaches the ultrasound piezo ceramic/piezo crystal and mist generator subassembly 82 and the ultrasound piezo ceramic/piezo crystal emission element 83, where the ultrasound waves 86 are generated and transmitted via the ultrasound transmission mist solution field pattern 85 towards the wound/lesion/tissue condition 19. The mist solution is produced by the mist solution nozzle/generator 84 that is connected through the mist solution tubing 81 (going alongside the combination power cable 34) to the fluid field generator 24 that is contained inside the control console/unit 22. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, the fluid field generator 24, the fluid pump (FP) 24A, and the ultrasound generator 88 are sharing a common power supply (PS) 23, which can be incorporated inside the control console/unit 22 (as presented in FIG. 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, two or more or multiple and different power supplies can be used for the shockwaves/pressure waves field generator 9, for the fluid field generator 24, for the fluid pump (FP) 24A, and for the ultrasound generator 88, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 8B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B. Also, the fluid field generator 24 directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050. As mentioned before in this invention, the fluid pump processor 2000 has a slave-master relationship with the control console/unit processor 1000, which means that the control console/unit processor 1000 controls the activity of the fluid pump processor 2000 via a continuous interaction process of sharing information and commands. As part of the fluid field generator 24, the fluid pump processor 2000 controls the activities of the fluid pump I/O (input/output) element 2010, the fluid pump memory 2020, the fluid pump Bluetooth module 2030, the fluid pump Wi-Fi module 2040, and the fluid pump RFID module 2050. In general, the purpose, structure, construction, usability, and functionality of these elements of the fluid field generator 24 are similar to the ones presented before for FIGS. 1A and 1B.

On its turn, the ultrasound generator processor 8000 also has a slave-master relationship with the control console/unit processor 1000, which means that the control console/unit processor 1000 controls the activity of the ultrasound generator processor 8000 via a continuous interaction process of sharing information and commands. Thus, the control console/unit processor 1000 dictates the functionality of the entire ultrasound generator 88. Through this process the control console/unit processor 1000 together with the fluid pump processor 2000 and the ultrasound generator processor 8000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution particle size, mist solution or solutions choice, mixing of different mist solutions and their sequence of pumping, and the ultrasound transmission mist solution field pattern 85.

The control console/unit processor 1000 practically controls the whole activity of any combination energy medical systems, including the combination shockwaves/pressure waves and non-contact ultrasound medical system 80 presented in FIGS. 8A and 8B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the ultrasound waves 86 that are delivered via ultrasound transmission mist solution field pattern 85 towards the wound/lesion/tissue condition 19. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution particle size, mist solution or solutions choice, mixing of different mist solutions and their sequence of pumping, ultrasound transmission mist solution field pattern 85, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the ultrasound generator 88 includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080.

In another embodiment, the ultrasound generator 88 contains only the ultrasound generator processor 8000. Additionally, in some embodiments depending on the capabilities of the ultrasound generator 88, fluid field generator 24 and control console/unit 22, the ultrasound generator 88 may also include the ultrasound generator Bluetooth (BT) module 8010, the ultrasound generator Wi-Fi module 8020, and the ultrasound generator RFID module 8030 to ensure a proper and rapid communication with the control console/unit 22 and fluid field generator 24. The ultrasound generator processor 8000 must work in tandem with the fluid pump processor 2000 and under the master control of the control console/unit processor 1000, to be able to properly synchronize the generation of mist solution with ultrasound waves 86 and formation of ultrasound transmission mist solution field pattern 85 towards the wound/lesion/tissue condition 19 and coordinate with the shockwaves/pressure waves 18 activation and propagation in the same wound/lesion/tissue condition 19. That means that for the good functionality of the combination shockwaves/pressure waves and non-contact ultrasound medical system 80 presented in FIGS. 8A and 8B, the fluid field generator 24 and its associated fluid pump (FP) 24A should function in total synchronicity with the ultrasound generator 88 to produce the ultrasound waves 86 and generate the mist solution in the same time that is the medium of transmission for the ultrasound waves 86 towards the wound/lesion/tissue condition 19 via mist solution field patterns 85. If the synchronicity is not there, then the ultrasound waves 86 will not be able to reach their target/wound/lesion/tissue condition 19 for the combination shockwaves/pressure waves and non-contact ultrasound medical system 80 presented in FIGS. 8A and 8B. In one embodiment, only one of these communication modules of the ultrasound generator 88 may be present, in another embodiment two of them, and in yet another embodiment all three of these communication modules may be present. Furthermore, in another embodiment, either the ultrasound generator I/O (input/output) element 8040 or ultrasound generator memory 8050 can be present or none of them are present in the construction of the ultrasound generator 88. The advantage of having the ultrasound generator I/O (input/output) element 8040 is that it offers the possibility to independently input or output data for the functionality of the ultrasound generator 88. Regarding the ultrasound generator memory 8050, this module allows to store the functionality of the ultrasound generator 88 separately from the control console/unit memory 1040 of the shockwaves/pressure waves field generator 9 or the fluid pump memory 2020 of the fluid field generator 24. The ultrasound generator 88 and its ultrasound generator processor 8000 control the ultrasound type (low, medium, or high frequency), mist and ultrasound time of activation, continuous or intermittent application of mist and ultrasound, ultrasound intensity/amplitude, ultrasound wavelength, mist pressure, mist particle size, mist solution or solutions choice, mixing of different mist solutions and their sequence of pumping, and the ultrasound transmission mist solution field pattern 85. For that, the ultrasound generator processor 8000 is using the ultrasound generator emission mechanism (EM) module 8060, the ultrasound generator timer module 8070, and the ultrasound field generator (USG) module 8080. The ultrasound field generator (USG) module 8080 may include hardware or components, which are well-known to those skilled in the art, for providing via the combination applicator 11 the proper ultrasound waves 86 towards the wound/lesion/tissue condition 19 using the associated ultrasound transmission mist solution field patterns 85. The ultrasound generator timer module 8070 provides timing sequence for emitting the one or more generated ultrasound waves 86 through mist solution and via ultrasound transmission mist solution field patterns 85 towards or into the wound/lesion/tissue condition 19, as dictated by the one or more energy combination treatment settings. The one or more ultrasound waves 86 are emitted in a manner controlled by the ultrasound generator emission mechanism (EM) module 8060 that transmits the emission commands, via a secured data transfer protocol, as mentioned before, in the form of electrical signals from the control console/unit 22 through the combination power cable 34 towards the combination applicator 11. The interaction of the shockwaves/pressure waves field generator 9, the fluid field generator 24, and ultrasound generator 88 is controlled and overseen by the control console/unit processor 1000, which works with the ultrasound generator processor 8000 and the fluid pump processor 2000 to be able to properly synchronize the generation of mist solution with ultrasound waves 86 and formation of ultrasound transmission mist solution field pattern 85 towards the wound/lesion/tissue condition 19 and finally coordinate with the shockwaves/pressure waves 18 activation and propagation in the same wound/lesion/tissue condition 19.

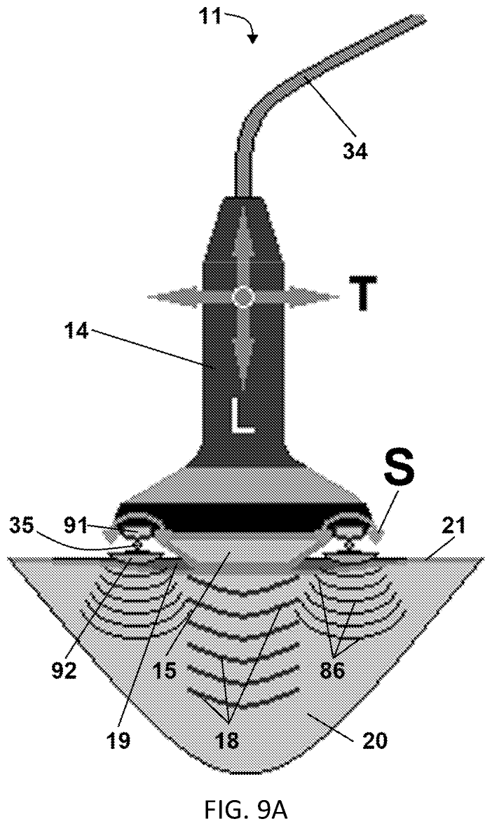

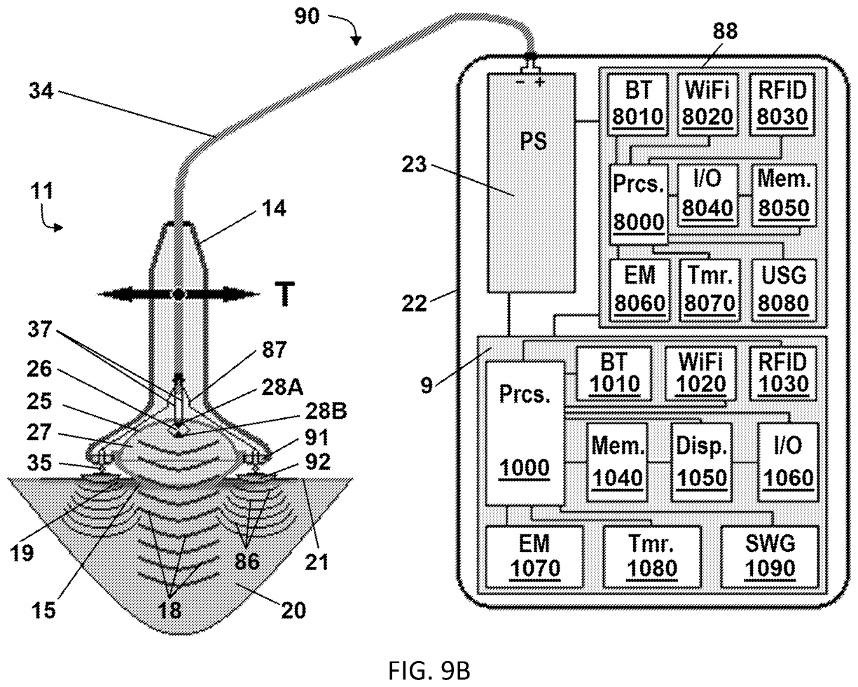

FIGS. 9A and 9B show the combination shockwaves/pressure waves and contact ultrasound medical system 90 that is capable in the same device to produce shockwaves/pressure waves in combination with contact ultrasound. The ultrasound waves 86 can be low frequency (20 kHz to 500 kHz), medium (501 kHz to 2 MHz), or high frequency ultrasound (2.1 MHz to 20 MHz). The higher the frequency of the ultrasound the higher is the probability to produce heating effects inside the wound/lesion/tissue condition 19. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous contact ultrasound waves 86 that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with contact ultrasound from the outside/exterior of the human or animal body (extracorporeally) and towards the targeted treatment zone.

As seen from FIGS. 9A and 9B, the combination shockwaves/pressure waves and contact ultrasound medical system 90 includes a combination applicator 11 that uses a combination power cable 34 to connect to the control console/unit 22 shown in FIG. 9B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 9B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more piezo ceramic/piezo crystal electric connection elements 91 are present that are connected via the swiveling hinge 35 to the ultrasound hinged piezo ceramic/piezo crystal emission elements 92, which are capable to create ultrasound waves 86 inside the wound/lesion/tissue condition 19. In FIGS. 9A and 9B there are two (2) piezo ceramic/piezo crystal electric connection elements 91, each of them with a dedicated ultrasound piezo ceramic/piezo crystal wiring 87. The swiveling movement S allows the correct position of the ultrasound hinged piezo ceramic/piezo crystal emission elements 92 and constant contact with the wound/lesion/tissue condition 19 or the skin 21. The combined effects of the shockwaves/pressure waves 18 and of the ultrasound waves 86 will enhance and expedite the healing of the wound/lesion/tissue condition 19, which is beneficial for a successful medical treatment and thus becomes more financially viable and less time consuming for both the patients and the medical personnel.

In FIG. 9B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the reflector 25, the applicator/coupling membrane 15, the ultrasound piezo ceramic/piezo crystal wiring 87 for the piezo ceramic/piezo crystal electric connection elements 91, the swiveling hinges 35, and the ultrasound hinged piezo ceramic/piezo crystal emission elements 92, which are placed inside or sit on or are attached to the applicator body 14.

The combination power cable 34 provides the connection in between the combination applicator 11 and the control console/unit 22 and it goes inside the applicator body 14, via the shockwave/pressure wave electrodes wiring 37, until it reaches the first electrode 28A and the second electrode 28B of the spark-gap 26, where the shockwaves/pressure waves 18 are generated inside the fluid-filled reflector and membrane cavity 27. Inside the applicator body 14, the combination power cable 34 branches into shockwave/pressure wave electrodes wiring 37 and also into the ultrasound piezo ceramic/piezo crystal wiring 87 for the piezo ceramic/piezo crystal electric connection element 91 to provide energy to the ultrasound hinged piezo ceramic/piezo crystal emission element 92. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, and the ultrasound generator 88 are sharing a common power supply (PS) 23, which can be incorporated inside the control console/unit 22 (as presented in FIG. 2B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and for the ultrasound generator 88, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 9B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The ultrasound generator processor 8000 also has a slave-master relationship with the control console/unit processor 1000, which means that the control console/unit processor 1000 controls the activity of the ultrasound generator processor 8000 via a continuous interaction process of sharing information and commands. Thus, the control console/unit processor 1000 dictates the functionality of the entire ultrasound generator 88. Through this process the control console/unit processor 1000 and the ultrasound generator processor 8000 control the ultrasound type (low, medium, or high frequency), ultrasound time of activation, continuous or intermittent application of ultrasound, ultrasound intensity/amplitude, and ultrasound wavelength.

The control console/unit processor 1000 practically controls the whole activity of any combination energy medical systems, including the combination shockwaves/pressure waves and contact ultrasound medical system 90 presented in FIGS. 9A and 9B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the ultrasound waves 86 that are delivered to the wound/lesion/tissue condition 19. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, ultrasound type (low, medium, or high frequency), ultrasound time of activation, continuous or intermittent application of ultrasound, ultrasound intensity/amplitude, ultrasound wavelength, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the ultrasound generator 88 includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080. The purpose, structure, construction, usability and functionality of these elements of the ultrasound generator 88 are similar to what was presented before for FIGS. 8A and 8B, for the ultrasound waves 86 generation and control. In the embodiment from FIGS. 9A and 9B, there is no mist solution involved and the ultrasound hinged piezo ceramic/piezo crystal emission elements 92 are in direct contact with the wound/lesion/tissue condition 19 or the skin 21 or a specially designed coupling bladder or spacer, which simplifies the generation and controlling of the ultrasound waves 86 when compared to the system/embodiment presented in FIGS. 8A and 8B.

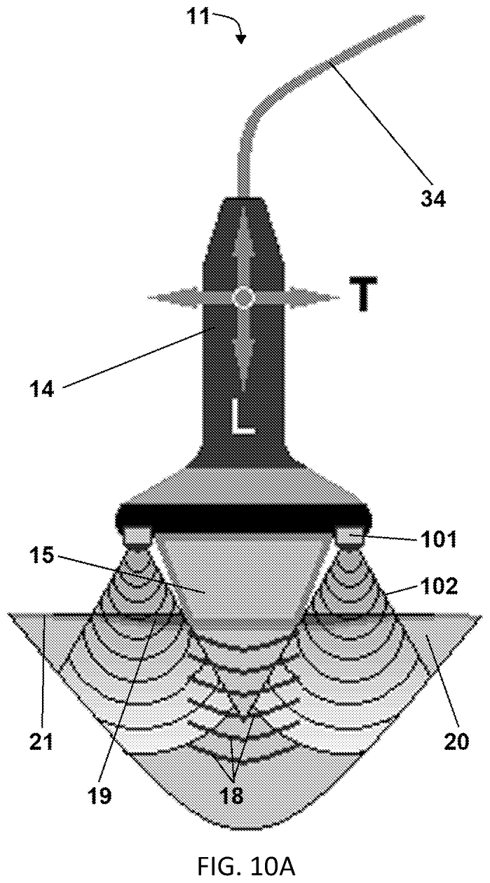

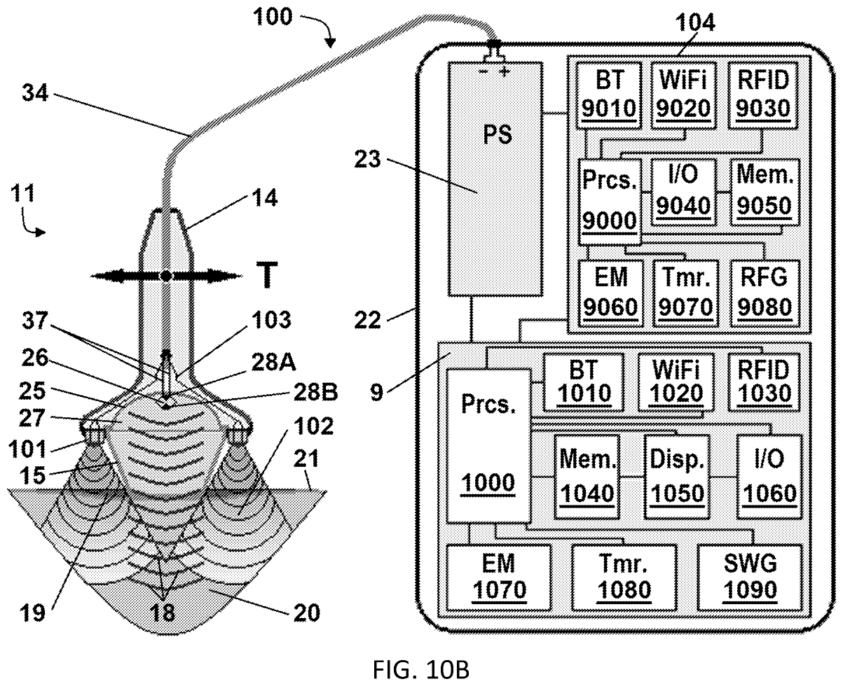

FIGS. 10A and 10B show the combination shockwaves/pressure waves and radio-frequency fields medical system 100 that is capable in the same device to produce shockwaves/pressure waves in combination with radio-frequency fields. These specially designed applicators and medical systems that use combination energy treatment regimens with energy-based medical technologies or combination devices/systems, which can deliver simultaneously multiple energy-based technologies, incorporate besides shockwaves/pressure waves generating design elements (shockwave/pressure wave generators, reflectors, membranes, etc.) also some specific components able to produce pulsed or continuous radio-frequency fields that are delivered simultaneously with the shockwaves/pressure waves 18 for stimulation, healing, and regeneration of cells, tissues, or organs. In this case, the combination applicators 11 will have a special design that can facilitate a medical treatment using the shockwaves/pressure waves 18 in combination with radio frequency fields from the outside/exterior of the human or animal body (extracorporeally) and towards the targeted treatment zone.

As seen from FIGS. 10A and 10B, the combination shockwaves/pressure waves and radio-frequency fields medical system 100 includes a combination applicator 11 that uses a combination power cable 34 to connect to the control console/unit 22 shown in FIG. 10B. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 that actuates an external motorized ancillary system (not specifically presented in any of the figures of this patent for simplicity) that also contains sensors, motors, cameras, and clamping mechanism for the combination applicator 11. The applicator/coupling membrane 15 sits on top of the reflector 25 (see FIG. 2B) that can have different geometries as ellipsoidal, parabolic, spherical, conical, pyramidal, or any combination of them, or in general any concave geometry. The applicator/coupling membrane 15 together with the reflector 25 form the fluid-filled reflector and membrane cavity 27, where the shockwaves/pressure waves 18 are generated via different principles using electrohydraulic generators using spark gap high voltage discharges, or electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil. The shockwaves/pressure waves 18 are transmitted inside the human/animal body 20 via applicator/coupling membrane 15 that is in direct contact with skin 21 or wound/lesion/tissue condition 19. In other embodiments, the applicator/coupling membrane 15 is not in direct contact with the skin 21 or wound/lesion/tissue condition 19, but rather in direct contact with a specially designed coupling bladder or spacer (not shown in the figures of this invention) that contains a fluid or a gel, to facilitate good propagation of the shockwaves/pressure waves 18 inside the human/animal body 20, with the specially designed coupling bladder or spacer sitting on top of the skin 21 or wound/lesion/tissue condition 19 and being in direct contact with the skin 21 or wound/lesion/tissue condition 19. The shockwaves/pressure waves 18 are passing through the wound/lesion/tissue condition 19 that can be positioned superficial, or immediately under the skin 21, or deep inside the human/animal body 20. On the peripheral part of the bulbous region of the applicator body 14 and next to the applicator/coupling membrane 15, at least two or three or four or more radio-frequency emission elements 101 are present, which are capable to create radio-frequency field patterns 102 inside the wound/lesion/tissue condition 19. In FIGS. 10A and 10B there are two (2) radio-frequency emission elements 101, each of them with a dedicated radio-frequency emission element wiring 103. The radio-frequency emission elements 101 can be at a certain distance from the skin 21 or wound/lesion/tissue condition 19 (as seen in FIGS. 10A and 10B) or from a specially designed coupling bladder or spacer (mentioned before in this paragraph) or in another embodiment the radio-frequency emission elements 101 can be modified to be positioned in direct contact with the skin 21 or wound/lesion/tissue condition 19 or with a specially designed coupling bladder or spacer. The combined effects of the shockwaves/pressure waves 18 and of the radio-frequency field patterns 102 will enhance and expedite the healing of the wound/lesion/tissue condition 19, which is beneficial for a successful medical treatment and thus becomes more financially viable and less time consuming for both the patients and the medical personnel.

In FIG. 10B, the focus is on the configuration of the control console/unit 22 and the internal structure of the combination applicator 11. The combination applicator 11 internal structure contains different elements as the combination power cable 34, the shockwave/pressure wave electrodes wiring 37 for the first electrode 28A and the second electrode 28B of the spark-gap 26, the radio-frequency emission element wiring 103 for the radio-frequency emission elements 101 (which are placed inside or sit on or are attached to the applicator body 14), the reflector 25, and the applicator/coupling membrane 15.

The combination power cable 34 provides the connection in between the combination applicator 11 and the control console/unit 22 and it goes inside the applicator body 14 until it reaches the first electrode 28A and the second electrode 28B of the spark-gap 26, where the shockwaves/pressure waves 18 are generated inside the fluid-filled reflector and membrane cavity 27. The high voltage that travels through combination power cable 34 is produced inside the control console/unit 22 by the power supply (PS) 23 that is controlled and directly connected to the shockwaves/pressure waves field generator 9. It is also important to note that the combination applicator 11, the control console/unit 22, the shockwaves/pressure waves field generator 9, and the radio-frequency generator 104 are sharing a common power supply (PS) 23, which can be incorporated inside the control console/unit 22 (as presented in FIG. 10B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the shockwaves/pressure waves field generator 9 and the radio-frequency generator 104, power supplies that can be internal to the control console/unit 22 or in other embodiment can be external to the control console/unit 22 as part of an independent module/ancillary system.

As presented before for FIGS. 1A and 1B, the shockwaves/pressure waves field generator 9 from FIG. 10B includes the control console/unit processor 1000, the control console/unit Bluetooth (BT) module 1010, the control console/unit Wi-Fi module 1020, the control console/unit RFID module 1030, the control console/unit memory 1040, the control console/unit display module 1050, the control console/unit I/O (input/output) element 1060, the control console/unit emission mechanism (EM) module 1070, the control console/unit timer module 1080, and the control console/unit shockwave or pressure wave generator (SWG) module 1090. The purpose, structure, construction, usability, and functionality of these elements of the shockwaves/pressure waves field generator 9 are similar to what was presented before for FIGS. 1A and 1B.

The radio-frequency field generator processor 9000 has a slave-master relationship with the control console/unit processor 1000, which means that the control console/unit processor 1000 controls the activity of the radio-frequency field generator processor 9000 via a continuous interaction process of sharing information and commands. Thus, the control console/unit processor 1000 dictates the functionality of the entire radio-frequency generator 104. Through this process the control console/unit processor 1000 together with the radio-frequency field generator processor 9000 control the radio-frequency type (non-thermal radio frequency or thermal radio-frequency), time of activation, intensity, frequency, continuous or intermittent application, and the radio-frequency field patterns 102.

The control console/unit processor 1000 practically controls the whole activity of any combination energy medical systems, including the combination shockwaves/pressure waves and radio-frequency fields medical system 100 presented in FIGS. 10A and 10B. Thus, the combination applicator 11 receives the system control information from the control console/unit processor 1000, via a secured data transfer protocol from the control console/unit 22, for generating a selected/optimized number of combination energy medical treatments, by utilizing a selected/optimized amount of energy, as determined by the one or more combination treatment settings for the shockwaves/pressure waves 18 and the radio-frequency field patterns 102. The data transfer protocol in between the control console/unit 22 and combination applicator 11 is secured via an authentication process that includes processor generated passcodes and passwords, authentication process that is performed when the combination power cable 34 is connected to the control console/unit 22 and the power is “On” for the control console/unit 22. The control console/unit processor 1000 will also monitor the treatment parameters for their consistency and repeatability during the actual treatment delivery. The final number of energy combination energy medical treatments, shockwaves/pressure waves input energy, frequency of shockwaves/pressure waves per second, radio-frequency type (non-thermal radio frequency or thermal radio-frequency), time of activation, intensity, frequency, continuous or intermittent application, radio-frequency field patterns 102, type and serial number of combination applicator 11, type and serial number of the control console/unit 22, date, timing, financial info about the treatment for reimbursement, name of facility performing the treatment, region where the treatment was performed, etc. are all processed by the control console/unit processor 1000 and then recorded in the control console/unit memory 1040, at the end of the treatment. The control console/unit 22 can also use its control console/unit processor 1000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In other embodiment, the treatment data and parameters monitored by the control console/unit processor 1000 and recorded in the control console/unit memory 1040 can be sent directly from the control console/unit 22 to the internet-based data storage/cloud database and its associated artificial intelligence (AI) server, via the control console/unit Bluetooth (BT) module 1010, or control console/unit Wi-Fi module 1020, or the control console/unit RFID module 1030. The functions of the artificial intelligence (AI) device are the same as the ones presented for FIGS. 1A and 1B.

Also, the radio-frequency generator 104 includes the radio-frequency field generator processor 9000, the radio-frequency field generator Bluetooth (BT) module 9010, the radio-frequency field generator Wi-Fi module 9020, the radio-frequency field generator RFID module 9030, the radio-frequency field generator I/O (input/output) element 9040, the radio-frequency field generator memory 9050, the radio-frequency field generator emission mechanism (EM) module 9060, the radio-frequency field generator timer module 9070, and the radio-frequency field generator (RFG) module 9080.

In another embodiment, the radio-frequency generator 104 contains only the radio-frequency field generator processor 9000. Additionally, in some embodiments depending on the capabilities of the radio-frequency generator 104 and control console/unit 22, the radio-frequency generator 104 may also include the radio-frequency field generator Bluetooth (BT) module 9010, the radio-frequency field generator Wi-Fi module 9020, and the radio-frequency field generator RFID module 9030 to ensure a proper and rapid communication with the control console/unit 22. In one embodiment, only one of these communication modules may be present, in another embodiment two of them, and in yet another embodiment all three of these communication modules may be present. Furthermore, in another embodiment, either the radio-frequency field generator I/O (input/output) element 9040 or the radio-frequency field generator memory 9050 can be present or none of them are present in the construction of the radio-frequency generator 104. The advantage of having the radio-frequency field generator I/O (input/output) element 9040 is that it offers the possibility to independently input or output data for the functionality of the radio-frequency generator 104. Regarding the radio-frequency field generator memory 9050, this module allows to store the functionality of the radio-frequency generator 104 separately from the control console/unit memory 1040 of the shockwaves/pressure waves field generator 9. The radio-frequency generator 104 and its radio-frequency field generator processor 9000 control the radio-frequency type (non-thermal radio frequency or thermal radio-frequency), time of activation, intensity, frequency, continuous or intermittent application, and the radio-frequency field patterns 102. For that, the radio-frequency field generator processor 9000 is using the radio-frequency field generator emission mechanism (EM) module 9060, the radio-frequency field generator timer module 9070, and the radio-frequency field generator (RFG) module 9080. The radio-frequency field generator (RFG) module 9080 may include hardware or components, which are well-known to those skilled in the art, for providing via the combination applicator 11 the proper radio-waves and the associated radio-frequency field pattern 102. The radio-frequency field generator timer module 9070 provides timing sequence for emitting the one or more generated radio-frequency field pattern 102 into the wound/lesion/tissue condition 19, as dictated by the one or more energy combination treatment settings. The one or more radio-frequency field pattern 102 are emitted in a manner controlled by the radio-frequency field generator emission mechanism (EM) module 9060 and transmitted from the control console/unit 22 through the combination power cable 34 towards the combination applicator 11, via a secured data transfer protocol, as mentioned before.

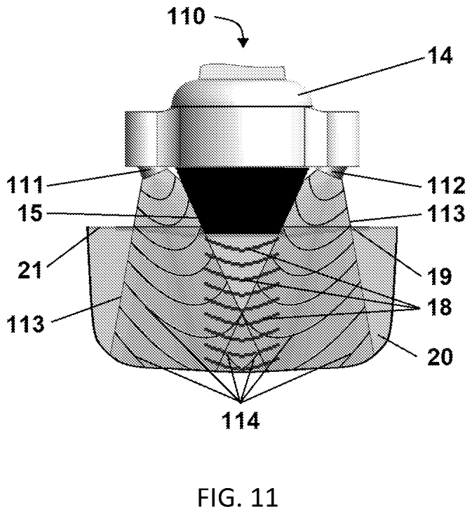

In the embodiments from FIGS. 1A-10B the energy-based medical technologies or combination devices/systems do not necessarily overlap their created shockwave/pressure waves or electric or electromagnetic or LED/OLED phototherapy or laser or ultrasound (contact or non-contact) or UV light or radio-frequency created fields in the wound/lesion/tissue condition 19 for a fixed position of the combination applicator 11. The overlap of effects from the combination energy medical technologies is realized by using the longitudinal movement L and transversal movement T of the combination applicator 11, and especially by using the transversal movement T along and across the wound/lesion/tissue condition 19, where the L and T movements can be done manually or automatically. In some situations, an overlap of the energy fields coming from different technologies is needed in one fixed position of the combination applicator 11, which enhance their effects on the wound/lesion/tissue condition 19. In FIG. 11 is presented an embodiment of a combination shockwaves/pressure waves and overlapping radio-frequency fields medical system 110 in one position of the combination applicator 11. The control console/unit 22 is not depicted in FIG. 11 for simplicity, since the control console/unit 22 has the same functionality and structure as the one presented in FIG. 10B. The combination shockwaves/pressure waves and overlapping radio-frequency fields medical system 110 is using a first angled radio-frequency emission element 111 and a second angled radio-frequency emission element 112 that are positioned on the peripheral part of the bulbous region of the applicator body 14 and next to the applicator/coupling membrane 15. As seen from FIG. 11, the angled orientation of the radio-frequency emission elements allows the orientation of the angled radio-frequency field patterns 113 in such way that creates the radio frequency fields 114 that overlap with the shockwaves/pressure waves 18 under the skin 21 and inside the wound/lesion/tissue condition 19 from the human/animal body 20. In FIG. 11 there are only two (2) angled radio-frequency emission elements 111 and 112, however at least two or three or four or more angled radio-frequency emission elements can be used, depending of the needs and targeted medical application of the combination shockwaves/pressure waves and overlapping radio-frequency fields medical system 110. The first angled radio-frequency emission element 111 and the second angled radio-frequency emission element 112 can have a hinge that allows the adjustment of their angled orientation relatively to the targeted treatment zone. The other elements and components associated with the embodiment from FIG. 11 that are related to the construction of the combination applicator 11 and the control console/unit 22 are the same in purpose, structure, construction, usability and functionality with the elements presented before for the embodiments from FIGS. 10A and 10B.

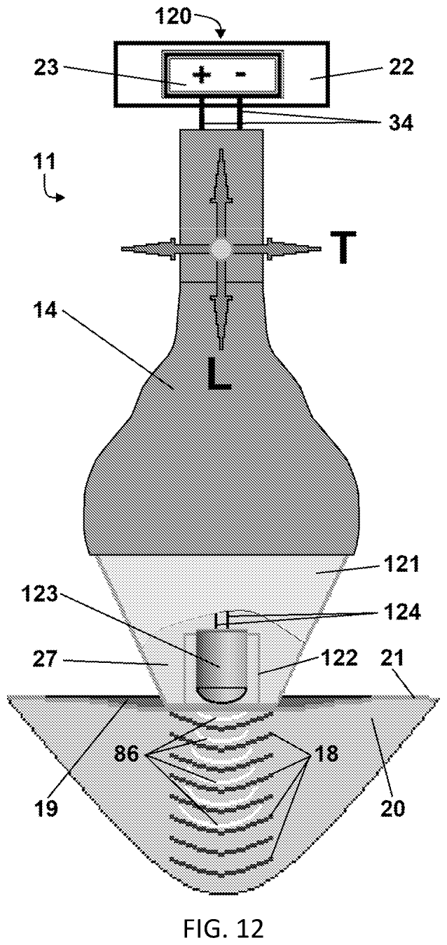

There are other ways to achieve an overlap of the energy fields coming from different technologies in one fixed position of the combination applicator 11. Thus, in FIG. 12 is presented an embodiment of a combination shockwaves/pressure waves and overlapping contact ultrasound medical system 120, where the membrane-integrated piezo ceramic/piezo crystal 123 is coaxial with the shockwaves/pressure waves 18 and their direction of propagation. For that, a specially designed central cylindrical dimple applicator/coupling membrane 121 is used, membrane that has a cylindrical dimple 122 at the contact with the wound/lesion/tissue condition 19 and which is symmetrically orientated around the longitudinal axis of the combination applicator 11. This design of the central cylindrical dimple applicator/coupling membrane 121 can accommodate membrane-integrated piezo ceramic/piezo crystal 123 that produces the direct contact ultrasound waves 86, which propagate in the same region of the wound/lesion/tissue condition 19 as the shockwaves/pressure waves 18 that produces their complete overlap, with increased benefits for treatment and healing of the wound/lesion/tissue condition 19 from the human/animal body 20. The combination applicator 11 is capable of producing simultaneously or sometimes subsequently the delivery of the coaxially shockwaves/pressure waves 18 and the ultrasound waves 86 into the wound/lesion/tissue condition 19, which is controlled by the control console/unit 22, in accordance of the user settings and preferences. The membrane-integrated piezo ceramic/piezo crystal 123 has its own piezo ceramic/piezo crystal wiring 124 that is connected inside the applicator body 14 to the combination power cable 34 and ultimately to the power supply (PS) 23 of the control console/unit 22. Of course, the combination power cable 34 is also connected to the first electrode 28A and the second electrode 28B of the spark-gap 26, via the shockwave/pressure wave electrodes wiring 37 (as seen in FIGS. 1A-10B) to produce the shockwaves/pressure waves 18 in the fluid-filled reflector and membrane cavity 27, and transmit the shockwaves/pressure waves 18 through the skin 21 and/or wound/lesion/tissue condition 19, as seen before in FIGS. 9A and 9B. Besides electrohydraulic generators using spark gap high voltage discharge in between the first electrode 28A and the second electrode 28B of the spark-gap 26, the shockwaves/pressure waves 18 can be also produced via electrohydraulic generators using one or multiple laser sources, or piezoelectric generators using piezo crystals/piezo ceramics, or piezoelectric generators using piezo fibers, or electromagnetic generators using a flat coil and an acoustic lens, or electromagnetic generators using a cylindrical coil, which will modify accordingly the construction of the combination applicator 11 with elements that were presented in detail in other inventions. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen in FIGS. 9A-9B) that actuates an external motorized ancillary system (not specifically presented in any of the figures of this patent for simplicity) that also contains sensors, motors, cameras, and clamping mechanism for the combination applicator 11. The other elements and components associated with the embodiment from FIG. 12 that are related to the construction of the combination applicator 11 and the control console/unit 22 are the same in purpose, structure, construction, usability and functionality with the elements presented before for the embodiments from FIGS. 9A and 9B.

For FIG. 11, the combination applicators 11 will have a special design that can facilitate a medical treatment using the shockwaves/pressure waves 18 in combination with radio frequency fields from the outside/exterior of the human or animal body (extracorporeally) and towards the targeted treatment zone. Likewise for FIG. 12, the combination applicators 11 will have a special design that can facilitate a medical treatment using the shockwaves/pressure waves 18 in combination with contact ultrasound from the outside/exterior of the human or animal body (extracorporeally) and towards the targeted treatment zone.

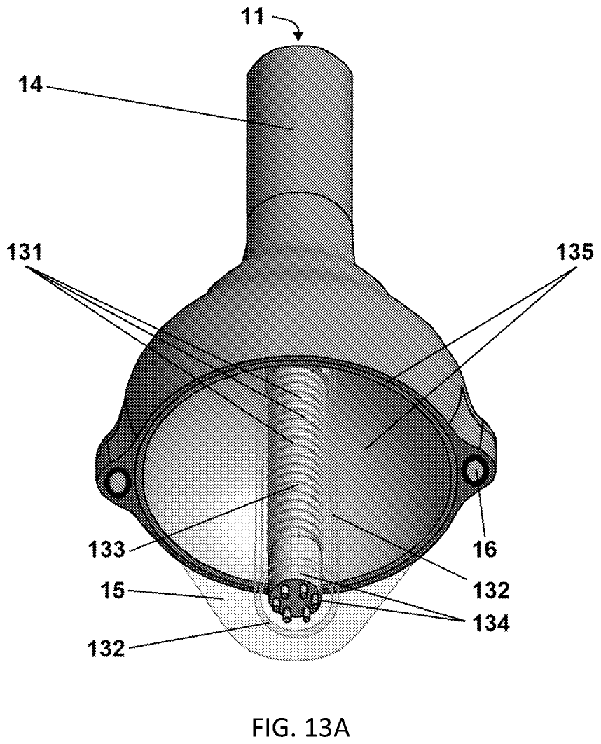

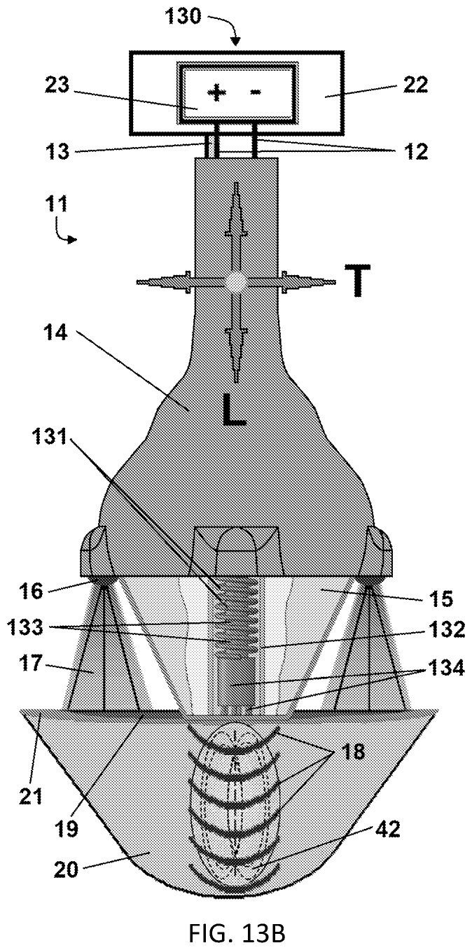

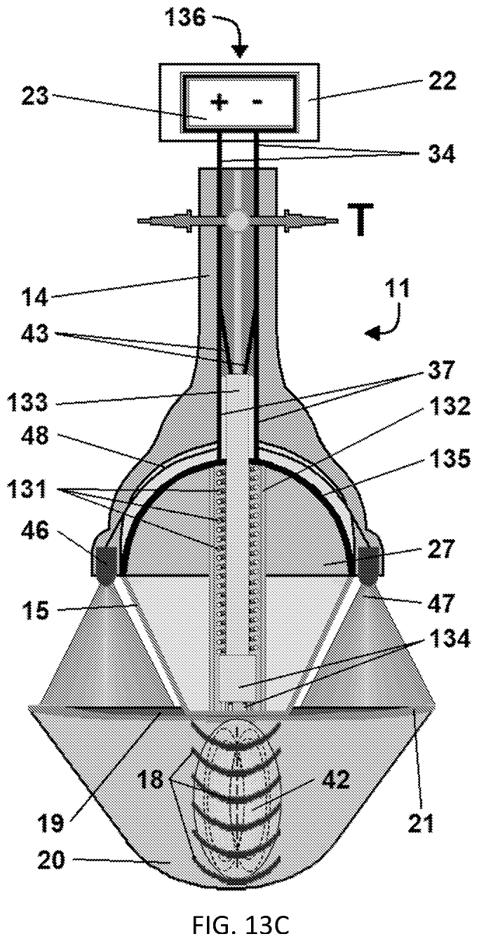

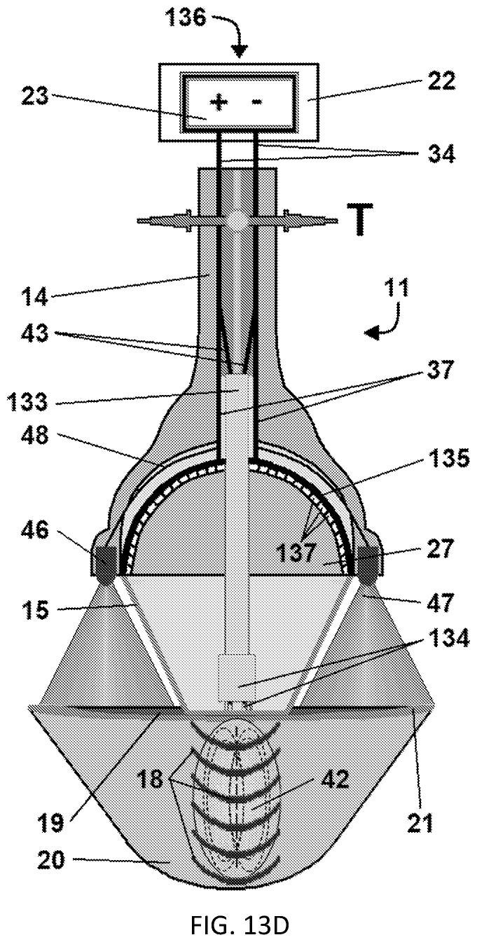

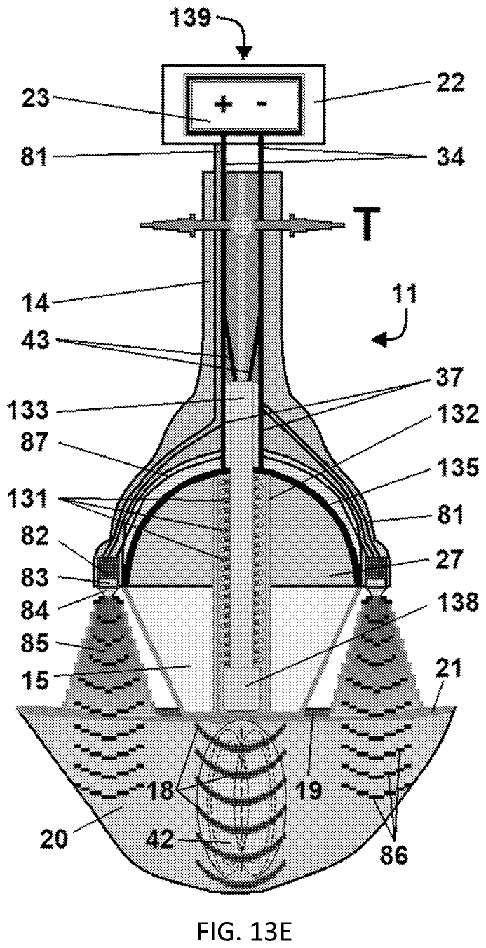

Furthermore, more than two energy technologies can be packages in the combination applicator 11 that enhances even more the efficacy of such systems when treating the wound/lesion/tissue condition 19 from the human/animal body 20, which is presented in FIGS. 13A-13E. Thus, FIGS. 13A and 13B show an embodiment of a combination energy system that simultaneously delivers shockwaves, a coaxial electromagnetic field and laterally from the bulbous part of the combination applicator 11 one or two different active substances from two (as seen in FIGS. 13A and 13B) or three or four or more spraying nozzle 16. The combination shockwaves/pressure waves, electromagnetic field, and active substance medical system 130 is using a combination applicator 11 that uses an applicator/coupling membrane 15 that together with the parabolic reflector 135 forms fluid-filled reflector and membrane cavity 27, which is an enclosed space filled with fluid. Inside this space there is a central cylindrical electromagnetic coil 131 that wraps around a central magnetic core 133, both of them being encased in a fluid-filled cylindrical encasing tube 132 to be able to propagate any waves produced by the central cylindrical electromagnetic coil 131 without any loses inside the parabolic reflector 135 and towards the parabolic reflector focal point, which is positioned outside the parabolic reflector 135 and inside the targeted wound/lesion/tissue condition 19. In another embodiment, the parabolic reflector 135 has its focal point inside the reflector, where the central cylindrical electromagnetic coil 131 is present, which creates pseudo-planar shockwaves through the wound/lesion/tissue condition 19. When a short electrical pulse provided by the power supply (PS) 23 (included in control console/unit 22) is transmitted via the high voltage cable 12 to the central cylindrical electromagnetic coil 131 it produces its excitation that is transmitted to the fluid-filled cylindrical encasing tube 132, which will results in a cylindrical wave (not shown in FIGS. 13A-13E) that can be focused by the parabolic reflector 135 towards the targeted wound/lesion/tissue condition 19 from the human/animal body 20, when the focal point of the parabolic reflector 135 is outside of reflector and positioned inside or overlapped with the wound/lesion/tissue condition 19. Conversely, when the focal point of the parabolic reflector 135 is inside the reflector, the pseudo planar shockwaves or pressure waves can be created and directed towards the wound/lesion/tissue condition 19. At its turn, the central magnetic core 133 has also the frontal electromagnetic antenna with props 134 that are used to produce coaxial electromagnetic field pattern 42 that overlaps with the field produced by the shockwaves/pressure waves 18 in the wound/lesion/tissue condition 19 and also deep in the human/animal body 20. Furthermore, using the spraying nozzles 16, one or more active substances are delivered superficially to the skin 21 or wound/lesion/tissue condition 19 via the active substance spraying patterns 17. In FIGS. 13A and 13B there are only two (2) spraying nozzles 16, however at least two or three or four or more spraying nozzles 16 can be used, depending of the needs and targeted medical application of the combination shockwaves/pressure waves, electromagnetic field, and active substance medical system 130. The active substance is brought to the spraying nozzle 16 using active substance tubing 13 (as independent elements or welded together to the high voltage cable 12 in a multi-capability cable). The active substance or multiple substances can be any of those presented for FIGS. 1A and 1B and their action on the wound/lesion/tissue condition 19 similar to those presented for the embodiments from FIGS. 1A and 1B. The combination applicator 11 is capable of producing simultaneously, intermittent, or sometimes subsequently the coaxially shockwaves/pressure waves 18, the electromagnetic field pattern 42, or the substance spraying patterns 17 at the surface or inside the wound/lesion/tissue condition 19. The activation of different technologies used by the combination shockwaves/pressure waves, electromagnetic field, and active substance medical system 130 can be done simultaneously or intermittent or subsequently and it is controlled by the control console/unit 22, in accordance of the user settings and preferences. The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen in FIGS. 1A, 1B, 3A, and 3B) that actuates an external motorized ancillary system (not specifically presented in any of the figures of this patent for simplicity) that also contains sensors, motors, cameras, and clamping mechanism for the combination applicator 11. For FIGS. 13A and 13B, the combination applicators 11 will have a special design that can facilitate a medical treatment using the shockwaves/pressure waves 18 in combination with electromagnetic fields and active medical substances (sprayed on the wound/lesion/tissue condition 19) from the outside/exterior of the human or animal body (extracorporeally) and towards the targeted treatment zone. The other elements and components associated with the embodiment from FIGS. 13A and 13B that are related to the construction of the combination applicator 11 and the control console/unit 22 are the same in purpose, structure, construction, usability and functionality with the elements presented before for the embodiments from FIGS. 1A, 1B, 3A, and 3B.

FIGS. 13C and 13D show embodiments of a combination shockwaves/pressure waves, electromagnetic field, and phototherapy medical system 136, which incorporates means to produce shockwaves/pressure waves 18, the electromagnetic field pattern 42, and the LED/OLED phototherapy field patterns 47. The central cylindrical electromagnetic coil 131 and the fluid-filled cylindrical encasing tube 132 (only for FIG. 13C) have the same roles and functionality in producing shockwaves/pressure waves 18 via the electromagnetic principle as presented in FIGS. 13A and 13B. Similarly, in FIGS. 13C and 13D the central magnetic core 133 and the frontal electromagnetic antenna with props 134 have the same roles and functionality in producing electromagnetic field pattern 42, as presented for FIGS. 13A and 13B. Also, the difference in between FIG. 13C and FIG. 13D is that the shockwaves/pressure waves 18 are produced via electromagnetic principle in FIG. 13C, which is similar to what was presented for the embodiment from FIGS. 13A and 13B and the shockwaves/pressure waves 18 from FIG. 13D are produced via the piezoelectric principle. Thus, in FIG. 13D, the central cylindrical electromagnetic coil 131 and the fluid-filled cylindrical encasing tube 132 are eliminated and the piezo ceramic/piezo crystal or piezo fibers elements 137 sitting on the parabolic reflector 135 are used to generate the shockwaves/pressure waves 18. In this case, the internal mechanical strain resulting from an applied electrical field to the piezo ceramic/piezo crystal or piezo fibers elements 137, which are uniformly placed on the parabolic reflector 135, generate the shockwaves/pressure waves 18, which are then focused by the parabolic reflector 135 (that has its focal point outside the reflector) towards the wound/lesion/tissue condition 19 from the human/animal body 20. The central magnetic core 133 and the frontal electromagnetic antenna with props 134 are electrically connected to the combination power cable 34 via the electromagnetic emission element wiring 43. In this way the frontal electromagnetic antenna with props 134 is capable to generate the electromagnetic field pattern 42 that overlaps with the shockwaves/pressure waves 18. The electrical current for the piezo ceramic/piezo crystal or piezo fibers elements 137, for the central magnetic core 133, and for the frontal electromagnetic antenna with props 134 is provided by the power supply (PS) 23, included in the control console/unit 22, via the combination power cable 34.

In FIGS. 13C and 13D on the peripheral part of the bulbous region of the applicator body 14 and next to the applicator/coupling membrane 15, at least two or three or four or more LEDs/OLEDs 46 are present, which are capable to create LED/OLED phototherapy field patterns 47 on the surface or inside the wound/lesion/tissue condition 19. For simplicity, in FIGS. 13C and 13D only two (2) LEDs/OLEDs 46 are shown, each of them with a dedicated LED/OLED wiring 48. The combined effects of the shockwaves/pressure waves 18, the electromagnetic field pattern 42, and of the LED/OLED phototherapy field patterns 47 will enhance and expedite the healing of the wound/lesion/tissue condition 19, which is beneficial for a successful medical treatment and thus becomes more financially viable and less time consuming for both the patients and the medical personnel. The activation of different energy technologies used by the combination shockwaves/pressure waves, electromagnetic field, and phototherapy field medical system 136 can be done simultaneously or intermittent or sometimes subsequently and is controlled by the control console/unit 22, in accordance of the user settings and preferences.

As seen from FIGS. 13C and 13D, the combination shockwaves/pressure waves, electromagnetic field, and phototherapy field medical system 136 includes a combination applicator 11 that uses a combination power cable 34 to connect to the control console/unit 22. The applicator body 14 is used by the user to move during treatment the combination applicator 11 along the longitudinal movement L (shown only on FIG. 13B, but also applicable to embodiment from FIGS. 13C and 13D) and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen in FIGS. 3A, 3B, 4A, and 4B) that actuates an external motorized ancillary system (not specifically presented in any of the figures of this patent for simplicity) that also contains sensors, motors, cameras, and clamping mechanism for the combination applicator 11. For FIGS. 13C and 13D, the combination applicators 11 will have a special design that can facilitate a medical treatment using the shockwaves/pressure waves 18 in combination with electromagnetic and phototherapy fields from the outside/exterior of the human or animal body (extracorporeally) and towards the targeted treatment zone. The other elements and components associated with the embodiment from FIGS. 13C and 13D that are related to the construction of the combination applicator 11 and the control console/unit 22 are the same in purpose, structure, construction, usability and functionality with the elements presented before for the embodiments from FIGS. 3A, 3B, 4A, 4B, 13A, and 13B.

Although the embodiments from FIGS. 13C and 13D show the use of LED/OLED phototherapy, similarly the laser phototherapy or the ultraviolet light therapy can be used as presented in FIGS. 5A-6B and 7A-7B. Also, in FIGS. 13C and 13D there are only two (2) LEDs/OLEDs 46, however at least two or three or four or more LEDs/OLEDs 46 or laser fiber optic cable exit lenses 52 or ultraviolet light lamps 71 can be used, depending of the needs and targeted medical application of the combination shockwaves/pressure waves, electromagnetic field, and phototherapy field medical system 136.

FIG. 13E shows an embodiment of a combination shockwaves/pressure waves, electromagnetic field, and non-contact ultrasound medical system 139, which incorporates means to produce shockwaves/pressure waves 18, the electromagnetic field pattern 42, and the ultrasound transmission mist solution field patterns 85 that are used to transmit the non-contact ultrasound waves 86 towards the wound/lesion/tissue condition 19 from the human/animal body 20. The central cylindrical electromagnetic coil 131 (sitting around the central magnetic core 133) and the fluid-filled cylindrical encasing tube 132 are used to producing shockwaves/pressure waves 18 via the electromagnetic principle, and the central magnetic core 133 and the frontal electromagnetic antenna with radial surface 138 are used to producing electromagnetic field pattern 42, as presented in the comments for FIGS. 13A and 13B. The frontal electromagnetic antenna with radial surface 138 is using the entire frontal surface to produce and spread/radiate a more powerful and larger electromagnetic field pattern 42 when compared to the pattern produced by the frontal electromagnetic antenna with props 134. The electromagnetic field pattern 42 produced by the props is limited in dimension by the relative positions of the props on the frontal part of the antenna, since the magnetic field can be developed only in between the props, which in the end creates a smaller and less powerful electromagnetic field pattern 42. The choice in between the frontal electromagnetic antenna with props 134 and the frontal electromagnetic antenna with radial surface 138 can be dictated by the amount of electromagnetic energy needed for a certain specific treatment or by the extension of the treatment region that is subjected to treatment, where a larger electromagnetic field pattern 42 can cover much easier the targeted wound/lesion/tissue condition 19 in conjunction with the shockwaves/pressure waves 18 and the ultrasound transmission mist solution field patterns 85 that are used to transmit the non-contact ultrasound waves 86 towards the wound/lesion/tissue condition 19 from the human/animal body 20. The mist solution that conducts the ultrasound towards the treatment targeted zone can be a saline solution or can incorporate different active fluid substances or specialized medications to help with the healing of the targeted area. The activation of different technologies used by the combination shockwaves/pressure waves, electromagnetic field, and non-contact ultrasound medical system 139 can be done simultaneously or intermittent or sometimes sequentially and it is controlled by the control console/unit 22, in accordance of the user settings and preferences.

As seen from FIG. 13E, the combination shockwaves/pressure waves, electromagnetic field, and non-contact ultrasound medical system 139 includes a combination applicator 11 that uses a combination power cable 34 and the mist solution tubing 81 to connect to the control console/unit 22. The applicator body 14 is used by the user to move during treatment the combination applicator 11 along the longitudinal movement L (shown only on FIG. 13B, but also applicable to embodiment from FIG. 13E) and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen in FIGS. 3A, 3B, 8A, and 8B) that actuates an external motorized ancillary system (not specifically presented in any of the figures of this patent for simplicity) that also contains sensors, motors, cameras, and clamping mechanism for the combination applicator 11. The applicator/coupling membrane 15 together with the parabolic reflector 135 form the fluid-filled reflector and membrane cavity 27, where the shockwaves/pressure waves 18 are generated via electromagnetic principle using a central cylindrical electromagnetic coil 131, sitting around the central magnetic core 133 and inside the fluid-filled cylindrical encasing tube 132 (see also explanations from FIGS. 13A, 13B, and 13C). The shockwaves/pressure waves 18 are transmitted inside the human/animal body 20 via applicator/coupling membrane 15 that is in direct contact with skin 21. The shockwaves/pressure waves 18 are passing through the wound/lesion/tissue condition 19 that can be positioned superficial, immediately under the skin 21, or deep inside the human/animal body 20. On the peripheral part of the bulbous region of the applicator body 14 and next to the applicator/coupling membrane 15, at least two or three or four or more ultrasound piezo ceramic/piezo crystal and mist generator subassemblies 82 are present, which are capable to create ultrasound transmission mist solution field patterns 85 to transmit the ultrasound waves 86 towards the wound/lesion/tissue condition 19. Each of the ultrasound piezo ceramic/piezo crystal and mist generator subassemblies 82 contain an ultrasound piezo ceramic/piezo crystal emission element 83 to produce ultrasound waves 86 and a mist solution nozzle/generator 84 that is used to generate the spraying pattern or ultrasound transmission mist solution field pattern 85, which is used to conduct the ultrasound waves 86 towards the wound/lesion/tissue condition 19. In FIG. 13E there are two (2) ultrasound piezo ceramic/piezo crystal and mist generator subassemblies 82, each of them with a dedicated mist solution tubing 81 and ultrasound piezo ceramic/piezo crystal wiring 87. However, other embodiments can use three (3) or four (4) or more ultrasound piezo ceramic/piezo crystal and mist generator subassemblies 82 that can increase the efficiency of the combination shockwaves/pressure waves, electromagnetic field, and non-contact ultrasound medical system 139. The combined effects of the shockwaves/pressure waves 18, the electromagnetic field pattern 42, and of the non-contact ultrasound waves 86 that are transmitted via ultrasound transmission mist solution field pattern 85 will enhance and expedite the healing of the wound/lesion/tissue condition 19, which is beneficial for a successful medical treatment and thus becomes more financially viable and less time consuming for both the patients and the medical personnel. For FIG. 13E, the combination applicators 11 will have a special design that can facilitate a medical treatment using the shockwaves/pressure waves 18 in combination with electromagnetic and non-contact ultrasound from the outside/exterior of the human or animal body (extracorporeally) and towards the targeted treatment zone. The other elements and components associated with the embodiment from FIG. 13E that are related to the construction of the combination applicator 11 and the control console/unit 22 are the same in purpose, structure, construction, usability and functionality with the elements presented before for the embodiments from FIGS. 3A, 3B, 8A, 8B, 13A, 13B, and 13C.

It is also important to note that for FIGS. 11-13E, the combination applicator 11 and the control console/unit 22 and its components are sharing a common power supply (PS) 23, which can be incorporated inside the control console/unit 22 or in some other embodiments can be an external power supply that is separate from the control console/unit 22. In other embodiments for FIGS. 11-13E, multiple and different power supplies can be used for the shockwaves/pressure waves field generator 9, or the electromagnetic generator 44, or photolight generator 49, or laser generator 54, or ultraviolet light generator 74, or ultrasound generator 88, and/or fluid field generator 24 (depending on what technologies are used by the combination applicator 11), the respective independent power supplies can be internal to the control console/unit 22 or in other embodiment can be external to the control console/unit 22 as part of an independent module/ancillary system. Although for embodiments from FIGS. 13A-13E a parabolic reflector 135 is used, there are other possible embodiments where other geometries can be used for the reflector as ellipsoidal, parabolic, spherical, conical, pyramidal or any combination of them, or in general any concave geometry, which can produce different forms of focused or unfocused shockwaves or pressure waves and can change the amount of energy that the shockwaves or pressure waves carry and deposit in each point of the wound/lesion/tissue condition 19. The other elements and components associated with the embodiments from FIGS. 11-13E that are related to the construction of the combination applicator 11 and the control console/unit 22 are the same in purpose, structure, construction, usability and functionality with the elements presented before for the embodiments from FIGS. 1A, 1B, 3A, 3B, 4A, 4B, 5A, 5B, 7A, 7B, 8A, and 8B.

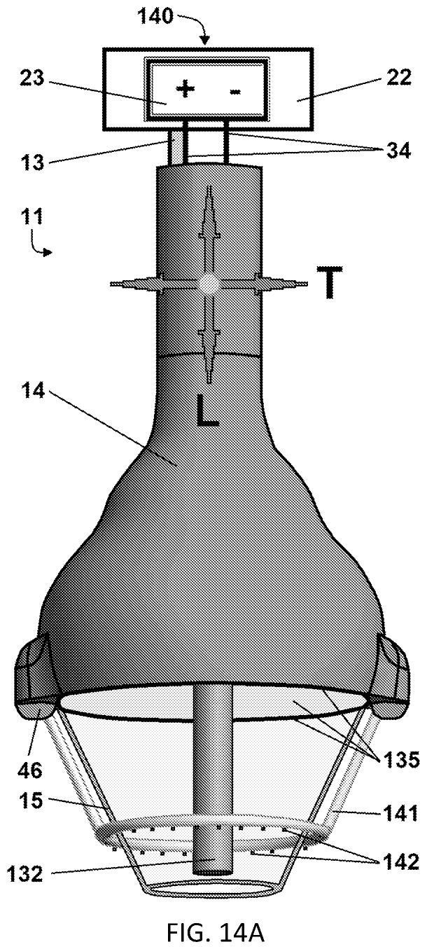

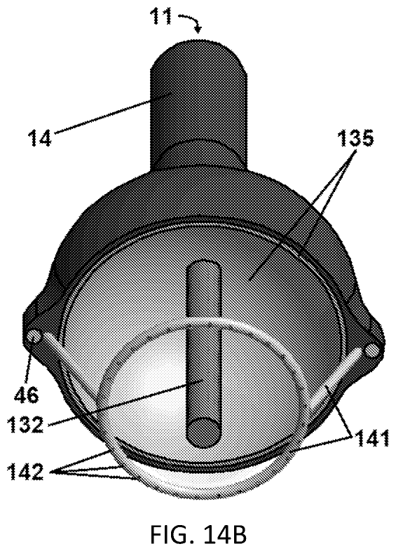

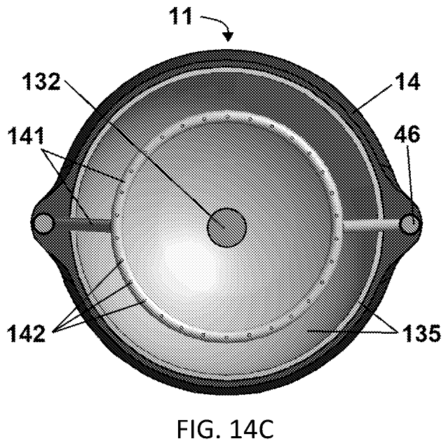

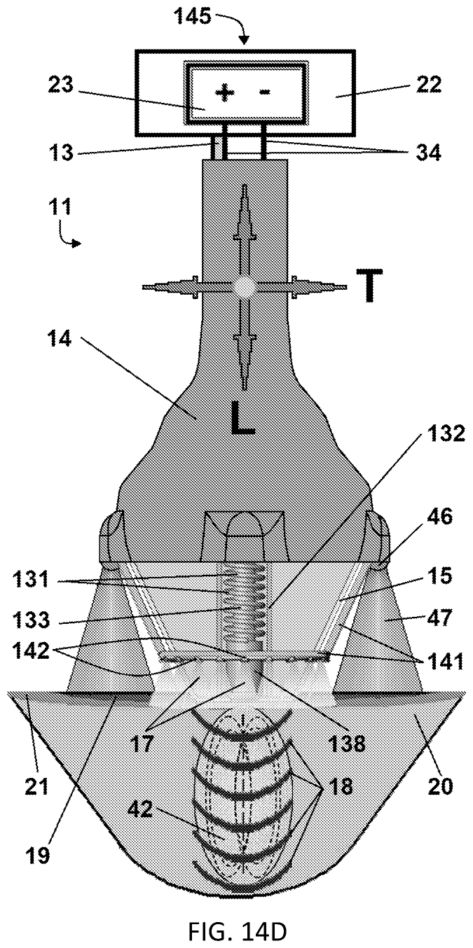

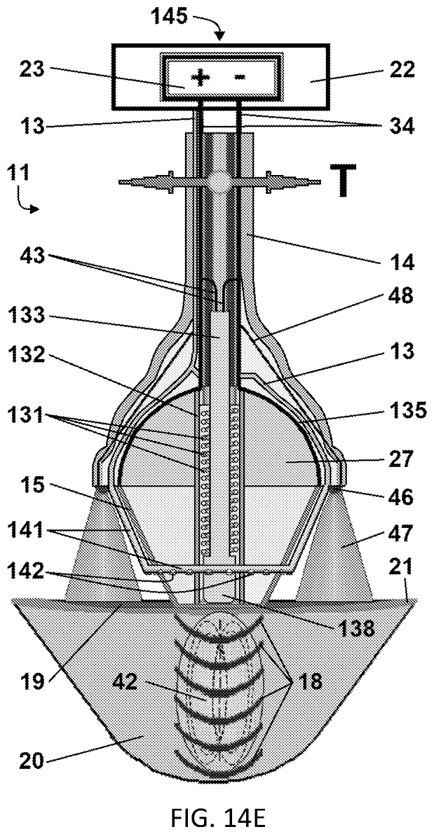

FIGS. 14A, 14B, and 14C show an embodiment of a combination shockwaves/pressure waves, photolight field, and active substance medical system 140 that can simultaneously deliver three types of medical energy modalities in the form of shockwaves/pressure waves 18, LED/OLED phototherapy field pattern 47 (not specifically shown in FIGS. 14A, 14B, and 14C, but shown in FIGS. 14D and 14E) via LED/OLED 46, and active substances from an active substance external spraying tubing 141. The applicator/coupling membrane 15 together with parabolic reflector 135 form the fluid-filled reflector and membrane cavity 27 (as seen in FIG. 14E), where the shockwaves/pressure waves 18 are generated via electromagnetic principle using a central cylindrical electromagnetic coil 131 (not specifically shown in FIGS. 14A, 14B, and 14C, but shown in FIGS. 14D and 14E) and the fluid-filled cylindrical encasing tube 132, shockwaves/pressure waves 18 that are then focused using the parabolic reflector 135 towards the wound/lesion/tissue condition 19. This is the case when the focal point of the parabolic reflector 135 is outside of the reflector. Conversely, when the focal point of the parabolic reflector 135 is inside the reflector, the pseudo planar shockwaves or pressure waves can be created and directed towards the wound/lesion/tissue condition 19. The embodiment from FIGS. 14A, 14B, and 14C cannot produce frontal electromagnetic fields for treatment purpose. The activation of different technologies used by the combination shockwaves/pressure waves, photolight field, and active substance medical system 140 can be done simultaneously or intermittent or sometimes subsequently and is controlled by the control console/unit 22, in accordance of the user settings and preferences.

The applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as presented for FIGS. 1A, 1B, 4A, and 4B) that actuates an external motorized ancillary system (not specifically presented in any of the figures of this patent for simplicity) that also contains sensors, motors, cameras, and clamping mechanism for the combination applicator 11.

The shockwaves/pressure waves 18 are transmitted inside the human/animal body 20 via applicator/coupling membrane 15 that is in direct contact with skin 21. The shockwaves/pressure waves 18 are passing through the wound/lesion/tissue condition 19 that can be positioned superficial, immediately under the skin 21, or deep inside the human/animal body 20. On the peripheral part of the bulbous region of the applicator body 14 and next to the applicator/coupling membrane 15, at least two or three or four or more LEDs/OLEDs 46 are present, which are capable to create LED/OLED phototherapy field patterns 47 (see FIGS. 14D and 14E) that are transmitted towards the wound/lesion/tissue condition 19. From the same peripheral part of the bulbous region of the applicator body 14 an active substance external spraying tubing 141 is connected to the active substance tubing 13 that comes along the combination power cable 34 from the control console/unit 22. The active substance external spraying tubing 141 has two tubes that go along the applicator/coupling membrane 15 and ends up in a circular/ring tubing that is positioned parallel and up/away from the wound/lesion/tissue condition 19. The circular/ring tubing of the active substance external spraying tubing 141 has multiple spraying tubing nozzles 142 equally distributed along the circumference of the circular/ring tubing of the active substance external spraying tubing 141. The spraying tubing nozzles 142 are used to spray an active substance and create active substance spraying patterns 17 (see FIG. 14D). The active substance that have medical effects on the wound/lesion/tissue condition 19 can be a fluid, as purified water, saline, isotonic solutions, hypotonic solutions, hypertonic solutions, etc., loaded with active liquid medicine that can be in the form of drugs, antibiotics, antioxidants, traditional natural medical substances, growth factors, proliferation factors, angiogenesis factors, cytokines, exosomes, enzymes, stem cells, bio-nanoparticles, genetic material, antibodies, vaccines, scaffolds or matrices to mimic extracellular matrices, nano-robots, specific proteins, etc. for healing of different afflictions or regeneration of cells, tissues, or organs for both human and animal subjects. The active substance can be sprayed for maximum efficiency against the wound/lesion/tissue condition 19 using a predetermined active substance spraying pattern 17 by each of the spraying tubing nozzles 142. In another embodiment the spraying tubing nozzles 142 can have different designs and thus multiple predetermined active substance spraying patterns 17 can be generated by one active substance external spraying tubing 141. Also, in another embodiment multiple active substances can be sprayed during treatment in a predetermined sequentially manner on the wound/lesion/tissue condition 19. The combined effects of the shockwaves/pressure waves 18, the LED/OLED phototherapy field patterns 47, and of the active substance that is sprayed through the active substance spraying patterns 17 via the spraying tubing nozzles 142 of the active substance external spraying tubing 141 will enhance and expedite the healing of the wound/lesion/tissue condition 19, which is beneficial for a successful medical treatment and thus becomes more financially viable and less time consuming for both the patients and the medical personnel.

The activation of different technologies used by the combination shockwaves/pressure waves, photolight field, and active substance medical system 140 can be done simultaneously or intermittent or sometimes sequentially and it is controlled by the control console/unit 22, in accordance of the user settings and preferences. For FIGS. 14A, 14B, and 14C, the combination applicators 11 will have a special design that can facilitate a medical treatment using the shockwaves/pressure waves 18 in combination with phototherapy fields and active medical substances (sprayed on the wound/lesion/tissue condition 19) from the outside/exterior of the human or animal body (extracorporeally) and towards the targeted treatment zone.

FIGS. 14D and 14E show an embodiment of a combination shockwaves/pressure waves, electromagnetic field, light field, and active substance medical system 145 that simultaneously delivers four types of medical energy modalities in the form of shockwaves/pressure waves 18, coaxial electromagnetic field pattern 42, peripheral LED/OLED phototherapy field pattern 47, and multiple active substance spraying patterns 17 produced by the multiple spraying tubing nozzles 142 that are equally distributed on the circular/ring tubing of the active substance external spraying tubing 141.

The applicator/coupling membrane 15 together with the parabolic reflector 135 form the fluid-filled reflector and membrane cavity 27, where the shockwaves/pressure waves 18 are generated via electromagnetic principle using a central cylindrical electromagnetic coil 131 and the fluid-filled cylindrical encasing tube 132, shockwaves/pressure waves 18 that are then focused using the parabolic reflector 135 towards the wound/lesion/tissue condition 19. This is the case when the focal point of the parabolic reflector 135 is outside of the reflector. Conversely, when the focal point of the parabolic reflector 135 is inside the reflector, the pseudo planar shockwaves or pressure waves can be created and directed towards the wound/lesion/tissue condition 19. The combination power cable 34 brings electric current to the central cylindrical electromagnetic coil 131 from the power supply (PS) 23 of the control console/unit 22. The shockwaves/pressure waves 18 are transmitted inside the human/animal body 20 via applicator/coupling membrane 15 that is in direct contact with skin 21 or the wound/lesion/tissue condition 19. The shockwaves/pressure waves 18 are passing through the wound/lesion/tissue condition 19 that can be positioned superficial, immediately under the skin 21 or deep inside the human/animal body 20. The central magnetic core 133 and the frontal electromagnetic antenna with radial surface 138 are used to producing electromagnetic field pattern 42 that is coaxial and overlaps with the shockwaves/pressure waves 18. As commented before for FIG. 13E, the frontal electromagnetic antenna with radial surface 138 is using the entire frontal surface to produce and spread/radiate a more powerful and larger electromagnetic field pattern 42 when compared to the pattern produced by the frontal electromagnetic antenna with props 134 (as seen in FIGS. 13A-13D). However, based on the specific energy needs of the treatment and the shape and/or positioning of the wound/lesion/tissue condition 19 relatively to the skin 21, the frontal electromagnetic antenna with radial surface 138 can be swapped with the frontal electromagnetic antenna with props 134 in the construction of the combination shockwaves/pressure waves, electromagnetic field, light field, and active substance medical system 145. The combination power cable 34 and the electromagnetic emission element wiring 43 brings electric current to the central magnetic core 133 and the frontal electromagnetic antenna with radial surface 138 from the power supply (PS) 23 of the control console/unit 22. On the peripheral part of the bulbous region of the applicator body 14 and next to the applicator/coupling membrane 15, at least two or three or four or more LEDs/OLEDs 46 are present, which are capable to create LED/OLED phototherapy field patterns 47 that are transmitted towards the wound/lesion/tissue condition 19. The combination power cable 34 and the LED/OLED wiring 48 brings electric current to the LEDs/OLEDs 46 from the power supply (PS) 23 of the control console/unit 22. From the same peripheral part of the bulbous region of the applicator body 14 an active substance external spraying tubing 141 is connected to the active substance tubing 13 that comes along the combination power cable 34 from the control console/unit 22. The active substance external spraying tubing 141 has two tubes that go along the applicator/coupling membrane 15 and ends up in a circular/ring tubing parallel and up/away from the wound/lesion/tissue condition 19. The circular/ring tubing of the active substance external spraying tubing 141 has multiple spraying tubing nozzles 142 equally distributed along the circumference of the circular/ring tubing. The spraying tubing nozzles 142 are used to spray an active substance and create active substance spraying patterns 17 (see FIG. 14D). The active substance that have medical effects on the wound/lesion/tissue condition 19 can be a fluid, as purified water, saline, isotonic solutions, hypotonic solutions, hypertonic solutions, etc., loaded with active liquid medicine that can be in the form of drugs, antibiotics, antioxidants, traditional natural medical substances, growth factors, proliferation factors, angiogenesis factors, cytokines, exosomes, enzymes, stem cells, bio-nanoparticles, antibodies, genetic material, vaccines, scaffolds or matrices to mimic extracellular matrices, nano-robots, specific proteins, etc. for healing of different afflictions or regeneration of cells, tissues, or organs for both human and animal subjects. The active substance can be sprayed for maximum efficiency against the wound/lesion/tissue condition 19 using a predetermined active substance spraying pattern 17 by each of the spraying tubing nozzles 142. In another embodiment the spraying tubing nozzles 142 can have different designs and thus multiple predetermined active substance spraying patterns 17 can be generated by one active substance external spraying tubing 141. Also, in another embodiment multiple active substances can be sprayed during treatment in a predetermined sequentially manner on the wound/lesion/tissue condition 19. The combined effects of the shockwaves/pressure waves 18, the electromagnetic field pattern 42, the LED/OLED phototherapy field patterns 47, and of the active substance or substances that are sprayed through the active substance spraying patterns 17 via the spraying tubing nozzles 142 of the active substance external spraying tubing 141 will enhance and expedite the healing of the wound/lesion/tissue condition 19, which is beneficial for a successful medical treatment and thus becomes more financially viable and less time consuming for both the patients and the medical personnel.

The activation of different technologies used by the combination shockwaves/pressure waves, electromagnetic field, light field, and active substance medical system 145 can be done simultaneously or intermittent or sometimes sequentially and it is controlled by the control console/unit 22, in accordance of the user settings and preferences. For FIGS. 14D, and 14E, the combination applicators 11 will have a special design that can facilitate a medical treatment using the shockwaves/pressure waves 18 in combination with electromagnetic fields, phototherapy fields, and active medical substances (sprayed on the wound/lesion/tissue condition 19) from the outside/exterior of the human or animal body (extracorporeally) and towards the targeted treatment zone.

In FIGS. 14D and 14E the applicator body 14 is used by the user to move during treatment the combination applicator 11 along the longitudinal movement L and transversal movement T, to allow the covering of the entire targeted treatment area or region or wound/lesion/tissue condition 19 of the human/animal body 20. The L and T movements can be done manually or automatically, and when they are performed automatically, these movements are controlled by the control console/unit processor 1000 (as seen in FIGS. 1A, 1B, 3A, 3B, 4A, and 4B) that actuates an external motorized ancillary system (not specifically presented in any of the figures of this patent for simplicity) that also contains sensors, motors, cameras, and clamping mechanism for the combination applicator 11.

Although the embodiments from FIGS. 14A-14E show the use of LED/OLED phototherapy, similarly the laser phototherapy or the ultraviolet light therapy can be used, as presented in FIGS. 5A-6B and 7A-7B. Also, in FIGS. 14A-14E there are only two (2) LEDs/OLEDs 46, however at least two or three or four or more LEDs/OLEDs 46 or laser fiber optic cable exit lenses 52 or ultraviolet light lamps 71 can be used, depending of the needs and targeted medical application of the combination shockwaves/pressure waves, electromagnetic field, and active substance medical system 130 or of the combination shockwaves/pressure waves, electromagnetic field, light field, and active substance medical system 145.

It is also important to note that for the FIGS. 14A-14E the combination applicator 11 and the control console/unit 22 and its components are sharing a common power supply (PS) 23, which can be incorporated inside the control console/unit 22 or in some other embodiments can be an external power supply that is separate from the control console/unit 22. In other embodiments for FIGS. 14A-14E, multiple and different power supplies can be used for the shockwaves/pressure waves field generator 9, or the electromagnetic generator 44, or photolight generator 49, or laser generator 54, or ultraviolet light generator 74, and/or fluid field generator 24 (depending on what technologies are used by the combination applicator 11), the respective independent power supplies can be internal to the control console/unit 22 or in other embodiment can be external to the control console/unit 22 as part of an independent module/ancillary system. Although for embodiments from FIGS. 14A-14E a parabolic reflector 135 is used, there are other possible embodiments where other geometries can be used for the reflector as ellipsoidal, parabolic, spherical, conical, pyramidal or any combination of them, or in general any concave geometry, which can produce different forms of focused or unfocused shockwaves or pressure waves and can change the amount of energy that the shockwaves or pressure waves carry and deposit in each point of the wound/lesion/tissue condition 19. The other elements and components associated with the embodiment from FIGS. 14A-14E that are related to the construction of the combination applicator 11 and the control console/unit 22 are the same in purpose, structure, construction, usability and functionality with the elements presented before for the embodiments from FIGS. FIGS. 1A, 1B, 3A, 3B, 4A, 4B, 5A, 5B, 7A, and 7B.

Shockwaves/pressure waves 18 can be also combined with delivery of nanoparticles in the same device/system, in the form of specially designed applicators and energy combination medical systems that incorporate besides shockwaves/pressure waves generating design elements (shockwave/pressure wave generators, reflectors, membranes, etc.) also some specific components able to deliver nanoparticles via a fluid solution simultaneously with the shockwaves/pressure waves 18 (similar to the system presented in FIGS. 1A and 1B) for stimulation, healing, and regeneration of cells, tissues, or organs. In this case, the combination applicators 11 will have a special design that allows the application of the shockwaves/pressure waves 18 in combination with a dispensing system for nanoparticles from the outside/exterior of the human or animal body (extracorporeally) and towards the targeted treatment zone.

Nanoparticles can be themselves active due to the material used to create them (silver, gold, zinc oxide, copper, ceramic, etc.) or can be hollow (become nanocarriers) and can carry different substances inside them as nitric oxide, antibiotics, antioxidants, growth factors, proliferation factors, angiogenesis factors, cytokines, vaccines, antibodies, exosomes, enzymes, drugs, different natural substances with active role in healing, genes, DNA, RNA, stem cells, etc. Thus, a localized delivery can be accomplished and, in this way, avoiding large dosages, provide high efficiency, and avoid the side effects when systemically approach for delivery is used. The nanoparticles properties can be also changed via their hydrophobicity (repelling water) or hydrophilicity (attracting water), which can have significant advantages in treatment of different afflictions and medical conditions. Also, the nanoparticles can be created with special three-dimensional shapes that are very effective in killing pathogens, prevent or break bacterial biofilms, or create immunological response to viruses or other invading micro-organisms.

In another embodiment, the nanoparticles can be delivered as a drug solution in a systemic way. By controlling the nanoparticle dimensions, certain types of tissues or organs can be targeted, based on the tissue or organ structural density and intercellular spaces, which allows the absorption of only nanoparticles of a certain dimension. Once the targeted tissue is reached and nanoparticles are absorbed inside the tissue, the nanoparticles can slowly deliver the active substances or a special extracorporeal energy combination system can be used to break the nanoparticles and locally deliver the active substances in sufficient high dosages for superior therapeutic results, without creating any systemic adverse reaction. That opens the door for the locally delivery of certain drug therapies that initially failed due to the adverse effects generated by the systemic delivery, although the compound effects could be beneficial for treating a certain disease, tissue, organ, etc. This approach of combining the systemic delivery of nano-particles combined with a special extracorporeal energy combination system could be used for delivering antibiotics, antioxidants, traditional natural medical substances, growth factors, proliferation factors, angiogenesis factors, cytokines, exosomes, enzymes, stem cells, genetic material, vaccines, antibodies, scaffolds or matrices to mimic extracellular matrices, nano-robots, specific proteins, etc. in a time/dose efficient way. For breaking the nanoparticles to deliver a specific active substance inside the human/animal body 20 in a certain human or animal tissue or organ, extracorporeal combination applicators 11 can be used, which produce shockwaves/pressure waves 18 in combination with electric current field patterns 33 (as presented in FIGS. 2A-2B), or electromagnetic field patterns 42 (as presented in FIGS. 3A-3B, 13A-13E, and 14D-14E), or LED/OLED phototherapy field patterns 47 (as presented in FIGS. 4A-4B, 13C-13D, and 14A-14E), or laser phototherapy field patterns 53 (as presented in FIGS. 5A-6B, 13C-13D, and 14A-14E), or ultraviolet light field patterns 72 (as presented in FIGS. 7A-7B, 13C-13D, and 14A-14E), or non-contact/contact ultrasound waves 86 (as presented in FIGS. 8A-9B, 12, and 13E), or radio-frequency field pattern 102 (as presented in FIGS. 10A-10B, and 11), to name a few. Such nanoparticles can be delivered systemically, injected locally or spread on the top of the targeted wound/lesion/tissue condition 19, or in the targeted treatment areas or regions. Also, based on different thickness of the nanoparticle wall, a selective delivery can be done in time due the use of different levels of energy needed for each type of nanoparticle to break a thinner or thicker nanoparticle wall.

Nanotechnology can also be used particularly in combination with energy combination systems that produce shockwaves/pressure waves 18 in combination with phototherapy, as presented in FIGS. 4A-4B, 5A-7B, 13C-13D, and 14A-14E. Special designed meshes are produced that have gold nanoparticles, which are activated by light and can produce localized heat. Such nanoparticle meshes can be used together with the energy combination systems and their combination applicators for different cancers or skin lesions. The same technology can be used to destroy planktonic bacteria and biofilms by activating the nanoparticles with short intense near-infrared light or lasers produced by the energy combination systems and their combination applicators. The same approach can make the bacteria more susceptible toward antibiotic therapy. Besides the combination energy systems of phototherapy with shockwaves/pressure waves (as presented in FIGS. 4A-4B, 5A-7B, 13C-13D, and 14A-14E), combination energy systems of phototherapy with ultrasound (as presented in FIGS. 8A-9B, 12, 13E, 17, 18, and 19) can also be used.

In another embodiment, the shockwaves/pressure waves 18 can also be combined with pneumatic compression in the same device (not specifically shown in any of the figures of this invention), in the form of specially designed combination applicators 11 and energy combination medical systems that incorporate besides shockwaves/pressure waves generating design elements (shockwave/pressure wave generators, reflectors, membranes, etc.) also some specific components able to deliver pneumatic pressure simultaneously with the shockwaves/pressure waves 18 for stimulation, healing, and regeneration of cells, tissues, or organs. In this case, the combination applicators 11 will have a special design that allows the application of shockwaves/pressure waves 18 and pneumatic compression from the outside/exterior of the human or animal body (extracorporeally) and towards the targeted treatment zone.

In another embodiment, the shockwaves/pressure waves 18 can be combined with hydrotherapy in the same device (not specifically shown in any of the figures of this invention), in the form of specially designed combination applicators 11 and energy combination medical systems that incorporate besides shockwaves/pressure waves generating design elements (shockwave/pressure wave generators, reflectors, membranes, etc.) also some specific components able to deliver hydrotherapy simultaneously with the shockwaves/pressure waves 18 for stimulation, healing, and regeneration of cells, tissues, or organs. In this case, the combination applicators 11 will have a special design that allows the application of the shockwaves/pressure waves 18 in combination with hydrotherapy from the outside/exterior of the human or animal body (extracorporeally) and towards the targeted treatment zone. In some embodiments where the hydrotherapy is used for cleaning of wound/lesion/tissue condition 19, the addition of the shockwaves/pressure waves 18 can create the movement of the fluid (due macro-streaming or micro-streaming) to produce whirlpools and pulsed lavage with concurrent suction, which will make the hydrotherapy more efficient in treating wound/lesion/tissue condition 19 from the human/animal body 20.

In another embodiment, the shockwaves/pressure waves 18 can be also combined with cold atmospheric plasma in the same device (not specifically shown in any of the figures of this invention), in the form of specially designed combination applicators 11 and energy combination medical systems that incorporate besides shockwaves/pressure waves generating design elements (shockwave/pressure wave generators, reflectors, membranes, etc.) also some specific components able to produce cold atmospheric plasma simultaneously with the shockwaves/pressure waves 18 for stimulation, healing, and regeneration of cells, tissues, or organs. In this case, the combination applicators 11 will have a special design that allows the application of the shockwaves/pressure waves 18 in combination with cold atmospheric plasma from the outside/exterior of the human or animal body (extracorporeally) and towards the targeted treatment zone.

In another embodiment, the shockwaves/pressure waves 18 can be also combined with topical negative pressures in the same device (not specifically shown in any of the figures of this invention), in the form of specially designed combination applicators 11 and energy combination medical systems that incorporate besides shockwaves/pressure waves generating design elements (shockwave/pressure wave generators, reflectors, membranes, etc.) also some specific components (foam bandage, negative pressure pumps, etc.) able to produce negative pressures simultaneously with the shockwaves/pressure waves 18 for stimulation, healing, and regeneration of cells, tissues, or organs. In this case, the combination applicators 11 will have a special design that allows the application of the shockwaves/pressure waves 18 in combination with topical negative pressures from the outside/exterior of the human or animal body (extracorporeally) and towards the targeted treatment zone. Furthermore, for this embodiment any of the combination applicators 11 presented in FIGS. 1A-14E should be able to have their action through the foam bandages that are used by the existing topical negative pressure systems, and in this way to have an additive effect besides the negative/vacuum pressures on stimulation healing, and regeneration of cells, tissues, or organs from the human/animal body 20.

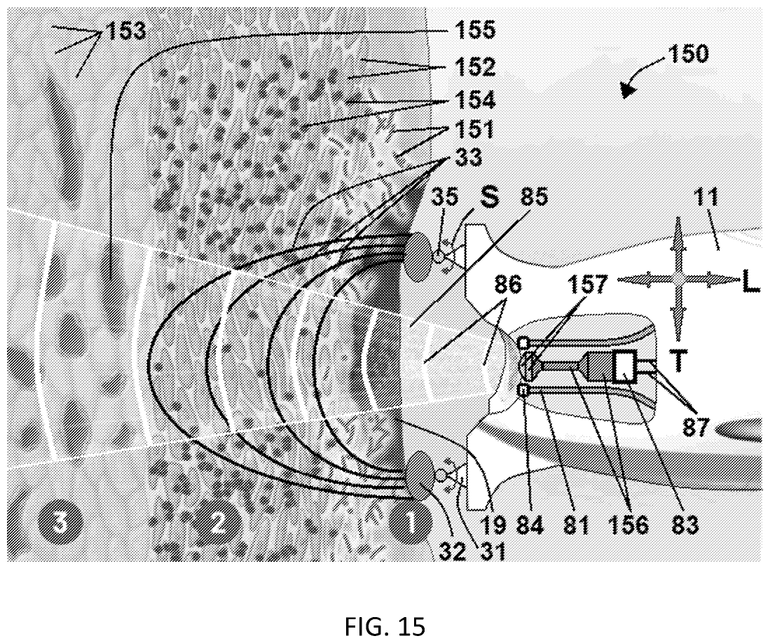

Furthermore in yet another embodiment, the shockwaves/pressure waves 18 can be combined with thermal therapy in the same device (not specifically shown in any of the figures of this invention), in the form of specially designed combination applicators 11 and energy combination medical systems that incorporate besides shockwaves/pressure waves generating design elements (shockwave/pressure wave generators, reflectors, membranes, etc.) also some specific components able to produce thermal therapy simultaneously with the shockwaves/pressure waves 18 for stimulation, healing, and regeneration of cells, tissues, or organs. The thermal effect can be done via specially designed external heating coils/resistors or heated fluids (inside the applicator/coupling membrane 15 or balloons) or can be produced via electric currents as the ones presented in FIGS. 2A-2B, and 15, electromagnetic fields as the ones presented in FIGS. 3A-3B, 13A-13E, 14D-14E, and 16, LEDs/OLEDs as the ones presented in FIGS. 4A-4B, 13C-13D, 14A-14E, and 17, lasers as the ones presented in FIGS. 5A-6B, 13C-13D, 14A-14E, and 18, contact ultrasound waves as the ones presented in FIGS. 9A-9B, 12, and 13E, ultraviolet light as the ones presented in FIGS. 7A-7B, 13C-13D, 14A-14E, and 19, and radio-frequency fields as the ones presented in FIGS. 10A-10B, 11, and 20, to name a few. The warming or heating of cells, tissues, or organs is used either to stimulate their functioning or for ablation of unwanted benign and non-benign tissues or cells. In this case, the combination applicators 11 will have a special design that allows the application of the combination energy therapies with the concomitant thermal therapy from the outside/exterior of the human or animal body (extracorporeally) and towards the targeted treatment zone.

The combination applicators 11 presented in FIGS. 1A-14E can be also combined with traditional Chinese and Indian medicine in the same device (not specifically shown in any of the figures of this invention), in the form of specially designed applicators and energy combination medical systems. The shockwaves/pressure waves 18 can also replace the acupuncture or can enhance the effect of the acupuncture that was applied before or during shockwaves application. Similar acupuncture effects can be obtained with using special electrodes to produce electrical currents. This is why the combination applicators 11 presented in FIGS. 1A-14E can be used to stimulate the cells, tissue, or organs and push more efficient different natural substances from traditional Chinese and Indian medicine inside the targeted wound/lesion/tissue condition 19 from the human/animal body 20.

Shockwaves/pressure waves 18 in combination with other energy technologies (pressurized active substances, electric currents, electromagnetic fields, LED/OLED phototherapy, lasers, no-contact or contact ultrasound waves, ultraviolet (UV) light fields, radio-frequency fields, topical negative pressures, energy-based nanotechnology, pneumatic compression, hydrotherapy, cold plasma, thermal therapy, energy-based oxygen, traditional Chinese and Indian medicine, etc.) can be used in combination applicators 11 presented in FIGS. 1A-14E for stimulation and healing of organ and tissue transplants or skin grafts and active skin substitutes (placental, dermal, fish skin, etc.). For all these cases, the combination applicators 11 will have a special design that allows the application of the energy combination therapy from the outside/exterior of the human or animal body (extracorporeally) and towards the targeted treatment zone.

FIG. 15 shows the combination non-contact ultrasound and electric current fields medical system 150 that is capable in the same device to produce non-contact ultrasound in combination with mobile electric fields. The ultrasound waves 86 can be low frequency (20 kHz to 500 kHz), medium (501 kHz to 2 MHz), or high frequency ultrasound (2.1 MHz to 20 MHz). The higher the frequency of the ultrasound the higher is the probability to produce heating effects inside the wound/lesion/tissue condition 19. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce pulsed or continuous electric fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with electric fields from the outside/exterior of the human or animal body (extracorporeally) and towards the targeted treatment zone.

As seen from FIG. 15, the combination non-contact ultrasound and electric current fields medical system 150 includes a combination applicator 11 that has a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 15, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 15, but seen in other figures). The L and T movements can be done manually or automatically, and when they are performed automatically, these movements are controlled by the control console/unit 22 that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial, can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. In FIG. 15, the ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 15, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or four or more electric current emission elements 31 are present and connected via the swiveling hinges 35 to the swiveling electric current electrodes 32, which are capable to create electric current field patterns 33 inside the wound/lesion/tissue condition 19. In FIG. 15 there are two (2) electric current emission elements 31, each of them with a dedicated electric current emission element wiring 36 (not shown specifically in FIG. 15, but shown in FIG. 2B). The swiveling movement S allows the correct position of the swiveling electric current electrode 32 and assures a constant contact with the wound/lesion/tissue condition 19. The combined effects of the non-contact ultrasound waves 86 that are transmitted via ultrasound transmission mist solution field pattern 85 and of the electric current field pattern 33 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and electric current fields medical system 150, the control console/unit 22 (not specifically shown in FIG. 15) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the electric current field generator (ECG) 38 that includes the electric current generator processor 3000, the electric current generator Bluetooth (BT) module 3010, the electric current generator Wi-Fi module 3020, the electric current generator RFID module 3030, the electric current generator I/O (input/output) element 3040, the electric current generator memory 3050, the electric current generator emission mechanism (EM) module 3060, the electric current generator timer module 3070, and the electric current field generator (ECG) module 3080, as seen in FIG. 2B. For FIG. 15, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the electric current generator processor 3000 has also a slave-master relationship with the ultrasound generator processor 8000, which means that the ultrasound generator processor 8000 controls also the activity of the electric current generator processor 3000. Through this process the ultrasound generator processor 8000 together with the fluid pump processor 2000 and the electric current generator processor 3000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, the ultrasound transmission mist solution field pattern 85, the electric current form (low intensity direct current (LIDC), high voltage pulsed current (HVPC), asymmetrical biphasic pulsed current (ABPC), alternative current (AC), etc.), time of activation, intensity, polarity, points of application, and the electric current field patterns 33. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 15. It is also important to note that the combination applicator 11, the control console/unit 22, the ultrasound generator 88, the fluid field generator 24 that directly controls the output of the fluid pump (FP) 24A, and the electric current field generator (ECG) 38 are sharing a common power supply (PS) 23, which can be incorporated inside the control console/unit 22 (as presented in FIG. 1B, 2B, or 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the electric current field generator (ECG) 38, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and electric current fields medical system 150 presented in FIG. 15. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the electric current field patterns 33. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, electric current form (low intensity direct current (LIDC), high voltage pulsed current (HVPC), asymmetrical biphasic pulsed current (ABPC), alternative current (AC), etc.), time of activation, intensity, polarity, points of application, the electric current field patterns 33, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 15, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the electric current field generator (ECG) 38 are similar to the ones presented before for FIGS. 1A-1B, 2A-2B, and 8A-8B.

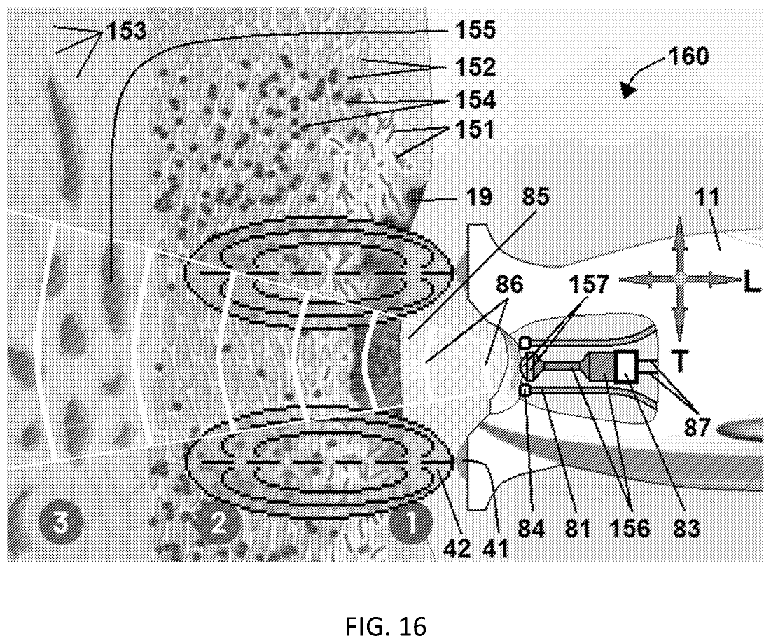

FIG. 16 shows the combination non-contact ultrasound and electromagnetic fields medical system 160 that is capable in the same device to produce non-contact ultrasound in combination with electromagnetic fields. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce electromagnetic fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with electromagnetic fields from the outside/exterior of the human or animal body (extracorporeally) and towards the targeted treatment zone.

As seen from FIG. 16, the combination non-contact ultrasound and electromagnetic fields medical system 160 includes a combination applicator 11 that uses a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 16, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 16, but seen in other figures). The L and T movements can be done manually or automatically, and when they are performed automatically, these movements are controlled by the control console/unit 22 (not shown in FIG. 16) that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. The ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 16, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or three or more electromagnetic emission elements 41 are present, which are capable to create electromagnetic field patterns 42 inside the wound/lesion/tissue condition 19. In FIG. 16 there are two (2) electromagnetic emission elements 41, each of them with a dedicated electric current emission element wiring 36 (not shown specifically in FIG. 16, but shown in FIG. 3B). The electromagnetic emission elements 41 can be at a certain distance from the skin 21 or wound/lesion/tissue condition 19 (as seen in FIG. 16) or in another embodiment the electromagnetic emission elements 41 can be modified/designed to be positioned in direct contact with the skin 21 or wound/lesion/tissue condition 19. The combined effects of the non-contact ultrasound waves 86 that are transmitted via ultrasound transmission mist solution field pattern 85 and of the electromagnetic field patterns 42 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and electromagnetic fields medical system 160, the control console/unit 22 (not specifically shown in FIG. 16) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the electromagnetic generator 44 that includes the electromagnetic generator processor 4000, the electromagnetic generator Bluetooth (BT) module 4010, the electromagnetic generator Wi-Fi module 4020, the electromagnetic generator RFID module 4030, the electromagnetic generator I/O (input/output) element 4040, the electromagnetic generator memory 4050, the electromagnetic generator emission mechanism (EM) module 4060, the electromagnetic generator timer module 4070, and the electromagnetic generator field (EMG) module 4080, as seen in FIG. 3B. For FIG. 16, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the electromagnetic generator processor 4000 has also a slave-master relationship with the ultrasound generator processor 8000, which means that the ultrasound generator processor 8000 controls also the activity of the electromagnetic generator processor 4000. Through this process the ultrasound generator processor 8000 together with the fluid pump processor 2000 and the electromagnetic generator processor 4000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, the ultrasound transmission mist solution field pattern 85, the type of electromagnetic fields (continuous electromagnetic fields (EMFs) or pulsed electromagnetic fields (PEMF)), electric current form intensity used to create electromagnetic fields, time of activation, intensity, polarity, points of application, and the electromagnetic field patterns 42. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 16. It is also important to note that the combination applicator 11, the control console/unit 22, the ultrasound generator 88, the fluid field generator 24 that directly controls the output of the fluid pump (FP) 24A, and the electromagnetic generator 44 are sharing a common power supply (PS) 23, which can be incorporated inside the control console/unit 22 (as presented in FIG. 1B, 3B, or 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the electromagnetic generator 44, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and electromagnetic fields medical system 160 presented in FIG. 16. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the electromagnetic field patterns 42. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, type of electromagnetic fields (continuous electromagnetic fields (EMFs) or pulsed electromagnetic fields (PEMF)), electric current form intensity used to create electromagnetic fields, time of activation, intensity, polarity, points of application, electromagnetic field patterns 42, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 16, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the electromagnetic generator 44 are similar to the ones presented before for FIGS. 1A-1B, 3A-3B, and 8A-8B.

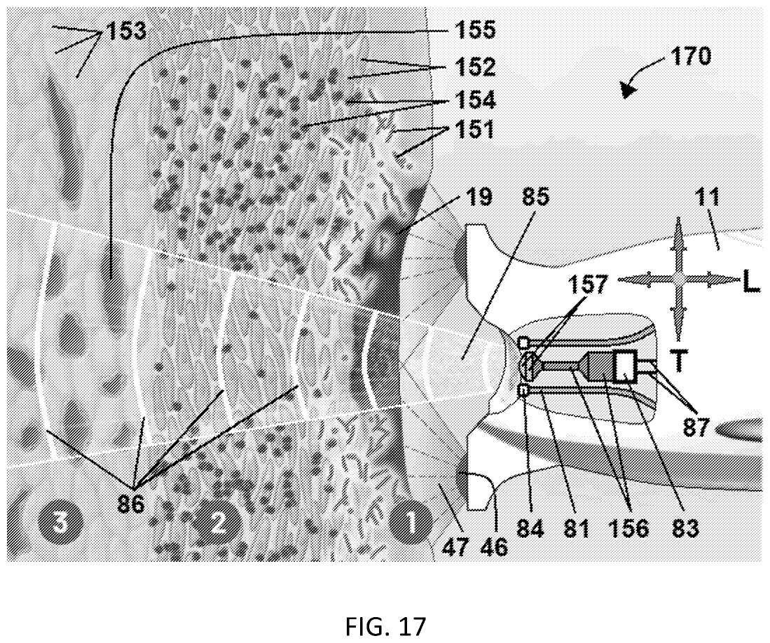

FIG. 17 shows the combination non-contact ultrasound and LED/OLED phototherapy medical system 170 that is capable in the same device to produce non-contact ultrasound in combination with LED/OLED phototherapy. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce LED/OLED phototherapy fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with LED/OLED phototherapy from the outside/exterior of the human or animal body (extracorporeally) and towards the targeted treatment zone.

As seen from FIG. 17, the combination non-contact ultrasound and LED/OLED phototherapy medical system 170 includes a combination applicator 11 that uses a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 17, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 17, but seen in other figures). The L and T movements can be done manually or automatically, and when they are performed automatically, these movements are controlled by the control console/unit 22 (not shown in FIG. 17) that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. The ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 17, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or three or more LEDs/OLEDs 46 are present, which are capable to create LED/OLED phototherapy field patterns 47 inside the wound/lesion/tissue condition 19. In FIG. 17 there are two (2) LEDs/OLEDs 46, each of them with a dedicated LED/OLED wiring 48 (not shown specifically in FIG. 17, but shown in FIG. 4B). The combined effects of the non-contact ultrasound waves 86 that are transmitted via ultrasound transmission mist solution field pattern 85 and of the LED/OLED phototherapy field patterns 47 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and LED/OLED phototherapy medical system 170, the control console/unit 22 (not specifically shown in FIG. 17) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the photolight generator 49 that includes the photolight generator processor 5000, the photolight generator Bluetooth (BT) module 5010, the photolight generator Wi-Fi module 5020, the photolight generator RFID module 5030, the photolight generator I/O (input/output) element 5040, the photolight generator memory 5050, the photolight generator emission mechanism (EM) module 5060, the photolight generator timer module 5070, and photolight field generator (PFG) module 5080, as seen in FIG. 4B. For FIG. 17, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the photolight generator processor 5000 has also a slave-master relationship with the ultrasound generator processor 8000, which means that the ultrasound generator processor 8000 controls also the activity of the photolight generator processor 5000. Through this process the ultrasound generator processor 8000 together with the fluid pump processor 2000 and the photolight generator processor 5000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, the ultrasound transmission mist solution field pattern 85, the type of light (white, blue, green, yellow, amber, red, or near-infrared), time of activation, intensity, points of application, levels of brightness, color-changing capability with broad spectral tunability and ultrafast responses, and the LED/OLED phototherapy field patterns 47. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 17. It is also important to note that the combination applicator 11, the control console/unit 22, the ultrasound generator 88, the fluid field generator 24 that directly controls the output of the fluid pump (FP) 24A, and the photolight generator 49 are sharing a common power supply (PS) 23, which can be incorporated inside the control console/unit 22 (as presented in FIG. 1B, 4B, or 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the photolight generator 49, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and LED/OLED phototherapy medical system 170 presented in FIG. 17. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the electric current field patterns 33. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, type of light (white, blue, green, yellow, amber, red, or near-infrared), time of activation, intensity, points of application, levels of brightness, color-changing capability with broad spectral tunability and ultrafast responses, LED/OLED phototherapy field patterns 47, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 17, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the photolight generator 49 are similar to the ones presented before for FIGS. 1A-1B, 4A-4B, and 8A-8B.

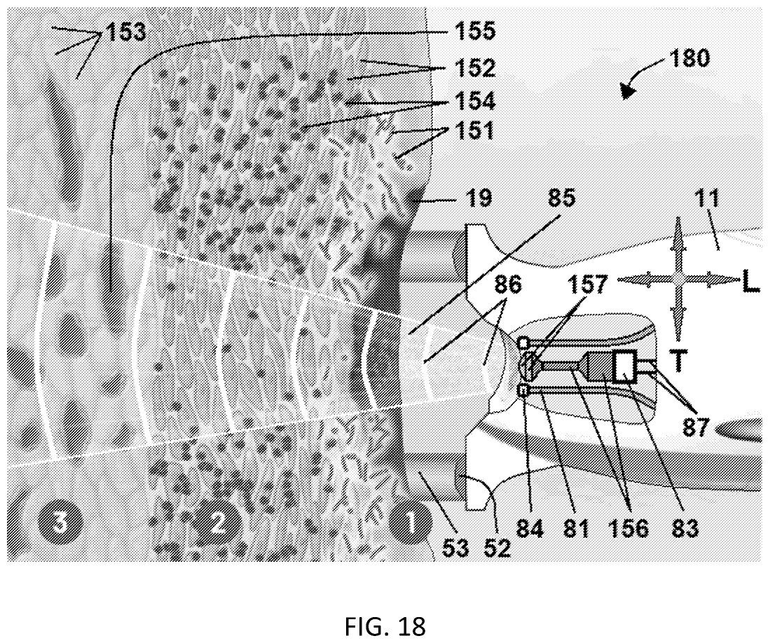

FIG. 18 shows the combination non-contact ultrasound and laser phototherapy medical system 180 that is capable in the same device to produce non-contact ultrasound in combination with laser phototherapy. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce laser phototherapy fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with laser phototherapy from the outside/exterior of the human or animal body (extracorporeally) and towards the targeted treatment zone.

As seen from FIG. 18, the combination non-contact ultrasound and laser phototherapy medical system 180 includes a combination applicator 11 that uses a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 18, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 18, but seen in other figures). The L and T movements can be done manually or automatically, and when they are performed automatically, these movements are controlled by the control console/unit 22 (not shown in FIG. 18) that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. The ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 18, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or three or more laser fiber optic cable exit lenses 52 are present, which are capable to create laser phototherapy field patterns 53 inside the wound/lesion/tissue condition 19. In FIG. 18 there are two (2) laser fiber optic cable exit lenses 52, each of them with a dedicated laser fiber optic cable 51 (not shown specifically in FIG. 18, but shown in FIG. 5B). The combined effects of the non-contact ultrasound waves 86 that are transmitted via ultrasound transmission mist solution field pattern 85 and of the laser phototherapy field patterns 53 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and laser phototherapy medical system 180, the control console/unit 22 (not specifically shown in FIG. 18) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the laser generator 54 that includes the laser generator processor 6000, the laser generator Bluetooth (BT) module 6010, the laser generator Wi-Fi module 6020, the laser generator RFID module 6030, the laser generator I/O (input/output) element 6040, the laser generator memory 6050, the laser generator emission mechanism (EM) module 6060, the laser generator timer module 6070, and the laser field generator (LaG) module 6080, as seen in FIG. 5B. For FIG. 18, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the laser generator processor 6000 has also a slave-master relationship with the ultrasound generator processor 8000, which means that the ultrasound generator processor 8000 controls also the activity of the laser generator processor 6000. Through this process the ultrasound generator processor 8000 together with the fluid pump processor 2000 and the laser generator processor 6000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, the ultrasound transmission mist solution field pattern 85, the type of laser (red or infrared), laser amplitude that dictates laser's color and penetration, duration of the pulse, time of activation, intensity, points of application, levels of brightness, continuous or intermittent application, and the laser phototherapy field patterns 53. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 18. It is also important to note that the combination applicator 11, the control console/unit 22, the ultrasound generator 88, the fluid field generator 24 that directly controls the output of the fluid pump (FP) 24A, and the laser generator 54 are sharing a common power supply (PS) 23, which can be incorporated inside the control console/unit 22 (as presented in FIG. 1B, 5B, or 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the laser generator 54, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and laser phototherapy medical system 180 presented in FIG. 18. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the laser phototherapy field patterns 53. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, type of laser (red or infrared), laser amplitude that dictates laser's color and penetration, duration of the pulse, time of activation, intensity, points of application, levels of brightness, continuous or intermittent application, laser phototherapy field patterns 53, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 18, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the laser generator 54 are similar to the ones presented before for FIGS. 1A-1B, 5A-5B, and 8A-8B.

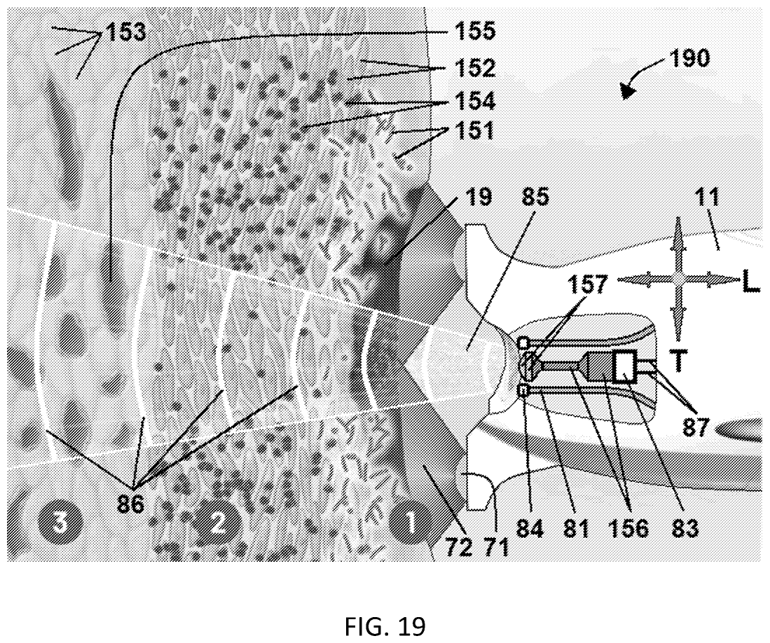

FIG. 19 shows the combination non-contact ultrasound and ultraviolet light medical system 190 that is capable in the same device to produce non-contact ultrasound in combination with ultraviolet light. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce ultraviolet light fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with ultraviolet light from the outside/exterior of the human or animal body (extracorporeally) and towards the targeted treatment zone.

As seen from FIG. 19, the combination non-contact ultrasound and ultraviolet light medical system 190 includes a combination applicator 11 that uses a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 19, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 19, but seen in other figures). The L and T movements can be done manually or automatically, and when they are performed automatically, these movements are controlled by the control console/unit 22 (not shown in FIG. 19) that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. The ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 19, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or three or more ultraviolet light lamps 71 are present, which are capable to create ultraviolet light field patterns 72 inside the wound/lesion/tissue condition 19. In FIG. 19 there are two (2) ultraviolet light lamps 71, each of them with a dedicated ultraviolet light lamp wiring 73 (not shown specifically in FIG. 19, but shown in FIG. 7B). The combined effects of the non-contact ultrasound waves 86 that are transmitted via ultrasound transmission mist solution field pattern 85 and of the ultraviolet light field patterns 72 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and ultraviolet light medical system 190, the control console/unit 22 (not specifically shown in FIG. 19) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the ultraviolet light generator (UVG) 74 that includes the ultraviolet light generator processor 7000, the ultraviolet light generator Bluetooth (BT) module 7010, the ultraviolet light generator Wi-Fi module 7020, the ultraviolet light generator RFID module 7030, the ultraviolet light generator I/O (input/output) element 7040, the ultraviolet light generator memory 7050, the ultraviolet light generator emission mechanism (EM) module 7060, the ultraviolet light generator timer module 7070, and the ultraviolet light field generator (UVG) module 7080, as seen in FIG. 7B. For FIG. 19, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the ultraviolet light generator processor 7000 has also a slave-master relationship with the ultrasound generator processor 8000, which means that the ultrasound generator processor 8000 controls also the activity of the ultraviolet light generator processor 7000. Through this process the ultrasound generator processor 8000 together with the fluid pump processor 2000 and the ultraviolet light generator processor 7000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, UV light type (UV light A (UVA), UV light B (UVB), UV light C (UVC) and vacuum UV), time of activation, intensity, distance and angle of the light source, and the ultraviolet light field patterns 72. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 19. It is also important to note that the combination applicator 11, the control console/unit 22, the ultrasound generator 88, the fluid field generator 24 that directly controls the output of the fluid pump (FP) 24A, and the ultraviolet light generator (UVG) 74 are sharing a common power supply (PS) 23, which can be incorporated inside the control console/unit 22 (as presented in FIG. 1B, 7B, or 8B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the ultraviolet light generator (UVG) 74, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and ultraviolet light medical system 190 presented in FIG. 19. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the ultraviolet light field patterns 72. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, UV light type (UV light A (UVA), UV light B (UVB), UV light C (UVC) and vacuum UV), time of activation, intensity, distance and angle of the light source, ultraviolet light field patterns 72, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 19, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the ultraviolet light generator (UVG) 74 are similar to the ones presented before for FIGS. 1A-1B, 7A-7B, and 8A-8B.

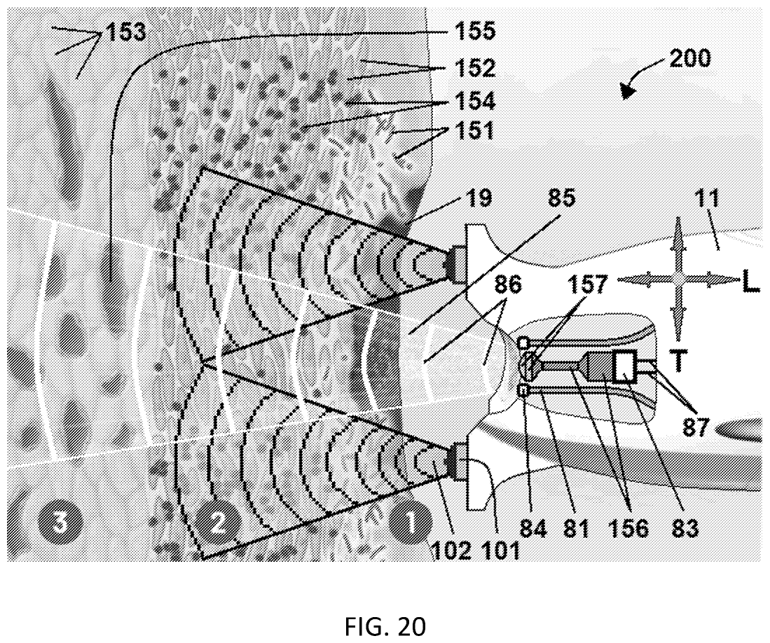

FIG. 20 shows the combination non-contact ultrasound and radio-frequency fields medical system 200 that is capable in the same device to produce non-contact ultrasound in combination with radio-frequency fields. These specially designed applicators and medical systems that use combination energy treatment regimens with energy-based medical technologies or combination devices/systems, can deliver simultaneously multiple energy-based technologies and incorporate besides non-contact ultrasound generating design elements (mist solution nozzle/generators, reflectors, ultrasound piezo ceramic/piezo crystal, acoustic horn, ultrasound-generating plate, etc.) also some specific components able to produce radio-frequency fields, which are delivered simultaneously with the ultrasound waves 86 for stimulation, healing, and regeneration of cells, tissues, or organs. In this case, the combination applicators 11 will have a special design that can facilitate a medical treatment using the non-contact ultrasound waves 86 in combination with radio-frequency fields from the outside/exterior of the human or animal body (extracorporeally) and towards the targeted treatment zone.

As seen from FIG. 20, the combination non-contact ultrasound and radio-frequency fields medical system 200 includes a combination applicator 11 that uses a combination power cable 34 together with a mist solution tubing 81 (where the mist solution tubing 81 is fused with or sits alongside the combination power cable 34) for connecting to the control console/unit 22 (where elements 22 and 34 are not shown for simplicity in FIG. 20, but they are similar to the ones shown in FIGS. 8A-8B). During treatment the user can move the combination applicator 11 along the longitudinal movement L and transversal movement T, to allow the covering of the entire targeted treatment area or region or wound/lesion/tissue condition 19 of the human/animal body 20 (not specifically identified in FIG. 20, but seen in other figures). The L and T movements can be done manually or automatically, and when they are performed automatically, these movements are controlled by the control console/unit 22 (not shown in FIG. 20) that actuates an external motorized ancillary system (not specifically presented in any of the figures of this patent for simplicity), which also contains sensors, motors, cameras, and clamping mechanism for the combination applicator 11. From the axial and central portion of the combination applicator 11, the ultrasound waves 86 are transmitted via the ultrasound transmission mist solution field pattern 85 towards the wound/lesion/tissue condition 19 from the human/animal body 20. The mist solution that conducts the ultrasound towards the treatment targeted zone can be a saline solution or can incorporate different active fluid substances or specialized fluid medicine to help with the healing of the targeted area. The wound/lesion/tissue condition 19 can be positioned superficial as a breach of the dermis 1 and epidermis 2, or immediately under the epidermis 1, or under dermis 2 that contains the dermis cells 152, or under subcutaneous fat tissue 3 that contains large subcutaneous tissue fat cells 153, or deep inside the human/animal body 20. In and around the wound/lesion/tissue condition 19 one can find bacteria 151, inflammatory cells 154, or blood vessels 155. When excited by the voltage transmitted through the piezo ceramic/piezo crystal wiring 87, the ultrasound piezo ceramic/piezo crystal emission element 83 produces acoustic vibrations through the acoustic horn 156 that amplifies these piezo vibrations and transmits those to the ultrasound-generating plate 157, which is used to produce ultrasound waves 86 of meaningful amplitude. The ultrasound-generating plate 157 has a convex surface that can be part of a sphere, paraboloid, ellipsoid, conical, pyramidal, etc., or a combination of these geometries, which will dictate the three-dimensional distribution of the ultrasound waves 86 via the ultrasound transmission mist solution field pattern 85 inside the wound/lesion/tissue condition 19. In another embodiment, the ultrasound-generating plate 157 can be replaced by a reflector that has any solid concave geometry as ellipsoidal, parabolic, spherical, conical, pyramidal or any combination of them, which will be able to direct and/or focus the ultrasound waves 86 in a precise location of the wound/lesion/tissue condition 19 and avoid the radial/divergent pattern produced by the ultrasound-generating plate 157. One or two or more-mist solution nozzles/generators 84 are used to generate the ultrasound transmission mist solution field pattern 85, which is used to conduct the ultrasound waves 86 towards the wound/lesion/tissue condition 19. Once reaching the skin 21 or human body 20 through the mist solution field pattern 85 (elements 20 and 21 are not specifically identified in FIG. 20, but they can be seen in other figures of this invention), the ultrasound waves 86 continue to propagate through the epidermis 1, dermis 2, and subcutaneous fat tissue 3, deep inside the human body 20. The same is valid for animal or plant tissues. The ultrasound piezo ceramic/piezo crystal emission element 83 has a dedicated ultrasound piezo ceramic/piezo crystal wiring 87 and each mist solution nozzle/generator 84 has its own dedicated mist solution tubing 81. On the peripheral part of the combination applicator 11, at least two or three or more radio-frequency emission elements 101 are present, which are capable to create radio-frequency field patterns 102 inside the wound/lesion/tissue condition 19. In FIG. 20 there are two (2) radio-frequency emission element 101, each of them with a dedicated radio-frequency emission element wiring 103 (not shown specifically in FIG. 20, but shown in FIG. 10B). The radio-frequency emission elements 101 can be at a certain distance from the skin 21 or wound/lesion/tissue condition 19 (as seen in FIG. 20) or in another embodiment the radio-frequency emission elements 101 can be modified/designed to be positioned in direct contact with the skin 21 or wound/lesion/tissue condition 19. The combined effects of the non-contact ultrasound waves 86 that are transmitted via ultrasound transmission mist solution field pattern 85 and of the radio-frequency field patterns 102 will enhance and expedite the healing of the wound/lesion/tissue condition 19, which is beneficial for a successful medical treatment and thus becomes more financially viable and less time consuming for both the patients and the medical personnel.

For the good functionality of the combination non-contact ultrasound and radio-frequency fields medical system 200, the control console/unit 22 (not specifically shown in FIG. 20) should contain the ultrasound generator 88 that includes the ultrasound generator processor 8000, the ultrasound generator Bluetooth (BT) module 8010, the ultrasound generator Wi-Fi module 8020, the ultrasound generator RFID module 8030, the ultrasound generator I/O (input/output) element 8040, the ultrasound generator memory 8050, the ultrasound generator emission mechanism (EM) module 8060, the ultrasound generator timer module 8070, and ultrasound field generator (USG) module 8080, as seen in FIG. 8B. Also, the control console/unit 22 should contain the fluid field generator 24 that directly controls the output of the fluid pump (FP) 24A via the fluid pump processor 2000, the fluid pump I/O (input/output) element 2010, the fluid pump memory 2020, the fluid pump Bluetooth module 2030, the fluid pump Wi-Fi module 2040, and the fluid pump RFID module 2050, as presented before in FIG. 1B. Finally, the control console/unit 22 should also have the radio-frequency generator 104 that includes the radio-frequency field generator processor 9000, the radio-frequency field generator Bluetooth (BT) module 9010, the radio-frequency field generator Wi-Fi module 9020, the radio-frequency field generator RFID module 9030, the radio-frequency field generator I/O (input/output) element 9040, the radio-frequency field generator memory 9050, the radio-frequency field generator emission mechanism (EM) module 9060, the radio-frequency field generator timer module 9070, and the radio-frequency field generator (RFG) module 9080, as seen in FIG. 10B. For FIG. 20, the fluid pump processor 2000 has a slave-master relationship with the ultrasound generator processor 8000, which means that the ultrasound generator processor 8000 controls the activity of the fluid pump processor 2000 via a continuous interaction process of sharing information and commands. Similarly, the radio-frequency field generator processor 9000 has also a slave-master relationship with the ultrasound generator processor 8000, which means that the ultrasound generator processor 8000 controls also the activity of the radio-frequency field generator processor 9000. Through this process the ultrasound generator processor 8000 together with the fluid pump processor 2000 and the radio-frequency field generator processor 9000 control the ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, radio-frequency type (non-thermal radio frequency or thermal radio-frequency), time of activation, intensity, frequency, continuous or intermittent application, and the radio-frequency field patterns 102. The voltage that travels through combination power cable 34 is produced inside the control console/unit 22 by the power supply (PS) 23 (see FIGS. 8A and 8B) that is controlled and directly connected to the ultrasound generator 88 for the embodiment from FIG. 20. It is also important to note that the combination applicator 11, the control console/unit 22, the ultrasound generator 88, the fluid field generator 24 that directly controls the output of the fluid pump (FP) 24A, and the radio-frequency generator 104 are sharing a common power supply (PS) 23, which can be incorporated inside the control console/unit 22 (as presented in FIG. 1B, 8B, or 10B) or in some other embodiments can be an external power supply that is separate from the control console/unit 22. The role of the power supply (PS) 23 is to convert and provide enough energy/power to activate both the combination applicator 11 and the control console/unit 22 and its respective components. In other embodiment, multiple and different power supplies can be used for the ultrasound generator 88, for the fluid field generator 24 that directly controls the output of the fluid pump (FP) 24A, and for the radio-frequency generator 104, power supplies that can be internal to the control console/unit 22 or in other embodiment can be external to the control console/unit 22 as part of an independent module/ancillary system.

The ultrasound generator processor 8000 practically controls the whole activity of the combination non-contact ultrasound and radio-frequency fields medical system 200 presented in FIG. 20. Thus, the combination applicator 11 receives the system control information from the ultrasound generator processor 8000, via a secured data transfer protocol from the control console/unit 22, for generating a selected/optimized number of combination energy medical treatments, by utilizing a selected/optimized amount of energy, as determined by the one or more combination treatment settings for the ultrasound transmission mist solution field pattern 85, non-contact ultrasound waves 86, and the radio-frequency field patterns 102. The data transfer protocol in between the control console/unit 22 and combination applicator 11 is secured via an authentication process that includes processor generated passcodes and passwords, authentication process that is performed when the combination power cable 34 and mist solution tubing 81 are connected to the control console/unit 22 and the power is “On” for the control console/unit 22. The ultrasound generator processor 8000 will also monitor the treatment parameters for their consistency and repeatability during the actual treatment delivery. The final number of energy combination energy medical treatments, ultrasound type (low, medium, or high frequency), mist solution and ultrasound time of activation, continuous or intermittent application of mist solution and ultrasound, ultrasound intensity/amplitude, ultrasound wavelength, mist solution pressure, mist solution spraying particle size, mist solution or solutions choice, mixing of different mist solutions and their sequence of pumping, ultrasound transmission mist solution field pattern 85, radio-frequency type (non-thermal radio frequency or thermal radio-frequency), time of activation, intensity, frequency, continuous or intermittent application, radio-frequency field patterns 102, type and serial number of combination applicator 11, type and serial number of the control console/unit 22, date, timing, financial info about the treatment for reimbursement, name of facility performing the treatment, region where the treatment was performed, etc. are all processed by the ultrasound generator processor 8000 and then recorded in the ultrasound generator memory 8050, at the end of the treatment. The control console/unit 22 can also use the ultrasound generator processor 8000 to transmit the treatment data and parameters to an artificial intelligence (A/I) device (not specifically shown in figures of patent invention) for processing by the artificial intelligence (A/I) device processor, or for storage into the artificial intelligence (A/I) device memory, or to be sent via an artificial intelligence (A/I) device Wi-Fi module towards the internet based data storage/cloud database or its associated artificial intelligence (AI) server. In another embodiment, the treatment data and parameters monitored by the ultrasound generator processor 8000 and recorded in the ultrasound generator memory 8050 can be sent directly from the control console/unit 22 to the internet-based data storage/cloud database and its associated artificial intelligence (AI) server, via the ultrasound generator Bluetooth (BT) module 8010, or the ultrasound generator Wi-Fi module 8020, or the ultrasound generator RFID module 8030, as presented in FIG. 8B. The functions of the artificial intelligence (AI) device are the same as the ones presented for FIGS. 1A and 1B.

Although not specifically shown in FIG. 20, for the embodiment presented in this figure the ultrasound generator 88 of the control console/unit 22 has the purpose, structure, construction, usability, and functionality similar to the shockwaves/pressure waves field generator 9 from the FIGS. 1A-10B. Also, in general, the purpose, structure, construction, usability, and functionality of the elements of the fluid field generator 24, of the ultrasound generator 88, and of the radio-frequency generator 104 are similar to the ones presented before for FIGS. 1A-1B, 8A-8B, and 10A-10B.

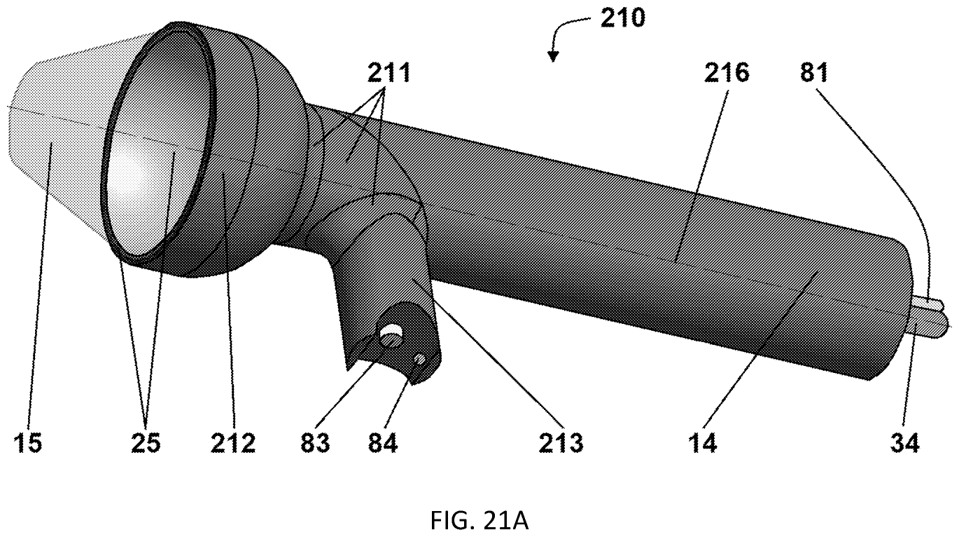

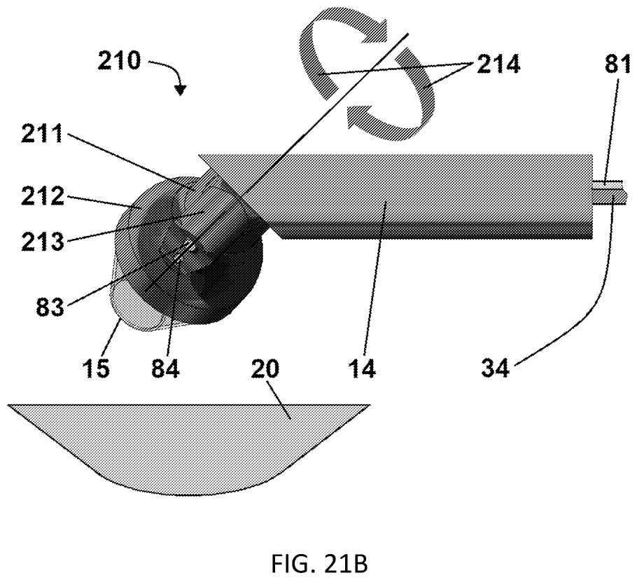

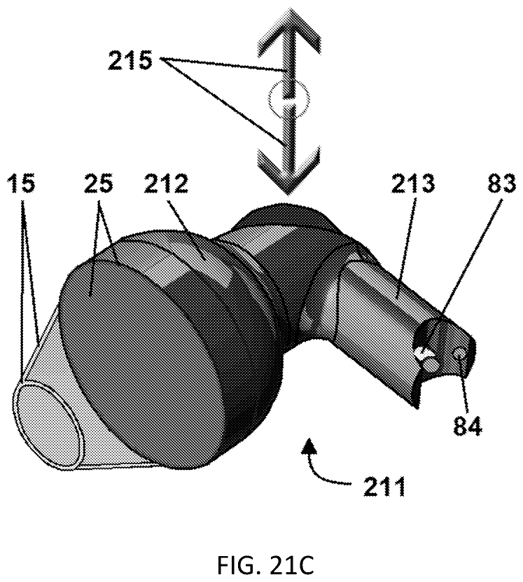

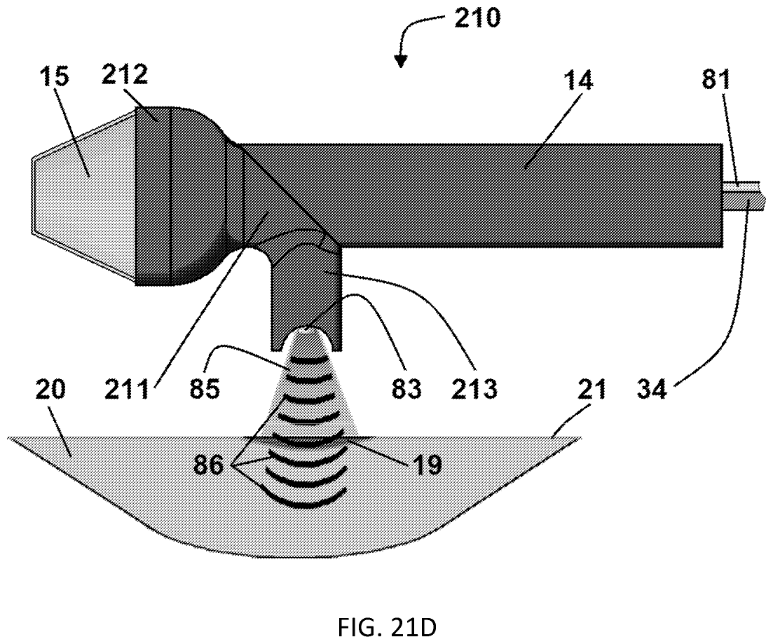

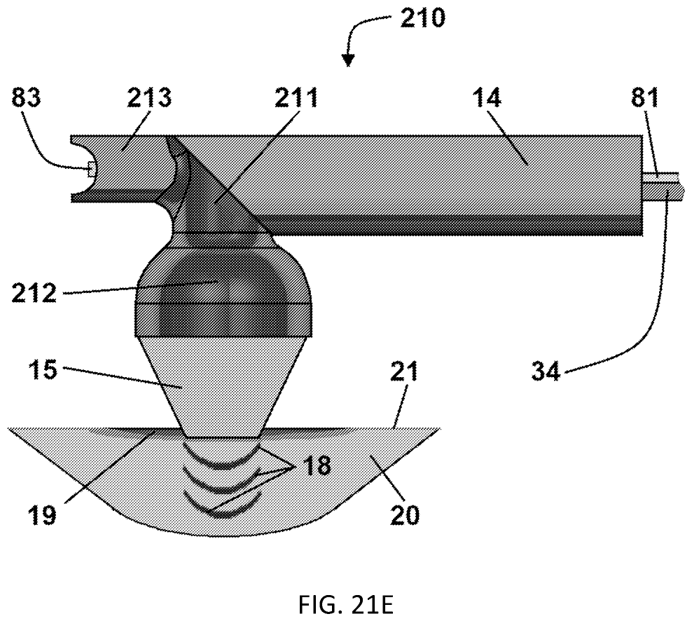

In embodiments from FIGS. 1A-20, the applicators and medical systems that use combination energy treatment regimens with energy-based medical technologies or combination devices/systems have their design elements for different energy technologies incorporated in a relative fixed position on the combination applicators 11 and could be used mostly in the same time or sometimes subsequently activated. In FIGS. 21A-21E is presented an embodiment that uses another approach, where two or more revolving and independent applicators/heads, capable of delivering different combination energy therapies, are mounted on one applicator body 14 and can be used only on subsequently manner. The revolving combination shockwaves/pressure waves and non-contact ultrasound medical system 210 has a revolving dual head 211 that includes a shockwave/pressure wave head 212 and a non-contact ultrasound head 213, which can deliver subsequently either shockwaves/pressure waves 18 (see FIG. 21E) or non-contact ultrasound waves 86 via an ultrasound transmission mist solution field pattern 85 (see FIG. 21D). The mist solution that conducts the ultrasound towards the treatment targeted zone can be a saline solution or can incorporate different active fluid substances or specialized fluid medicine to help with the healing of the targeted area. The desired position of different heads relatively to the applicator body 14 is accomplished using the revolving movement 214, as seen in FIG. 21B. In FIG. 21A the shockwave/pressure wave head 212 is locked in a position along/coaxially with the longitudinal axis 216 of the applicator body 14 of the revolving combination shockwaves/pressure waves and non-contact ultrasound medical system 210. In this position the non-contact ultrasound head 213 is oriented perpendicularly to the longitudinal axis 216 of the applicator body 14. For the embodiments presented in FIGS. 21A-21E, the combination shockwaves/pressure waves and non-contact ultrasound medical system 210 is used to treat wound/lesion/tissue condition 19 positioned laterally from the longitudinal axis 216 of the applicator body 14. During the treatment, the combination shockwaves/pressure waves and non-contact ultrasound medical system 210 will be held in a horizontal position relatively to the human/animal body 20 and wound/lesion/tissue condition 19 (see FIGS. 21D and 21E). However, in other embodiments the treatments could be applied with the head that is coaxial with the longitudinal axis 216 of the applicator body 14, situation where the combination shockwaves/pressure waves and non-contact ultrasound medical system 210 will be held vertically and perpendicularly to the wound/lesion/tissue condition 19 from the human/animal body 20. Due the versatility and symmetry of the combination shockwaves/pressure waves and non-contact ultrasound medical system 210, in yet another embodiment it can be used in either horizontal or vertical position during treatment, based on the position of the wound/lesion/tissue condition 19 from the human/animal body 20, to allow the choosing of the easiest access to the wound/lesion/tissue condition 19 for the medical personnel/healthcare worker performing the procedure or therapy. For all the embodiments presented in FIGS. 21A-21E the combination energy treatments are performed using different energy-based technologies present on one device/systems, as the combination shockwaves/pressure waves and non-contact ultrasound medical system 210, but they can be performed only in a subsequent and independent manner. This is different from the systems presented in FIGS. 1A-20, where multiple energy therapies could be used mostly concurrently and sometimes subsequently.

The revolving dual head 211 of the revolving combination shockwaves/pressure waves and non-contact ultrasound medical system 210 is designed to be mechanically snapped in place on the applicator body 14 via the revolving dual head assembly snapping motion 215, as seen in FIG. 21C. When snapped in place on the applicator body 14, the revolving dual head 211 will also have means to electrically connect to the combination power cable 34 and the mist solution tubing 81 (see FIGS. 21A-21B and 21D-21E) that are connected to a control console/unit 22 similar in functionality and construction to the one presented in FIG. 8B.

Although not specifically shown in FIGS. 21A-21E, to accomplish the proper coverage of the wound/lesion/tissue condition 19 the combination shockwaves/pressure waves and non-contact ultrasound medical system 210 is moved during treatment along the longitudinal movement L and transversal movement T (similarly to the embodiments presented in FIGS. 1A-20), to allow the covering of the entire targeted treatment area or region or wound/lesion/tissue condition 19 of the human/animal body 20. The L and T movements can be done manually or automatically, and when they are performed automatically, these movements are controlled by the control console/unit 22 (not shown in FIGS. 21A-21E) that actuates an external motorized ancillary system (not specifically presented in any of the figures of this patent for simplicity), which also contains sensors, motors, cameras, and clamping mechanism for the combination applicator 11.

FIGS. 21D-21E show how the revolving combination shockwaves/pressure waves and non-contact ultrasound medical system 210 is used to perform a treatment. In the sequence presented in these figures, first the non-contact ultrasound head 213 is used to produce non-contact ultrasound waves 86 that are transmitted via the ultrasound transmission mist solution field pattern 85 (see FIG. 21D). The non-contact ultrasound waves 86 are produced by the ultrasound piezo ceramic/piezo crystal emission element 83 and the ultrasound transmission mist solution field pattern 85 is produced by the mist solution nozzle/generator 84 that are similar in structure and functionality to the ones presented in FIGS. 8A-8B and 15-20 (when an acoustic horn 156 and ultrasound-generating plate 157 are present). After finishing the treatment of the wound/lesion/tissue condition 19 with the non-contact ultrasound waves 86, the operator will use the revolving movement 214 to put in the right position the shockwave/pressure wave head 212. At this point in time, the applicator/coupling membrane 15 is in contact (via an ultrasound gel or a hydrogel or a liquid substance, which are not specifically shown in any of the figures of this invention) with the wound/lesion/tissue condition 19 or the skin 21 of the human/animal body 20, and the revolving combination shockwaves/pressure waves and non-contact ultrasound medical system 210 is ready to perform the subsequent treatment with the shockwaves/pressure waves 18 that are directed towards the wound/lesion/tissue condition 19 using the reflector 25 (see FIG. 21E). In this way a combination energy treatment or therapy is performed in a sequentially, which allows the application of the shockwaves/pressure waves 18 in combination and sequentially with non-contact ultrasound waves 86 from the outside/exterior of the human or animal body (extracorporeally) and towards the wound/lesion/tissue condition 19 from the human/animal body 20. In another embodiment, the shockwaves/pressure waves 18 could be applied first and then followed by the non-contact ultrasound waves 86.

Although in FIGS. 21A-21E the two heads/stations/spots were used to deliver shockwaves/pressure waves 18 or non-contact ultrasound waves 86 via an ultrasound transmission mist solution field pattern 85, other combinations of energy technologies can be used in a revolving combination system. Thus, in such combination systems/devices on the applicator body 14 it is possible to have two or more heads/stations/spots that can deliver shockwaves, or pressurized active substances, or electric currents, or electromagnetic fields, or ultrasound (contact or non-contact), or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or acupuncture, or cold atmospheric plasma, to name a few.

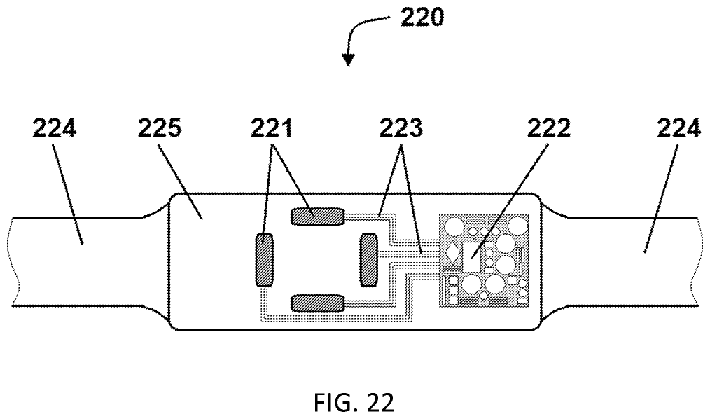

In another embodiment or embodiments, combination treatments with energy-based medical technologies are combined in the form of a combination energy therapy protocol that is using different energy-based technologies in a subsequent and independent manner, to maintain the upregulation/boost of benefic factors or inhibition of harmful factors in between different treatment sessions produced by a main energy system that is used for stimulation, healing, and regeneration of cells, tissues, or organs. For that, any of the embodiments presented in FIGS. 1A-21E can be used in different days or separate treatment sessions, which requires the patients to come often to the medical facilities or hospitals. For the convenience of the patients and of the medical staff, new and different energy generating medical devices can be used in the form of smart bandages that can be applied immediately after the main treatment sessions performed with any of the systems/devices presented in FIGS. 1A-21E and can stay in place for days or a week or many weeks. The smart bandages could be kept in place in the comfort of the home environment for days or a week or many weeks, until the next subsequent treatment session is scheduled to be perform at the medical office/hospital with any of the main systems/devices presented in FIGS. 1A-21E. This is convenient for the patients (avoids going to the medical facility too often) and still accomplishes to maintain the upregulation/boost of benefic factors or inhibition of harmful factors produced by subsequent main energy treatment sessions performed at intervals of few days, or a week or multiple weeks with any of the systems/devices presented in FIGS. 1A-21E, for stimulation, healing, and regeneration of cells, tissues, or organs. Such smart bandages can deliver their energy therapy continuously or more preferably intermittently to the wound/lesion/tissue condition 19 or organs. Preferably, all the smart bandage embodiments associated with FIGS. 22-27 must be battery operated and should not require any charging or recharging of the batteries or exchange of such batteries by the patient when such smart bandages are used at home. The batteries can also be made to recharge and produce small therapeutic electrical pulses and electric fields necessary to operate the smart bandage, from converting normal body parts or skin movements that occur during usual day-to-day activities or during normal breathing (kinetic recharging), or from using body heat through a responsive resistor/thermistor and skin exudates to generate chemical reactions in electrochemically active materials capable of producing small therapeutic electrical pulses and electric fields (electrochemical recharging). This type of bandage or patch can be used for treating skin and tissues underneath the skin for acute or chronic lesions or for cosmetic purposes and to fight bacterial biofilm infections. In general, this type of bandage is appropriate to treat any type of soft, semi-soft, and hard tissues that are superficial or deep inside the human or animal body.

In an alternative embodiment where the smart bandage associated with FIGS. 22-27 might be used intensively for long periods of time, they can be provided with simple charging means or use wireless charging to replenish rechargeable batteries during rest periods for the patient, when such activity is not a big inconvenience. Alternatively, when such bandages are used intensively for long periods of time, the patient can use subsequently two or more bandages that are battery-operated and do not require charging means. Regardless of the approach taken by the health care professionals and the patient, the smart bandage associated with FIGS. 22-27 should have means (optical or auditive) to indicate to the patient that the respective smart bandages have their batteries depleted.

Also, the smart bandages should be made with mechanical, electric, or electronic components that are able to sustain one sterilization process (gamma radiation, Ethylene Oxide gas, etc.) and should be a single use device/bandage. Other requirements for smart bandages are related to the materials used in their construction that should be hypoallergenic, biocompatible, resistant to fluids, self-cleaning, antibacterial, with mechanical resistance and elasticity, electrical conductance, resistance to electromagnetic interference, reduced or controlled liquid absorption, good adherence to the skin, and recyclable, to name a few. All the smart bandages should be extracorporeal and designed to be easily and temporarily attached or secured to the patient skin or bodily limbs or any appendages, depending on the position of the targeted wound/lesion/tissue condition 19. These smart bandages should also have easy and intuitive means to activate them for the delivery of their energy therapy to the wound/lesion/tissue condition 19. In the same way, one or multiple light emitting diodes (LEDs) or organic light emitting diodes (OLEDs) should be incorporated into the smart bandage to indicate that the bandage is functionally active or into inactive periods and also to show its state during the time of the energy delivery to the wound/lesion/tissue condition 19. Overall, the electronics incorporated in such smart bandages should be relatively simple, reliable, and efficient in delivery the proper energy therapy. Although not shown in any of the FIGS. 22-27, some embodiments for the smart bandages can contain Bluetooth or Wi-Fi or radio frequency identification device (RFID) that are able to communicate with a specially designed, independent, and external associated monitoring device, or a tablet, or a laptop/computer/workstation, or a cellphone, or an app, etc., to download treatment functionality parameters that can be copied by the medical personnel when the smart bandage is removed after its usage. The Bluetooth or Wi-Fi or radio frequency identification device (RFID) associated with smart bandages can also be used by the patient to live-monitoring of the active periods of such bandages. The embodiments of smart bandages with external communication means should also have the capacity to be preprogramed before their usage with different possible protocols (based on the specific targeted wound/lesion/tissue condition 19), which can be stored in a library of protocols on the specially designed, independent, and external associated monitoring device, or on a tablet, or on a laptop/computer/workstation, or on a cellphone, or on an app, etc. To do such preprogramming the Bluetooth or Wi-Fi or radio frequency identification device (RFID) are necessary to be present on such smart bandage.

FIGS. 22-27 will only show the construction and functionality of different smart bandages that incorporate diverse energy technologies. These figures do not show the actual usage and application of the smart bandages to the skin 21 or targeted wound/lesion/tissue condition 19 and the position of the wound/lesion/tissue condition 19 relatively to the skin 21 or inside the human/animal body 20. However, these elements that were seen in other embodiments presented in FIGS. 1A-21E will be mentioned in the following paragraphs that describe the construction and functionality of the smart bandages presented in FIGS. 22-27.

Thus, in FIG. 22 is presented a smart bandage with electrical currents 220 that is capable to produce electrical stimulation with low intensity direct current—LIDC, or high voltage pulsed current—HVPC, or asymmetrical biphasic pulsed current—ABPC, or alternative current—AC, or transcutaneous electrical nerve stimulation (TENS), or interferential current therapy (IFC), etc., in between main/primary treatment sessions that are performed with any of the energy systems/embodiments presented in this invention or by using independent energy devices (incorporating only one energy technology that are presented in other patents). The electrical current activation for treatment can be done continuously or intermittently following a certain pre-programmed protocol that assures sufficient wound/lesion/tissue condition 19 stimulation and which is also efficiently covered by the capacity of the batteries incorporated in such bandages. This smart bandage with electrical currents 220 can be used in between the main/primary treatment sessions with shockwave/pressure waves (focused, unfocused, planar, pseudo-planar, or radial), or pressurized active substances, or electromagnetic fields, or ultrasound waves (contact or non-contact), or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the electric currents, the smart bandage with electrical currents 220 contains the electrical current micro-electrodes 221 that are activated continuously or intermittently by the electrical current generator and electronics 222 via the electrical connectors 223. In the embodiment from FIG. 22, the number of electrical current micro-electrodes 221 is four (4). However, two (2), or four (4), or more paired (in pairs of one anode and one cathode) electrical current micro-electrodes 221 can be used, depending on the size and specific targeted wound/lesion/tissue condition 19 and its position inside the human/animal body 20. The electrical current generator and electronics 222 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these possible elements of the electrical current generator and electronics 222 and the electrical connectors 223 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the electrical current micro-electrodes 221 must be exposed during usage to the direct contact with the skin 21 or targeted wound/lesion/tissue condition 19. Before the usage of the bandage, the electrical current micro-electrodes 221 can be protected with a protective adherent foil that can be removed before the activation and usage of the smart bandage with electrical currents 220. For the easy-use and attachment to the skin or around a bodily limbs or appendances, the smart bandage with electrical currents 220 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain at least two, four or more electrical current micro-electrodes 221, the electrical current generator and electronics 222, and the associated electrical connectors 223. The wound/lesion/tissue condition smart bandage coverage area 225 is the region or area of the bandage where the generated electrical currents can be active and effective in treating the wound/lesion/tissue condition 19. The smart bandage has specific markings (not shown in the FIG. 22 for simplicity of the figure) to clearly indicate the position of the smart bandage active elements relatively to the smart bandage coverage area 225, to allow the correct position of the electrical current micro-electrodes 221 in such way to provide complete/full overlap of the produced electric fields with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage with electrical currents 220, based on the specific medical treatment, or the size of the limb, or appendage, or wound/lesion/tissue condition 19.

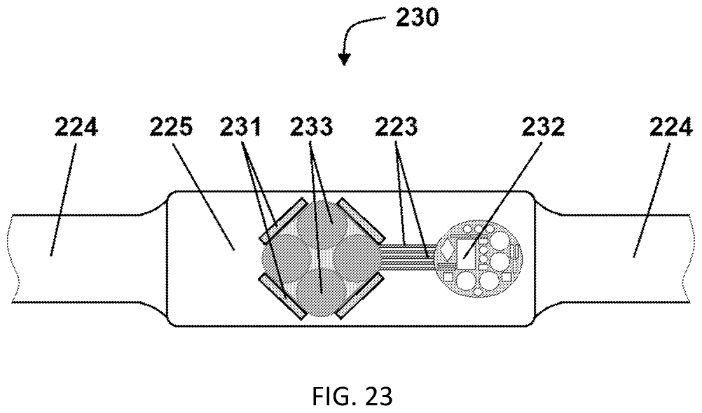

In FIG. 23 is presented a smart bandage with electrolysis electrodes for producing oxygen/oxygen species 230 that is capable to produce low intensity direct currents to generate oxygen and oxygen species in oxygen delivery patches 233 that are initially filled with an aqueous solution with sodium hydroxide (NaOH), or sodium chloride, or salts, or baking soda, etc., to assure a good current conduction in water/aqueous solution from the oxygen delivery patches 233, where the electrolytic process takes place. The oxygen and oxygen species generated by the smart bandage with electrolysis electrodes for producing oxygen/oxygen species 230 are used in between main/primary treatment sessions that are performed with any of the energy systems/embodiments presented in this invention or using independent energy devices (incorporating only one energy technology that are presented in other patents). Such smart bandages with electrolysis electrodes for producing oxygen/oxygen species 230 can produce and deliver continue or intermittent oxygen or oxygen species to a wound/lesion/tissue condition 19 in between visits to the physician's office where other energy treatments can be applied as main/primary treatments. This provides a new mechanism of action and practically a mini-hyperbaric therapy. The oxygen and oxygen species can migrate or being absorbed inside the tissue. In other cases, the oxygen can be pushed inside the desired areas (skin, wounds, different tissues under the skin or deeper, etc.) using ultrasound, shockwaves, pressure, mechanical force, radio frequency, currents, electromagnetic fields, etc. Such smart bandages/dressings with a continuous or intermittent diffusion of oxygen can be effective to improve the chance of successful wound/lesion/tissue condition 19 healing and to fight bacteria or bacterial biofilm infections.

For this specific smart bandage, it is interesting to note that the electric currents used for the electrolysis process can also do an additional overlapping electric stimulation of the wound/lesion/tissue condition 19, which is dependent on the current intensity used and the position of the wound/lesion/tissue condition 19 relatively to the skin 21. The electrical stimulation, for producing via electrolysis process the oxygen/oxygen species, can be generated continuously or intermittently following a certain pre-programmed protocol that assures sufficient wound/lesion/tissue condition 19 stimulation and which is also efficiently covered by the capacity of the batteries incorporated in such bandages. This smart bandage with electrolysis electrodes for producing oxygen/oxygen species 230 can be used in between the main/primary treatment sessions with shockwave/pressure waves (focused, unfocused, planar, pseudo-planar, or radial), or pressurized active substances, or electric current fields, or electromagnetic fields, or ultrasound waves (contact or non-contact), or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the electric currents used for the electrolysis process, the smart bandage with electrolysis electrodes for producing oxygen/oxygen species 230 contains the electrolysis micro-electrodes 231 that are activated continuously or most likely intermittently by the electrolysis currents generator and electronics 232, via the electrical connectors 223. The electrolysis micro-electrodes 231 are used to initiate the electrolysis processes in the oxygen delivery patches 233 from where the oxygen is released in the wound/lesion/tissue condition 19. In the embodiment from FIG. 23, the number of electrolysis micro-electrodes 231 is four (4). However, two (2), or four (4), or more paired (in pairs of one anode and one cathode) electrolysis micro-electrodes 231 can be used, depending on the size and specific targeted wound/lesion/tissue condition 19 and its position inside the human/animal body 20. Similarly, in the embodiment from FIG. 23 the number of oxygen delivery patches 233 is four (4). However, two (2), or four (4), or more oxygen delivery patches 233 can be used, depending on the number of paired electrolysis micro-electrodes 231, the size and specific targeted wound/lesion/tissue condition 19, and its position inside the human/animal body 20. The electrolysis currents generator and electronics 232 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these elements of the electrolysis currents generator and electronics 232, the electrical connectors 223, and the electrolysis micro-electrodes 231 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the oxygen delivery patches 233 must be exposed during usage to the direct contact with the skin 21 or targeted wound/lesion/tissue condition 19, to be able to release oxygen or oxygen species in the targeted treatment region/area. Before the usage of the bandage, the oxygen delivery patches 233 can be protected with a protective adherent foil that can be removed before the activation and usage of the smart bandage with electrolysis electrodes for producing oxygen/oxygen species 230. For the easy-use and attachment to the skin or around a bodily limbs or appendances, the smart bandage with electrolysis electrodes for producing oxygen/oxygen species 230 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain at least two, four or more electrolysis micro-electrodes 231, the electrolysis currents generator and electronics 232, the oxygen delivery patches 233, and the associated electrical connectors 223. The wound/lesion/tissue condition smart bandage coverage area 225 is the region or area of the bandage where the generated oxygen or oxygen species can be active and effective in treating the wound/lesion/tissue condition 19. For the smart bandage with electrolysis electrodes for producing oxygen/oxygen species 230, the wound/lesion/tissue condition smart bandage coverage area 225 should have adhesive all around its perimeter to adhere to the skin 21, which will prevent the oxygen or oxygen species to escape in the exterior/outside of the bandage instead of being absorbed by the wound/lesion/tissue condition 19. The adhesive portion of the wound/lesion/tissue condition smart bandage coverage area 225 should be kept to the minimal size, to avoid any allergenic reaction and easy detachment from the skin at the end of the treatment. The smart bandage has specific markings (not shown in the FIG. 23 for simplicity of the figure) to clearly indicate the position of the smart bandage active elements relatively to the smart bandage coverage area 225, to allow the correct position of the oxygen delivery patches 233 in such way to provide complete/full overlap of the oxygen delivery area with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage with electrolysis electrodes for producing oxygen/oxygen species 230, based on the specific medical treatment, or the size of the limb, or appendage, or wound/lesion/tissue condition 19.

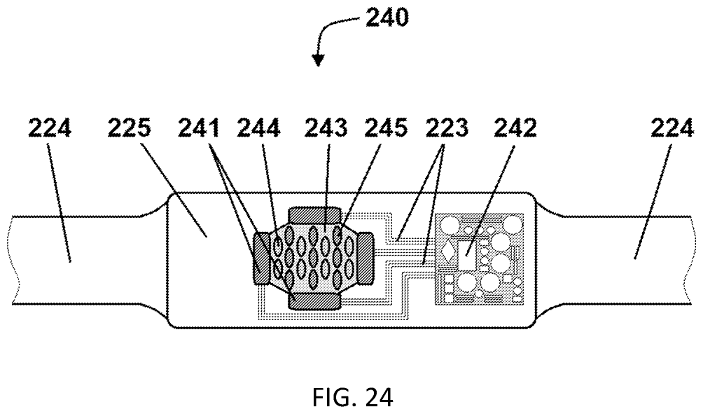

In FIG. 24 is presented a smart bandage for drug delivery using electric currents 240 that is capable to produce electrical stimulation with low intensity direct current—LIDC, or high voltage pulsed current—HVPC, or asymmetrical biphasic pulsed current—ABPC, or alternative current—AC, etc., to activate the drug delivery, from the drug A small pockets 244 and the drug B small pockets 245, in between main/primary treatment sessions that are performed with any of the energy systems/embodiments presented in this invention or using independent energy devices (incorporating only one energy technology that are presented in other patents). For this specific smart bandage, it is interesting to note that the electric currents used for the drug delivery activation process can also do an additional overlapping electric stimulation of the wound/lesion/tissue condition 19, which is dependent on the current intensity used and the position of the wound/lesion/tissue condition 19 relatively to the skin 21. The electrical stimulation for drug activation can be generated continuously or intermittently following a certain pre-programmed protocol that assures sufficient wound/lesion/tissue condition 19 stimulation and which is also efficiently covered by the capacity of the batteries incorporated in such bandages. This smart bandage for drug delivery using electric currents 240 can be used in between the main/primary treatment sessions with shockwave/pressure waves (focused, unfocused, planar, pseudo-planar, or radial), or pressurized active substances, or electric current fields, or electromagnetic fields, or ultrasound waves (contact or non-contact), or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the electric currents used for the drug delivery process, the smart bandage for drug delivery using electric currents 240 contains the drug delivery electric micro-electrodes 241 that are activated continuously or most likely intermittently by the drug delivery current generator and electronics 242, via the electrical connectors 223. The drug delivery electric micro-electrodes 241 are used to initiate the drug delivery processes into the wound/lesion/tissue condition 19, by melting/puncturing different drug A small pockets 244 and/or drug B small pockets 245 from the drug delivery patch 243. In the embodiment from FIG. 24, the number of drug delivery electric micro-electrodes 241 is four (4). However, two (2), or four (4), or more paired (in pairs of one anode and one cathode) drug delivery electric micro-electrodes 241 can be used, depending on the size and specific targeted wound/lesion/tissue condition 19 and its position inside the human/animal body 20. Similarly, in the embodiment from FIG. 24 there are two different drugs (drug A and drug B) that can be delivered from drug A small pockets 244 and/or drug B small pockets 245. However, one (1), or two (2), or three (3), or four (4), or more different drugs can be used, depending on the dimension of the drug delivery patch 243, type of affliction, the size and specific targeted wound/lesion/tissue condition 19, and its position inside the human/animal body 20. The drug delivery current generator and electronics 242 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these elements of the drug delivery current generator and electronics 242, the electrical connectors 223, and the drug delivery electric micro-electrodes 241 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the drug delivery patch 243 must be exposed during usage to the direct contact with the skin 21 or targeted wound/lesion/tissue condition 19, to be able to activate the drug(s) delivery in the targeted treatment region/area. Before the usage of the bandage, the drug delivery patch 243 can be protected with a protective adherent foil that can be removed before the activation and usage of the smart bandage for drug delivery using electric currents 240. For the easy-use and attachment to the skin or around a bodily limbs or appendances, the smart bandage for drug delivery using electric currents 240 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain at least two, four or more drug delivery electric micro-electrodes 241, the drug delivery current generator and electronics 242, the drug delivery patch 243, and the associated electrical connectors 223. The wound/lesion/tissue condition smart bandage coverage area 225 is the region or area of the bandage where the drug delivery is produced and is effective in treating the wound/lesion/tissue condition 19. For the smart bandage for drug delivery using electric currents 240, the wound/lesion/tissue condition smart bandage coverage area 225 should have adhesive all around its perimeter to adhere to the skin 21, which will prevent the drug(s) to escape in the exterior/outside of the bandage instead of being absorbed by the wound/lesion/tissue condition 19. The adhesive portion of the wound/lesion/tissue condition smart bandage coverage area 225 should be kept to the minimal size, to avoid any allergenic reaction and easy detachment from the skin at the end of the treatment. The smart bandage has specific markings (not shown in the FIG. 24 for simplicity of the figure) to clearly indicate the position of the smart bandage active elements relatively to the smart bandage coverage area 225, to allow the correct position of the drug delivery patch 243 in such way to provide complete/full overlap of the drug delivery area with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage for drug delivery using electric currents 240, based on the specific medical treatment, or the size of the limb, or appendage, or wound/lesion/tissue condition 19.

For the smart bandage for drug delivery using electric currents 240, specially designed sensors might be used in the smart bandage that measure the skin temperature, pH, sweat, blood oxygen saturation, etc., which transmit their readings to a microprocessor/processor, which may trigger an electric current through drug delivery electric micro-electrodes 241 or in other designs for the smart bandage through heating elements. or high intensity heating ultrasound or radio-frequency pulses or electromagnetic fields to produce moderate heat that is used to release a drug or an active substance from the smart bandage.

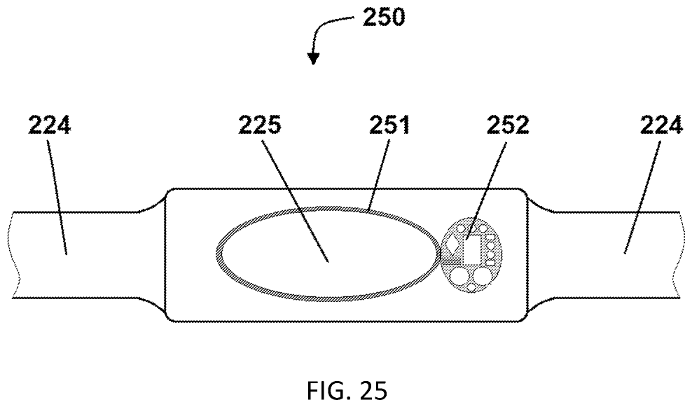

In FIG. 25 is presented a smart bandage with electromagnetic field(s) 250 that is capable to produce electromagnetic fields, which are well known to induce pain modulation and tissue regeneration. The electromagnetic fields generated by the smart bandage with electromagnetic field(s) 250 are used in between main/primary treatment sessions that are performed with any of the energy systems/embodiments presented in this invention or using independent energy devices (incorporating only one energy technology that are presented in other patents). The electromagnetic fields can be generated continuously or intermittently following a certain pre-programmed protocol that assures sufficient wound/lesion/tissue condition 19 stimulation and which is also efficiently covered by the capacity of the batteries incorporated in such bandages. This smart bandage with electromagnetic field(s) 250 can be used in between the main/primary treatment sessions with shockwave/pressure waves (focused, unfocused, planar, pseudo-planar, or radial), or pressurized active substances, or electric current fields, or ultrasound waves (contact or non-contact), or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the electromagnetic fields, the smart bandage with electromagnetic field(s) 250 contains the electromagnetic loop(s) 251 that are activated continuously or most likely intermittently by the electromagnetic field generator and electronics 252. In the embodiment from FIG. 25, is presented only one (1) electromagnetic loop 251. However, two (2), or three (3), or more electromagnetic loop(s) 251 can be used, depending on the size and specific targeted wound/lesion/tissue condition 19 and its position inside the human/animal body 20. The electromagnetic field generator and electronics 252 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these elements of the electromagnetic field generator and electronics 252 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the electromagnetic loop(s) 251 must be exposed during usage to the direct contact with the skin 21 or targeted wound/lesion/tissue condition 19, to be able to produce the associated electromagnetic fields in the targeted treatment region/area. Before the usage of the bandage, the electromagnetic loop(s) 251 can be protected with a protective adherent foil that can be removed before the activation and usage of the smart bandage with electromagnetic field(s) 250. For the easy-use and attachment to the skin or around a bodily limbs or appendances, the smart bandage with electromagnetic field(s) 250 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain the electromagnetic field generator and electronics 252 and the electromagnetic loop(s) 251. The wound/lesion/tissue condition smart bandage coverage area 225 is the region or area of the bandage where the generated electromagnetic fields can be active and effective in treating the wound/lesion/tissue condition 19. The smart bandage has specific markings (not shown in the FIG. 25 for simplicity of the figure) to clearly indicate the position of the smart bandage active elements relatively to the smart bandage coverage area 225, to allow the correct position of the electromagnetic loop(s) 251 in such way to provide complete/full overlap of the electromagnetic fields with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage with electromagnetic field(s) 250, based on the specific medical treatment, or the size of the limb, or appendage, or wound/lesion/tissue condition 19.

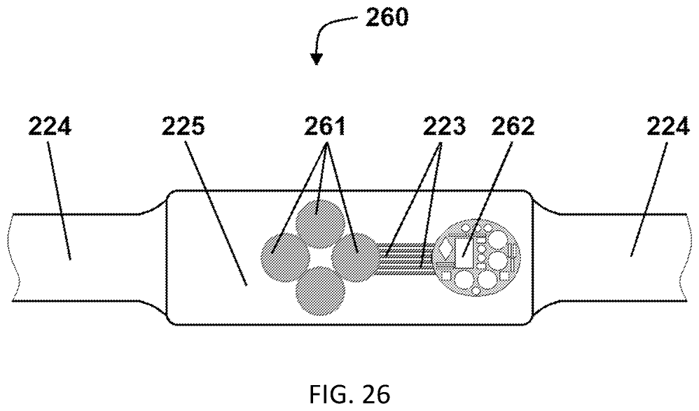

In FIG. 26 is presented a smart bandage with ultrasound fields 260 that is capable to produce direct contact ultrasound fields. The direct contact ultrasound fields generated by the smart bandage with ultrasound fields 260 are used in between main/primary treatment sessions that are performed with any of the energy systems/embodiments presented in this invention or using independent energy devices (incorporating only one energy technology that are presented in other patents). The direct contact ultrasound fields, can be generated continuously or intermittently following a certain pre-programmed protocol that assures sufficient wound/lesion/tissue condition 19 stimulation and which is also efficiently covered by the capacity of the batteries incorporated in such bandages. This smart bandage with ultrasound fields 260 can be used in between the main/primary treatment sessions with shockwave/pressure waves (focused, unfocused, planar, pseudo-planar, or radial), or pressurized active substances, or electric current fields, or electromagnetic fields, or non-contact ultrasound waves, or LED/OLED phototherapy, or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the direct contact ultrasound fields, the smart bandage with ultrasound fields 260 contains the ultrasound micro-piezo crystals/micro-piezo ceramics/micro-piezo fibers 261 that are activated continuously or most likely intermittently by the piezo ceramics/crystals/fibers current generator and electronics 262, via the electrical connectors 223. In the embodiment from FIG. 26, the number of ultrasound micro-piezo crystals/micro-piezo ceramics/micro-piezo fiber areas 261 is four (4). However, one (1), or two (2), or three (3), or four (4), or more ultrasound micro-piezo crystals/micro-piezo ceramics/micro-piezo fiber areas 261 can be used, depending on the size and specific targeted wound/lesion/tissue condition 19 and its position inside the human/animal body 20. The piezo ceramics/crystals/fibers current generator and electronics 262 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these elements of the piezo ceramics/crystals/fibers current generator and electronics 262 and the electrical connectors 223 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the ultrasound micro-piezo crystals/micro-piezo ceramics/micro-piezo fiber areas 261 must be exposed during usage to the direct contact with the skin 21 or targeted wound/lesion/tissue condition 19, to be able to produce direct contact ultrasound fields in the targeted treatment region/area. Before the usage of the bandage, the ultrasound micro-piezo crystals/micro-piezo ceramics/micro-piezo fiber areas 261 can be protected with a protective adherent foil that can be removed before the activation and usage of the smart bandage with ultrasound fields 260. For the easy-use and attachment to the skin or around a bodily limbs or appendances, the smart bandage with ultrasound fields 260 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain at least one (1), or two (2), or three (3), or four (4), or more ultrasound micro-piezo crystals/micro-piezo ceramics/micro-piezo fiber areas 261, the piezo ceramics/crystals/fibers current generator and electronics 262, and the associated electrical connectors 223. The wound/lesion/tissue condition smart bandage coverage area 225 is the region or area of the bandage where the generated direct contact ultrasound fields can be active and effective in treating the wound/lesion/tissue condition 19. The smart bandage has specific markings (not shown in the FIG. 26 for simplicity of the figure) to clearly indicate the position of the smart bandage active elements relatively to the smart bandage coverage area 225, to allow the correct position of the ultrasound micro-piezo crystals/micro-piezo ceramics/micro-piezo fiber areas 261 in such way to provide complete/full overlap of the direct contact ultrasound fields with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage with ultrasound fields 260, based on the specific medical treatment, or the size of the limb, or appendage, or wound/lesion/tissue condition 19.

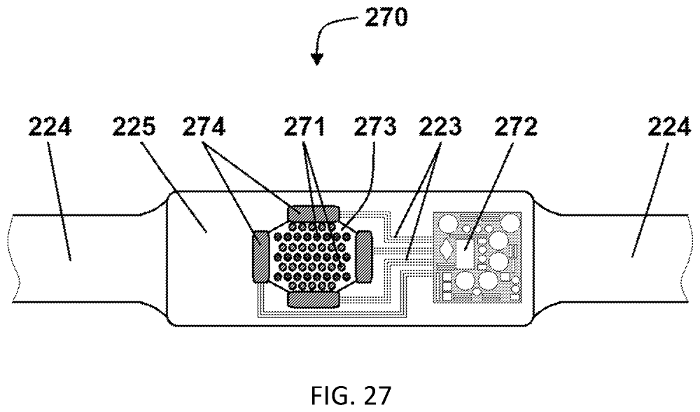

In FIG. 27 is presented a smart bandage with LED (light emitting diode)/OLED (organic light emitting diode) phototherapy fields 270 that is capable to produce LED/OLED phototherapy fields. The LED/OLED phototherapy fields generated by the smart bandage with LED/OLED phototherapy fields 270 are used in between main/primary treatment sessions that are performed with any of the energy systems/embodiments presented in this invention or using independent energy devices (incorporating only one energy technology that are presented in other patents). The LED/OLED phototherapy fields can be generated continuously or intermittently following a certain pre-programmed protocol that assures sufficient wound/lesion/tissue condition 19 stimulation and which is also efficiently covered by the capacity of the batteries incorporated in such bandages. This smart bandage with LED/OLED phototherapy fields 270 can be used in between the main/primary treatment sessions with shockwave/pressure waves (focused, unfocused, planar, pseudo-planar, or radial), or pressurized active substances, or electric current fields, or electromagnetic fields, or ultrasound waves (contact or non-contact), or photodynamic therapy, or laser phototherapy, or ultraviolet light, or radio frequency, or energy-activated nanotechnology, or hydrotherapy, or pneumatic compression, or topical negative pressures, or thermal therapy, or energy technology-generated acupuncture, or cold atmospheric plasma, or similar to any other embodiments mentioned in this invention, to keep the necessary upregulation/boost of benefic factors or inhibition of harmful factors for stimulation, healing, and regeneration of cells, tissues, or organs from the human/animal body 20.

To produce the LED/OLED phototherapy fields, the smart bandage with LED/OLED phototherapy fields 270 contains the LED/OLED actuators 274 that are activated continuously or most likely intermittently by the LED/OLED electrical current generator and electronics 272, via the electrical connectors 223. The LED/OLED actuators 274 are used to actuate the LED/OLED elements 271 from the phototherapy patch 273, and thus producing phototherapy fields in the wound/lesion/tissue condition 19. It is important to mention that the LED/OLED elements 271 can produce a certain light (white, blue, green, yellow, amber, red, or near infrared) or multiple colors in the same time or subsequently based on a certain pre-programmed protocol. In the embodiment from FIG. 27, the number of LED/OLED actuators 274 is four (4). However, two (2), or four (4), or more paired (in pairs of one anode and one cathode) LED/OLED actuators 274 can be used, depending on the size and specific targeted wound/lesion/tissue condition 19 and its position inside the human/animal body 20. The LED/OLED electrical current generator and electronics 272 includes the necessary batteries and other electronic elements as memories, one or more processors, amplifiers, resistors, transducers, transistors, capacitors, timers/clocks, feedback loops, electrical connections, heat sinks, LED/OLEDs functional indicators, audible means, Bluetooth or WiFi antennas, or an RFID chip, etc., depending on the complexity of such smart bandage and the specific energy technology or possible technologies used in its construction. All these elements of the LED/OLED electrical current generator and electronics 272, the electrical connectors 223, and the LED/OLED actuators 274 must be potted with flexible polymers or covered with Parylene or any other flexible plastic or composite coatings to protect them against any dust or external fluids, as bodily fluids, exudates, water, etc. and thus providing mechanical resistance, reduced electromagnetic interference, and prevent any electrical shorts. In contrast, the phototherapy patch 273 and its associated LED/OLED elements 271 must be exposed during usage to the direct contact with the skin 21 or targeted wound/lesion/tissue condition 19, to be able to completely illuminate/create the phototherapy fields in the targeted treatment region/area. Before the usage of the bandage, the phototherapy patch 273 and its associated LED/OLED elements 271 can be protected with a protective adherent foil that can be removed before the activation and usage of the smart bandage with LED/OLED phototherapy fields 270. For the easy-use and attachment to the skin or around a bodily limbs or appendances, the smart bandage with LED/OLED phototherapy fields 270 has the smart bandage straps 224, which are the portions of the bandage that have the smaller width and specific lengths, dimensions that are dictated by the type of treatment and by the size of the limb or appendage or by the location of the wound/lesion/tissue condition 19. It is important that the smart bandage straps 224 have enough area to assure secured adhesion of the smart bandage to the skin or around a bodily limbs or appendances, but in the same time the adhesion area should be kept to the minimal size, to avoid any allergenic reaction and easy detachment from the skin or limb or appendance at the end of the treatment. The wound/lesion/tissue condition smart bandage coverage area 225 is the area of the bandage that is larger in width and with a length dictated by the dimensions of the wound/lesion/tissue condition 19. The wound/lesion/tissue condition smart bandage coverage area 225 is designed in such way to contain at least two, four or more LED/OLED actuators 274, the LED/OLED electrical current generator and electronics 272, the phototherapy patch 273 and its associated LED/OLED elements 271, and the electrical connectors 223. The wound/lesion/tissue condition smart bandage coverage area 225 is the region or area of the bandage where the generated LED/OLED phototherapy fields can be active and effective in treating the wound/lesion/tissue condition 19. The smart bandage has specific markings (not shown in the FIG. 27 for simplicity of the figure) to clearly indicate the position of the smart bandage active elements relatively to the smart bandage coverage area 225, to allow the correct position of the phototherapy patch 273 in such way to provide complete/full overlap of the LED/OLED phototherapy fields with the wound/lesion/tissue condition 19. The overall length of the bandage, which includes the wound/lesion/tissue condition smart bandage coverage area 225 and the smart bandage straps 224, can be different based on the size of the limb or appendage or wound/lesion/tissue condition 19, which creates the necessity to have multiple sizes for such smart bandage with LED/OLED phototherapy fields 270, based on the specific medical treatment, or the size of the limb, or appendage, or wound/lesion/tissue condition 19.

In another embodiment, the LED/OLED actuators 274 can be replaced by actuators that use lasers, photodynamic therapy, or ultraviolet light. The type of ancillary light therapy systems (similar in construction to the smart bandage with LED/OLED phototherapy fields 270 presented in FIG. 27) used in conjunction with the main/primary treatments will be dictated by the type of cells, tissues, or individual organs, by the specific disease, or patient characteristics and their collateral comorbidities.

Although in the embodiments presented in FIGS. 22-27, the smart bandages incorporate only one energy technology in them, it is also possible to have other embodiments that are capable to use two or more energy technologies incorporated in one smart bandages. Such designs could be used for more complex and/or large wound/lesion/tissue condition 19, which will need large bandages that are capable to incorporate more batteries, multiple electronic circuits, connectors and energy delivery elements electric electrodes or electromagnetic loops or patches or piezo ceramics/crystals/fibers or LED/OLED actuators, etc. The smart bandages that incorporate multiple energy technologies can activate those concomitantly or subsequently following a certain pre-programmed protocol that assures sufficient treatment of wound/lesion/tissue condition 19 and which is efficiently covered by the capacity of the batteries incorporated in such bandages.

Besides of using smart bandages in between energy-based main/primary treatment sessions (as presented in FIGS. 22-27), in other embodiments different energy-based treatments can be used in combination energy therapy protocols that use alternately different energy-based technologies in separate treatment sessions performed in different days, which will keep the necessary upregulation/boost of benefic factors or inhibition of harmful factors for stimulation, healing, and regeneration of cells, tissues, tissue transplants or organ transplants or skin grafts or active skin substitutes (placental, dermal, fish skin, etc.), and organs. When the treatments with shockwaves/pressure waves 18 are the main/primary ones, they can be alternated in a combination energy therapy protocol with secondary/subordinate treatments performed with dedicated devices/systems in different days, in between the main/primary treatments, using pressurized active substances, or electric current fields, or electromagnetic fields, or LED/OLED phototherapy, or laser phototherapy, or ultraviolet light, or non-contact and/or contact ultrasound waves, or radio frequency therapy, or energy-driven nanotechnology therapy, or pneumatic compression, or hydrotherapy, or cold atmospheric plasma therapy, or topical negative pressure therapy, or thermal therapy, and energy-driven Chinese or Indian Medicine. Interesting to note, when the topical negative pressures/vacuum suction and shockwaves/pressure waves 18 are used in an energy combination protocol therapy, the shockwaves/pressure waves 18 can be transmitted through the foam patch used by the negative pressure/suction devices to absorb exudates. That means that the shockwave treatment can be delivered without any disturbance to the negative pressure/suction. Alternatively, the shockwave treatments can be done before installation of the foam bandage.

When the treatments with non-contact ultrasound waves 86 transmitted via a mist solution are the main/primary treatments, they can be alternated in a combination energy therapy protocol with secondary/subordinate treatments performed with dedicated devices/systems in different days, in between the main/primary treatments, using pressurized active substances, or electric current fields, or electromagnetic fields, or LED/OLED phototherapy, or laser phototherapy, or ultraviolet light, or contact ultrasound waves, or shockwaves/pressure waves, or radio frequency therapy, or energy-driven nanotechnology therapy, or pneumatic compression, or hydrotherapy, or cold atmospheric plasma therapy, or topical negative pressure therapy, or thermal therapy, and energy-driven Chinese or Indian Medicine. These energy therapy protocols, which use alternately different energy-based technologies in separate treatment sessions performed in different days, will keep the necessary upregulation/boost of benefic factors or inhibition of harmful factors for stimulation, healing, and regeneration of cells, tissues, tissue transplants or organ transplants or skin grafts or active skin substitutes (placental, dermal, fish skin, etc.), and organs.

Alternatively, any of the energy therapies (electric stimulation, electromagnetic fields, phototherapy, contact ultrasound, energy-driven nanotechnology, pneumatic compression therapy, hydrotherapy, radio frequency, and cold atmospheric plasma) can be used as main/primary treatment and any of the energy-driven treatments that are not used as primary, can be applied as the secondary treatments in between the main/primary treatments. For all these potential energy-based combination protocols or for any of the embodiments presented in FIGS. 1A-27, the main reason to combine different energy technologies is given by the overlap of different mechanism of actions provided by each individual energy technology, which assures that a certain affliction is attacked from multiple angles to assure a higher rate of success in healing the affected cells, tissues, or organs. Also, the combination energy-based therapies or protocols can be used not only for treatment, but also for prevention, pre-conditions for other medical procedures, and maintenance of the targeted cells, tissues, or organs.

Another important embodiment of this invention is the use of an à la carte system that is able to provide multiple energy-based medical treatments tuned on the particularities of each medical case, type of disease, grade of chronicity, patient comorbidities, etc., via a special algorithm created through deep analysis by artificial intelligence (AI) of the medical historical data related to different medical treatments for the type of disease that is targeted. The à la carte system will help the medical personnel in choosing of the proper treatment regimen, the appropriate technology or combination of multiple technologies to be used for the treatment, based on the specific affliction and also on the evolution of the disease or progress in healing, which can dictate a total change of the regimen used previously. This system should be also capable to issue the financial statements for the treatment, payment options, and connect to the reimbursement department of the respective medical establishment or with an ERP (Enterprise Resource Planning) system where the treatment was performed. This will simplify the office or hospital or medical facility procedures for tracking and recording of medical treatments and in the end can reduce the time spent on administrative matters, which translates in significant savings. Furthermore, an à la carte medical system, which employs multiple energy medical technologies used in combination with energy-based devices or combination energy-based treatment protocols (that use combination energy-based devices or independent energy devices incorporating only one energy technology), should be able to connect to the electronic medical records software packages to collect and send back information about the patient treatment and progress towards healing, as part of the national electronic medical records system.

Such à la carte/menu-based system 280 with three different energy combination applicators, which incorporate multiple energy technologies, is presented in the embodiment from FIGS. 28A and 28B. FIG. 28A shows a three-dimensional view of the à la carte/menu-based system 280 with the energy combination applicators 282A, 282B, and 282C connected to the multi-applicator control console 281. In FIG. 28B the à la carte/menu-based system 280 is viewed from the front and with the energy combination applicators 282A, 282B, and 282C removed. Such system is formed of two main components, the multi-applicator control console 281 and the energy combination applicators 282A, 282B, and 282C. In general, the energy technologies incorporated in the first energy combination applicator 282A are different from the energy technologies incorporated in the second energy combination applicator 282B and the energy technologies incorporated in the third energy combination applicator 282C. Any embodiments or combination of embodiments, as the ones presented in FIGS. 1-21E, can be used in the construction of the energy combination applicators 282A, 282B, and 282C. This assures that these energy combination applicators 282A, 282B, and 282C have complementary energy technologies incorporated in them, to give a large range of options for the healthcare user to perform specific treatments. The energy combination applicators 282A, 282B, and 282C are connected to the multi-applicator control console 281 via the combination power cables 34, which have at one end the energy combination applicators 282A, 282B, and 282C and at the opposite end the combination applicator power connectors 285. The multi-applicator control console 281 has dedicated console power slots 286 (see FIG. 28B) in which the combination applicator power connectors 285 of the combination power cables 34 are introduced. The combination applicator power connectors 285 have RFID (Radio Frequency Identification Devices) tags/buttons, or SIM (Subscriber Identity Module) cards, or other means to authenticate the energy combination applicators 282A, 282B, and 282C with the multi-applicator control console 281. This assures the correct pairing of the console with the applicators and overall application of an accurate treatment to the patient. The RFID tags/buttons or SIM cards or any other electronic devices used for authentication are also capable to store information about treatments (time of the treatment, type and serial number of energy combination applicator used, the console type and serial number used for the treatment, settings, type of disease/targeted treatment region, etc.), console counters to track the use of the system, financial records, software errors, or any information that can be transmitted or received in between the energy combination applicators 282A, 282B, and 282C and the multi-applicator control console 281. Although in FIGS. 28A and 28B there are three combination applicator support slot 283 and three associated and dedicated console power slots 286, in some embodiments only two or four or more such elements (283 and 286) can be used, based on the customer's requirements for the complexity of the custom-made multi-applicator control console 281 of the à la carte/menu-based system 280. The customization can be dictated by the targeted use of such custom made à la carte/menu-based system 280 for explicit treatments of specific afflictions.

The combination power cables 34 provide power/energy from the power source or sources and generators incorporated in the multi-applicator control console 281 (not specifically shown in FIGS. 28A and 28B, but shown in detail in FIGS. 1B, 2B, 3B, 4B, 5B, 7B, 8B, 9B, and 10B) and transfer info from the energy combination applicators 282A, 282B, and 282C to the multi-applicator control console 281 and vice versa. In between their use, the energy combination applicators 282A, 282B, and 282C can be safely secured/stored in dedicated combination applicator support slots 283 that are present on the multi-applicator control console 281. When one of the energy combination applicators 282A, 282B, and 282C is used for treatment, the corresponding functionality LED indicator 284 will be lit. The treatment parameters setting, options for treatment, treatment algorithm, real-time display of parameter during treatment, pictures of the targeted treatment area/region, name of targeted treatment zone/region, treatment progress, real-time videos of the targeted area/region during treatment, financial info about the treatment for reimbursement, name of facility performing the treatment, errors, cautions, tutorials, trouble-shooting, patient info, comorbidities, medical history, treatment history, etc., are shown on the console integrated display 288. The functionality and internal structure of the multi-applicator control console 281 can be any of the ones presented in detail for the embodiments from FIGS. 1B, 2B, 3B, 4B, 5B, 7B, 8B, 9B, and 10B or any combination of those, depending on the type of energy combination technologies that are used for the energy combination applicators 282A, 282B, and 282C. On their turn, the energy combination applicators 282A, 282B, and 282C can be any of the embodiments presented in FIGS. 1-21E or any combination of those embodiments.

For the ease of operation, the multi-applicator control console 281 can also have an upper handle 287A, to help with transportation from one location to another, and also a storage drawer 289 with its own drawer handle 289A (see FIG. 28B), where the energy combination applicators 282A, 282B, and 282C or ancillary devices or any extra components associated with the à la carte/menu-based system 280 can be stored for longer period of times in between the use of the multi-applicator control console 281 and of the à la carte/menu-based system 280.

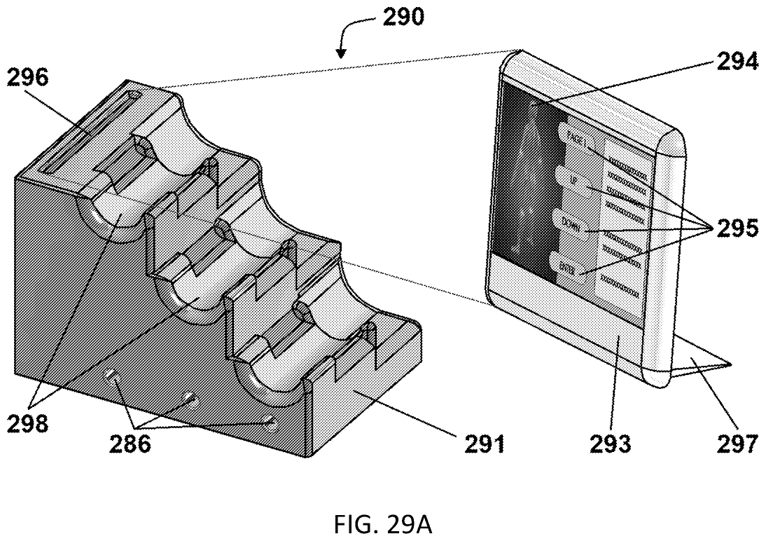

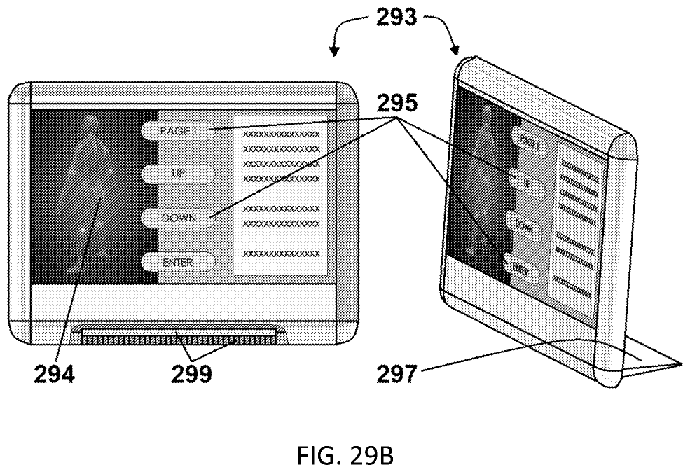

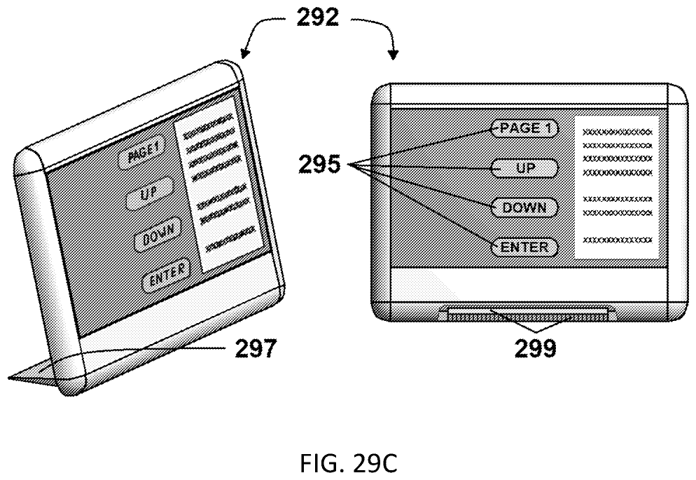

In FIG. 29A is presented a three-dimensional view of another embodiment that is a modular à la carte/menu-based system 290 that uses a modular control console 291 in combination with a control tablet with orthopedic menu 293 (as presented in FIGS. 29A and 29B) or a control tablet with general menu 292 (as presented in FIG. 29C). When compared to the multi-applicator control console 281 from FIGS. 28A and 28B, the modular control console 291 has a totally different design, which has three combination applicator cradles 298 instead of the combination applicator support slots 283 from FIGS. 28A and 28B. Furthermore, the modular control console 291 does not have an incorporated display in it, as seen in the multi-applicator control console 281 from FIGS. 28A and 28B, where a console integrated display 288 is present. Instead, the modular control console 291 from FIG. 29A has a tablet connector slot 296 where an independent control tablet with orthopedic menu 293 can be inserted, if the healthcare users choose to do that. In another embodiment, the control tablet with orthopedic menu 293 from FIG. 29B, can be replaced by a control tablet with general menu 292, as the one presented in FIG. 29C. The design of the control tablet with orthopedic menu 293 or of the control tablet with general menu 292 includes a tablet adjustable stand 297, which allows the use of these tablets as independent displays/components and be completely separated from the modular control console 291. This permits the medical personnel to place the control tablet with orthopedic menu 293 or of the control tablet with general menu 292 in positions that are easier to view them and perform the treatment in the same time, without the need to turn their head towards the modular control console 291, which usually sits away from the patient and the targeted treatment zone.

The modular control console 291 of the modular à la carte/menu-based system 290, besides the “step” construction of the three combination applicator cradles 298, incorporate three dedicated console power slots 286 and the tablet connector slot 296. Although in FIG. 29A there are three combination applicator cradles 298 and three associated and dedicated console power slots 286, in some embodiments only two or more than three such elements (298 and 286) can be used, based on the customer's requirements for the complexity of the custom-made modular control console 291 of the modular à la carte/menu-based system 290. The customization can be dictated by the targeted use of such custom made modular à la carte/menu-based system 290 for explicit treatments of specific afflictions.

In FIG. 29B is presented a control tablet with orthopedic menu 293, which has on its display a human body pictorial with treatment targeted regions 294 and the main menu possible selections 295. The human body pictorial and main menu general selections should be simple (“UP”, “DOWN”, “ENTER”, and treatment info separated in different pages “PAGE 1”, PAGE 2”, etc.), intuitive, with minimum verbiage, and enough information to perform the right treatment selection, monitor the actual treatment, and have safe-fail features that allow the stoppage or restart of the treatment when needed. Also, the control tablet with orthopedic menu 293, should be able to display on its pages the treatment parameters setting, options for treatment, treatment algorithm, real-time display of parameter during treatment, pictures of the targeted treatment area/region, name of targeted treatment zone/region, treatment progress, real-time videos of the targeted area/region during treatment, financial info about the treatment for reimbursement, name of facility performing the treatment, errors, cautions, tutorials, trouble-shooting, patient info, comorbidities, medical history, treatment history, etc., and tutorials related to the use of the modular à la carte/menu-based system 290 and its trouble-shooting. The control tablet with orthopedic menu 293 can be used as a stand-alone component and for that the tablet has a tablet adjustable stand 297, or the tablet can be used as part of the modular control console 291, when the tablet is dropped in the tablet connector slot 296, with the electrical and power and digital connections accomplished via the tablet connector 299.

Furthermore, in FIG. 29C is presented a control tablet with general menu 292, which has on its display the main menu possible selections 295. The main menu general selections should be simple (“UP”, “DOWN”, “ENTER”, and treatment info separated in different pages “PAGE 1”, PAGE 2”, etc.), intuitive, with minimum verbiage, and enough information to perform the right treatment selection, monitor the actual treatment, and have safe-fail features that allow the stoppage or restart of the treatment when needed. Also, the control tablet with general menu 292, should be able to display the medical branch selection (cardiovascular, orthopedic, urology, gastroenterology, sport medicine, nephrology, wound care, etc., to name a few), targeted tissue or organ or region, treatment parameters setting, options for treatment, treatment algorithm, real-time display of parameter during treatment, pictures of the targeted treatment area/region, treatment progress, real-time videos of the targeted area/region during treatment, financial info about the treatment for reimbursement, name of facility performing the treatment, errors, cautions, tutorials, trouble-shooting, patient info, comorbidities, medical history, treatment history, etc., and tutorials related to the use of the modular à la carte/menu-based system 290 and its trouble-shooting. The control tablet with general menu 292 can be used as a stand-alone component and for that the tablet has a tablet adjustable stand 297, or the tablet can be used as integral part of the modular control console 291, when the tablet is dropped in the tablet connector slot 296, with the electrical and power and digital connections accomplished via the tablet connector 299.

In FIGS. 30A and 30B is presented an à la carte/menu-based modular system with tablet attached 300, which is a similar system to the one presented in FIGS. 29A, 29B, and 29C, but this time with the control tablet with general menu 292 or the control tablet with orthopedic menu 293 being permanently attached/incorporated to the modular control console 291 (non-detachable) using the tablet connector slot 296, without the possibility to remove the tablet from its position. However, in such design the control tablet with orthopedic menu 293 or of the control tablet with general menu 292 should have a hinge at their lower part (not shown in FIGS. 30A and 30B), which will allow the folding of the tablets to a position that can protect their displays in between treatments, if the medical personnel choose to do that. Furthermore, the control tablet with orthopedic menu 293 or of the control tablet with general menu 292 can be designed to be able to swivel/rotate around the mid-point of their lower part (not shown in FIGS. 30A and 30B), which will allow the healthcare users/medical personnel to rotate the tablets in any position that allows an optimum view for them during the treatment.

In this embodiment from FIGS. 30A and 30B, the à la carte/menu-based modular system with tablet attached 300 is shown in combination with three different energy combination applicators connected to the modular control console 291. The à la carte/menu-based modular system with tablet attached 300 shown in FIGS. 30A and 30B has a first energy combination applicator as the one presented for the combination shockwaves/pressure waves and overlapping radio-frequency fields medical system 110 (see FIG. 11), a second energy combination applicator as the one presented for the a combination shockwaves/pressure waves and overlapping contact ultrasound medical system 120 (see FIG. 12), and a third energy combination applicator as the one presented for the a combination shockwaves/pressure waves, electromagnetic field, and active substance medical system 130 (see FIG. 13A-13B), just as an exemplification of the use of different embodiments presented before in this invention. However, any embodiments or combination of embodiments, as the ones presented in FIGS. 1-21E, can be used in the construction of the energy combination applicators shown in FIGS. 30A and 30B. This assures that these energy combination applicators have complementary energy technologies incorporated in them, to give a large range of options for the healthcare user to perform specific treatments. The energy combination applicators are connected to the modular control console 291 via the combination applicator power cables 301, which have at one end the energy combination applicators and at the opposite end the combination applicator power connectors 285 that are introduced in the console power slots 286 (not specifically shown in FIGS. 30A and 30B, but similar to those shown in FIG. 28B). In the embodiment presented in FIGS. 30A and 30B, the console power slots 286 that are used for the combination applicator power connectors 285 are positioned on the lateral side of the modular control console 291, which is different from the embodiment presented in FIGS. 28A and 28B, where the console power slots 286 were positioned on the frontal side of the multi-applicator control console 281. Also, to assure a stable positioning on a table, medical cart, etc. of the multi-applicator control consoles 281 and of the modular control consoles 291, these consoles have the control console supports/legs 302, as seen in FIGS. 28B and 30B. The combination applicator power connectors 285, from FIGS. 30A and 30B, have RFID (Radio Frequency Identification Devices) tags/buttons, or SIM (Subscriber Identity Module) cards, or other electronic means/devices to authenticate the energy combination applicators with the modular control console 291. This assures the correct pairing of the console with the applicators and overall performing of an accurate treatment to the patient. The RFID tags/buttons or SIM cards or any other electronic devices used for authentication are also capable to store information about treatments (time of the treatment, type and serial number of energy combination applicator used, the console type and serial number used for the treatment, settings, type of disease/targeted treatment region, etc.), console counters to track the use of the system, financial records, software errors, or any information that can be transmitted or received in between the energy combination applicators and the modular control console 291. The combination applicator power cables 301 provide power/energy from the power source or sources and generators incorporated in the multi-applicator control console 281 (not specifically shown in FIGS. 28A and 28B, but shown in detail in FIGS. 1B, 2B, 3B, 4B, 5B, 7B, 8B, 9B, and 10B). In some embodiments, the combination applicator power cables 301 can have along their length one or more active substance tubing 13 (see FIGS. 1A, 1B, 13B, 14A, 14D, and 14E) or one or more laser fiber optic cable 51 (see FIGS. 5A, 5B, and 6A) or one or more mist solution tubing 81 (see FIGS. 8A, 8B, 13E, 21A, 21B, 21D, and 21E). The same combination applicator power cables 301 can be used to transfer info from the energy combination applicators to the modular control console 291 and vice versa. In between their use, the energy combination applicators can be safely secured/stored in dedicated combination applicator cradles 298 that are present on the modular control console 291. Although not specifically shown in FIGS. 30A and 30B, when one of the energy combination applicators is used for treatment, a corresponding functionality LED indicator 284 will be lit, as seen in FIGS. 28A and 28B. The medical branch selection (cardiovascular, orthopedic, urology, gastroenterology, sport medicine, nephrology, wound care, etc., to name a few), targeted tissue or organ or region treatment parameters setting, options for treatment, treatment algorithm, real-time display of parameter during treatment, pictures of the targeted treatment area/region, treatment progress, real-time videos of the targeted area/region during treatment, financial info about the treatment for reimbursement, name of facility performing the treatment, errors, cautions, tutorials, trouble-shooting, patient info, comorbidities, medical history, treatment history, etc., are shown on the control tablet with general menu 292 (not specifically shown in FIGS. 30A and 30B) or on the control tablet with orthopedic menu 293 (as seen in FIGS. 30A and 30B). The functionality and internal structure of the modular control console 291 can be any of the ones presented in detail for the embodiments from FIGS. 1B, 2B, 3B, 4B, 5B, 7B, 8B, 9B, and 10B or any combination of those, depending on the type of energy combination technologies that are used for the energy combination applicators. On their turn, the energy combination applicators can be any of the embodiments presented in FIGS. 1-21E or any combination of those embodiments.

From the manufacturing point of view, all the energy combination systems or devices or energy combination treatment protocols that were presented before for the embodiments from FIGS. 1-30B are designed to completely eliminate or reduce moving parts, which assures increased reliability. Also, these systems and devices should be easy to assemble, made of compatible materials with medical use and recyclable, and should be built with as much as possible off-the-shelf parts.

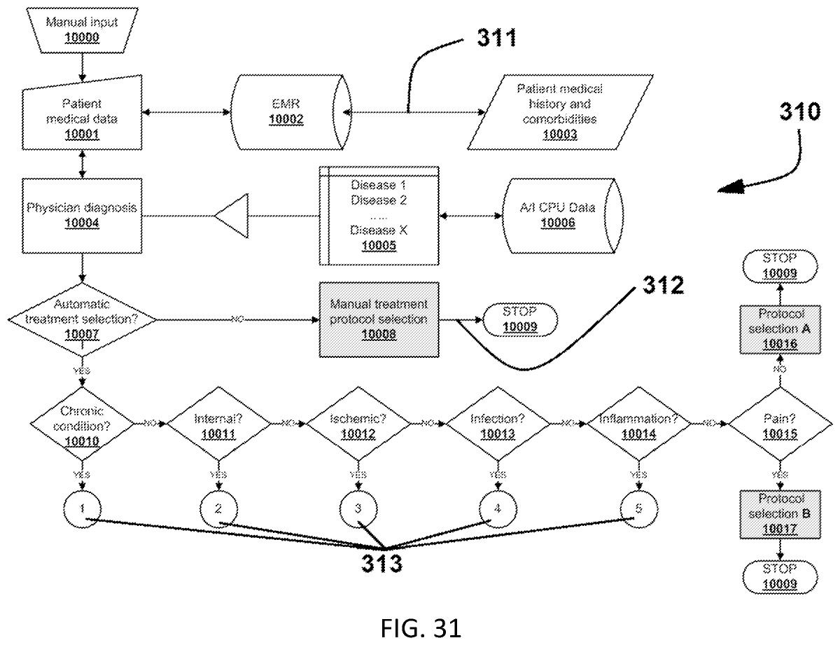

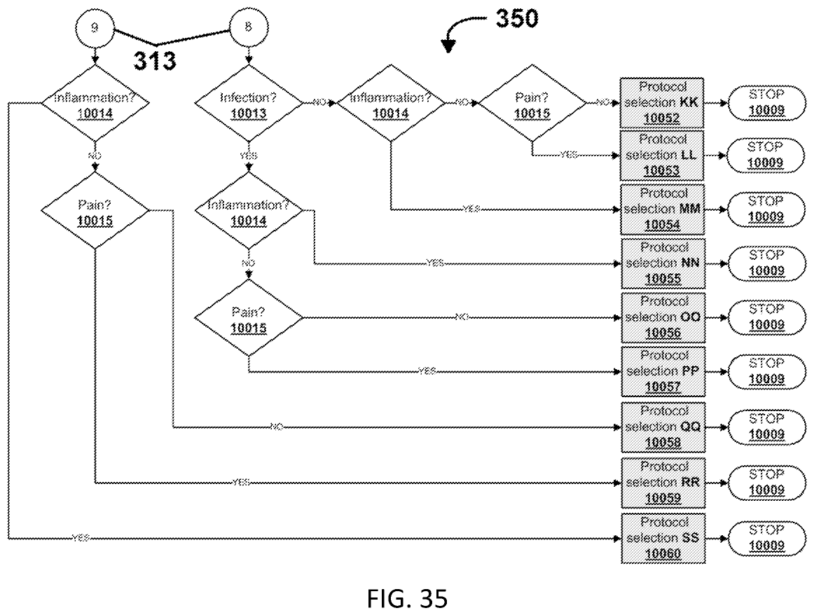

The à la carte systems described in FIGS. 28A-30B are capable to perform different energy combination treatment algorithms that are based on the type of affliction/disease and its chronicity, tissue/organ targeted, and its position on the surface or inside the human or animal body (superficial or internal), the situation of blood circulation in the targeted area or region for treatment (ischemic or non-ischemic), the presence of infection or not, inflammation situation, and if the pain is present or not. All these factors are dictated by the type of disease/affliction and its manifestation, stage of the disease, patient comorbidities, life style, hygiene, sex, age, race, genetic make-up, and other intrinsic factors that dictate the acute or chronic manifestation of the symptoms for the respective disease/affliction. An example of the algorithm used to determine the combination protocol that employs multiple energy medical technologies is presented in FIGS. 31-35.

In general, regardless of the disease or affliction or the type of cells, tissues, or organs affected by the disease or affliction, for both humans and animals, the main characteristics that are dictating the diagnosis and treatment are related to the following elements:
a. Chronic or not chronic condition of the respective disease.
b. Internal/deep or superficial positioning of the targeted treatment zone that can be a cellular structure, tissue(s), tissue transplants, organ transplants, skin grafts, active skin substitutes (placental, dermal, fish skin, etc.), or organs.
c. The ischemic or non-ischemic situation (sufficient or insufficient blood supply) to the treatment zone or organ or a pre-existing condition related to chronic cardiovascular problems.
d. The presence of infection or not in the treatment zone, tissue, or organ, as local or systemic infection.
e. Inflammation or lack of inflammation in the treatment zone, tissue, organ, or chronic systemic inflammation throughout the body.
f. The presence of pain or not associated with the treatment zone, tissue, organ, or chronic pain throughout the body related with a pre-existing condition.

Once the treatment regimen/protocol is determined, then other adjustments can be applied to the treatment input parameters as energy-input setting, time of the treatment, frequency, number of pulses, number of treatment sessions, etc., to customize the medical treatment based on specific patient comorbidities, physical characteristic, life-style, etc., as it is detailed in the U.S. Pat. No. 10,888,715.

One of the common characteristics for any medical treatment across any possible technology is the treatment time duration. This parameter can be calculated by combination of multiple parameters or can be an independent one. For example, in the case of shockwaves the treatment time is equal with the total number of shockwaves for a treatment session divided with the number of shockwaves per second, which is the frequency of shockwave pulses. For ultrasound, phototherapy, electromagnetic, electrical stimulation, hydrotherapy, negative pressure, thermal therapy, etc., to name a few, the treatment time is an independent parameter that is calculated based on the size/area of wound, tissue, or skin condition, the extend of local infection and its severity (grade of infection—I to V, the type of pathogen(s), and the organ or type of tissue), the volume of tissues or organs that need treatment for bacterial infection or biofilm infection or fungal infection or parasite infection or harmful micro-organisms' infection, the volume of necrotic skin or necrotic tissue or necrotic bone or necrotic organ, the area of the burned tissue, the volume of hard (bone), semi-hard (ligaments or tendons) or soft tissue (skin, muscle, tissues, organs, etc.) that requires regeneration, the length of skin or ligament or tendon tears, the volume of muscle that exhibits contraction, the gap and location of bone non-unions, the volume of an organ affected by anormal functioning, the size of bone fissure or fracture, the location and size of backbone that requires fusion, the size of a bone spur, the size of heterotopic ossification or calcification, the size of dental plaque, the area affected by pain or inflammation or edema, the grade of obstruction and size of the plaque/calcification, vulnerable plaque, or restenosis of a blood vessel, the percentage/size of an occlusion of a blood vessel, the size of a blood clot (thrombus or embolus) that blocks a blood vessel, the diameter and length of a blood vessel aneurysm, the size of the tissue inflammation produced by blood vessels stenting (metal or drug eluting stents) or by angioplasty with balloons or drug eluting balloons, the length of in-stent restenosis of blood vessels, the length of a blood vessel that have spasms, the volume of muscle spasm and contraction, the area of tissue that requires capillaries' formation, the diameter and size of a lymphatic vessel that needs repair, the size of heart muscle ischemia or in general tissue ischemia, the size of area around the heart valves that requires their actuation muscles regrowth and revascularization, the size of heart muscle regeneration, the area of the heart sack that requires treatment for fluid accumulation, the area that requires treatments using any type of human or animal or artificial grafts and biologics from placenta or other human harvested tissues, the size of area affected by lymphedema, the grade/amount of organ or tissue or vessel hyperplasia, the size of chronic tissue or organ inflammation, the area of organ adhesions produced by surgeries, the length of a surgical incision, the extend and severity of capsular contraction around implants, the severity of implant infection (area of infection, grade of infection—I to V, and the type of pathogen(s)), the extend of area of cellulite, the volume or area of body sculpting, the volume or area that needs liposuction, area affected by spider veins, area of skin that requires rejuvenation, volume or area of the zone that needs collagen formation, the size/area of a scar or fibrotic tissue, the severity and size of tissue spasm/contraction, the area surrounding the painful peripheral nerves, the location and size of peripheral nerve that requires regeneration, the area of ischemic tissue that requires blood vessel growth and/or angiogenesis or vasculogenesis, the length and width (area) of veins that needs treatment, the area of tissue/skin affected by autoimmune diseases, the size/volume of cancer tumors or unwanted benign tissue, the grade and severity of bacterial or abacterial prostatitis (chronic pelvic syndrome), the area of the bladder affected by interstitial cystitis, the volume of tissue or organ and the quantity of stem cells' solution or DNA/RNA/mRNA fragments or gene cocktails or proliferation factors or growth factors or angiogenesis factors used for the treatment, the volume of tissue that requires stimulation of dormant stem cells, the area of tissue or organ that requires in vivo stem cell proliferation and differentiation, the volume of a blood vessel or of the tissue or of the organ that needs localized drug delivery, the location and size of a patch or implant or any biodegradable structure that delivers localized drugs or active substances or cellular materials or vaccines, the location and the volume of tissue or organ that is affected by an autoimmune disease, the size of pacemaker leads or any implant or prosthesis that requires loosening and extraction, the size of the implant that needs treatment for aseptic loosening, and the like. The algorithm and principles that employ energy combination systems or devices or treatment energy protocols presented into this patent apply for this general parameter, which is the treatment time, a characteristic across any medical technology applied for treatment of human or animal subjects for healing or alleviate a certain medical condition. The same principle of treatment time applies also for plant treatment using energy combination systems or devices or treatment energy protocols.

The energy combination systems or devices presented in this invention can be used independently for treatments or for combination energy treatment protocols that consist of main treatments and secondary treatments performed in between the main treatment sessions. Also, although the energy combination systems or devices presented in this invention were presented as being extracorporeal, these energy combination systems can also be used for an intracorporeal approach, by reducing their size and changing some design features to accommodate the intracorporeal treatment. Such intracorporeal energy combination systems and combination energy treatment protocols using energy delivery devices (employing one energy technology) can use the natural human and animal conduits or artificial conduits, can be minimal invasive as part of open surgeries, can be used for prevention/prophylactic treatment or for an active treatment of a specific disease or affliction or following other medical interventions, can be performed in medical offices, hospitals, long care facilities, nursing homes, or hospices.

The total number of energy treatment protocols options presented in the general embodiment from FIGS. 31-35 is forty-five (45). These 45 energy treatment protocols can be performed only with the individual/distinctive energy systems/devices (using only one form of energy technology) or by using energy combination systems (as the ones presented in this patent) or by using specific non-energy devices or any possible combination of these different systems/devices that employ energy or not, which is dictated by the characteristics of the required treatment needed for a specific human or animal affliction or sometimes for treatment of plants. Of course, this is a general approach, which fits for any kind of disease that is directed to treat cells, tissues, or organs from human or animal bodies or plants. Furthermore, this general approach algorithm can be designed as an artificial intelligence (A/I) software system, in which the algorithm is continuously improved based on new available research data or through information given by the experience in treating a specific disease of the human or animal body or from plants. However, the examples presented in FIGS. 31-35 are presented for human afflictions or disease and sometimes for animal afflictions or disease.

To understand the differences in between these treatment protocols, without any limitations to the use of this algorithm, specifically detailed explanations are presented for same specific cases of general diseases that affects humans and/or animals and fit for a certain treatment protocol. For optimum energy combination systems or devices used for treatment, or for optimum combination energy treatment protocols described in FIGS. 31-35, the actual artificial intelligence (A/I)-based algorithm uses certain common elements to explain pathway, connections from one page to the next. Thus, the bidirectional communication 311 (as seen in FIG. 31) defines a connection that goes both ways in between two distinctive parts of the algorithm, where the flow of the algorithm or information can go in both directions from one part of the algorithm to the second part and vice versa. In contrast, the unidirectional communication 312 (as seen in FIG. 31) functions in only one direction, which means that the flow of the algorithm or information can go only in one direction in between two distinctive parts of the algorithm. To show the connection in between different pages of the artificial intelligence (A/I)-based algorithm that are in different figures, the connectors to the following pages 313 are used, as seen in FIGS. 31-35.

Each of the treatment protocols described by the FIGS. 31-35 will have at least one or multiple treatment sessions. The characteristics that are specific for each treatment session are the treatment time duration and energy level or wattage level delivered to the body/tissue/organ, which overall gives the dosage of the treatment session. For energy devices/systems or energy protocols a treatment session the time duration should be in the range of 2 minutes to 180 minutes, with a preferable range of 5 minutes to 60 minutes. Also, the energy level should be 10 millijoule to 1 joule, preferable 20 millijoule to 500 millijoule and the wattage level should be 5 watts to 200 watts, preferable 10 watt to 100 watt. These are base settings, which can be altered to create an individualized treatment, as presented in U.S. Pat. No. 10,888,715. Thus, for individualized treatment session of a tissue/organ condition with energy devices/systems, the personalized determination and automatic adjustment of a treatment session dosage (treatment time duration and energy level or wattage level) to be administered for a personalized treatment is based on factors such as a patient's comorbidities, state of the tissue condition, individual physical characteristics, and lifestyle parameters.

As seen in FIG. 31, the artificial intelligence (AI)-based algorithm starts with the manual input 10000 that is done by a physician or a nurse or a qualified medical health worker, based on the physical characteristics, living habits, comorbidities and symptoms of the patient, defined as patient medical data/records 10001, chronicity of the affliction, location (internal or external) of the affected cells, or tissue(s), or organ(s), the integrity of blood supply (ischemic or non-ischemic condition), presence or not of infection, or inflammation, or pain. Some or all of the patient medical data/records 10001 can be extracted from the patient medical history and comorbidities 10003 that already exists in the electronic medical records (EMR) 10002 that can be extracted from a national health records database or from the data existing at the respective health facility where the treatment takes place. This information is usually given by the patient via different questionnaires that are completed at the medical health facilities and it is updated periodically based in changes of the health status of the patient. That means that the information can flow in both directions, as needed for each particular situation, and thus the use of the bidirectional communication 311.

Based on the patient medical data/records 10001, the physician can provide a physician diagnosis 10004. In some specific situation, a diagnosis can also be provided by a nurse or a qualified medical health worker. When a physician or a nurse or a qualified medical health worker is asked the question about preference on automatic treatment selection 10007, they can choose to follow the manual treatment option or the automatic treatment option. If the diagnosis is a simple or clear one and has well-known treatment options for which the physician or a nurse or a qualified medical health worker is knowledgeable that will bring success, then a manual treatment protocol selection 10008 is made and the algorithm will have the algorithm stop 10009. The connection in between manual treatment protocol selection 10008 and the algorithm stop 10009 can go in only an unidirectional way and thus the use of the unidirectional communication 312. However, in most situation the automatic treatment selection of the artificial intelligence (AI)-based algorithm can be chosen. For that, the provider of the algorithm will have an artificial intelligence (A/I) central processor unit (CPU) data 10006 that stores the software on which the algorithm is based and also the database information and pathways that needed to be taken to provide a specific treatment protocol based on the specific disease from a list of possible diseases 10005. The connection in between the list of possible diseases 10005 and artificial intelligence (A/I) central processor unit (CPU) data 10006 is a bidirectional communication 311, since the information goes back and forth all the time for continuous improvement of the artificial intelligence (AI) process. As mentioned before, the algorithm is continuously improved based on new available research data or through information given by the medical personnel experiences in treating a specific disease of the human or animal body. Thus, the algorithm developer can modify the number of diseases that are tackled by the algorithm, which can be continually increased. Furthermore, the algorithm developer can continuously improve the algorithm based on different options to treat the respective affliction, based on complex and new data related to available energy-based on non-energy treatment options and also based on the physical characteristics, living habits, comorbidities, and symptoms of the patient. Since the artificial intelligence (A/I)-based algorithm is a proprietary one for the developer, the direction of information from the artificial intelligence (A/I) central processor unit (CPU) data 10006 and the list of possible diseases 10005 is only unidirectional towards the physician diagnosis 10004 via the unidirectional and protected connection to artificial intelligence (A/I) 314. Periodic updates of the artificial intelligence (A/I)-based algorithm can be done by the developer to the health facility for free or for a certain subscription fee, based on the contract in between the two parties.

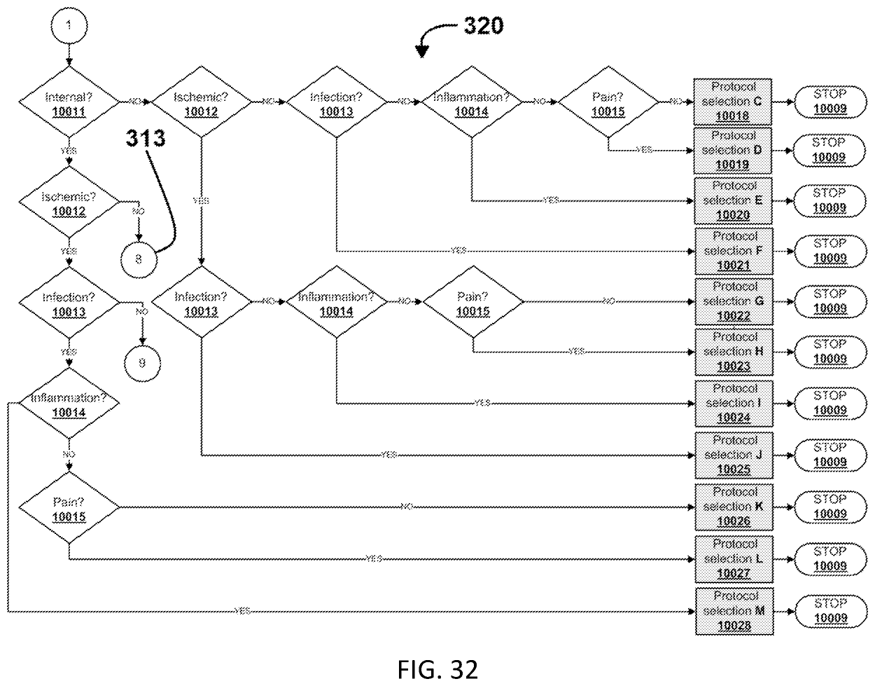

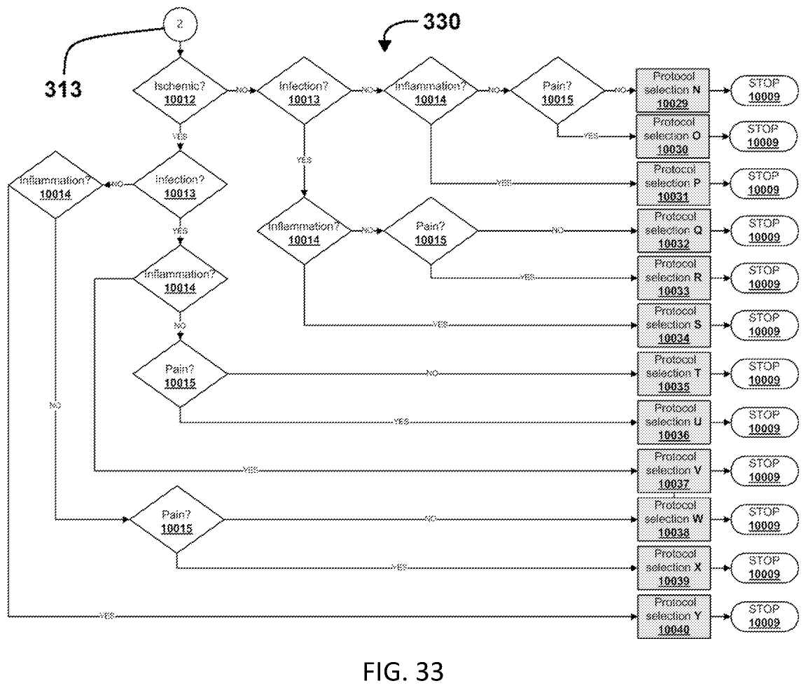

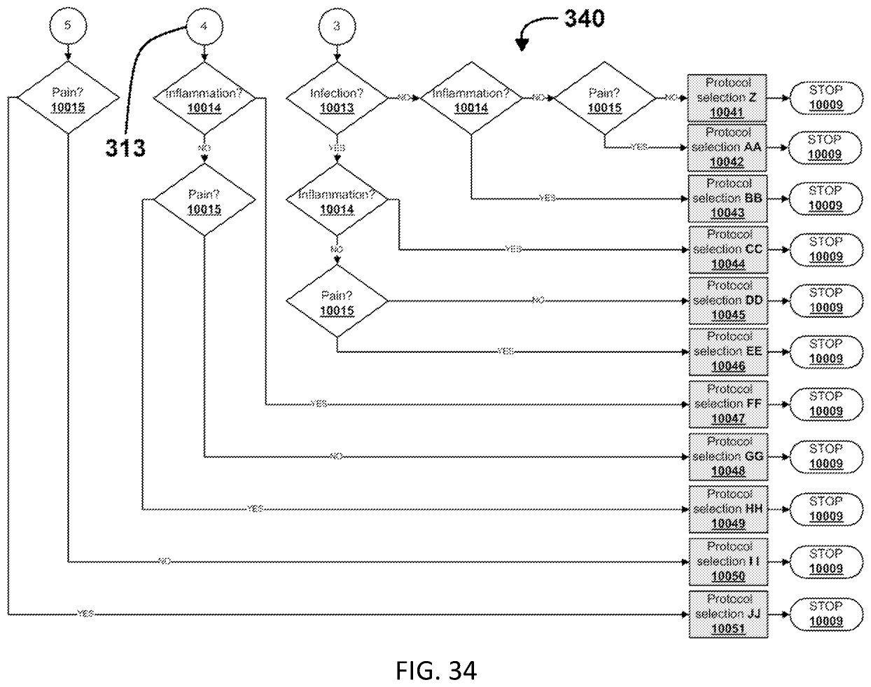

If the automatic treatment selection was chosen by the physician or a nurse or a qualified medical health worker, then the automatic artificial intelligence (A/I)-based algorithm continues with the question about presence of chronic condition or not 10010. If the answer about condition's chronicity is YES, then the algorithm continues in FIG. 32 following the connector to the following pages 313 numbered as 1. If the answer about condition chronicity is NO, then the question about internal condition or not 10011 is asked. If the answer about internal condition is YES, then the algorithm continues in FIG. 33 following the connector to the following pages 313 numbered as 2. If the answer about internal condition is NO (that means that the condition is superficial), then the question about ischemic condition or not 10012 is asked. If the answer about ischemic condition is YES, then the algorithm continues in FIG. 34 following the connector to the following pages 313 numbered as 3. If the answer about ischemic condition is NO, then the question about presence of infection or not 10013 is asked. If the answer about presence of infection is YES, then the algorithm continues in FIG. 34 following the connector to the following pages 313 numbered as 4. If the answer about presence of infection is NO, then the question about presence of inflammation or not 10014 is asked. If the answer about presence of inflammation is YES, then the algorithm continues in FIG. 34 following the connector to the following pages 313 numbered as 5. If the answer about presence of inflammation is NO, then the question about presence of pain or not 10015 is asked. If the answer about presence of pain is NO, then the Protocol A 10016 will be used for the treatment and the algorithm will have an algorithm stop 10009. If the answer about presence of pain is YES, then the Protocol B 10017 will be used for the treatment and the algorithm will have an algorithm stop 10009.

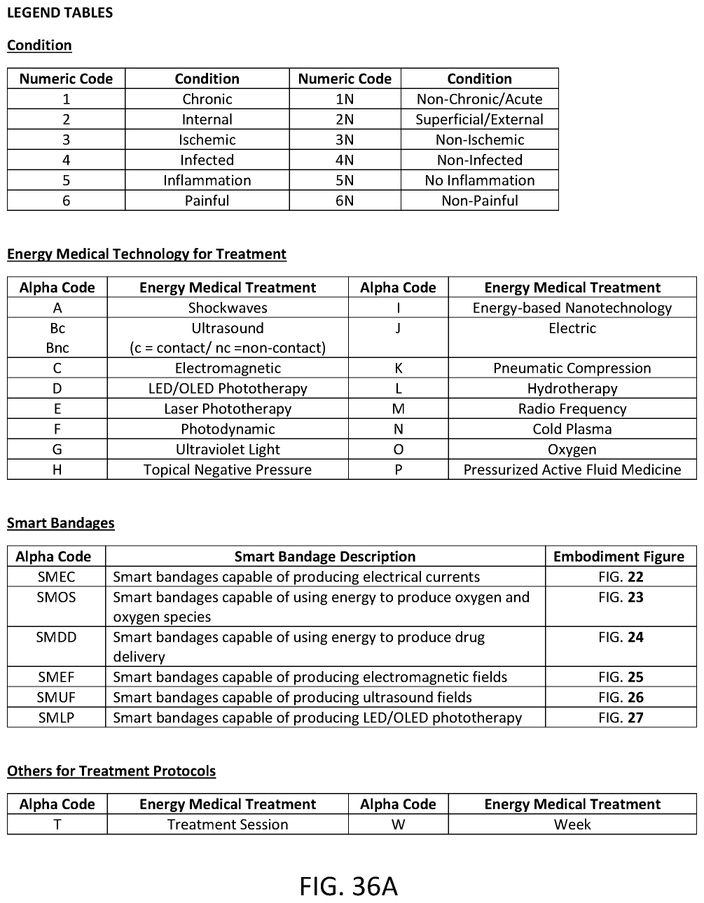

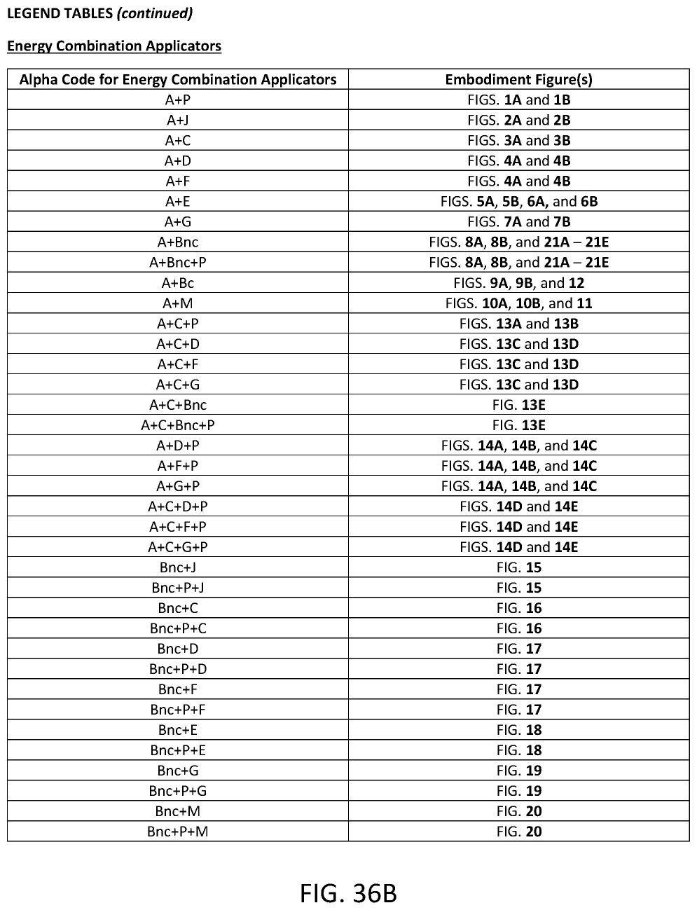

For an easy understanding of the conditions and energy medical treatment options, legend tables are presented in FIG. 36A that show the numeric codes for different conditions related to an affliction or the alpha codes for different energy medical treatments or energy technologies and smart bandages that can be used to perform the protocols presented in FIGS. 31-35 that are using an artificial intelligence (A/I)-based algorithm. In FIG. 36B is presented the alpha code for the energy combination applicators associated with the embodiments presented in this invention and also the corresponding figures where the construction and functionality of such energy combination applicators were described in detail.

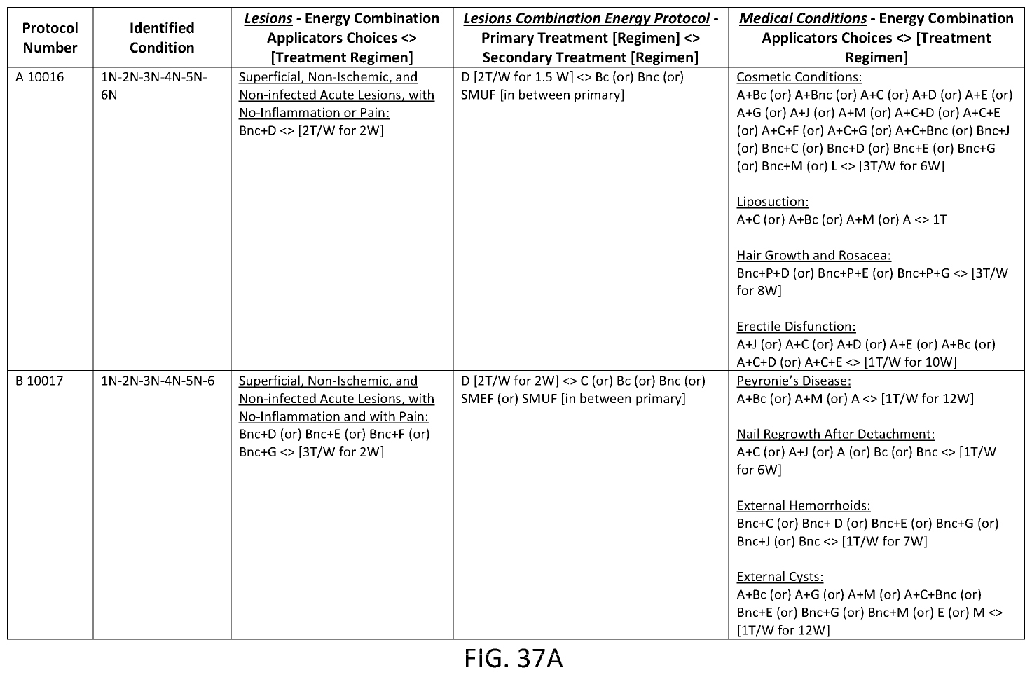

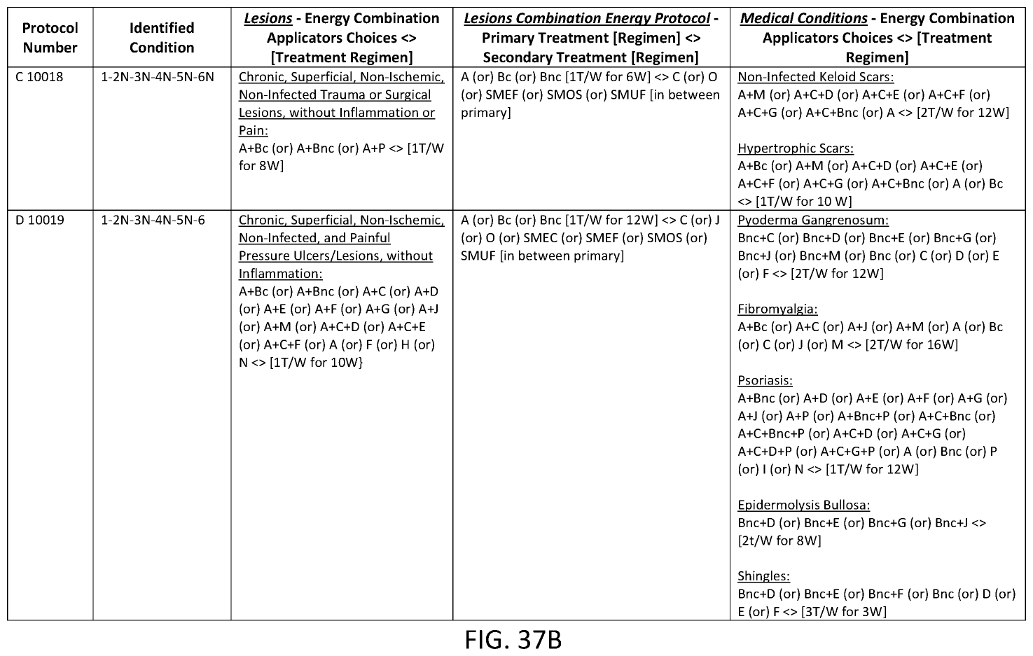

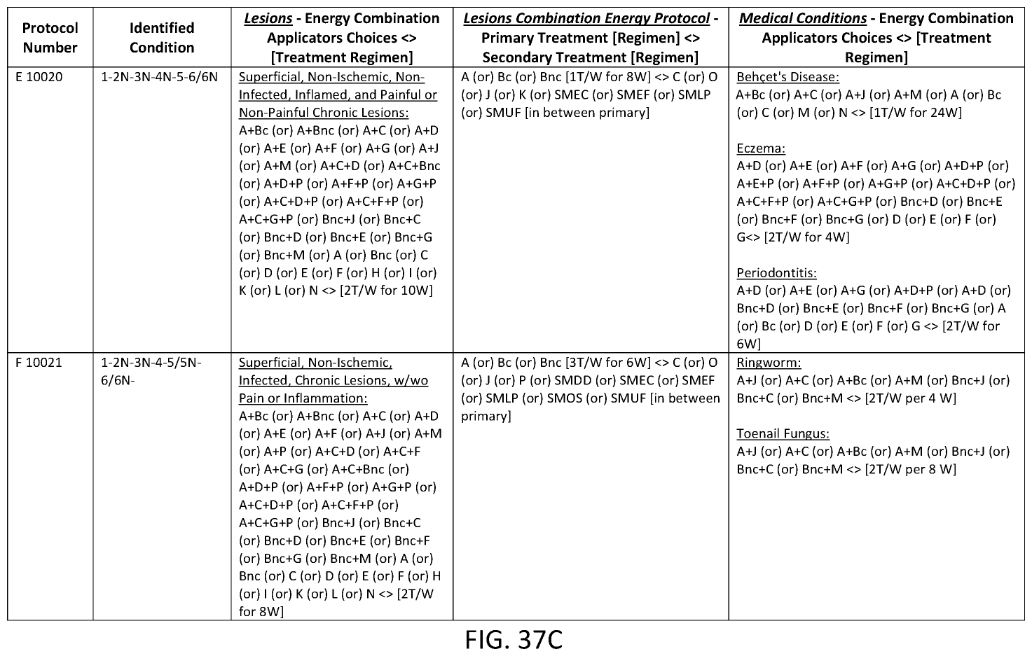

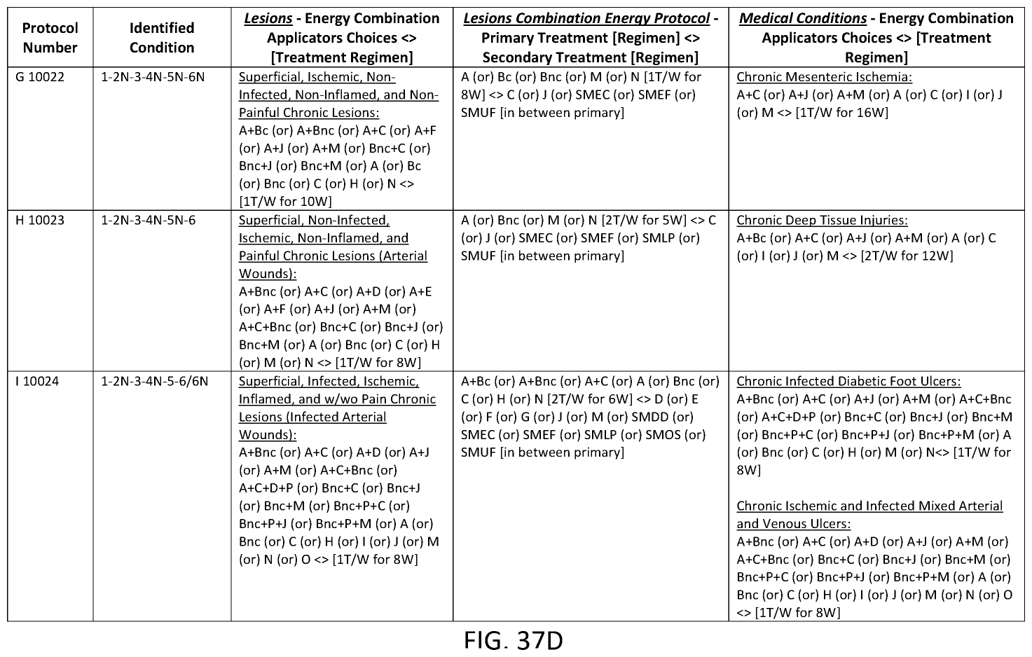

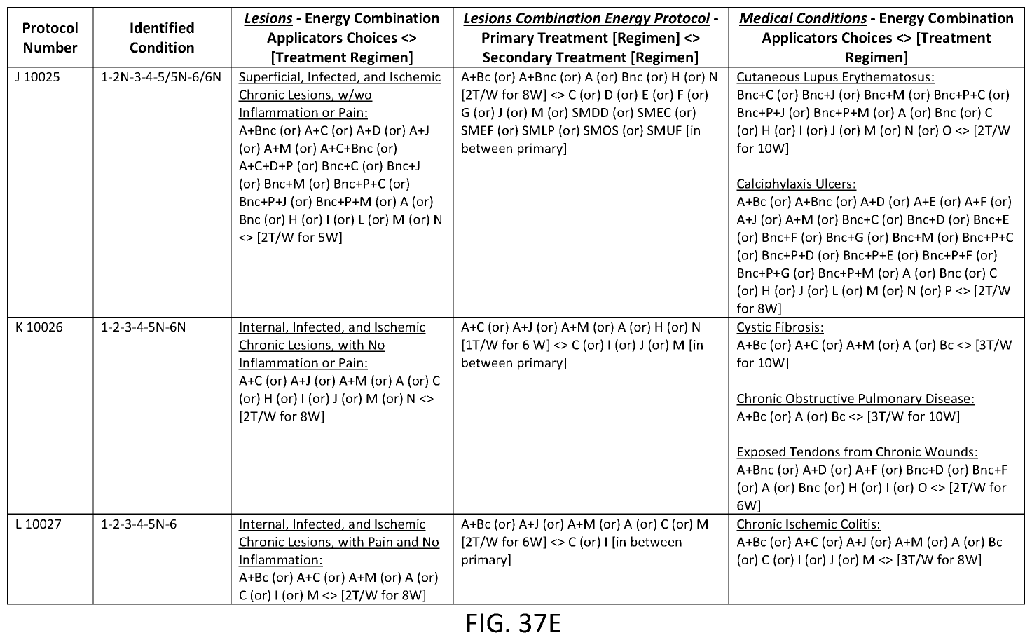

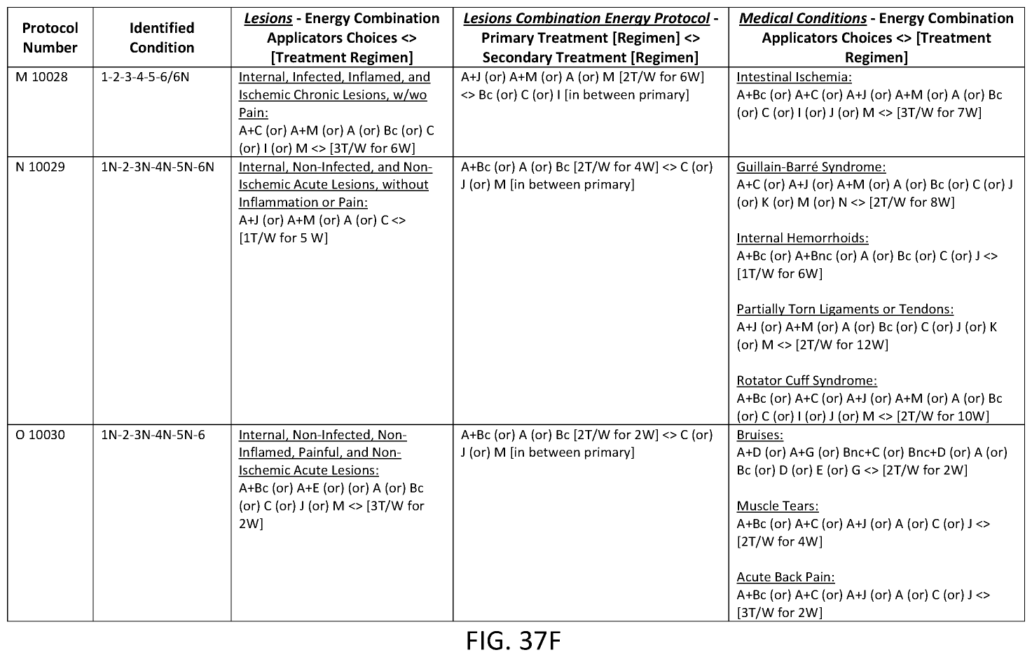

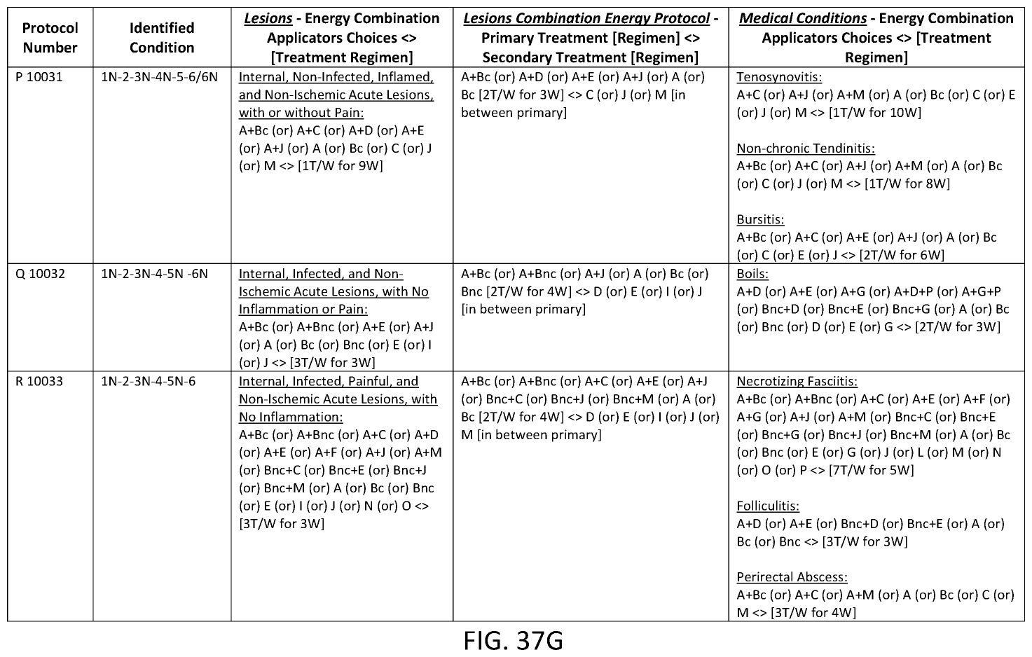

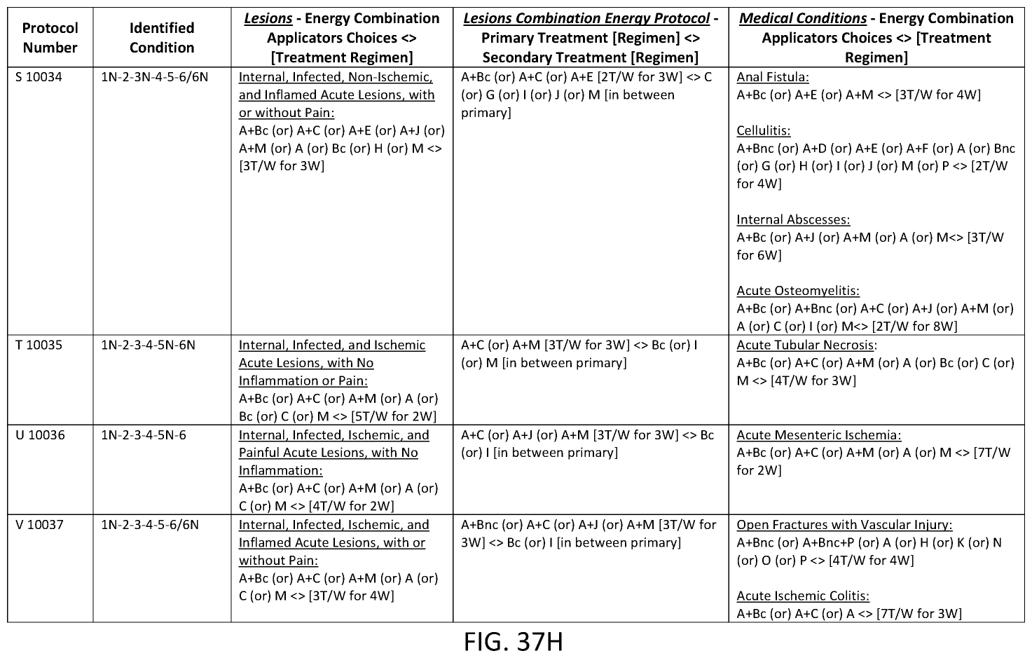

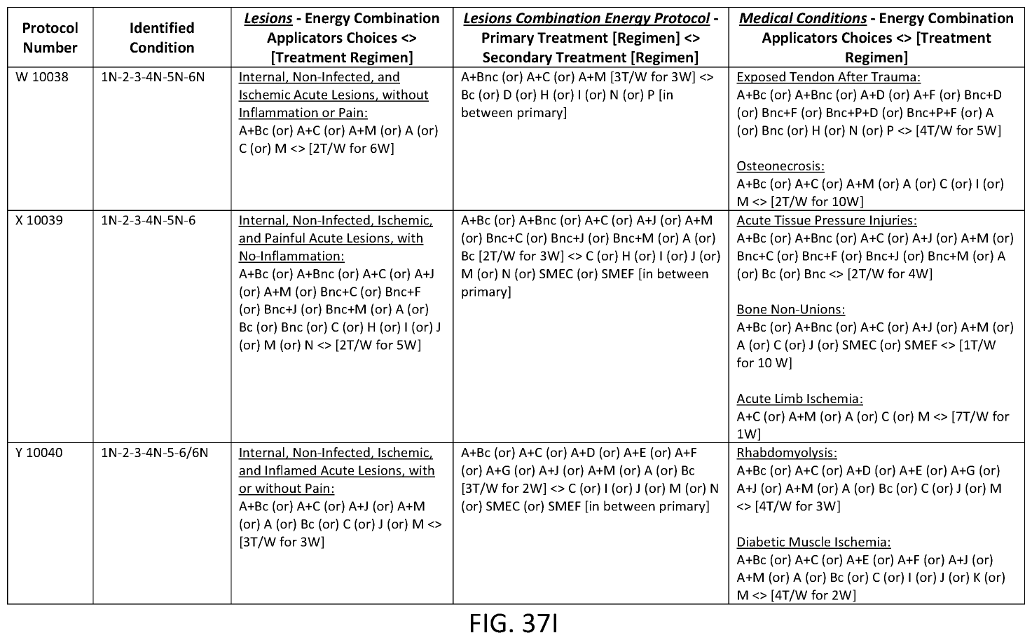

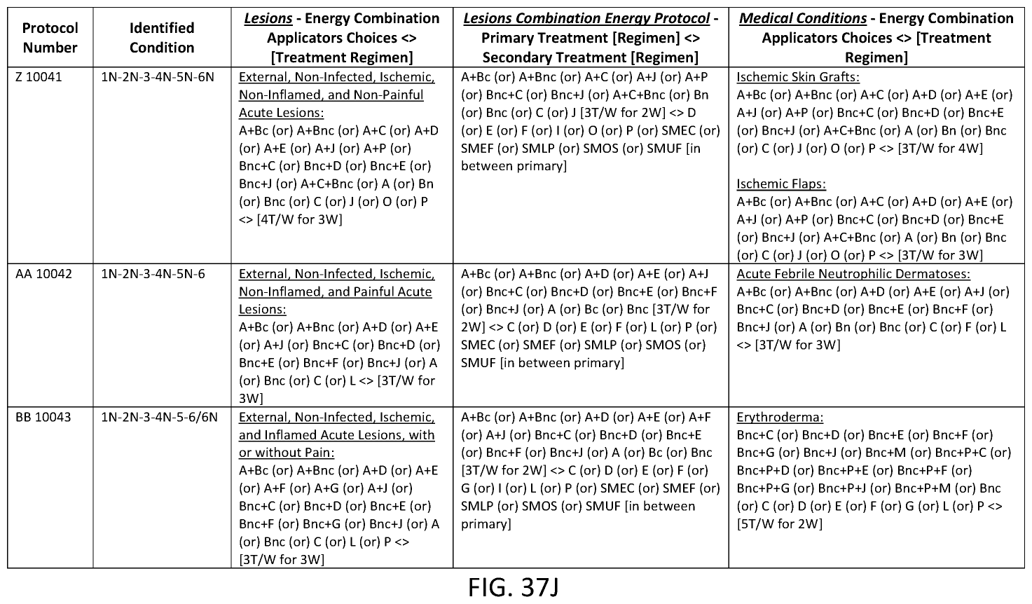

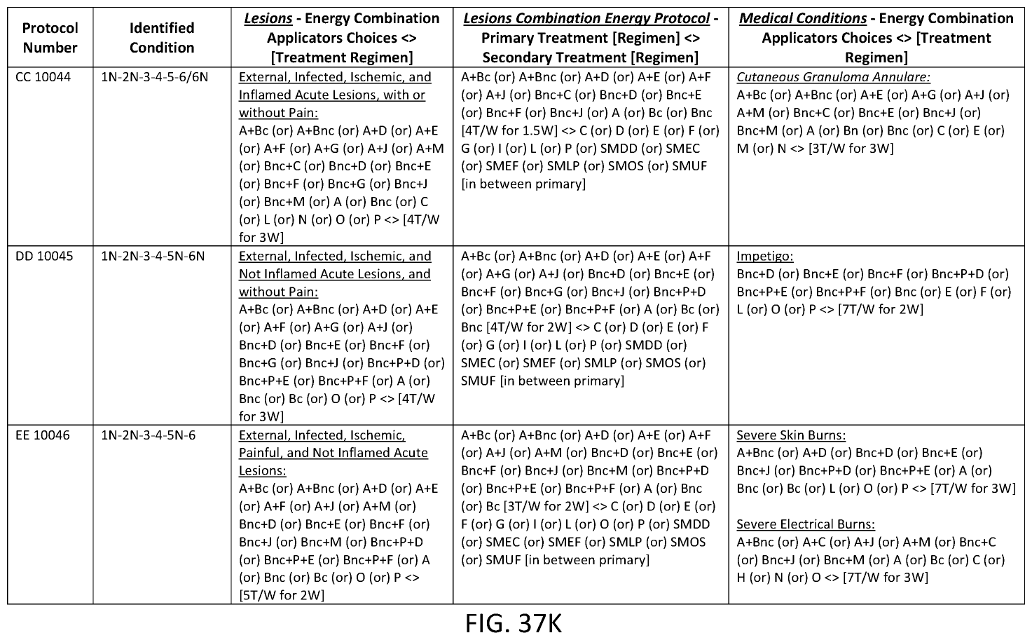

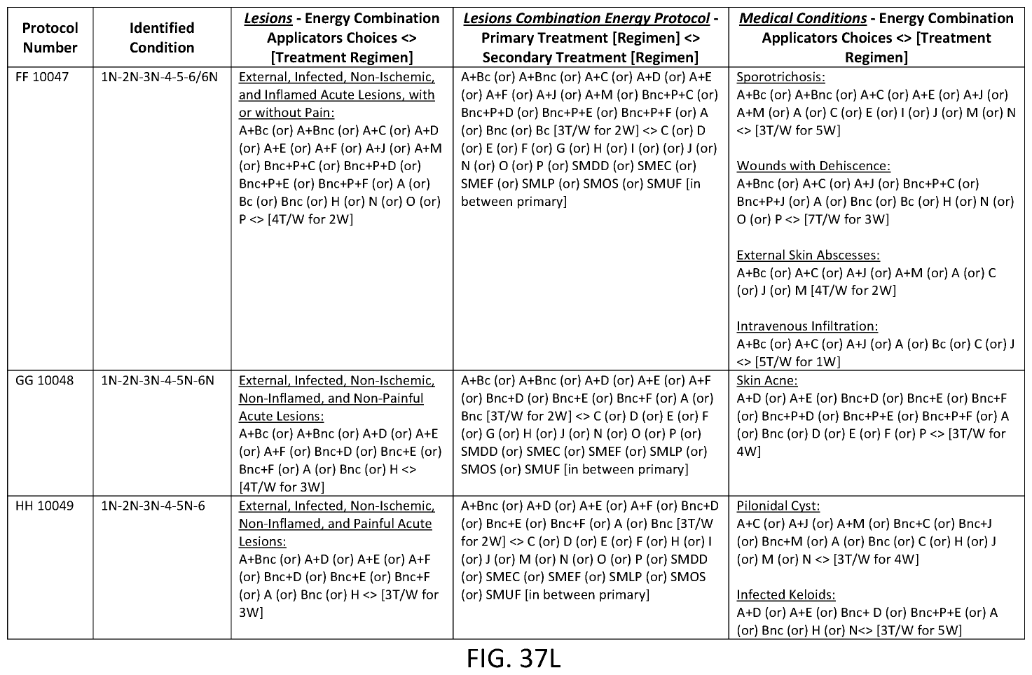

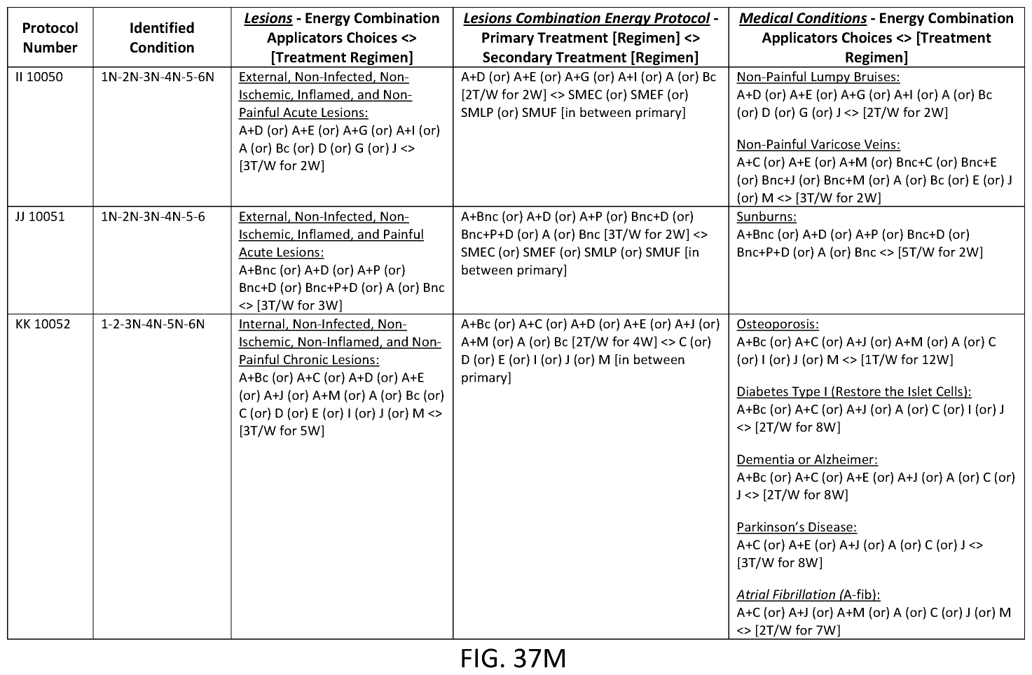

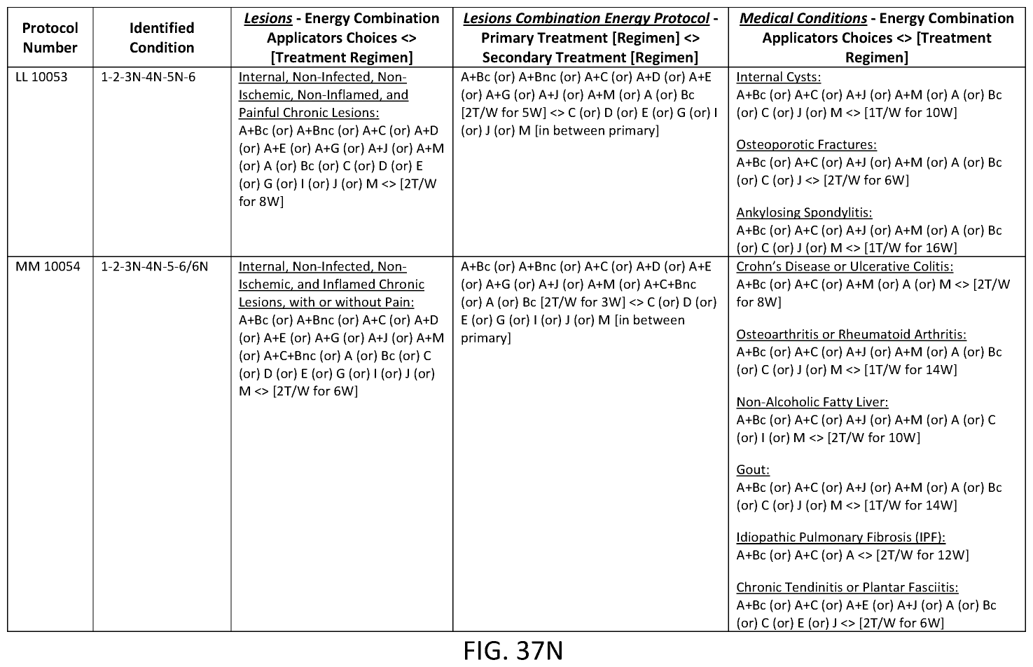

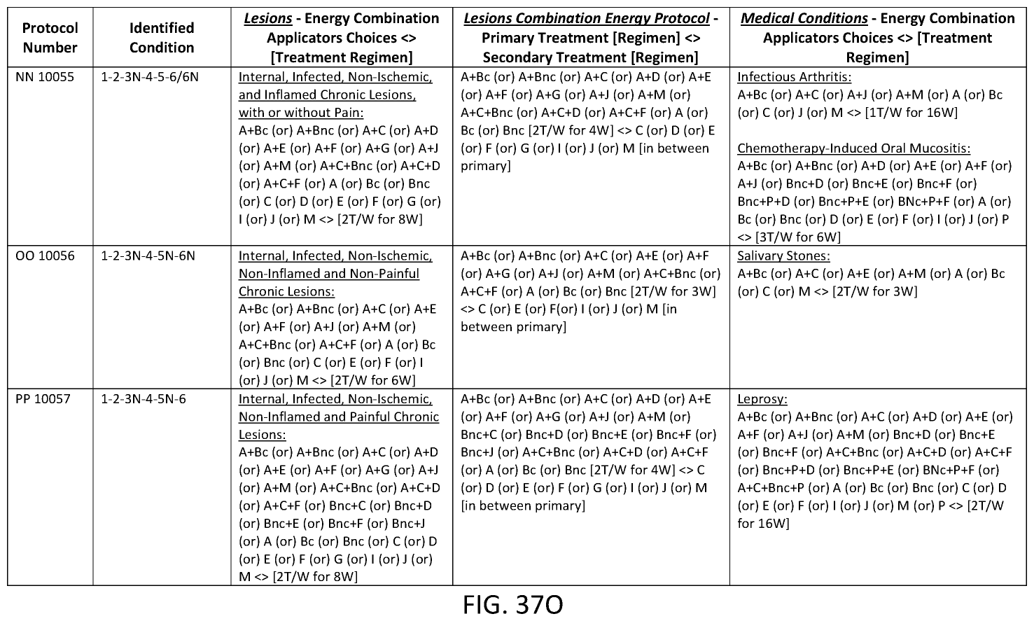

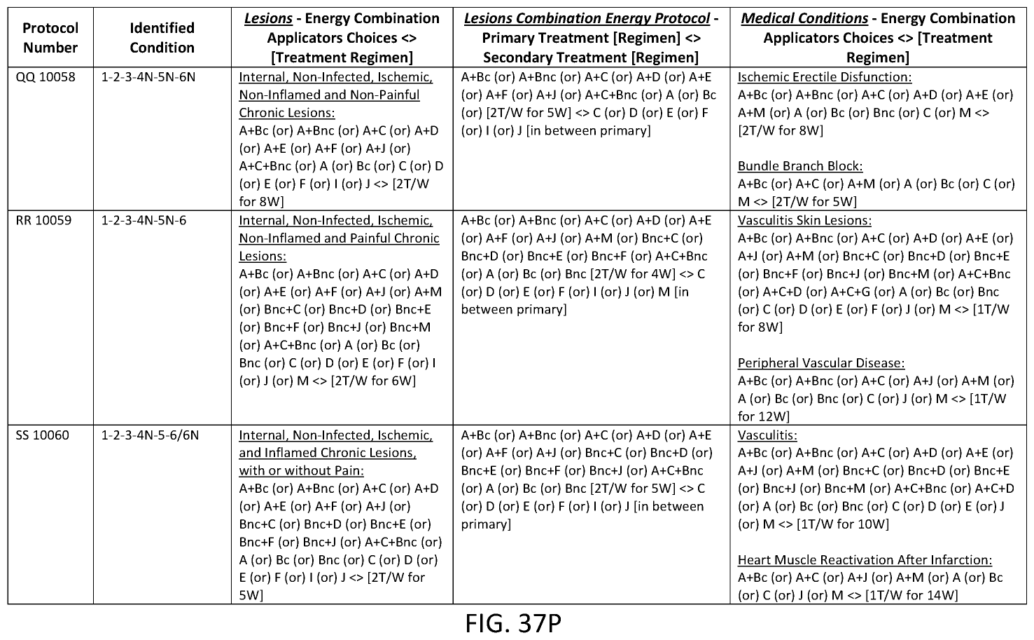

The first treatment selection that is determined via the algorithm from FIGS. 31-35 is Protocol A 10016, which refers to the treatment of non-chronic, superficial/external, non-ischemic, non-infected, with no inflammation, and no painful medical conditions. This is the case of benign superficial acute tissue lesions. For the options of the energy combination systems and their associated energy combination applicators together with treatment regimens of such lesions using Protocol A 10016 see FIG. 37A and also FIGS. 36A-36B for the symbols used. From FIG. 37A, the energy combination applicators option is Bnc+D, which according to the legend tables from FIGS. 36A-36B translates in an energy combination applicator that incorporates a non-contact ultrasound and LED/OLED phototherapy technologies. From the same FIG. 37A, the treatment regimen is [2T/W for 2 W] that according to the legend tables from FIGS. 36A-36B translates in 2 treatment sessions per week (2T/W) for 2 weeks (2 W). When multiple energy combination applicator options are available, they are separated by (or) that indicates that there is option 1 or an option 2 or etc. For example, that can be seen in FIG. 37A for Protocol B 10017.

Equally, a combination energy protocol is a protocol that is using independent energy devices (incorporating only one energy technology, as presented in other patents as U.S. Pat. Nos. 10,888,715, 11,224,767 and 11,331,520) or energy combination applicators and systems (incorporating two or more energy technologies, as presented in this invention) to perform main treatment sessions, which are combined with secondary treatment sessions that can be performed in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol A 10016 see FIG. 37A and FIGS. 36A-36B for the symbols used.

Examples of other non-chronic, superficial, non-ischemic, non-infected, with no inflammation and no painful medical conditions that can use the energy combination systems or combination energy protocols corresponding in general to the selection of Protocol A 10016 are cosmetic conditions (cellulite, body sculpting, skin rejuvenation, spider veins, scar prevention, wrinkles, skin roughness, actinic elastosis, mottled hyperpigmentation, etc.), liposuction, hair regrowth, rosacea, erectile disfunction based on neurologic etiology, and others.

For the energy treatment of cosmetic conditions in accordance with Protocol A 10016, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

For the energy treatment of liposuction in accordance with Protocol A 10016, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used). These options can be used as extracorporeal solutions, depending on the condition and preference of the medical staff. The same type of combination applicators can be used together with the subcutaneous wands that use radio-frequency, laser, or ultrasound technologies. The liposuction is done in one (1) treatment session. Complications from liposuction can be treated with other protocols presented in these inventions, based on the type of complications (bruising, fluid accumulation, infection, damage to nerves, blood vessels, muscles, etc.). To avoid such complications generated by invasive treatments, the non-invasive approach using energy combination applicators, as presented in these inventions, is much more indicated.

For the energy treatment of hair regrowth and rosacea in accordance with Protocol A 10016, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used). For the energy combination applicators/systems that use the non-contact ultrasound, the mist solution that conduct the ultrasound towards the treatment targeted zone can incorporate different active substances to help with reducing the action of different factors that generate rosacea or scalp irritation or bring necessary vitamins to hair follicles that will promote hair regrowth.

For the energy treatment of erectile disfunction based on neurologic etiology in accordance with Protocol A 10016, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

Note that there are different solutions of energy combination applicators or systems (incorporating two or more energy technologies, as presented in this invention) or independent energy devices (incorporating only one energy technology) that can be used for treatment sessions of a tissue/organ condition according to Protocol A 10016. Using artificial intelligence (AI) algorithm(s), these different solutions can be chosen to be specifically suitable for a personalized treatment based on factors such as a patient's comorbidities, state of the tissue condition, individual physical characteristics, and lifestyle parameters. That is how differentiation is done in between different options for the medical devices or systems or applicators used for a treatment session. Also, depending on the medical facility inventory of devices/systems/applicators, different solutions can be chosen based on the artificial intelligence (AI) algorithm(s) that knows the availability of such medical devices or systems or energy applicators for that respective facility. Furthermore, the same artificial intelligence (AI) algorithm(s) can provide an automatic adjustment of each individual treatment session dosage (treatment time duration and energy level or wattage level) to be administered. This approach is valid not only for Protocol A 10016, but also for all the other protocols mentioned in FIGS. 31-35.

Depending on the affliction and as needed for each case/patient, all treatment embodiments mentioned above for Protocol A 10016 might be repeated after a period of pause of at least one month.

The next treatment selection that is determined via the algorithm from FIGS. 31-35 is Protocol B 10017, which refers to the treatment of non-chronic, superficial/external, non-ischemic, non-infected, with no inflammation, and painful medical conditions. This is the case of a painful superficial acute tissue lesion. For the options of the energy combination systems and their associated energy combination applicators together with treatment regimens of such lesions using Protocol B 10017 see FIG. 37A and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol B 10017 see FIG. 37A and FIGS. 36A-36B for the symbols used.

Examples of other non-chronic, superficial, non-ischemic, non-infected, with no inflammation and painful medical conditions that can use the energy combination systems or combination energy protocols corresponding in general to the selection of Protocol B 10017 are Peyronie's disease, nail regrowth after detachment, external hemorrhoids, external cysts (sebaceous, breast, Bartholin's cysts from vagina entrance, etc.), and others.

For the energy treatment of Peyronie's disease (fibrous scar tissue inside the penis that causes curved and painful erections) in accordance with Protocol B 10017, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

For the energy treatment of nail regrowth after detachment in accordance with Protocol B 10017, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

For the energy treatment of external hemorrhoids in accordance with Protocol B 10017, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

For the energy treatment of external cysts in accordance with Protocol B 10017, different options of energy combination systems (with applicators incorporating multiple energy technologies and their control consoles, as presented in this invention) or independent energy devices (incorporating only one energy technology) are available, which are presented together with the associated treatment regimen in FIG. 37A (see also FIGS. 36A-36B for the symbols used).

Depending on the affliction and as needed for each case/patient, all treatment embodiments mentioned above for Protocol B 10017 might be repeated after a period of pause of at least one month.

The next treatment selection that is determined via the algorithm from FIGS. 31-35 is Protocol C 10018, which refers to the treatment of chronic, superficial, non-ischemic, non-infected, with no inflammation, and no painful medical conditions. This is the case of a non-painful superficial chronic/non-healing surgical or trauma tissue lesion. For the options of the energy combination systems and their associated energy combination applicators together with treatment regimens of such lesions using Protocol C 10018 see FIG. 37B and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol C 10018 see FIG. 37B and FIGS. 36A-36B for the symbols used.

Examples of other chronic, superficial, non-ischemic, non-infected, with no inflammation and no painful medical conditions that can use the energy combination systems or combination energy protocols using different energy products corresponding in general to the selection of Protocol C 10018 are non-infected keloid scars, hypertrophic scars, and others. In this case, one treatment session or multiple treatment sessions can be applied, depending on the type of condition treated.

A non-infected keloid scar is a thick raised scar caused by an excess protein (collagen) in the skin during healing after an injury. They can grow to be much larger than the original injury that caused the scar and are more likely for people who have dark skin. Treatment is usually done via surgical intervention and radiation. For the energy treatment of non-infected keloid scar condition in accordance with Protocol C 10018, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The radiation regimen can be continued concurrently with the Protocol C 10018 presented in this invention, at the physician's discretion.

A hypertrophic scar is a thick raised scar that is an abnormal response to wound healing following skin trauma, burns, or surgical incisions. Treatments include medication, freezing, injections, lasers, and surgery. For the energy treatment of hypertrophic scar condition in accordance with Protocol C 10018, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The specialized medication regimen can be continued concurrently with the Protocol C 10018 presented in this invention, at the physician's discretion.

Depending on the affliction and as needed for each case/patient, all treatment embodiments mentioned above for Protocol C 10018 might be repeated after a period of pause of at least one month.

The next treatment selection that is determined via the algorithm from FIGS. 31-35 is Protocol D 10019, which refers to the treatment of chronic, superficial, non-ischemic, non-infected, with no inflammation, and painful medical conditions. This is the case of superficial chronic pressure ulcers/tissue lesions. Superficial chronic pressure ulcers (also known as pressure sores or bedsores) are injuries to the skin and underlying tissue, primarily caused by prolonged pressure on the skin. They can happen to anyone, but usually affect people confined to bed or who sit in a chair or wheelchair for long periods of time. For the options of the energy combination systems and their associated energy combination applicators together with treatment regimens of such lesions using Protocol D 10019 see FIG. 37B and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol D 10019 see FIG. 37B and FIGS. 36A-36B for the symbols used.

Examples of other chronic, superficial, non-ischemic, non-infected, with no inflammation and painful medical conditions that can use the energy combination systems or combination energy protocols using different energy products corresponding in general to the selection of Protocol D 10019 are pyoderma gangrenosum, fibromyalgia, psoriasis, epidermolysis bullosa, shingles, and others.

Pyoderma gangrenosum is a rare condition of the immune system defined as noninfectious neutrophilic dermatosis of the skin that causes large, painful sores (ulcers) to develop on the skin, most often on legs. Treatment of pyoderma gangrenosum is aimed at reducing inflammation, controlling pain, promoting wound healing, and controlling any underlying disease, with a combination of pills, creams or injections. For the energy treatment of pyoderma gangrenosum condition in accordance with Protocol D 10019, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used).

Fibromyalgia is a disorder characterized by widespread musculoskeletal pain accompanied by fatigue, sleep, memory, and mood issues. With fibromyalgia, a mix of treatments often works best. Medicine may be prescribed to ease pain and improve sleep. Physical therapy and counseling also can reduce pain and boost quality of life. Lifestyle changes are important too. It's key to eat healthy, get regular exercise and get quality sleep. For the energy treatment of fibromyalgia condition in accordance with Protocol D 10019, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The specialized medication regimen can be continued concurrently with the Protocol D 10019 presented in this invention, at the physician's discretion.

Psoriasis is a skin chronic disease that causes red, itchy scaly patches, most commonly on the knees, elbows, trunk, and scalp. Topical ointments, light therapy, and medications can offer relief. For the energy treatment of psoriasis condition in accordance with Protocol D 10019, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The specialized medication regimen can be continued concurrently with the Protocol D 10019 presented in this invention, at the physician's discretion.

Epidermolysis bullosa is a group of rare diseases that cause fragile, blistering skin in response to minor injury, even from heat, rubbing, scratching, or adhesive tape. Treatment consists of topical gel medication that can help manage epidermolysis bullosa wounds. Medication is often needed to relieve the pain as antidepressants and acetaminophen. If the pain is severe, medicine like fentanyl, morphine, or ketamine can be prescribed. For the energy treatment of epidermolysis bullosa condition in accordance with Protocol D 10019, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The specialized topical medication regimen can be continued concurrently with the Protocol D 10019 presented in this invention, at the physician's discretion.

Shingles is a viral infection that causes a painful rash that may appear as a stripe of blisters on the trunk of the body. Anyone who's had chickenpox may develop shingles, which is a reactivation of the virus. Treatments include pain relief and antiviral medications such as acyclovir or valacyclovir. A chickenpox vaccine in childhood or a shingles vaccine as an adult can minimize the risk of developing shingles. For the energy treatment of shingles condition in accordance with Protocol D 10019, different options of energy combination systems (with applicators incorporating multiple energy technologies and their control consoles, as presented in this invention) or independent energy devices (incorporating only one energy technology) are available, which are presented together with the associated treatment regimen in FIG. 37B (see also FIGS. 36A-36B for the symbols used). The specialized antiviral medication regimen can be continued concurrently with the Protocol D 10019 presented in this invention, at the physician's discretion.

Depending on the affliction and as needed for each case/patient, all treatment embodiments mentioned above for Protocol D 10019 might be repeated after a period of pause of at least one month.

The next treatment selection that is determined via the algorithm from FIGS. 31-35 is Protocol E 10020, which refers to the treatment of chronic, superficial, non-ischemic, non-infected, with inflammation, and painful or non-painful medical conditions. This is the case of a superficial chronic tissue lesion that present significant inflammation (for example a venous wound). For the options of the energy combination systems and their associated energy combination applicators together with treatment regimens of such lesions using Protocol E 10020 see FIG. 37C and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol E 10020 see FIG. 37C and FIGS. 36A-36B for the symbols used.

Examples of other chronic, superficial, non-ischemic, non-infected, with inflammation and painful or non-painful medical conditions that can use the energy combination systems or combination energy protocols using different energy products corresponding in general to the selection of Protocol E 10020 are Behçet's disease, eczemas, periodontitis, and others.

Behçet's disease is a rare disorder causing inflammation in blood vessels. The cause of Behçet's disease is unknown, but it may be the body's immune system attacking healthy cells (autoimmune disorder). Symptoms include mouth and genital sores, inflamed eyes, and rashes. Treatment consists of medicines that control the symptoms, as steroids and other anti-inflammatory medicines, immunosuppressants to reduce the activity of the immune system, which overall reduce inflammation in the affected parts of the body. For the energy treatment of Behçet's disease condition in accordance with Protocol E 10020, different options of energy combination systems (with applicators incorporating multiple energy technologies and their control consoles, as presented in this invention) or independent energy devices (incorporating only one energy technology) are available, which are presented together with the associated treatment regimen in FIG. 37C (see also FIGS. 36A-36B for the symbols used). The specialized medication regimen can be continued concurrently with the Protocol E 10020 presented in this invention, at the physician's discretion.

Eczema, which is also called dermatitis, is a term for several different types of skin swelling. It causes dry, itchy skin and rashes on the face, inside the elbows and behind the knees, and on the hands and feet. Scratching the skin can cause it to turn red, to swell, and itch even more. Treatment varies and may include creams and ointments to control inflammation or treat any infectious causes. For the energy treatment of eczema condition in accordance with Protocol E 10020, different options of energy combination systems (with applicators incorporating multiple energy technologies and their control consoles, as presented in this invention) or independent energy devices (incorporating only one energy technology) are available, which are presented together with the associated treatment regimen in FIG. 37C (see also FIGS. 36A-36B for the symbols used).

Periodontitis also called gum disease, is a serious gum infection that damages the soft tissue and, without treatment, can destroy the bone that supports your teeth. Periodontitis can cause teeth to loosen or lead to tooth loss. It produces inflammation of the gum, bleeding, pain, bead breath, etc. The treatment for periodontitis consists of topical or oral antibiotics to control bacterial infection and also professionally cleaning of the pockets around teeth to prevent damage to surrounding bone. Advanced cases may require surgery. For the energy treatment of periodontitis condition in accordance with Protocol E 10020, different options of energy combination systems (with applicators incorporating multiple energy technologies and their control consoles, as presented in this invention) or independent energy devices (incorporating only one energy technology) are available, which are presented together with the associated treatment regimen in FIG. 37C (see also FIGS. 36A-36B for the symbols used). The antibiotics regimen can be continued concurrently with the Protocol E 10020 presented in this invention, at the physician discretion.

Depending on the affliction and as needed for each case/patient, all treatment embodiments mentioned above for Protocol E 10020 might be repeated after a period of pause of at least one month.

The next treatment selection that is determined via the algorithm from FIGS. 31-35 is Protocol F 10021, which refers to the treatment of chronic, superficial, non-ischemic, infected medical conditions, which might present or not inflammation and/or pain. This is the case of a superficial infected chronic tissue lesion. For the options of the energy combination systems and their associated energy combination applicators together with treatment regimens of such lesions using Protocol F 10021 see FIG. 37C and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol F 10021 see FIG. 37C and FIGS. 36A-36B for the symbols used.

Examples of other chronic, superficial, non-ischemic, infected medical conditions, which might present or not inflammation and/or pain that can use the energy combination systems or combination energy protocols using different energy products corresponding in general to the selection of Protocol F 10021 are ringworm infections, toenail fungus, and others.

Ringworm is a common skin or scalp highly contagious infection that is caused by a fungus. It is called “ringworm” because it can cause a circular rash (shaped like a ring) that is usually scaly, red, and itchy. The infection is spread by skin-to-skin contact or by touching an infected animal or object. Ringworm of the scalp is common in children, where it may cause bald patches. The treatment for ringworm is antifungal medications. For the energy treatment of ringworms condition in accordance with Protocol F 10021, different options of energy combination systems (with applicators incorporating multiple energy technologies and their control consoles, as presented in this invention) or independent energy devices (incorporating only one energy technology) are available, which are presented together with the associated treatment regimen in FIG. 37C (see also FIGS. 36A-36B for the symbols used). The antifungal medication regimen can be continued concurrently with the Protocol F 10021 presented in this invention, at the physician's discretion.

Toenail fungus is a nail fungus causing thickened, brittle, crumbly, or ragged nails. Usually, the problems caused by this condition are cosmetic. The main symptoms are changes in the appearance of nails and rarely, the condition causes pain or a slightly foul odor. Treatments include oral antifungal drugs, medicated nail polish or cream, or nail removal. For the energy treatment of toenail fungus condition in accordance with Protocol F 10021, different options of energy combination systems (with applicators incorporating multiple energy technologies and their control consoles, as presented in this invention) or independent energy devices (incorporating only one energy technology) are available, which are presented together with the associated treatment regimen in FIG. 37C (see also FIGS. 36A-36B for the symbols used). The antifungal medication regimen can be continued concurrently with the Protocol F 10021 presented in this invention, at the physician's discretion.

Depending on the affliction and as needed for each case/patient, all treatment embodiments mentioned above for Protocol F 10021 might be repeated after a period of pause of at least one month.

The next treatment selection that is determined via the algorithm from FIGS. 31-35 is Protocol G 10022, which refers to the treatment of chronic, superficial, ischemic, non-infected, with no inflammation, and no painful medical conditions. This is the case of a superficial non-infected and ischemic chronic tissue lesion. For the options of the energy combination systems and their associated energy combination applicators together with treatment regimens of such lesions using Protocol G 10022 see FIG. 37D and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol G 10022 see FIG. 37D and FIGS. 36A-36B for the symbols used.

Examples of other chronic, superficial, ischemic, non-infected, with no inflammation and no painful medical conditions that can use the energy combination systems or combination energy protocols using different energy products corresponding in general to the selection of Protocol G 10022 are chronic mesenteric ischemia and others.

Chronic mesenteric ischemia, also referred to as intestinal ischemia occurs when plaque builds up in the major arteries that supply blood to the small intestine or small bowel. When left untreated, the blockage can decrease blood flow so much that the tissues in the intestines die. Chronic mesenteric ischemia is treated with open surgery or a procedure called angioplasty. For the energy treatment of chronic mesenteric ischemia in accordance with Protocol G 10022, different options of energy combination systems (with applicators incorporating multiple energy technologies and their control consoles, as presented in this invention) or independent energy devices (incorporating only one energy technology) are available, which are presented together with the associated treatment regimen in FIG. 37D (see also FIGS. 36A-36B for the symbols used).

Depending on the affliction and as needed for each case/patient, all treatment embodiments mentioned above for Protocol G 10022 might be repeated after a period of pause of at least one month.

The next treatment selection that is determined via the algorithm from FIGS. 31-35 is Protocol H 10023, which refers to the treatment of chronic, superficial, ischemic, non-infected, with no inflammation, and painful medical conditions. This is the case of a superficial non-infected and ischemic chronic tissue lesion (arterial wounds). Chronic ischemic (arterial) ulcers can occur when there is poor blood flow to the legs and on feet. Poor blood flow causes cells to die and damages tissue. This type of wounds can be slow to heal. For the options of the energy combination systems and their associated energy combination applicators together with treatment regimens of such lesions using Protocol H 10023 see FIG. 37D and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol H 10023 see FIG. 37D and FIGS. 36A-36B for the symbols used.

Examples of other chronic, superficial, ischemic, non-infected, with no inflammation and painful medical conditions that can use the energy combination systems or combination energy protocols using different energy products corresponding in general to the selection of Protocol H 10023 are chronic deep tissue injuries, and others.

Chronic deep tissue injuries are injuries are defined as ‘purple or maroon localized area of discolored intact skin or blood-filled blister due to damage of underlying soft tissue from localized intense pressure and/or shear. Treatment consists of frequent repositioning off the site of injury, good skin care, proper support surface selection, as well as correcting any systemic issues or nutritional deficiencies. For the energy treatment of chronic deep tissue injuries in accordance with Protocol H 10023, different options of energy combination systems (with applicators incorporating multiple energy technologies and their control consoles, as presented in this invention) or independent energy devices (incorporating only one energy technology) are available, which are presented together with the associated treatment regimen in FIG. 37D (see also FIGS. 36A-36B for the symbols used).

Depending on the affliction and as needed for each case/patient, all treatment embodiments mentioned above for Protocol H 10023 might be repeated after a period of pause of at least one month.

The next treatment selection that is determined via the algorithm from FIGS. 31-35 is Protocol I 10024, which refers to the treatment of chronic, superficial, ischemic, non-infected, with inflammation medical conditions, which might have pain or not. This is the case of a superficial non-infected and ischemic chronic tissue lesion that has significant inflammation, as infected arterial ulcers. Chronic ischemic (arterial) ulcers that present infection can occur when there is poor blood flow to the legs and on feet. Poor blood flow causes cells/tissue to die and makes the area prone for infections. This type of wounds can be very slow to heal. For the options of the energy combination systems and their associated energy combination applicators together with treatment regimens of such lesions using Protocol I 10024 see FIG. 37D and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol I 10024 see FIG. 37D and FIGS. 36A-36B for the symbols used.

Examples of other chronic, superficial, ischemic, non-infected, with inflammation medical conditions that might present or not pain, which can use the energy combination systems or combination energy protocols using different energy products corresponding in general to the selection of Protocol I 10024 are chronic infected diabetic foot ulcers (DFUs), chronic ischemic and infected mixed arterial and venous ulcers, and others.

Chronic infected diabetic foot ulcers (DFUs) are open sores or wounds that occur in approximately 15 percent of patients with diabetes, and are commonly located on the bottom of the foot. The standard practices in DFU management include surgical debridement, dressings to facilitate a moist wound environment and exudate control, wound off-loading, vascular assessment, and infection and glycemic control. For the energy treatment of chronic infected diabetic foot ulcers in accordance with Protocol I 10024, different options of energy combination systems (with applicators incorporating multiple energy technologies and their control consoles, as presented in this invention) or independent energy devices (incorporating only one energy technology) are available, which are presented together with the associated treatment regimen in FIG. 37D (see also FIGS. 36A-36B for the symbols used). The antibiotic regimen (for infected DFUs) can be continued concurrently with the Protocol I 10024 presented in this invention, at the physician's discretion.

Chronic ischemic and infected mixed arterial and venous ulcers are also known as combined arterial and venous insufficiency ulcers. The patients presenting with such ulcers combine chronic venous insufficiency and peripheral arterial occlusive disease. To ensure healing, such wounds must be vascularized, free of necrotic and devitalized tissue, clear of infection, and kept moist. Ideal wound dressings absorb excess fluid while maintaining a moist environment, control bacterial invasion and/or proliferation, eliminate dead space, debride necrotic tissue, avoid maceration of healthy surrounding tissue, and minimize pain with dressing changes. For the energy treatment of chronic ischemic and infected mixed arterial and venous ulcers in accordance with Protocol I 10024, different options of energy combination systems (with applicators incorporating multiple energy technologies and their control consoles, as presented in this invention) or independent energy devices (incorporating only one energy technology) are available, which are presented together with the associated treatment regimen in FIG. 37D (see also FIGS. 36A-36B for the symbols used). The antibiotic regimen (for infected ulcers) can be continued concurrently with the Protocol I 10024 presented in this invention, at the physician's discretion.

Depending on the affliction and as needed for each case/patient, all treatment embodiments mentioned above for Protocol I 10024 might be repeated after a period of pause of at least one month.

The next treatment selection that is determined via the algorithm from FIGS. 31-35 is Protocol J 10025, which refers to the treatment of chronic, superficial, ischemic, infected medical conditions, which might present or not inflammation and pain. This is the case of a superficial infected and ischemic chronic tissue lesion that has or not inflammation and pain. For the options of the energy combination systems and their associated energy combination applicators together with treatment regimens of such lesions using Protocol J 10025 see FIG. 37E and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol J 10025 see FIG. 37E and FIGS. 36A-36B for the symbols used.

Examples of other chronic, superficial, ischemic, infected medical conditions, which might present or not inflammation and pain, which can use the energy combination systems or combination energy protocols using different energy products corresponding in general to the selection of Protocol J 10025 are Cutaneous Lupus Erythematosus, calciphylaxis ulcers, and others.

Cutaneous Lupus Erythematosus is caused by an autoimmune response, meaning the body attacks its own tissues and organs. In cutaneous lupus, the immune system targets skin cells, causing inflammation that leads to red, thick, and often scaly rashes and sores that may burn or itch or can get infected. Treatment for cutaneous lupus erythematosus include pain relievers, medicines for possible infections, corticosteroids, biologics and medicines that lower immune system responses. For the energy treatment of Cutaneous Lupus Erythematosus in accordance with Protocol J 10025, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used). The antibiotic regimen (for infected lesions) can be continued concurrently with the Protocol J 10025 presented in this invention, at the physician's discretion.

Calciphylaxis ulcers are produced by Calciphylaxis that is a serious, uncommon disease, which generates calcium accumulation in small blood vessels of the fat and skin tissues. Calciphylaxis causes blood clots, painful skin ulcers and may cause serious infections that can lead to death. Treatment consists of optimizing wound management with judicious use of surgical debridement and hyperbaric oxygen therapy, halting progression of vessel wall calcification by ameliorating risk factors of calciphylaxis, and reversing vessel wall calcification with sodium thiosulfate. For the energy treatment of calciphylaxis ulcers in accordance with Protocol J 10025, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used). The antibiotic regimen (for infected ulcers) and the administration of sodium thiosulfate can be continued concurrently with the Protocol J 10025 presented in this invention, at the physician's discretion.

Depending on the affliction and as needed for each case/patient, all treatment embodiments mentioned above for Protocol J 10025 might be repeated after a period of pause of at least one month.

The next treatment selection that is determined via the algorithm from FIGS. 31-35 is Protocol K 10026, which refers to the treatment of chronic, internal, ischemic, infected medical conditions, with no inflammation or pain. This is the case of an internal infected and ischemic chronic tissue lesion, with no inflammation or pain. For the options of the energy combination systems and their associated energy combination applicators together with treatment regimens of such lesions using Protocol K 10026 see FIG. 37E and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol K 10026 see FIG. 37E and FIGS. 36A-36B for the symbols used.

Examples of other chronic, internal, ischemic, infected medical conditions, with no inflammation or pain, which can use the energy combination systems or combination energy protocols using different energy products corresponding in general to the selection of Protocol K 10026 are cystic fibrosis, chronic obstructive pulmonary disease, exposed tendons from chronic wounds, and others.

Cystic fibrosis (CF) is an inherited disorder that causes severe damage to the lungs, digestive system, and other organs in the body. This disorder affects the cells that produce mucus, sweat, and digestive juices, which are normally thin and slippery. Cystic fibrosis causes these fluids to become thick and sticky, which then plug up tubes, ducts, and passageways. Symptoms vary and can include cough, repeated lung infections, inability to gain weight, and fatty stools. The treatments are used to ease symptoms, and include chest wall oscillation, postural drainage, antibiotics for infections, and cough medicine. For the energy treatment of cystic fibrosis in accordance with Protocol K 10026, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used). The antibiotic regimen (for infected CF) can be continued concurrently with the Protocol K 10026 presented in this invention, at the physician's discretion.

Chronic obstructive pulmonary disease, or COPD, refers to a group of diseases that cause airflow blockage and breathing-related problems. Emphysema and chronic bronchitis are the most common conditions that make up COPD. Damage to the lungs from COPD cannot be reversed. Symptoms include shortness of breath, wheezing, or a chronic cough. Vaccination, rescue inhalers, nebulizers, and inhaled or oral steroids can help control symptoms and minimize further damage. For more complicated cases oxygen therapy is needed. For the energy treatment of COPD in accordance with Protocol K 10026, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used).

Exposed tendons for severe chronic wounds as Stage IV pressure ulcers and diabetic ulcers. Tendons are nourished by blood vessels and diffusion of nutrients from synovial fluid. The exposure of tendons to air will cause desiccation and subsequent tissue necrosis and infection. For tendon-exposed wounds, flap prostheses or skin grafts are used after long-term drug replacement. Some wounds require secondary flap prostheses because the thick flap partially affects appearance and function. For the energy treatment of exposed tendons from chronic wounds in accordance with Protocol K 10026, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used). The antibiotic regimen (for infected tendons) can be continued concurrently with the Protocol K 10026 presented in this invention, at the physician's discretion.

Depending on the affliction and as needed for each case/patient, all treatment embodiments mentioned above for Protocol K 10026 might be repeated after a period of pause of at least one month.

The next treatment selection that is determined via the algorithm from FIGS. 31-35 is Protocol L 10027, which refers to the treatment of chronic, internal, ischemic, infected, painful, and without inflammation medical conditions. This is the case of an internal infected and ischemic chronic tissue lesion, with significant pain and no inflammation. For the options of the energy combination systems and their associated energy combination applicators together with treatment regimens of such lesions using Protocol L 10027 see FIG. 37E and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol L 10027 see FIG. 37E and FIGS. 36A-36B for the symbols used.

Examples of other chronic, internal, ischemic, infected, painful and without inflammation medical conditions, which can use the energy combination systems or combination energy protocols using different energy products corresponding in general to the selection of Protocol L 10027 are chronic ischemic colitis, and others.

Chronic ischemic colitis is inflammation of the large intestine, or colon due to the lack of blood flow to the area, usually because an artery is chronically blocked or narrowed. If the blood does not get to the colon, then the tissue dies and produce life threating infections. Treatment includes antibiotics to prevent infections, intravenous fluids for dehydration, treatment for any underlying medical condition, such as congestive heart failure or an irregular heartbeat, and avoiding medications that constrict blood vessels, such as migraine drugs, hormone medications and some heart drugs. For the energy treatment of chronic ischemic colitis in accordance with Protocol L 10027, different options of energy combination systems (with applicators incorporating multiple energy technologies and their control consoles, as presented in this invention) or independent energy devices (incorporating only one energy technology) are available, which are presented together with the associated treatment regimen in FIG. 37E (see also FIGS. 36A-36B for the symbols used). The antibiotic regimen for infected ischemic colitis must be continued concurrently with the Protocol L 10027 presented in this invention.

Depending on the affliction and as needed for each case/patient, all treatment embodiments mentioned above for Protocol L 10027 might be repeated after a period of pause of at least one month.

The next treatment selection that is determined via the algorithm from FIGS. 31-35 is Protocol M 10028, which refers to the treatment of chronic, internal, ischemic, infected, and with inflammation medical conditions, with or without pain. This is the case of an internal chronic, infected and ischemic chronic tissue lesion, with significant inflammation. For the options of the energy combination systems and their associated energy combination applicators together with treatment regimens of such lesions using Protocol M 10028 see FIG. 37F and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol M 10028 see FIG. 37F and FIGS. 36A-36B for the symbols used.

Examples of other chronic, internal, ischemic, infected, with inflammation medical conditions, and with or without pain, which can use the energy combination systems or combination energy protocols using different energy products corresponding in general to the selection of Protocol M 10028 are intestinal ischemia, and others.

Intestinal ischemia describes a variety of conditions that occur when blood flow to intestines decreases. Ischemia can be due to a fully or partially blocked blood vessel, usually an artery, or low blood pressure leading to an overall reduced blood flow. Intestinal ischemia can affect the small intestine, the large intestine (colon) or both. Treatment may include antibiotics against infection, blood thinners or a surgical procedure to restore blood flow to the small intestine. For the energy treatment of intestinal ischemia in accordance with Protocol M 10028, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used). The aim of the energy treatment is the restoration of blood flow and elimination of infection. The antibiotic regimen must be continued concurrently with the Protocol M 10028 presented in this invention.

Depending on the affliction and as needed for each case/patient, all treatment embodiments mentioned above for Protocol M 10028 might be repeated after a period of pause of at least one month.

The next treatment selection that is determined via the algorithm from FIGS. 31-35 is Protocol N 10029, which refers to the treatment of non-chronic, internal, non-ischemic, non-infected, with no inflammation, and no painful medical conditions. This is the case of an internal non-infected and non-ischemic acute tissue lesion, with no inflammation or pain. For the options of the energy combination systems and their associated energy combination applicators together with treatment regimens of such lesions using Protocol N 10029 see FIG. 37F and also FIGS. 36A-36B for the symbols used.

Equally, a combination energy protocol is a protocol that is using independent energy devices (incorporating only one energy technology, which are presented in other patents) or energy combination applicators and systems (incorporating two or more energy technologies, as presented in this invention) to perform main treatment sessions that are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol N 10029 see FIG. 37F and FIGS. 36A-36B for the symbols used.

Examples of other non-chronic, internal, non-ischemic, non-infected, with no inflammation, and no painful medical conditions, which can use the energy combination systems or combination energy protocols using different energy products corresponding in general to the selection of Protocol N 10029 are Guillain-Barré syndrome, internal hemorrhoids, partially torn ligaments or tendons (grade 1 or 2), rotator cuff syndrome, and others.

Guillain-Barré syndrome (GBS) is a rare, autoimmune disorder in which a person's own immune system damages the nerves, causing muscle weakness and sometimes paralysis. GBS can cause symptoms that last for a few weeks to several years. Most people recover fully, but some have permanent nerve damage. Special blood treatments (plasma exchange and immunoglobulin therapy) can relieve symptoms and also physical therapy is needed. For the energy treatment of Guillain-Barré syndrome in accordance with Protocol N 10029, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Internal hemorrhoids lie inside the rectum and they rarely cause discomfort. However, due straining or irritation when passing stool, the hemorrhoids can bleed during bowel movements and are typically painless, even when they produce bleeding. Treatment consists of rubber band ligation, sclerotherapy (injection of solution into an internal hemorrhoid), infrared photocoagulation, and electrocoagulation. For the energy treatment of internal hemorrhoids in accordance with Protocol N 10029, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Partially torn ligaments or tendons (grade 1 or 2) can heal themselves in a long period of time. Mild ligament sprains can take from two to four weeks to heal, and moderate sprains may take more than 10 weeks. The torn ligaments need proper care and doctor's supervision for better and quick healing. The treatment consists of rest, ice, compression, and elevation. For the energy treatment of partially torn ligaments or tendons in accordance with Protocol N 10029, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Rotator cuff syndrome is a tear in the tissues connecting muscle to bone (tendons) around the shoulder joint. A rotator cuff tear often occurs in people who repeatedly perform the same shoulder motions. Symptoms include shoulder pain and weakness. Treatment includes rest, medication, physical therapy, corticosteroid injections, and possibly surgery. For the energy treatment of rotator cuff syndrome in accordance with Protocol N 10029, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used). The corticosteroid regimen can be continued with the Protocol N 10029 presented in this invention, at the physician's discretion.

Depending on the affliction and as needed for each case/patient, all treatment embodiments mentioned above for Protocol N 10029 might be repeated after a period of pause of at least one month.

The next treatment selection that is determined via the algorithm from FIGS. 31-35 is Protocol O 10030, which refers to the treatment of non-chronic, internal, non-ischemic, non-infected, with no inflammation, and painful medical conditions. This is the case of an internal non-infected and non-ischemic acute tissue lesion, with no inflammation but with significant pain. For the options of the energy combination systems and their associated energy combination applicators together with treatment regimens of such lesions using Protocol O 10030 see FIG. 37F and also FIGS. 36A-36B for the symbols used.

Equally, a combination energy protocol is a protocol that is using independent energy devices (incorporating only one energy technology, which are presented in other patents) or energy combination applicators and systems (incorporating two or more energy technologies, as presented in this invention) to perform main treatment sessions that are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol O 10030 see FIG. 37F and FIGS. 36A-36B for the symbols used.

Examples of other non-chronic, internal, non-ischemic, non-infected, with no inflammation, and painful medical conditions, which can use the energy combination systems or combination energy protocols using different energy products corresponding in general to the selection of Protocol O 10030 are bruises, muscle tears, acute low back pain, and others.

Bruises are blood or bleeding under the skin due to trauma of any kind. Typically, black and blue at first, with color changes as healing progresses. They are usually treated with cold compresses, raise of the bruised area, pain medicine, etc. For the energy treatment of bruises in accordance with Protocol O 10030, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Muscle tears represent a stretching or tearing of a muscle or a tissue connecting muscle to bone (tendon). Strains often occur in the lower back and in the muscle in the back of the thigh. Symptoms include pain, swelling, muscle spasms, and limited ability to move the muscle. Treatment may include pain relievers, ice, or splinting. For the energy treatment of muscle tears in accordance with Protocol O 10030, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Acute low back pain is most often caused by a sudden injury to the muscles and ligaments supporting the back. Common causes include improper lifting, poor posture, lack of regular exercise, fracture, ruptured disk, or arthritis. The pain may be caused by muscle spasms or a strain or tear in the muscles and ligaments. Most low back pain goes away on its own in two to four weeks. Physical therapy and pain relievers can help. A few cases may require surgery. For the energy treatment of acute low back pain in accordance with Protocol O 10030, different options of energy combination systems (with applicators incorporating multiple energy technologies and their control consoles, as presented in this invention) or independent energy devices (incorporating only one energy technology) are available, which are presented together with the associated treatment regimen in FIG. 37F (see also FIGS. 36A-36B for the symbols used).

Depending on the affliction and as needed for each case/patient, all treatment embodiments mentioned above for Protocol O 10030 might be repeated after a period of pause of at least one month.

The next treatment selection that is determined via the algorithm from FIGS. 31-35 is Protocol P 10031, which refers to the treatment of non-chronic, internal, non-ischemic, non-infected, and with inflammation medical conditions, with or without pain. This is the case of an internal non-infected and non-ischemic acute tissue lesion, with inflammation, and with or without pain. For the options of the energy combination systems and their associated energy combination applicators together with treatment regimens of such lesions using Protocol P 10031 see FIG. 37G and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol P 10031 see FIG. 37G and FIGS. 36A-36B for the symbols used.

Examples of other non-chronic, internal, non-ischemic, non-infected, and inflamed medical conditions, with or without pain, which can use the energy combination systems or combination energy protocols using different energy products corresponding in general to the selection of Protocol P 10031 are tenosynovitis, non-chronic tendonitis, bursitis, and others.

Tenosynovitis is inflammation of the lining of the sheath that surrounds a tendon, which is a painful condition. The hands, wrists, and feet are often affected. Common causes are injury and repetitive use. Symptoms include pain, swelling, and difficulty moving the affected joint. Treatment includes pain relievers, ice, and joint immobilization. In rare cases, treatment consists of corticosteroid injections, physical therapy, or surgery. For the energy treatment of tenosynovitis in accordance with Protocol P 10031, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used).

Non-chronic tendonitis is the inflammation or irritation of a tendon, which is the thick fibrous cords that attach muscle to bone. The condition causes pain and tenderness just outside a joint. While non-chronic tendinitis can occur in any of tendons, it is most common around the shoulders, elbows, wrists, knees, and heels. Recommended treatment is for pain relief by resting the joint, applying ice packs, compressing the area with an elastic bandage to ease soreness and inflammation, keeping the joint elevated and using over-the-counter pain relievers, such as aspirin (in adults), naproxen, or ibuprofen. For the energy treatment non-chronic tendonitis in accordance with Protocol P 10031, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used).

Bursitis is inflammation of the bursae, which are small fluid-filled sacs that reduce friction between moving parts in the body's joints. Shoulder, elbow, hip, and trochanteric bursitis are the most known. Symptoms include pain that is worse with activities such as standing, walking, or running, depending on the joint. Treatments include ice, anti-inflammatory medications, steroid injections, and physical therapy. For the energy treatment of bursitis in accordance with Protocol P 10031, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used).

Depending on the affliction and as needed for each case/patient, all treatment embodiments mentioned above for Protocol P 10031 might be repeated after a period of pause of at least one month.

The next treatment selection that is determined via the algorithm from FIGS. 31-35 is Protocol Q 10032, which refers to the treatment of non-chronic, internal, non-ischemic, infected, with no inflammation, and no painful medical conditions. This is the case of an internal infected and non-ischemic acute tissue lesion, with no inflammation or pain. For the options of the energy combination systems and their associated energy combination applicators together with treatment regimens of such lesions using Protocol Q 10032 see FIG. 37G and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol Q 10032 see FIG. 37G and FIGS. 36A-36B for the symbols used.

Examples of other non-chronic, internal, non-ischemic, infected, with no inflammation and no painful medical conditions, which can use the energy combination systems or combination energy protocols using different energy products corresponding in general to the selection of Protocol Q 10032 are boils and others.

A boil is a pus-filled bump that forms under the skin when bacteria infect and inflame one or more hair follicles. A carbuncle is a cluster of boils that form a connected area of infection under the skin. Boils start as red, tender lumps and fill with pus, grow, rupture and finally drain. Treatment includes washing with antiseptic soap, bathe or shower every day, wash hands regularly, and covering the area with a dressing or gauze until the boil heals. For the energy treatment of boils in accordance with Protocol Q 10032, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used).

Depending on the affliction and as needed for each case/patient, all treatment embodiments mentioned above for Protocol Q 10032 might be repeated after a period of pause of at least one month.

The next treatment selection that is determined via the algorithm from FIGS. 31-35 is Protocol R 10033, which refers to the treatment of non-chronic, internal, non-ischemic, infected, with no inflammation, and painful medical conditions. This is the case of an internal, infected and non-ischemic acute painful tissue lesion, with no inflammation. For the options of the energy combination systems and their associated energy combination applicators together with treatment regimens of such lesions using Protocol R 10033 see FIG. 37G and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol R 10033 see FIG. 37G and FIGS. 36A-36B for the symbols used.

Examples of other non-chronic, internal, non-ischemic, infected, with no inflammation, and painful medical conditions, which can use the energy combination systems or combination energy protocols using different energy products corresponding in general to the selection of Protocol R 10033 are necrotizing fasciitis, folliculitis, perirectal abscesses, and others.

Necrotizing fasciitis or flesh-eating disease is a rare bacterial infection through a break in the skin that destroys tissue under the skin, which spreads quickly in the body and can cause death. Symptoms include blisters, fever, fatigue, and pain worse than a person would expect based on the wound's appearance. Accurate diagnosis, rapid IV antibiotic treatment, and prompt surgery are important to stopping this infection. For the energy treatment of necrotizing fasciitis in accordance with Protocol R 10033, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used). The antibiotic regimen must be continued with the Protocol R 10033 presented in this invention.

Folliculitis is a common skin condition in which hair follicles become infected. It is usually caused by a bacterial or fungal infection. At first it may look like small red bumps or white-headed pimples around hair follicles. The treatment with topical mupirocin or clindamycin is generally effective. Alternatively, benzoyl peroxide 5% wash may be used for 5 to 7 days when showering. For the energy treatment of folliculitis in accordance with Protocol R 10033, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used).

Perirectal abscess is an infection that is deep and tracks up along the rectum into the pelvis and are very rare in healthy newborns and children. These deep abscesses can be found in various locations in the pelvis and can be associated with inflammatory bowel disease, such as Crohn's disease. Perirectal abscesses often require surgical drainage even if they have ruptured or not. Antibiotics are necessary to prevent infections. For the energy treatment of perirectal abscesses in accordance with Protocol R 10033, different options of energy combination systems (with applicators incorporating multiple energy technologies and their control consoles, as presented in this invention) or independent energy devices (incorporating only one energy technology) are available, which are presented together with the associated treatment regimen in FIG. 37G (see also FIGS. 36A-36B for the symbols used). The antibiotic regimen can be continued concurrently with the Protocol R 10033 presented in this invention, at the physician's discretion.

Depending on the affliction and as needed for each case/patient, all treatment embodiments mentioned above for Protocol R 10033 might be repeated after a period of pause of at least one month.

The next treatment selection that is determined via the algorithm from FIGS. 31-35 is Protocol S 10034, which refers to the treatment of non-chronic, internal, non-ischemic, infected, with inflammation, and with or without pain present medical conditions. This is the case of an internal infected and non-ischemic acute tissue lesion, with significant inflammation and with or without pain present. For the options of the energy combination systems and their associated energy combination applicators together with treatment regimens of such lesions using Protocol S 10034 see FIG. 37H and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol S 10034 see FIG. 37H and FIGS. 36A-36B for the symbols used.

Examples of other non-chronic, internal, non-ischemic, infected, with inflammation, and with or without pain present medical conditions, which can use the energy combination systems or combination energy protocols using different energy products corresponding in general to the selection of Protocol S 10034 are anal fistula, cellulitis, internal abscesses, acute osteomyelitis, and others.

An anal fistula is a small tunnel that develops between the end of the bowel and the skin near the anus. They are usually the result of an infection in an anal gland that spreads to the skin. near the anus causing a collection of pus (abscess) in the nearby tissue. When the pus drains away, it can leave a small channel behind. Symptoms include pain, swelling, and discharge of blood or pus from the anus. A fistulotomy is the most effective treatment for many anal fistulas, which involves cutting along the whole length of the fistula to open it up, so it heals as a flat scar. For the energy treatment of anal fistulas in accordance with Protocol S 10034, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used).

Cellulitis is a common bacterial skin infection that causes redness, swelling, and pain in the infected area of the skin. Affected skin appears swollen and red and may be hot and tender. If untreated, cellulitis can spread rapidly and cause serious health problems. Good wound care and hygiene are important for preventing cellulitis. Without treatment with an antibiotic, cellulitis can be life-threatening. For the energy treatment of cellulitis in accordance with Protocol S 10034, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used). The antibiotic regimen must be continued concurrently with the Protocol S 10034 presented in this invention.

An abscess is a painful collection of pus, usually caused by a bacterial infection. Abscesses can develop anywhere in the body. Internal abscesses, which develop inside the body, in an organ or in the spaces between organs. An intra-abdominal abscess is a collection of pus or infected fluid that is surrounded by inflamed tissue inside the belly. It can involve any abdominal organ, or it can settle in the folds of the bowel. An intra-abdominal abscess often will need to be drained of fluid in order to heal. Typically, however, antibiotics are given along with draining the abscess. For the energy treatment of internal abscesses in accordance with Protocol S 10034, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used). The antibiotic regimen must be continued concurrently with the Protocol S 10034 presented in this invention, at the physician's discretion.

Acute osteomyelitis is inflammation or swelling that occurs in the bone. It can result from an infection somewhere else in the body that has spread to the bone, or it can start in the bone often as a result of an injury. Osteomyelitis is more common in younger children (five and under) but can happen at any age. Treatment is usually surgery to remove portions of bone that have died. This is followed by strong antibiotics, often by an intra-venous treatment, for at least six weeks. Amputation may be needed, especially in people with diabetes or poor blood circulation. For the energy treatment of acute osteomyelitis in accordance with Protocol S 10034, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used). The antibiotic regimen must be continued concurrently with the Protocol S 10034 presented in this invention.

Depending on the affliction and as needed for each case/patient, all treatment embodiments mentioned above for Protocol S 10034 might be repeated after a period of pause of at least one month.

The next treatment selection that is determined via the algorithm from FIGS. 31-35 is Protocol T 10035, which refers to the treatment of non-chronic, internal, ischemic, infected, without inflammation, and no painful medical conditions. This is the case of an internal infected and ischemic acute tissue lesion, with no inflammation or pain present. For the options of the energy combination systems and their associated energy combination applicators together with treatment regimens of such lesions using Protocol T 10035 see FIG. 37H and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol T 10035 see FIG. 37H and FIGS. 36A-36B for the symbols used.

Examples of other non-chronic, internal, ischemic, infected, without inflammation, and with no pain present medical conditions, which can use the energy combination systems or combination energy protocols using different energy products corresponding in general to the selection of Protocol T 10035 are acute tubular necrosis and others.

Acute tubular necrosis is a kidney disorder involving damage to the tubule cells of the kidneys due to a poison or harmful medication, which can lead to acute kidney failure. The tubules are tiny ducts in the kidneys that help filter the blood when it passes through the kidneys. Treatment is directed at the cause, for example, stopping medications that are damaging the kidneys, giving intravenous fluids to raise blood pressure, and giving antibiotics to treat infection. For the energy treatment of acute tubular necrosis to relax the kidney structure and improve blood circulation in accordance with Protocol T 10035, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used). The intravenous fluids and antibiotic regimen must be continued concurrently with the Protocol T 10035 presented in this invention.

Depending on the affliction and as needed for each case/patient, all treatment embodiments mentioned above for Protocol T 10035 might be repeated after a period of pause of at least one month.

The next treatment selection that is determined via the algorithm from FIGS. 31-35 is Protocol U 10036, which refers to the treatment of non-chronic, internal, ischemic, infected, without inflammation, and painful medical conditions. This is the case of an internal infected, ischemic, and painful acute tissue lesion, with no inflammation. For the options of the energy combination systems and their associated energy combination applicators together with treatment regimens of such lesions using Protocol U 10036 see FIG. 37H and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol U 10036 see FIG. 37H and FIGS. 36A-36B for the symbols used.

Examples of other non-chronic, internal, ischemic, infected, without inflammation, and painful medical conditions, which can use the energy combination systems or combination energy protocols using different energy products corresponding in general to the selection of Protocol T 10035 are acute mesenteric ischemia and others.

Acute mesenteric ischemia is a condition that happens when sudden narrowed or blocked arteries restrict blood flow to the small intestine. Decreased blood flow can permanently damage the small intestine. Treatment may include blood thinners or a surgical procedure to restore blood flow to the small intestine. For the energy treatment of acute mesenteric ischemia in accordance with Protocol U 10036, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used).

Depending on the affliction and as needed for each case/patient, all treatment embodiments mentioned above for Protocol U 10036 might be repeated after a period of pause of at least one month.

The next treatment selection that is determined via the algorithm from FIGS. 31-35 is Protocol V 10037, which refers to the treatment of non-chronic, internal, ischemic, infected, with inflammation, and with or without pain medical conditions. This is the case of an internal infected and ischemic acute tissue lesion, with significant inflammation, which might have or not pain present. For the options of the energy combination systems and their associated energy combination applicators together with treatment regimens of such lesions using Protocol V 10037 see FIG. 37H and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol V 10037 see FIG. 37H and FIGS. 36A-36B for the symbols used.

Examples of other non-chronic, internal, ischemic, infected wound, with inflammation, and with or without pain medical conditions, which can use the energy combination systems or combination energy protocols using different energy products corresponding in general to the selection of Protocol V 10037 are the open fractures with vascular injuries, ischemic colitis, and others.

Open fractures with vascular injury are bone fractures that produce concomitantly also vascular injuries. For example, the displaced fractures of the distal femur and proximal tibia and particularly dislocation of the knee are associated with the highest risk of concomitant vascular injury and the poorest collateral circulation to support the distal limb. The treatment consists of fracture stabilization followed by surgery for vascular repair. For the energy treatment of open fractures with vascular injury in accordance with Protocol V 10037, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used).

Acute ischemic colitis occurs when a part of the colon experiences a decrease in blood flow due to a blood clot. The treatment consists of antibiotics to prevent infections, intravenous fluids for resuscitation/dehydration, optimizing cardiac output, use of supplemental oxygen, and avoiding medications that constrict blood vessels. For the energy treatment of acute ischemic colitis in accordance with Protocol V 10037, different options of energy combination systems (with applicators incorporating multiple energy technologies and their control consoles, as presented in this invention) or independent energy devices (incorporating only one energy technology) are available, which are presented together with the associated treatment regimen in FIG. 37H (see also FIGS. 36A-36B for the symbols used). The antibiotic regimen can be continued concurrently with the Protocol V 10037 presented in this invention, at the physician's discretion.

Depending on the affliction and as needed for each case/patient, all treatment embodiments mentioned above for Protocol V 10037 might be repeated after a period of pause of at least one month.

The next treatment selection that is determined via the algorithm from FIGS. 31-35 is Protocol W 10038, which refers to the treatment of non-chronic, internal, ischemic, non-infected, without inflammation or pain medical conditions. This is the case of an internal non-infected and ischemic acute tissue lesion, with no inflammation or pain present. For the options of the energy combination systems and their associated energy combination applicators together with treatment regimens of such lesions using Protocol W 10038 see FIG. 37I and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol W 10038 see FIG. 37I and FIGS. 36A-36B for the symbols used.

Examples of other non-chronic, internal, ischemic, non-infected, without inflammation or pain medical conditions, which can use the energy combination systems or combination energy protocols using different energy products corresponding in general to the selection of Protocol W 10038 are the exposed tendon after trauma, osteonecrosis, and others.

Tendons may be exposed in trauma wounds such as massive crush injuries or fractures and contaminated or infected surgical wounds. Tendons are nourished by blood vessels and diffusion of nutrients from synovial fluid, exposure of this structure to air will cause desiccation and subsequent tissue necrosis and infection. For exposed tendons, flap prostheses or skin grafts are used after long-term drug replacement. For the energy treatment of exposed tendon after trauma in accordance with Protocol W 10038, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Osteonecrosis, also known as avascular necrosis (AVN), aseptic necrosis or ischemic bone necrosis, is a disease resulting in the death of bone cells, due to ischemia. If the process involves the bones near a joint, it often leads to collapse of the joint surface and subsequent arthritis due to an irregular joint surface. The treatments for osteonecrosis are core decompression (removal of a part of the inner layer of bone), bone transplant (graft), bone reshaping (osteotomy), joint replacement, and regenerative medicine treatment. For the energy treatment of osteonecrosis in accordance with Protocol W 10038, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Depending on the affliction and as needed for each case/patient, all treatment embodiments mentioned above for Protocol W 10038 might be repeated after a period of pause of at least one month.

The next treatment selection that is determined via the algorithm from FIGS. 31-35 is Protocol X 10039, which refers to the treatment of non-chronic, internal, ischemic, non-infected, without inflammation, and painful medical conditions. This is the case of an internal non-infected and ischemic acute tissue lesion, with no inflammation, and significant pain present. For the options of the energy combination systems and their associated energy combination applicators together with treatment regimens of such lesions using Protocol X 10039 see FIG. 37I and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol X 10039 see FIG. 37I and FIGS. 36A-36B for the symbols used.

Examples of other non-chronic, internal, ischemic, non-infected, without inflammation, and painful medical conditions, which can use the energy combination systems or combination energy protocols using different energy products corresponding in general to the selection of Protocol X 10039 are acute tissue pressure injuries, bone non-unions, acute limb ischemia, and others.

Acute tissue pressure injuries are persistent non-blanchable deep red, purple or maroon areas of intact skin, non-intact skin or blood-filled blisters. Tissue pressure injury (DTI) is an injury to the soft tissue under the skin due to pressure and is usually over boney prominence. This injury is commonly seen in bedridden patients in hospitals and nursing homes. These wounds are most commonly left intact and dry, with careful offloading at all times, and the use direct foam padding dressings. For the energy treatment of acute tissue pressure injuries in accordance with Protocol X 10039, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Bone non-union is the body's inability to heal a fracture that persists for a minimum of nine months without signs of healing for three months. The treatment consists of grafting bone to the fracture site followed by surgical procedure for stabilization, or the non-surgical treatment using a bone stimulator using ultrasound or pulsed magnetic waves. For the energy treatment of bone non-unions in accordance with Protocol X 10039, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Acute limb ischemia is a sudden decrease in limb perfusion that threatens limb viability, due to presence of the thrombus. The sudden ischemia affects all the metabolically active tissues of the limb: skin, muscles, and nerves. Treatment methods for acute limb ischemia include surgical treatment (such as thrombo-embolectomy and bypass surgery), endovascular treatment (such as catheter-directed thrombolysis, percutaneous thrombus aspiration, and stent placement), and hybrid treatment that combines both therapies. For the energy treatment of acute limb ischemia in accordance with Protocol X 10039, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Depending on the affliction and as needed for each case/patient, all treatment embodiments mentioned above for Protocol X 10039 might be repeated after a period of pause of at least one month.

The next treatment selection that is determined via the algorithm from FIGS. 31-35 is Protocol Y 10040, which refers to the treatment of non-chronic, internal, ischemic, non-infected, with inflammation, and with or without pain medical conditions. This is the case of an internal non-infected and ischemic acute tissue lesion, with significant inflammation, and with or without pain present. For the options of the energy combination systems and their associated energy combination applicators together with treatment regimens of such lesions using Protocol Y 10040 see FIG. 37I and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol Y 10040 see FIG. 37I and FIGS. 36A-36B for the symbols used.

Examples of other non-chronic, internal, ischemic, non-infected, without inflammation, and painful medical conditions, which can use the energy combination systems or combination energy protocols using different energy products corresponding in general to the selection of Protocol Y 10040 are deep rhabdomyolysis, diabetic muscle ischemia, and others.

Rhabdomyolysis can be a life-threatening condition caused by muscle breakdown and muscle death, which may be caused by trauma or crush injuries, overexertion, toxic substances, or disease. As muscle cells disintegrate, they release a protein called myoglobin into the blood. The treatment is based on the use of drugs such as cocaine, amphetamines, statins, heroin, or phencyclidine. For the energy treatment of rhabdomyolysis in accordance with Protocol Y 10040, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used). The drug regimen can be continued concurrently with the Protocol Y 10040 presented in this invention, at the physician's discretion.

Diabetic muscle ischemia or diabetic muscle infarction/necrosis occurs from thrombosis of medium and small arterioles in patients with atherosclerosis and poorly controlled diabetes. Clinical symptoms consist of severe pain with a history of a palpable mass with or without swelling. Pain management and activity restriction in the acute phase followed by gentle physical therapy is the treatment of choice. For the energy treatment of diabetic muscle ischemia in accordance with Protocol Y 10040, different options of energy combination systems (with applicators incorporating multiple energy technologies and their control consoles, as presented in this invention) or independent energy devices (incorporating only one energy technology) are available, which are presented together with the associated treatment regimen in FIG. 37I (see also FIGS. 36A-36B for the symbols used).

Depending on the affliction and as needed for each case/patient, all treatment embodiments mentioned above for Protocol Y 10040 might be repeated after a period of pause of at least one month.

The next treatment selection that is determined via the algorithm from FIGS. 31-35 is Protocol Z 10041, which refers to the treatment of non-chronic, external, ischemic, non-infected, without inflammation or pain medical conditions. This is the case of an external non-infected and ischemic acute tissue lesion, with no inflammation or pain present. For the options of the energy combination systems and their associated energy combination applicators together with treatment regimens of such lesions using Protocol Z 10041 see FIG. 37J and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol Z 10041 see FIG. 37J and FIGS. 36A-36B for the symbols used.

Examples of other non-chronic, external, ischemic, non-infected, without inflammation or pain medical conditions, which can use the energy combination systems or combination energy protocols using different energy products corresponding in general to the selection of Protocol Z 10041 are ischemic skin grafts, ischemic flaps, and others.

Ischemic skin grafts are produced by the lack of oxygenation (blood circulation) for few days. A graft can tolerate an ischemic interval when placed on a poorly vascularized bed. Thick full thickness skin grafts appear to tolerate ischemia for up to 3 days while thin full thickness skin grafts survive for up to 5 days. Split-thickness grafts take well even after 4 days of ischemia. The treatment of ischemic skin grafts consists of hyperbaric oxygen therapy, dressings, different creams, etc. For the energy treatment of ischemic skin grafts in accordance with Protocol Z 10041, different options of energy combination systems (with applicators incorporating multiple energy technologies and their control consoles, as presented in this invention) or independent energy devices (incorporating only one energy technology) are available, which are presented together with the associated treatment regimen in FIG. 37J (see also FIGS. 36A-36B for the symbols used). The main scope of the energy treatment is to reduce or eliminate ischemia.

A “flap” consists of one or more tissue components including skin, deeper tissues, muscle and bone. Ischemic flaps have a poor blood circulation and lack proper oxygenation. Treatments used for this condition are different drugs, hyperbaric oxygen therapy, etc. The treatment of ischemic flaps consists of hyperbaric oxygen therapy, dressings, different creams, etc. For the energy treatment of ischemic flaps in accordance with Protocol Z 10041, different options of energy combination systems (with applicators incorporating multiple energy technologies and their control consoles, as presented in this invention) or independent energy devices (incorporating only one energy technology) are available, which are presented together with the associated treatment regimen in FIG. 37J (see also FIGS. 36A-36B for the symbols used). The main scope of the energy treatment is to reduce or eliminate ischemia.

Depending on the affliction and as needed for each case/patient, all treatment embodiments mentioned above for Protocol Z 10041 might be repeated after a period of pause of at least one month.

The next treatment selection that is determined via the algorithm from FIGS. 31-35 is Protocol AA 10042, which refers to the treatment of non-chronic, external, ischemic, non-infected, without inflammation, and painful medical conditions. This is the case of an external non-infected and ischemic acute tissue lesion, with no inflammation, and with significant pain present. For the options of the energy combination systems and their associated energy combination applicators together with treatment regimens of such lesions using Protocol AA 10042 see FIG. 37J and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol AA 10042 see FIG. 37J and FIGS. 36A-36B for the symbols used.

Examples of other non-chronic, external, ischemic, non-infected, without inflammation and painful medical conditions, which can use the energy combination systems or combination energy protocols using different energy products corresponding in general to the selection of Protocol AA 10042 are acute febrile neutrophilic dermatosis and others.

Acute febrile neutrophilic dermatosis (AFND), also known as Sweet syndrome or Gomm Button disease, consists of erythematous plaques and papules along with constitutional symptoms such as fever and malaise. The cutaneous lesions present as tender, edematous, erythematous plaques and papules, which may occasionally be bullous. The most effective treatment is prednisone or other drugs as dapsone, colchicine, and potassium iodide. For the energy treatment of acute febrile neutrophilic dermatosis in accordance with Protocol AA 10042, different options of energy combination systems (with applicators incorporating multiple energy technologies and their control consoles, as presented in this invention) or independent energy devices (incorporating only one energy technology) are available, which are presented together with the associated treatment regimen in FIG. 37J (see also FIGS. 36A-36B for the symbols used). The drug regimen can be continued concurrently with the Protocol AA 10042 presented in this invention, at the physician's discretion.

Depending on the affliction and as needed for each case/patient, all treatment embodiments mentioned above for Protocol AA 10042 might be repeated after a period of pause of at least one month.

The next treatment selection that is determined via the algorithm from FIGS. 31-35 is Protocol BB 10043, which refers to the treatment of non-chronic, external, ischemic, non-infected, with inflammation, and with or without pain medical conditions. This is the case of an external non-infected and ischemic acute tissue lesion, with significant inflammation, and with or without pain present. For the options of the energy combination systems and their associated energy combination applicators together with treatment regimens of such lesions using Protocol BB 10043 see FIG. 37J and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol BB 10043 see FIG. 37J and FIGS. 36A-36B for the symbols used.

Examples of other non-chronic, external, ischemic, non-infected, with inflammation, and with or without pain medical conditions, which can use the energy combination systems or combination energy protocols using different energy products corresponding in general to the selection of Protocol BB 10043 are erythroderma and others.

Erythroderma is a severe and potentially life-threatening inflammation of most of the body's skin surface. It is also called generalized exfoliative dermatitis. A very large area of the body, if not most of the body, is bright red and inflamed. The body can appear to be covered in a peeling red rash that usually itches or burns. The treatment is done with emollients and colloidal oatmeal baths, weak topical corticosteroids or systemic corticosteroids. For the energy treatment of erythroderma in accordance with Protocol BB 10043, different options of energy combination systems (with applicators incorporating multiple energy technologies and their control consoles, as presented in this invention) or independent energy devices (incorporating only one energy technology) are available, which are presented together with the associated treatment regimen in FIG. 37J (see also FIGS. 36A-36B for the symbols used). The corticosteroids regimen can be continued concurrently with the Protocol BB 10043 presented in this invention, at the physician's discretion.

Depending on the affliction and as needed for each case/patient, all treatment embodiments mentioned above for Protocol BB 10043 might be repeated after a period of pause of at least one month.

The next treatment selection that is determined via the algorithm from FIGS. 31-35 is Protocol CC 10044, which refers to the treatment of non-chronic, external, ischemic, infected, with inflammation, and with or without pain medical conditions. This is the case of an external infected and ischemic acute tissue lesion, with significant inflammation, and with or without pain present. For the options of the energy combination systems and their associated energy combination applicators together with treatment regimens of such lesions using Protocol CC 10044 see FIG. 37K and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol CC 10044 see FIG. 37K and FIGS. 36A-36B for the symbols used.

Examples of other non-chronic, external, ischemic, infected, with inflammation, and with or without pain medical conditions, which can use the energy combination systems or combination energy protocols using different energy products corresponding in general to the selection of Protocol CC 10044 are the cutaneous granulomas annulare and others.

Cutaneous granuloma annulare is a skin condition that causes a raised rash or bumps (lesions) in a ring pattern. The most common type affects young adults and usually affects the hands and feet. The treatment is done with medicated creams or ointments, corticosteroid injections, and by applying liquid nitrogen to the affected area may help remove the bumps. For the energy treatment of cutaneous granulomas annulare in accordance with Protocol CC 10044, different options of energy combination systems (with applicators incorporating multiple energy technologies and their control consoles, as presented in this invention) or independent energy devices (incorporating only one energy technology) are available, which are presented together with the associated treatment regimen in FIG. 37K (see also FIGS. 36A-36B for the symbols used). The corticosteroids regimen can be continued concurrently with the Protocol CC 10044 presented in this invention, at the physician's discretion.

Depending on the affliction and as needed for each case/patient, all treatment embodiments mentioned above for Protocol CC 10044 might be repeated after a period of pause of at least one month.

The next treatment selection that is determined via the algorithm from FIGS. 31-35 is Protocol DD 10045, which refers to the treatment of non-chronic, external, ischemic, infected, without inflammation or pain medical conditions. This is the case of an external infected and ischemic acute tissue lesion, with no inflammation or pain present. For the options of the energy combination systems and their associated energy combination applicators together with treatment regimens of such lesions using Protocol DD 10045 see FIG. 37K and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol DD 10045 see FIG. 37K and FIGS. 36A-36B for the symbols used.

Examples of other non-chronic, external, ischemic, infected, without inflammation or pain medical conditions, which can use the energy combination systems or combination energy protocols using different energy products corresponding in general to the selection of Protocol DD 10045 are impetigo and others.

Impetigo is a common and highly contagious skin infection that mainly affects infants and young children. It usually appears as reddish sores on the face, especially around the nose and mouth and on the hands and feet. Over about a week, the sores burst and develop honey-colored crusts. Impetigo treatment involves antibiotics. For the energy treatment of impetigo in accordance with Protocol DD 10045, different options of energy combination systems (with applicators incorporating multiple energy technologies and their control consoles, as presented in this invention) or independent energy devices (incorporating only one energy technology) are available, which are presented together with the associated treatment regimen in FIG. 37K (see also FIGS. 36A-36B for the symbols used). The antibiotic regimen can be continued concurrently with the Protocol DD 10045 presented in this invention, at the physician's discretion.

Depending on the affliction and as needed for each case/patient, all treatment embodiments mentioned above for Protocol DD 10045 might be repeated after a period of pause of at least one month.

The next treatment selection that is determined via the algorithm from FIGS. 31-35 is Protocol EE 10046, which refers to the treatment of non-chronic, external, ischemic, infected, without inflammation, and painful medical conditions. This is the case of an external infected and ischemic acute tissue lesion, with no inflammation, and significant pain present. For the options of the energy combination systems and their associated energy combination applicators together with treatment regimens of such lesions using Protocol EE 10046 see FIG. 37K and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol EE 10046 see FIG. 37K and FIGS. 36A-36B for the symbols used.

Examples of other non-chronic, external, ischemic, infected, without inflammation, and painful medical conditions, which can use the energy combination systems or combination energy protocols using different energy products corresponding in general to the selection of Protocol EE 10046 are severe skin burns, severe electrical burns, and others.

Severe skin burns are a damage to the skin or deeper tissues caused by sun, hot liquids, fire, or chemicals. The degree of severity of most burns is based on the size and depth of the burn. Symptoms range from a feeling of minor discomfort to a life-threatening emergency, depending on the size and depth (degree) of the burn. Sunburn and small scalds can often be treated at home. Deep or widespread skin burns and chemical burns need immediate medical care, often at specialized burn units. The treatment for the skin burns is done by cooling the area with cold water, apply specialized creams and bandages, use painkillers for pain, and administer antibiotics against possible infection. For the energy treatment of skin burns in accordance with Protocol EE 10046, different options of energy combination systems (with applicators incorporating multiple energy technologies and their control consoles, as presented in this invention) or independent energy devices (incorporating only one energy technology) are available, which are presented together with the associated treatment regimen in FIG. 37K (see also FIGS. 36A-36B for the symbols used). The antibiotic regimen can be continued concurrently with the Protocol EE 10046 presented in this invention, at the physician's discretion.

Severe electrical burns are generated by electricity and are more difficult to diagnose because they are capable of causing significant injury beneath the skin without showing any signs of damage on the surface. Deep or widespread electrical burns need immediate medical care at specialized burn units. The treatment for serious electrical burns is done by applying specialized creams and bandages, use painkillers for pain, surgery to repair the burned area, treatment of internal trauma, and administer antibiotics against possible infection. For the energy treatment of electrical burns in accordance with Protocol EE 10046, different options of energy combination systems (with applicators incorporating multiple energy technologies and their control consoles, as presented in this invention) or independent energy devices (incorporating only one energy technology) are available, which are presented together with the associated treatment regimen in FIG. 37K (see also FIGS. 36A-36B for the symbols used). The antibiotic regimen can be continued concurrently with the Protocol EE 10046 presented in this invention, at the physician's discretion.

Depending on the affliction and as needed for each case/patient, all treatment embodiments mentioned above for Protocol EE 10046 might be repeated after a period of pause of at least one month.

The next treatment selection that is determined via the algorithm from FIGS. 31-35 is Protocol FF 10047, which refers to the treatment of non-chronic, external, non-ischemic, infected, with inflammation, and with or without pain medical conditions. This is the case of an external infected and non-ischemic acute tissue lesion, with significant inflammation, and with or without pain present. For the options of the energy combination systems and their associated energy combination applicators together with treatment regimens of such lesions using Protocol FF 10047 see FIG. 37L and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol FF 10047 see FIG. 37L and FIGS. 36A-36B for the symbols used.

Examples of other non-chronic, external, non-ischemic, infected, with inflammation, and with or without pain medical conditions, which can use the energy combination systems or combination energy protocols using different energy products corresponding in general to the selection of Protocol FF 10047 are sporotrichoses, wounds with dehiscence, external skin abscesses, intravenous infiltration, and others.

Sporotrichosis is a subcutaneous mycosis that has long been recognized as the gardener's disease, because the infection is in most cases a result of the inoculation of the fungus Sporothrix spp. by thorns, stings, scrapes, and minor injuries. The first symptom of cutaneous (skin) sporotrichosis is usually a small, painless bump that can develop any time from 1 to 12 weeks after exposure to the fungus. The bump can be red, pink, or purple, and usually appears on the finger, hand, or arm where the fungus has entered through a break in the skin. The treatment for this type of sporotrichosis is itraconazole taken by mouth for 3 to 6 months. Supersaturated potassium iodide (SSKI) is another treatment option for skin sporotrichosis. For the energy treatment of sporotrichoses in accordance with Protocol FF 10047, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen must be continued concurrently with the Protocol FF 10047 presented in this invention.

Wound dehiscence is a surgery complication where the incision, a cut made during a surgical procedure, reopens. It is sometimes called wound breakdown, wound disruption, or wound separation. Partial dehiscence means that the edges of an incision have pulled apart in one or more small areas. The treatment for wounds with dehiscence includes pain medication, antibiotics for infection, surgery to remove dead tissue, wet-and-dry dressing, and vacuum-assisted closure of the wound. For the energy treatment of wound dehiscence in accordance with Protocol FF 10047, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen must be continued concurrently with the Protocol FF 10047 presented in this invention.

External skin abscess is a painful collection of pus, usually caused by a bacterial infection, which develops under the skin. When an area becomes infected, the body's immune system sends white blood cells to fight the infection. These cells collect and combine with the damaged tissue and germs, creating liquid called pus. An abscess is characterized by a painful, swollen lump that is filled with pus. Treatment consists of drainage of the pus and antibiotics. For the energy treatment of external skin abscesses in accordance with Protocol FF 10047, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen can be continued concurrently with the Protocol FF 10047 presented in this invention, at the physician's discretion.

Intravenous infiltration is a common complication of intravenous therapy. Infiltration occurs when intravenous (IV) fluid or medications slips out of the vein lumen, due to improper placement or dislodgment of the catheter, and leaks into the surrounding tissue. Treatment consists in stopping the infusion, removal of the IV, and inject different mediation into the subcutaneous tissue. For the energy treatment of intravenous infiltration in accordance with Protocol FF 10047, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used).

Depending on the affliction and as needed for each case/patient, all treatment embodiments mentioned above for Protocol FF 10047 might be repeated after a period of pause of at least one month.

The next treatment selection that is determined via the algorithm from FIGS. 31-35 is Protocol GG 10048, which refers to the treatment of non-chronic, external, non-ischemic, infected, without inflammation or pain medical conditions. This is the case of an external infected and non-ischemic acute tissue lesion, with no inflammation or pain present. For the options of the energy combination systems and their associated energy combination applicators together with treatment regimens of such lesions using Protocol GG 10048 see FIG. 37L and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol GG 10048 see FIG. 37L and FIGS. 36A-36B for the symbols used.

Examples of other non-chronic, external, non-ischemic, infected, without inflammation or pain medical conditions, which can use the energy combination systems or combination energy protocols using different energy products corresponding in general to the selection of Protocol GG 10048 are skin acnes and others.

Acne is a skin condition that occurs when the hair follicles become plugged with oil and dead skin cells. Acne is most common among teenagers, though it affects people of all ages. Symptoms range from uninflamed blackheads to pus-filled pimples or large, red, and tender bumps. The treatment for skin acne includes a combination of benzoyl peroxide and a topical antibiotic (erythromycin or clindamycin), topical retinoid, or both. For the energy treatment of skin acnes in accordance with Protocol GG 10048, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen can be continued concurrently with the Protocol GG 10048 presented in this invention, at the physician discretion.

Depending on the affliction and as needed for each case/patient, all treatment embodiments mentioned above for Protocol GG 10048 might be repeated after a period of pause of at least one month.

The next treatment selection that is determined via the algorithm from FIGS. 31-35 is Protocol HH 10049, which refers to the treatment of non-chronic, external, non-ischemic, infected, without inflammation, and painful medical conditions. This is the case of an external infected and non-ischemic acute tissue lesion, with no inflammation, and significant pain present. For the options of the energy combination systems and their associated energy combination applicators together with treatment regimens of such lesions using Protocol HH 10049 see FIG. 37L and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol HH 10049 see FIG. 37L and FIGS. 36A-36B for the symbols used.

Examples of other non-chronic, external, non-ischemic, infected, without inflammation, and painful medical conditions, which can use the energy combination systems or combination energy protocols using different energy products corresponding in general to the selection of Protocol HH 10049 are pilonidal cysts, infected keloids, and others.

A pilonidal cyst is an abnormal pocket in the skin that usually contains hair and skin debris. A pilonidal cyst is almost always located near the tailbone at the top of the cleft of the buttocks. Symptoms of an infected cyst include pain, reddened skin, or drainage of pus or blood. Treatment may include antibiotics, hot compresses, topical treatment with depilatory creams, cyst drainage, or surgery to remove completely the cyst. For the energy treatment of pilonidal cysts in accordance with Protocol HH 10049, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen can be continued concurrently with the Protocol HH 10049 presented in this invention, at the physician's discretion.

An infected keloid will need to be attended to urgently. Such infections not only cause pain and discomfort, they can also result in systemic/bloodstream infections. An infected keloid is tender, painful and warmer than the surrounding normal skin. A course of oral antibiotics can usually resolve this complication. For the energy treatment of infected keloids in accordance with Protocol HH 10049, different options of energy combination systems (with applicators incorporating multiple energy technologies and their control consoles, as presented in this invention) or independent energy devices (incorporating only one energy technology) are available, which are presented together with the associated treatment regimen in FIG. 37L (see also FIGS. 36A-36B for the symbols used). The antibiotic regimen must be continued concurrently with the Protocol HH 10049 presented in this invention.

Depending on the affliction and as needed for each case/patient, all treatment embodiments mentioned above for Protocol HH 10049 might be repeated after a period of pause of at least one month.

The next treatment selection that is determined via the algorithm from FIGS. 31-35 is Protocol II 10050, which refers to the treatment of non-chronic, external, non-ischemic, non-infected, with inflammation, and no painful medical conditions. This is the case of an external non-infected and non-ischemic acute tissue lesion, with inflammation, and no pain present. For the options of the energy combination systems and their associated energy combination applicators together with treatment regimens of such lesions using Protocol II 10050 see FIG. 37M and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol II 10050 see FIG. 37M and FIGS. 36A-36B for the symbols used.

Examples of other non-chronic, external, non-ischemic, non-infected, with inflammation, and non-painful medical conditions, which can use the energy combination systems or combination energy protocols using different energy products corresponding in general to the selection of Protocol II 10050 are non-painful lumpy bruises, non-painful varicose veins, and others.

Non-painful lumpy bruises have swelling around the bruised skin or under the bruise and create a loss of function in the affected area (joint, limb, or muscle). The treatment consists in applying cold compresses and taken pain medicine. For the energy treatment of non-painful lumpy bruises in accordance with Protocol II 10050, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used).

Non-painful varicose veins that are twisted and enlarged veins. Any vein that is close to the skin's surface (superficial) can become varicosed. Varicose veins most commonly affect the veins in the legs. The treatment for varicose veins is the use of compression stocking, thermal ablation, vein stripping, and sclerotherapy (using special foam to close the veins). For the energy treatment of non-painful varicose veins in accordance with Protocol II 10050, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used).

Depending on the affliction and as needed for each case/patient, all treatment embodiments mentioned above for Protocol II 10050 might be repeated after a period of pause of at least one month.

The next treatment selection that is determined via the algorithm from FIGS. 31-35 is Protocol JJ 10051, which refers to the treatment of non-chronic, external, non-ischemic, non-infected, with inflammation, and painful medical conditions. This is the case of an external non-infected and non-ischemic acute tissue lesion, with inflammation, and significant pain present. For the options of the energy combination systems and their associated energy combination applicators together with treatment regimens of such lesions using Protocol JJ 10051 see FIG. 37M and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using smart bandages, they can be applied immediately after the main treatment sessions and kept for the whole time period in between main treatments. In other cases where independent energy devices are used, based on the specifics of each medical affliction, one or multiple secondary treatment sessions can be done during the time period in between the main treatment sessions. For the combination energy protocol options and treatment regimen using Protocol JJ 10051 see FIG. 37M and FIGS. 36A-36B for the symbols used.

Examples of other non-chronic, external, non-ischemic, non-infected, with inflammation, and painful medical conditions, which can use the energy combination systems or combination energy protocols using different energy products corresponding in general to the selection of Protocol JJ 10051 are sunburns and others.

A sunburn is red, painful skin, itchy area that feels hot to the touch. Skin may also blister. It usually appears within a few hours after too much exposure to ultraviolet (UV) light from sunshine or artificial sources, such as sunlamps. Treatment usually includes hydration, different creams as aloe vera and moisturizers, coconut oil, and painkillers. For the energy treatment of sunburns in accordance with Protocol JJ 10051, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used).

Depending on the affliction and as needed for each case/patient, all treatment embodiments mentioned above for Protocol JJ 10051 might be repeated after a period of pause of at least one month.

The next treatment selection that is determined via the algorithm from FIGS. 31-35 is Protocol KK 10052, which refers to the treatment of chronic, internal, non-ischemic, non-infected, without inflammation or pain medical conditions. This is the case of an internal non-infected and non-ischemic chronic tissue lesion, with no inflammation or pain present. For the options of the energy combination systems and their associated energy combination applicators together with treatment regimens of such lesions using Protocol KK 10052 see FIG. 37M and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol KK 10052 see FIG. 37M and FIGS. 36A-36B for the symbols used.

Examples of other chronic, internal, non-ischemic, non-infected, without inflammation or pain medical conditions, which can use the energy combination systems or combination energy protocols using different energy products corresponding in general to the selection of Protocol KK 10052 are osteoporosis, diabetes type IA (restore the islet cells), dementia, Alzheimer, Parkinson's disease, atrial fibrillation (A-fib), and others.

Osteoporosis is a disease that thins and weakens the bones, which become fragile and fracture (break) easily, especially the bones in the hip, spine, and wrist. The treatment consists in special osteoporosis medication, healthy diet, and weight-bearing exercise to help prevent bone loss or strengthen already weak bones. For the energy treatment of osteoporosis in accordance with Protocol KK 10052, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used). The specialized medication regimen can be continued concurrently with the Protocol KK 10052 presented in this invention, at the physician's discretion.

Diabetes type 1A is a chronic condition in which the pancreas produces little or no insulin. It typically appears in adolescence. Symptoms include increased thirst, frequent urination, hunger, fatigue, and blurred vision. Treatment aims at maintaining normal blood sugar levels through regular monitoring, insulin therapy, diet, and exercise. For the energy treatment of Diabetes type 1 A (restore the islet cells) in accordance with Protocol KK 10052, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used).

Dementia is a general term for loss of memory and judgement, language, problem-solving, and other thinking abilities that are severe enough to interfere with daily life. Symptoms include forgetfulness, limited social skills, and thinking abilities so impaired that it interferes with daily functioning. The treatment consists of cognition-enhancing medication, rehabilitation, and occupational therapy.

Alzheimer is a progressive disease that destroys memory and other important mental functions. Brain cell connections and the cells themselves degenerate and die, eventually destroying memory and other important mental functions. Memory loss and confusion are the main symptoms. For treatment cognition-enhancing medications, physical exercise, and management strategies may temporarily improve symptoms. For the energy treatment of dementia or Alzheimer in accordance with Protocol KK 10052, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used). The specialized medication regimen for dementia or Alzheimer can be continued concurrently with the Protocol KK 10052 presented in this invention, at the physician's discretion.

Parkinson's disease is a brain disorder that leads to shaking, stiffness, and difficulty with walking, balance, and coordination. Parkinson's symptoms usually begin gradually and get worse over time. Nerve cell damage in the brain causes dopamine levels to drop, leading to the symptoms of Parkinson's. As the disease progresses, people may have difficulty walking and talking. Treatments for Parkinson's disease consist of specialized medications, surgery, complementary and supportive therapies, such as diet, exercise, physical therapy, occupational therapy, and speech therapy. For the energy treatment of Parkinson's disease in accordance with Protocol KK 10052, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used). The specialized medication regimen can be continued concurrently with the Protocol KK 10052 presented in this invention, at the physician's discretion.

Atrial fibrillation (A-fib) is an irregular and often very rapid heart rhythm (arrhythmia) that can lead to blood clots in the heart. The heart's upper chambers (atria) beat out of coordination with the lower chambers (ventricles). This condition may have no symptoms, but when symptoms do appear they include palpitations, shortness of breath, and fatigue. Treatments include drugs, electrical shock (cardioversion), and minimally invasive surgery (ablation). For the energy treatment of atrial fibrillation (A-fib) in accordance with Protocol KK 10052, different options of energy combination systems (with applicators incorporating multiple energy technologies and their control consoles, as presented in this invention) or independent energy devices (incorporating only one energy technology) are available, which are presented together with the associated treatment regimen in FIG. 37M (see also FIGS. 36A-36B for the symbols used). The drug regimen can be continued concurrently with the Protocol KK 10052 presented in this invention, at the physician's discretion.

Depending on the affliction and as needed for each case/patient, all treatment embodiments mentioned above for Protocol KK 10052 might be repeated after a period of pause of at least one month.

The next treatment selection that is determined via the algorithm from FIGS. 31-35 is Protocol LL 10053, which refers to the treatment of chronic, internal, non-ischemic, non-infected, without inflammation, and painful medical conditions. This is the case of an internal non-infected and non-ischemic chronic tissue lesion, with no inflammation, and significant pain present. For the options of the energy combination systems and their associated energy combination applicators together with treatment regimens of such lesions using Protocol LL 10053 see FIG. 37N and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol LL 10053 see FIG. 37N and FIGS. 36A-36B for the symbols used.

Examples of other chronic, internal, non-ischemic, non-infected, without inflammation, and painful medical conditions, which can use the energy combination systems or combination energy protocols using different energy products corresponding in general to the selection of Protocol LL 10053 are internal cysts, osteoporotic fractures, ankylosing spondylitis, and others.

Many internal cysts, such as those that occur in the kidneys or the liver, may not cause any symptoms at all. If they are large, a dull or sharp pain can be felt on one side of pelvis or abdomen and in some cases, one feels bloated or a heaviness in the lower abdomen. If the cyst ruptures, a sudden sharp pain and severe back pain is felt that gets worse when stand or walk. Trouble bending or twisting your body is another indication of an internal cyst rupture. The treatment of internal cysts consists of different supplements to drain and shrink the cyst and surgery for complete removal. For the energy treatment of internal cysts in accordance with Protocol LL 10053, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used).

Osteoporotic fractures are a result of osteoporosis, a condition in which the bones become more fragile due to bone deterioration or low bone mass. Particularly the fractures in the spine or hip are the most serious complications of osteoporosis and can result in disability and even an increased risk of death within the first year after the injury. The treatment is the same as for a non-osteoporotic fracture and involves immobilization, fixation, nutrition, and physical therapy. For the energy treatment of osteoporotic fractures to accelerate the healing, after immobilization and fixation, in accordance with Protocol LL 10053, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used).

Ankylosing spondylitis is an inflammatory disease that, over time, can cause some of the bones in the spine (vertebrae) to fuse. This fusing makes the spine less flexible and can result in a hunched posture. If ribs are affected, it can be difficult to breathe deeply. Pain in the back and joints is also common. This condition is more common among men and usually begins in early adulthood. Treatment includes medication, physical therapy, and in rare cases surgery. For the energy treatment of ankylosing spondylitis in accordance with Protocol LL 10053, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The specialized medication regimen can be continued concurrently with the Protocol LL 10053 presented in this invention, at the physician's discretion.

Depending on the affliction and as needed for each case/patient, all treatment embodiments mentioned above for Protocol LL 10053 might be repeated after a period of pause of at least one month.

The next treatment selection that is determined via the algorithm from FIGS. 31-35 is Protocol MM 10054, which refers to the treatment of chronic, internal, non-ischemic, non-infected, with inflammation, and with or without pain medical conditions. This is the case of an internal non-infected and non-ischemic chronic tissue lesion, with significant inflammation, and with or without pain present. For the options of the energy combination systems and their associated energy combination applicators together with treatment regimens of such lesions using Protocol MM 10054 see FIG. 37N and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol MM 10054 see FIG. 37N and FIGS. 36A-36B for the symbols used.

Examples of other chronic, internal, non-ischemic, non-infected, with inflammation, and with or without pain medical conditions, which can use the energy combination systems or combination energy protocols using different energy products corresponding in general to the selection of Protocol MM 10054 are Crohn's disease, ulcerative colitis, osteoarthritis, rheumatoid arthritis (RA) of the joints, non-alcoholic fatty liver, gout, idiopathic pulmonary fibrosis (IPF), chronic tendinitis, plantar fasciitis, and others.

Crohn's disease is a type of inflammatory bowel disease. It causes inflammation of the digestive tract, which can lead to abdominal pain, severe diarrhea, fatigue, weight loss, and malnutrition. Inflammation caused by Crohn's disease can involve different areas of the digestive tract in different people. Most people with Crohn's disease need to take steroids (such as prednisolone) from time to time.

Ulcerative colitis is an inflammatory bowel disease that causes inflammation and ulcers (sores) in the digestive tract. Ulcerative colitis affects the innermost lining of the large intestine (colon) and rectum. Symptoms usually develop over time, rather than suddenly. Ulcerative colitis treatment usually involves either medication therapy or surgery. For the energy treatment of Crohn's disease or ulcerative colitis in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The special medication regimen for Crohn's disease or ulcerative colitis can be continued concurrently with the Protocol MM 10054 presented in this invention, at the physician's discretion.

Osteoarthritis is the most common form of arthritis, which occurs when the protective cartilage that cushions the ends of the bones wears down over time. Although osteoarthritis can damage any joint, the disorder most commonly affects joints from hands, knees, hips, and spine. Treatment includes medicines, physical therapy and, sometimes, surgery to reduce pain and maintain joint movement.

Rheumatoid arthritis (RA) of the joints is a form of arthritis when the body's immune system attacks its own tissue, including joints that causes pain, swelling, stiffness, and loss of function in joints. In severe cases, it attacks internal organs. Rheumatoid arthritis affects joint linings, causing painful swelling. Over long periods of time, the inflammation associated with rheumatoid arthritis can cause bone erosion and joint deformity. Treatment includes nonsteroidal anti-inflammatory drugs (NSAIDs), steroids, corticosteroid medications, such as prednisone, and biologic agents to reduce inflammation, pain and slow joint damage. For the energy treatment of osteoarthritis or rheumatoid arthritis of the joints in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The specialized drug regimen for osteoarthritis or rheumatoid arthritis can be continued concurrently with the Protocol MM 10054 presented in this invention, at the physician's discretion.

Non-alcoholic fatty liver it is an increased buildup of fat in the liver. Major risk factors include obesity and type 2 diabetes, though it is also associated with excessive alcohol consumption. It usually causes no symptoms, but when symptoms occur, they include fatigue, weight loss, and abdominal pain. Treatment involves reducing the risk factors such as obesity through a diet and exercise program. It is generally a benign condition, but in a minority of patients, it can progress to liver failure (cirrhosis). For the energy treatment of non-alcoholic fatty liver in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used).

Gout is a form of arthritis characterized by severe pain, redness, swelling in joints, and tenderness in joints. Pain and inflammation occur when too much uric acid crystallizes and deposits in the joints. Attacks can come suddenly, often at night. During an acute attack, anti-inflammatory medications can help relieve pain and shorten the length of the attack. For the energy treatment of gout in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The medication regimen can be continued concurrently with the Protocol MM 10054 presented in this invention, at the physician's discretion.

Idiopathic pulmonary fibrosis (IPF) is a progressive interstitial lung disease that develops as a result of overexuberant remodeling following pulmonary epithelial damage, and which is characterized by chronic inflammation, alveolar epithelial hyperplasia, and deposition of extracellular matrix leading to development of a permanent “scar”. Treatment includes medications that are called anti-fibrotic agents to slow down the rate of fibrosis or scarring in the lungs. For the energy treatment of idiopathic pulmonary fibrosis (IPF) in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The medication regimen can be continued concurrently with the Protocol MM 10054 presented in this invention, at the physician's discretion.

Chronic tendinitis is the chronic inflammation or irritation of a tendon, which is the thick fibrous cords that attach muscle to bone. The condition causes pain and tenderness just outside a joint. While tendinitis can occur in any of the tendons, it is most common around the shoulders, elbows, wrists, knees, and heels. The treatment consists in the joint rest, nonsteroidal anti-inflammatory drugs (NSAIDs), over-the-counter NSAIDs like ibuprofen (Advil®, Motrin®) can help with inflammation and swelling, corticosteroid injections, orthotics, splints and braces, physical therapy, massage, acupuncture, and platelet-rich plasma.

Plantar fasciitis is an inflammation of a thick band of tissue that connects the heel bone to the toes. The inflamed tissue runs across the bottom of the foot. Symptoms include stabbing pain near the heel. Pain might be worst in the morning. Treatments include physical therapy, shoe inserts, corticosteroid injections, and surgery. For the energy treatment of chronic tendinitis or plantar fasciitis in accordance with Protocol MM 10054, different options of energy combination systems (with applicators incorporating multiple energy technologies and their control consoles, as presented in this invention) or independent energy devices (incorporating only one energy technology) are available, which are presented together with the associated treatment regimen in FIG. 37N (see also FIGS. 36A-36B for the symbols used). The corticosteroids regimen for chronic tendinitis or plantar fasciitis can be continued concurrently with the Protocol MM 10054 presented in this invention, at the physician's discretion.

Depending on the affliction and as needed for each case/patient, all treatment embodiments mentioned above for Protocol MM 10054 might be repeated after a period of pause of at least one month.

The next treatment selection that is determined via the algorithm from FIGS. 31-35 is Protocol NN 10055, which refers to the treatment of chronic, internal, non-ischemic, infected, with inflammation, and with or without pain medical conditions. This is the case of an internal infected and non-ischemic chronic tissue lesion, with significant inflammation, and with or without pain present. For the options of the energy combination systems and their associated energy combination applicators together with treatment regimens of such lesions using Protocol NN 10055 see FIG. 37O and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol NN 10055 see FIG. 37O and FIGS. 36A-36B for the symbols used.

Examples of other chronic, internal, non-ischemic, infected, with inflammation, and with or without pain medical conditions, which can use the energy combination systems or combination energy protocols using different energy products corresponding in general to the selection of Protocol NN 10055 are infectious arthritis, chemotherapy-induced oral mucositis, and others.

Infectious arthritis is an infection in the joints. The infection comes from a bacterial, viral, or fungal infection that spreads from another part of the body. Symptoms of infectious arthritis include intense pain in the joint, redness, and swelling. Treatment of infectious arthritis comprises antimicrobial therapy and joint fluid drainage (arthrotomy, arthroscopy, or daily needle aspiration), followed by antimicrobial therapy. Other treatments include medicines for pain and fever, physical therapy, etc. For the energy treatment of infectious arthritis in accordance with Protocol NN 10055, different options of energy combination systems (with applicators incorporating multiple energy technologies and their control consoles, as presented in this invention) or independent energy devices (incorporating only one energy technology) are available, which are presented together with the associated treatment regimen in FIG. 37O (see also FIGS. 36A-36B for the symbols used). The medication regimen must be continued concurrently with the Protocol NN 10055 presented in this invention.

Chemotherapy-induced oral mucositis causes the mucosal lining of the mouth to atrophy and break down, and thus generating erythema, edema, and ulcerations of the oral mucosa, with debilitating consequences related to pain and the inability to proper eat food or drink liquids. The most common treatments for cancer treatments induced mucositis are antifungals, mucosal protectants, antihistamines, proton-pump inhibitors, etc. For the energy treatment of chemotherapy-induced oral mucositis in accordance with Protocol NN 10055, different options of energy combination systems (with applicators incorporating multiple energy technologies and their control consoles, as presented in this invention) or independent energy devices (incorporating only one energy technology) are available, which are presented together with the associated treatment regimen in FIG. 37O (see also FIGS. 36A-36B for the symbols used). The medication regimen can be continued concurrently with the Protocol NN 10055 presented in this invention, at the physician's discretion.

Depending on the affliction and as needed for each case/patient, all treatment embodiments mentioned above for Protocol NN 10055 might be repeated after a period of pause of at least one month.

The next treatment selection that is determined via the algorithm from FIGS. 31-35 is Protocol OO 10056, which refers to the treatment of chronic, internal, non-ischemic, infected, without inflammation or pain medical conditions. This is the case of an internal infected and non-ischemic chronic tissue lesion, with no inflammation or pain present. For the options of the energy combination systems and their associated energy combination applicators together with treatment regimens of such lesions using Protocol OO 10056 see FIG. 37O and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol OO 10056 see FIG. 37O and FIGS. 36A-36B for the symbols used.

Examples of other chronic, internal, non-ischemic, infected, without inflammation or pain medical conditions, which can use the energy combination systems or combination energy protocols using different energy products corresponding in general to the selection of Protocol OO 10056 are salivary stones and others.

Salivary stones are small stones that form in the glands of the mouth. They can block the flow of saliva and cause pain or discomfort. The treatment consists of applying moist heat and gentle massage to the salivary gland. Staying well-hydrated is important. Lemon drops or other tart candies can help stimulate salivation. Ibuprofen or NSAIDs can be used to reduce pain. In some cases, surgery is needed to remove the stones from the salivary glands. For the energy treatment of salivary stones in accordance with Protocol OO 10056, different options of energy combination systems (with applicators incorporating multiple energy technologies and their control consoles, as presented in this invention) or independent energy devices (incorporating only one energy technology) are available, which are presented together with the associated treatment regimen in FIG. 37O (see also FIGS. 36A-36B for the symbols used). The prescribed medication or antibiotic (in case of infection) regimen can be continued concurrently with the Protocol OO 10056 presented in this invention, at the physician's discretion.

Depending on the affliction and as needed for each case/patient, all treatment embodiments mentioned above for Protocol OO 10056 might be repeated after a period of pause of at least one month.

The next treatment selection that is determined via the algorithm from FIGS. 31-35 is Protocol PP 10057, which refers to the treatment of chronic, internal, non-ischemic, infected, without inflammation, and painful medical conditions. This is the case of an internal infected and non-ischemic chronic tissue lesion, with no inflammation, and significant pain present. For the options of the energy combination systems and their associated energy combination applicators together with treatment regimens of such lesions using Protocol PP 10057 see FIG. 37O and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol PP 10057 see FIG. 37O and FIGS. 36A-36B for the symbols used.

Examples of other chronic, internal, non-ischemic, infected, without inflammation, and painful medical conditions, which can use the energy combination systems or combination energy protocols using different energy products corresponding in general to the selection of Protocol PP 10057 are leprosy and others.

Leprosy, also known as Hansen's disease, is a chronic infectious disease caused by Mycobacterium leprae. The disease mainly affects the skin, the peripheral nerves, mucosal surfaces of the upper respiratory tract and the eyes. Symptoms include light colored or red skin patches with reduced sensation, numbness, and weakness in hands and feet. Leprosy can be cured with 6-12 months of multi-drug therapy. Early treatment avoids disability. For the energy treatment of leprosy in accordance with Protocol PP 10057, different options of energy combination systems (with applicators incorporating multiple energy technologies and their control consoles, as presented in this invention) or independent energy devices (incorporating only one energy technology) are available, which are presented together with the associated treatment regimen in FIG. 37O (see also FIGS. 36A-36B for the symbols used). The multi-drug therapy regimen can be continued concurrently with the Protocol PP 10057 presented in this invention, at the physician's discretion.

Depending on the affliction and as needed for each case/patient, all treatment embodiments mentioned above for Protocol PP 10057 might be repeated after a period of pause of at least one month.

The next treatment selection that is determined via the algorithm from FIGS. 31-35 is Protocol QQ 10058, which refers to the treatment of chronic, internal, ischemic, non-infected, without inflammation or pain medical conditions. This is the case of an internal ischemic and non-infected chronic tissue lesion, with no inflammation, and with no pain present. For the options of the energy combination systems and their associated energy combination applicators together with treatment regimens of such lesions using Protocol QQ 10058 see FIG. 37P and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol QQ 10058 see FIG. 37P and FIGS. 36A-36B for the symbols used.

Examples of other chronic, internal, ischemic, non-infected, without inflammation or pain medical conditions, which can use the energy combination systems or combination energy protocols using different energy products corresponding in general to the selection of Protocol QQ 10058 are ischemic erectile disfunction, bundle branch block, and others.

Ischemic erectile disfunction happens when arteries are blocked by plaque or fatty deposits, the condition can make it impossible for blood to travel to the penis. Treatment consists of phosphodiesterase type 5 (PDE5) inhibitors as sildenafil (Viagra), tadalafil (Cialis), vardenafil (Levitra), and avanafil (Stendra), testosterone, vacuum devices and other therapies. For the energy treatment of ischemic erectile disfunction in accordance with Protocol QQ 10058, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used). The phosphodiesterase type 5 (PDE5) inhibitor regimen can be continued concurrently with the Protocol QQ 10058 presented in this invention, at the physician's discretion.

Bundle branch block is a condition in which there's a delay or blockage along the pathway that electrical impulses travel to make the heart beating. Left bundle branch block sometimes makes it harder for the heart to pump blood efficiently through the circulatory system. Most people do not have symptoms and when symptoms occur, they include fainting or a slow heart rate. In patients with heart failure, a pacemaker also can relieve symptoms as well as prevent death. For the energy treatment of bundle branch block in accordance with Protocol QQ 10058, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used).

Depending on the affliction and as needed for each case/patient, all treatment embodiments mentioned above for Protocol QQ 10058 might be repeated after a period of pause of at least one month.

The next treatment selection that is determined via the algorithm from FIGS. 31-35 is Protocol RR 10059, which refers to the treatment of chronic, internal, ischemic, non-infected, without inflammation, and painful medical conditions. This is the case of an internal non-infected and ischemic chronic tissue lesion, with no inflammation, and significant pain present. For the options of the energy combination systems and their associated energy combination applicators together with treatment regimens of such lesions using Protocol RR 10059 see FIG. 37P and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol RR 10059 see FIG. 37P and FIGS. 36A-36B for the symbols used.

Examples of other chronic, internal, ischemic, non-infected, without inflammation, and painful medical conditions, which can use the energy combination systems or combination energy protocols using different energy products corresponding in general to the selection of Protocol RR 10059 are vasculitis skin lesions, peripheral vascular disease (PVD), and others.

Common vasculitis skin lesions are red or purple dots (petechiae), usually most numerous on the legs. In other cases, larger spots are produced, about the size of the end of a finger (purpura), some of which look like large bruises. The treatment consists of medication for pain and immunosuppressants for several years. For the energy treatment of vasculitis skin lesions in accordance with Protocol RR 10059, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used). The immunosuppressants regimen can be continued concurrently with the Protocol RR 10059 presented in this invention, at the physician's discretion.

Peripheral vascular disease (PVD) is a slow and progressive blood circulation disorder, due to the narrowing, blockage, or spasms in a blood vessel. PVD may affect any blood vessel outside of the heart including the arteries, veins, or lymphatic vessels. Treatment may include procedures to widen the artery, medications to reduce the build-up of fatty deposits within blood vessels, and lifestyle changes such as weight loss and regular exercise. For the energy treatment of peripheral vascular disease (PVD in accordance with Protocol RR 10059, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used).

Depending on the affliction and as needed for each case/patient, all treatment embodiments mentioned above for Protocol RR 10059 might be repeated after a period of pause of at least one month.

The next treatment selection that is determined via the algorithm from FIGS. 31-35 is Protocol SS 10060, which refers to the treatment of chronic, internal, ischemic, non-infected, with inflammation, and with or without pain medical conditions. This is the case of an internal infected and non-ischemic chronic tissue lesion, with significant inflammation, and with or without pain present. For the options of the energy combination systems and their associated energy combination applicators together with treatment regimens of such lesions using Protocol SS 10060 see FIG. 37P and also FIGS. 36A-36B for the symbols used.

Equally, a combination energy protocol is a protocol that is using independent energy devices (incorporating only one energy technology that are presented in other patents) or energy combination applicators and systems (incorporating two or more energy technologies, as presented in this invention) to perform main treatment sessions, which are combined with secondary treatment sessions that can be applied in between the main treatment sessions. If secondary treatment is using independent energy devices, based on the specifics of each medical affliction, one or multiple secondary treatment sessions can be done during the time period in between the main treatment sessions. For the combination energy protocol options and treatment regimen using Protocol SS 10060 see FIG. 37P and FIGS. 36A-36B for the symbols used.

Examples of other chronic, internal, ischemic, non-infected, with inflammation, and with or without pain medical conditions, which can use the energy combination systems or combination energy protocols using different energy products corresponding in general to the selection of Protocol SS 10060 are vasculitis, heart muscle reactivation after heart infarction, and others.

Vasculitis involves inflammation of the blood vessels. The inflammation can cause the walls of the blood vessels to thicken, which reduces the width of the passageway through the vessel. If blood flow is restricted, it can result in organ and tissue damage. The key to lessening the symptoms of the many types of vasculitis is to reduce inflammation. Anti-inflammatory medications, notably glucocorticoids such as prednisone or methylprednisolone, are the most common first-line treatments. For the energy treatment of vasculitis in accordance with Protocol SS 10060, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used). The anti-inflammatory medication regimen can be continued concurrently with the Protocol SS 10060 presented in this invention, at the physician's discretion.

Reactivation of heart muscle after heart infarction, which is tissue death or necrosis due to inadequate blood supply to the affected area. Once disease reduces blood flow, the heart muscle may hibernate. It reduces its function to the point where it's barely keeping itself alive. Cardiac heart muscle rehabilitation involves exercise training, emotional support, and education about a heart-healthy lifestyle. Healthy lifestyle habits include eating a nutritious diet, managing weight, and quitting smoking. For the energy treatment of reactivation of heart muscle after heart infarction in accordance with Protocol SS 10060, different options of energy combination systems (with applicators incorporating multiple energy technologies and their control consoles, as presented in this invention) or independent energy devices (incorporating only one energy technology) are available, which are presented together with the associated treatment regimen in FIG. 37P (see also FIGS. 36A-36B for the symbols used).

Depending on the affliction and as needed for each case/patient, all treatment embodiments mentioned above for Protocol SS 10060 might be repeated after a period of pause of at least one month.

For the energy combination systems embodiments from FIGS. 1A-14E, 21A-21E, when the applicator/coupling membrane 15 is in direct contact with the wound/lesion/tissue condition 19, if the distal end of the applicator/coupling membrane 15 is in the form of a suction cup that creates an enclosure in between the applicator/coupling membrane 15 and the skin 21 that can be filled with saline or any other fluid and thus be able to eliminate the gel and produce cavitation at the surface of the wound/lesion/tissue condition 19, which can destroy biofilms or prevent their formation with benefic effects on healing. The same suction cup design for applicator/coupling membrane 15 can be also used to simultaneously apply a negative pressure treatment in combination with any of the embodiments from FIGS. 1A-14E, 21A-21E.

Furthermore, the energy combination systems embodiments from FIGS. 1A-21E, 28A-30B can be used for the delivery of drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, etc., which are in a fluid form or are incorporated into a surrounding delivery fluid that creates an active medication. For such purpose, the energy combination systems embodiments from FIGS. 1A-21E, 28A-30B should be placed on top of a patch containing the respective fluid or fluids or active medications and use their energy delivery mechanisms to push them into the tissue from the targeted treatment zone. In another embodiment, the liquid active substances mentioned before can be spread on top of the wound/lesion/tissue condition 19 or are injected in the targeted region and then the energy combination systems embodiments from FIGS. 1A-21E, 28A-30B can be used to push such liquid active substances into the tissue from the targeted treatment zone.

Moreover, sometimes the delivery of a certain liquid active substance (drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., and the like) in the desired tissue or organ is hindered by the excessive inflammation and/or scar tissue or due to non-adequate blood circulation. Energy combination systems embodiments from FIGS. 1A-30B can be used to increase blood circulation via blood vessels dilatation, or to modulate inflammation and to reduce or eliminate of scarring, or to prep the targeted treatment zone before the specific treatment. This will enhance the uptake of the drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., and the like. Furthermore, after the injection or systemic oral administration or local delivery of drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., energy combination systems embodiments from FIGS. 1A-21E, 28A-30B can be used to promote the precise delivery inside the human or animal tissues or organs of the treating substances/medicines and facilitate their proper activation in the desired/targeted regions of the human or animal body.

Also, energy combination systems embodiments from FIGS. 1A-30B can be periodically used to treat the targeted region after initial injection or oral administration or local delivery of the drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., to continue improving blood circulation and tissue/cellular activation and stimulation, to be sure that the treatment is effective and properly sustained by enough oxygenation, nutrients, and other healing factors. In the case of the stem cells, the fast differentiation of the stem cells in the type of cells need for repair can be promoted and sustained by such energy combination systems embodiments from FIGS. 1A-21E, 28A-30B.

The big drawback for many drugs/medications is represented by the adverse effects generated when the dosages are enhanced to provide a better therapeutically effect. Many drugs/medications that must be delivered systemically, via gastro-intestinal or blood circulatory routes, are dropped out during research phase due to their delirious side effect at the effective dosages. The energy combination systems embodiments from FIGS. 1A-21E, 28A-30B can play an important role for such drugs/medications that produce significant side effects, when delivered systemically. If such drugs/medications in a liquid form are encapsulated in liposomes, lipid nano-particles or any other artificially or natural envelopes, they can be delivered to the specific tissue via a locally or systemically approaches without adverse effects. When these drugs encapsulated in liposomes, lipid nano-particles or any other artificially or natural envelopes reach the desired tissue, energy combination systems embodiments from FIGS. 1A-21E, 28A-30B can be applied extracorporeally to the specific tissue or organ, to break these envelopes and deliver safely a high dosage of the respective active substance where is needed, without systemic side effects in other parts of the body. The diametral size of the envelopes used will dictate the type of tissue that is targeted, where such enveloped-drug should get in interstitially and concentrate. The intact envelopes that did not reached the desired tissue will be safely eliminated via urinary or gastric tracts. The extracorporeal approach and the possibility to penetrate deep inside the human and animal body, makes the energy combination systems embodiments from FIGS. 1A-21E, 28A-30B the ideal delivery systems for such drugs/medications.

By using the energy combination systems embodiments from FIGS. 1A-21E, 28A-30B as adjunct systems for proper delivery and penetration and activation of the drugs, antibiotics, antioxidants, traditional natural medical substances, vaccines, growth factors, proliferation factors, angiogenesis factors, cytokines, exosomes, enzymes, stem cells, antibodies, osteoclasts, fibroblast, keratinocytes, endothelial cells, bio-nanoparticles, nano-robots, genetic material (DNA/RNA/mRNA fragments), scaffolds or matrices to mimic extracellular matrices, nano-robots, specific proteins, or mixtures/cocktails of multiple active ingredients, etc., (denoted in general as active substances or medicines) there is a distinct possibility to significantly reduce the quantity of active substances or medicines needed for successful outcome. Such reduced dosages will have a significant impact on the manufacturing process for these active substances or medicines, which will allow more doses to be available for the same quantity produced and also on diminished toxicity and side effects associated with it. Furthermore, by having a lower dosage of the active substances or medicines for a certain treatment, will reduce the possibility of generating side effects and rejection, which will make the treatment more tolerable and successful. Even more, the active substances or medicines that were initially rejected for side effects may be brought back into the game due to the enhanced and targeted delivery produced by the energy combination systems embodiments from FIGS. 1A-21E, 28A-30B.

The energy combination systems embodiments from FIGS. 1A-30B can be also used to deliver cellular material from placenta or umbilical cord tissues, or from human, fish or animal skin, to form skin substitutes or to help with the integration of scaffolds made of placental or umbilical cord tissues, human, animal or fish skin, etc. in acute or chronic wounds or different lesions or tissue conditions, etc.

In another embodiment, the energy combination systems embodiments from FIGS. 1A-30B can be used to deliver growth factors (FGF—fibroblast growth factor, IGFBP-3—insulin-like growth factor-binding protein, IGF-a—insulin-like growth factor, EGF—epidermal growth factor, VEGF—vascular endothelial growth factor, natural fibrin, GM-CSF—granulocyte/macrophage colony-stimulating factor, which is a hematopoietic growth factor and immune modulator, BMP—bone morphogenetic protein, a group of signaling proteins that belongs to the TGF-β—Transforming Growth Factor-β, PDGF—platelet-derived growth factor, etc.) in an autologous form that is extracted from patients own platelets, or PRP—platelet-rich plasma, or rhVEGF—recombinant vascular endothelial growth factor, or PDGF—platelet derived growth factor, which includes the natural growth factors produced by the patient own body or in a recombinant form, which is in a molecular biologic form.

In another embodiment, the energy combination systems embodiments from FIGS. 1A-30B can be also used to generate the change in shape of the nanoparticles (shape-shifting nanoparticles similar to origami), which allows these shape-shifting nanoparticles to release active agents after they are subject to the energy action. After activation with energy combination systems from FIGS. 1A-30B, the shape-shifting nanoparticles also can act as anti-bacterial or anti-viral or antibody particles that fight bacterial or viral or micro-organisms infections or bacterial biofilms, due to their specific shape that produce the breach the integrity of bacterial, viral, or micro-organisms membrane/envelopes. The shape-shifting nanoparticles that specifically activated in specific shapes (due to the energy combination systems from FIGS. 1A-30B action) can also poke the biofilms to allow their disintegration/breach, which facilitates the penetration of different drugs that will destroy the bacterial biofilms. The shape-shifting nanoparticles can be designed to have minimal size for delivery inside the tissues/organs/human or animal bodies and after activation, via energy combination systems embodiments from FIGS. 1A-30B, can turn in three dimensional shapes with different prongs or spikes that will make the nanoparticles much larger in size. Having the poking means (spears-like prongs, spikes, thorns, pointy cones, cylindrical prongs or any other form of prongs, etc.) will allow the shape-shifting nanoparticles (after their activation with the energy combination systems embodiments from FIGS. 1A-30B) to be used to breach the membrane/external envelope of bacteria, viruses, micro-organisms, etc. or scrape the biofilms.

In yet another embodiment, the energy combination systems embodiments from FIGS. 1A-30B can be used to push stem cells (adult stem cells, embryonic stem cells, or pluripotent stem cells—see. U.S. Pat. No. 8,728,809) inside the wounds/lesions/tissue conditions 19 or organs and produce their differentiation in the desired type of tissues. The same energy combination systems embodiments from FIGS. 1A-30B can be used first to prep the area (to create new blood vessels and thus enhancing nutrition to the targeted cellular material or tissues or organs). The energy combination systems embodiments from FIGS. 1A-30B can also be used after the stem cell delivery to the targeted region, to help “open” stem cells and neighboring cell walls and thus stimulate inter-cellular communication to facilitate the proper stem cell differentiation into the right type of cells and tissues that is needed for the cure or treatment and to enhance stem cells integration with the surrounding tissues or organs.

Due to the use of the energy combination systems embodiments from FIGS. 1A-30B, a reduction of the number of stem cells needed for the medical treatment can be accomplished with the advantages mentioned before for active medical substances in general. Such advantages are less adverse events and less stem cells used for each treatment, which reduces the manufacturing cycle for each dose and maximizes the number of dosages per each batch of stem cells that were stimulated and multiplied in a bioreactor. The reduction of the necessary dosage of the stem cells needed for a treatment is accomplished due to the targeted and facilitated more efficient differentiation produced by the combination energy treatments. Also, by applying the energy combination systems embodiments from FIGS. 1A-30B to cells or tissues or organs, an enhanced blood supply is produced immediately due to blood vessels dilation and in the long run due to formation of new small blood vessels (angiogenesis). This means that when the energy combination systems embodiments from FIGS. 1A-30B are used in conjunction with the stem cell therapy, they will produce an accelerated differentiation and enhanced survivability of the newly differentiated stem cells in the body due the proper nutrients being brought to the area/region by sufficient blood circulation, which enhances the healing and the stem cells treatment benefic results. Based on the same rationale, as presented in this paragraph, the same action principles and benefic effects can be used for delivering reduce dosages of active substances/drugs or ingredients or materials/nano-materials or genetic materials or mixture/cocktail of multiple active ingredients, for various treatments of human or animal cells/tissues/organs or for plants.

It will also be appreciated that in some embodiments, the energy combination systems embodiments from FIGS. 1A-30B can be used in an ex vivo (out of body) environment in combination with stem cell therapy, gene therapies, other active substances, and the like, to generate a desired type of cell, tissue, organ or similar body element that is subsequently introduced into the body or transplanted in an in vivo environment (inside the body). Before implantation of such transplant, the energy combination systems embodiments from FIGS. 1A-30B can be used to prep the area for proper blood circulation and to enhance cellular communication. After transplant implantation in the desired targeted region, the energy combination systems embodiments from FIGS. 1A-30B can be also used to stimulate the surrounding tissues for easy integration and increased chance of survivability of the transplant.

Similar energy combination systems from FIGS. 1A-21E, 28A-30B can be used when the stem cells or DNA, or RNA, or proteins, or active ingredients, or drugs are delivered via bio scaffolds/implants, injection, or systemic approach. Such energy combination systems embodiments from FIGS. 1A-21E, 28A-30B can be used to prep the treatment targeted area/region for increase blood circulation and to open the pores on the cell membrane (via induced cellular mechanotransduction). The same energy combination systems from FIGS. 1A-21E, 28A-30B are used to push/deliver the active DNA, RNA, proteins, and other active ingredients or drugs to the treatment targeted region from the human or animal body. After successful delivery of active DNA, RNA, proteins, other active ingredients, or drugs to the targeted cellular region or tissues or organs, the energy combination systems embodiments from FIGS. 1A-30B can be also utilized for stimulation in situ of such active ingredients for proper integration and therapeutical effect. This post implantation stimulation via energy combination systems embodiments from FIGS. 1A-30B, it will provide positive effects in the upregulation of benefic healing factors that accelerate the cellular or tissue or organ regeneration.

In general, energy combination systems embodiments from FIGS. 1A-30B can open pores in the membranes of the mammalian cells through a mechanotransduction mechanism, which is produced by rapid variation from high compressive positive pressures to negative pressure in the targeted treatment region generated by different acoustic waves, or by heat stimulation, or by electromagnetic activation, or by radio-frequency initiation, or by electric currents stimulation, or by photo-modulation, etc., which are generated by treatments using the energy combination systems embodiments from FIGS. 1A-30B. Thus, combination energy medical treatments can be applied with the energy combination systems embodiments from FIGS. 1A-30B to stimulate the targeted tissue or cells via mechanotransduction, and produce the opening of the cell's membrane vesicles, which assures a rapid absorption into the cells of the subsequently injected genetic material, or viruses, or different vectors during a gene therapy. The energy combination systems embodiments from FIGS. 1A-30B facilitating such absorption, allow an enhanced targeted delivery of the genetic treatment (normal genes or genetic modified material or the base editing means/apparatus) inside the targeted tissue or cells. Furthermore, the same energy combination systems embodiments from FIGS. 1A-30B can be used after genetic material injection/delivery, to enhance body absorption and reengineering of the tissue, due to formation of new blood vessels and enhanced growth factors that help with the desired changes in cells or tissues or organs. Additionally, these combination energy medical treatments can help reduce the dosages of gene therapy needed for a successful treatment, since the facilitation of absorption should require a reduction in the dosage of vectors and genetic material required to produce the desired genetic modification. Furthermore, the reduced dosage needed for the gene treatments, can also significantly diminish the side effects of such therapies.

In certain embodiments, the energy combination systems presented in FIGS. 1A-21E, 28A-30B may be used to facilitate genetic modifications (such as in conjunction with gene therapies) and also to enable stem cell therapies (such as promotion and acceleration of differentiation of stem cells), and thus producing a combination of genes and stem cells therapies. These energy combination systems presented in FIGS. 1A-21E, 28A-30B can promote the gene therapies and/or stem cells therapies, or combination of them, and can enhance the absorption of genetic material inside the cells/tissues/organs or produce gene modification of the cells or expedite the action of the stem cell treatment. These effects produced by the addition of energy combination systems presented in FIGS. 1A-21E, 28A-30B for genetic treatments or stem cells treatments or combination of them can reduce the number of genetic material or stem cells needed for one dosage, with significant implication in reducing possible adverse reactions at the recipient, which can enhance the adoption of such treatments that are now plagued with such adverse events. Also, from the manufacturing point of view, if one injection or dosage requires less genetic load (DNA, RNA, mRNA, gRNA) or stem cells, this will allow the production of much more dosages with the same amount of genetic or stem cell material that was produced in one batch from one bioreactor.

The energy combination systems from FIGS. 1A-30B can be used to facilitate the combination of gene therapy (and or other active substance therapies) with stem cell therapy in different ways, as follows:
initially introducing gene therapy to a treatment site using the energy combination systems embodiments from FIGS. 1A-30B to facilitate the gene therapy and subsequently introducing stem cells, preferably using the same energy combination systems embodiments from FIGS. 1A-30B to accelerate processes, to differentiate the stem cells into cells that mimic cells that have been “repaired’ by gene therapy;
simultaneously introducing both the genes and stem cells to a treatment site in conjunction with the use of energy combination systems embodiments from FIGS. 1A-30B to facilitate both genetic modification and stem cell differentiation in the targeted tissue, as an enhanced treatment modality;
initially introducing stem cells to a treatment site using the energy combination systems embodiments from FIGS. 1A-30B to facilitate stem cell differentiation in “healthy” cells necessary to create a proper environment for the surviving of the gene modified cells introduced subsequently, preferably using the same energy combination systems embodiments from FIGS. 1A-30B, to accelerate absorption of the genetically modify cells as desired for the treatment.

Regardless of the administration method chosen for the combination of gene therapy with the stem cells therapy, it is preferable to continue periodically (at least two times per week) the treatment with the energy combination systems embodiments from FIGS. 1A-30B post-implantation of the stem cells and genetic material, to increase new blood vessels creation (neovascularization into the targeted region), which will give enhanced oxygenation and nutrients to assure proper integration of the new stem cells and genetically modified cells inside the tissues or organs and ultimately to produce functionality regeneration of the targeted human or animal tissues or organs. The same method can be applied to plant materials for enhanced hybridization to create plants that are more resistant to diseases and predators and simultaneously have enhanced productivity.

Furthermore, drugs or nanobots or nanoparticles, and other active substances or medicines could be used in conjunction with gene and/or stem cell therapies when the energy combination systems embodiments from FIGS. 1A-30B are used to facilitate their enhanced delivery and integration in the cellular material or tissues or organs to obtain the desired outcomes for the treatments of human or animal or plants.

The first generation of gene editing was CRISPR (Clustered Regularly Interspaced Short Palindromic Repeats), a technology developed in 2012 that can target and cut sections of DNA like a pair of scissors. CRISPR gene editing is a genetic engineering technique in molecular biology by which the genomes of living organisms may be modified. It is based on a simplified version of the bacterial CRISPR-Cas9 antiviral defense system. By delivering the Cas9 nuclease complexed with a synthetic guide RNA (gRNA) into a cell, the cell's genome can be cut at a desired location, allowing existing genes to be removed and/or new ones added in vivo. Another technique for gene editing is known as Base Editing technology that works more like a pencil, which can target a single misspelling in the DNA code allowing for much greater precision. These technologies could theoretically cure thousands of the genetic diseases caused by single letter misspellings, known as point mutations.

Examples of rare genetic disease that might benefit from CRISPR or Base Editing technologies and associated genetic treatments are presented below.

Sickle Cell Disease, which is an inherited blood disorder causes severe pain. Abnormal hemoglobin molecules—hemoglobin S—stick to one another and form long, rod-like structures. These structures cause red blood cells to become stiff, assuming a sickle shape. Their shape causes these red blood cells to pile up, causing blockages and damaging vital organs and tissue.

T-Cell Acute Lymphoblastic Leukemia, which can produce fast-growing blood cancer. Acute lymphocytic leukemia (ALL) is also called acute lymphoblastic leukemia. “Acute” means that the leukemia can progress quickly, and if not treated, would probably be fatal within a few months. “Lymphocytic” means it develops from early (immature) forms of lymphocytes, a type of white blood cell.

Acute Myeloid Leukemia is another disorder that produces fast-growing blood cancer. Acute myeloid leukemia (AML) is a cancer of the myeloid line of blood cells, characterized by the rapid growth of abnormal cells that build up in the bone marrow.

Alpha-1 antitrypsin (AAT) deficiency is an under-recognized hereditary disorder associated with the premature onset of chronic obstructive pulmonary disease, liver cirrhosis in children and adults, and less frequently, relapsing panniculitis, systemic vasculitis, and other inflammatory, autoimmune, and neoplastic diseases.

Glycogen Storage Disorder 1a Inherited, known also as Von Gierke disease, is a disorder characterized by the body inability to store sugar. Glycogen storage disease type 1 is an inherited disorder caused by the buildup of a complex sugar called glycogen in the body's cells. The accumulation of glycogen in certain organs and tissues, especially the liver, kidneys, and small intestines, impairs their ability to function normally.

Stargardt disease is also called Stargardt macular dystrophy, juvenile macular degeneration, or fundus flavimaculatus. The disease causes progressive damage or degeneration of the macula, which is a small area in the center of the retina that is responsible for sharp, straight-ahead vision.

Angelman Syndrome is a genetic condition in which most people have a gene called UBE3A that is absent or faulty. When this gene is faulty or missing, nerve cells in the brain are unable to work properly, causing a range of physical and intellectual problems.

Ankylosing Spondylitis is a kind of arthritis that affects the joints and ligaments of the spine, but can affect other large joints, and can be related to problems in the eyes, skin, bowel, and heart.

Apert Syndrome is a rare condition, usually evident at birth, that causes an abnormally shaped skull and fused fingers and toes. Other body parts and organs are also affected.

Charcot-Marie-Tooth (CMT) Disease is an inherited neurological condition that causes problems with the muscles of the feet, legs, arms, and hands.

Cystic Fibrosis is a genetic disease that mostly affects the lungs and digestive system. It results from a fault in a particular gene. As a result, the mucus produced by the lungs and intestines becomes thick and sticky.

Duchenne Muscular Dystrophy (DMD) is a severe type of muscular dystrophy that primarily affects boys. Muscle weakness usually begins around the age of four, and worsens quickly. Muscle loss typically occurs first in the thighs and pelvis followed by the arms. This can result in trouble standing up. Most are unable to walk by the age of 12. Affected muscles may look larger due to increased fat content. Scoliosis is also common. Some may have intellectual disability. Females with a single copy of the defective gene may show mild symptoms.

Haemochromatosis is an inherited condition that causes the body to absorb too much iron. In some cases of haemochromatosis, the extra iron can lead to organ damage.

Hemophilia is a bleeding disorder caused by a gene mutation. Due to hemophilia, the blood does not clot properly, which makes it difficult to control bleeding. When a blood vessel is injured, special proteins in the blood called ‘clotting factors’ act to control blood loss by plugging or patching up the injury. People with hemophilia have lower than normal levels of a clotting factor.

Huntington's Disease is an inherited condition that affects the nervous system. Although Huntington's disease can occur at any age, symptoms often don't appear until middle age. Main symptoms are stiffness, involuntary movements, changes in balance and coordination, loss of control of bodily functions such as swallowing and speaking, fatigue, difficulty concentrating, deterioration of memory, judgement and speed of thought.

Klinefelter Syndrome is a genetic condition affecting males. It occurs if a man is born with an extra X chromosome. It can cause a variety of problems, including a small penis, small testes, and infertility.

Marfan Syndrome is caused by a gene abnormality, specifically a change (mutation) in the gene that affects the proteins that help make healthy connective tissue that holds together bones, muscles, organs, and tissues in the body muscles and joints. It affects the heart, eyes, blood vessels and skeleton. People with Marfan Syndrome are usually tall and thin with unusually long arms, legs, fingers and toes. The damage caused by Marfan syndrome can be mild or severe.

Neurofibromatosis is a genetic condition characterized by the growth of benign tumors. The symptoms include light brown spots on the skin, freckles in the armpit and groin, small bumps within nerves, and scoliosis. Also, there may be hearing loss, cataracts at a young age, balance problems, flesh colored skin flaps, and muscle wasting.

Prader-Willi Syndrome is a rare genetic disorder that causes a range of physical, intellectual, and behavioral problems. A key feature of Prader-Willi Syndrome is a constant sense of hunger that usually begins at about 2 years of age.

Rett Syndrome is a rare genetic disorder that mainly affects the nervous system of girls, causing intellectual and physical disability. It is caused by a mutation in a gene on the X chromosome that usually results from a random genetic mutation rather than being inherited.

Tay-Sachs Disease also known as Progeria is a genetic disorder that leads to the premature death of young children. Babies with infantile Tay-Sachs Disease appear healthy at birth, but by the time they are 6 months old, their development is slowing. They gradually lose power and movement in their limbs, and gradually lose their vision. Over time the children regress in other ways, losing the power of speech and many other functions. Most children with infantile Tay-Sachs disease die before getting to school age.

Thalassemia is an inherited genetic disorder that affects the blood. People with thalassemia do not produce enough healthy hemoglobin. Inherited blood disorder causes severe anemia.

Von Willebrand Disease is an inherited bleeding disorder. People with von Willebrand Disease have problems controlling their bleeding.

For the treatment of many of these above-mentioned genetic diseases, one of the big technical challenges of CRISPR and Base Editing technologies is the refining of the successfully delivery methods of modified genetic material into the patient's body. While for some of modified genetic material treatments, the modification can be done outside the body and inserted, for other diseases, like Progeria, the base editor will have to be directly inserted into the patient. The challenge is the creation of a delivery system that is going to take the genetic material or a Base Editing apparatus efficiently and safely to the cells where it needs to do their work. For that the cells need to open membrane pores and allow the selectively delivery of the genetic material undamaged inside the mammalian cells, via viruses or other vectors. The energy combination systems from FIGS. 1A-30B can be successfully used to open pores in the mammalian cells based on mechanotransduction that is the process by which physical forces created by energy combination systems embodiments from FIGS. 1A-21E are sensed and converted into biochemical and electrical signals that then result in cellular responses in creating pores on their outer membrane. These pores can make the delivery of the genetic material, via viruses or other vectors, undamaged inside the mammalian cells, and better control the CRISPR and Base Editing processes for genetic modifications.

Traditional Chinese medicine is using body's energy points or acupuncture points that regulate the energy balance of the tissues and organs for healing and for reducing or eliminating pain and inflammation. The needles used in the Traditional Chinese medicine acupuncture can be replaced by shockwaves, ultrasound, electric currents, non-ablation lasers, or a combination of them, as presented in the energy combination systems embodiments from FIGS. 2A-2B, 5A-6B, 8A-9B, 12, 13E, 15, 18, 21A-21E of this invention. In this case, the combination energy applicators of the before mentioned embodiments must be moved along the body's energy lines in different parts of the body. The advantage is that the application of combination energy systems instead of acupuncture needles is less painful and much easier accepted by the patients, and in the same time might stimulate via growth factors the regeneration of the cells, or tissues, or organs, or to generate new blood vessels in the ischemic regions, which can enhance the healing, reduce pain and inflammation of the targeted tissue or lesion or organ.

For energy combination systems that use sound waves (as shockwaves or ultrasound), which are known to produce cavitation, coupling fluids or injected fluids with air nuclei can be used to create more cavitation. Such sterilized fluids that are used to enhance cavitation on top of the wound/lesion/tissue condition 19 can be used to fill-in specially designed coupling bladders or spacers (not shown in the figures of this invention). These bladders and spacers can seep such fluids on top of the wound/lesion/tissue condition 19 through specially designed pores, which can open only through the action of the energy combination systems presented in the embodiments from FIGS. 1A-21E, 28A-30B. In other embodiments, the coupling bladders or spacers can be designed to adhere tightly to the skin 21 of the human/animal body 20 and thus creating a tight and enclosed space in which the fluid has direct contact with the wound/lesion/tissue condition 19, which will enhance the action of cavitation. The sterilized coupling fluids or injected fluids with air nuclei can be spread on top of the wound/lesion/tissue condition 19 or injected inside the wound/lesion/tissue condition 19 at the point-of-care by the medical personnel, or can be prefilled in the specially designed coupling bladders or spacers by specialized medical companies. The specially designed coupling bladders or spacers, which have means to adhere to the skin 21 of the human/animal body 20 and thus creating a tight and enclosed space in which the fluid has direct contact with the wound/lesion/tissue condition 19, can be used for all the energy combination systems presented in the embodiments from FIGS. 1A-21E, 28A-30B.

Furthermore, miniaturized energy systems producing electromagnetic fields, or radio-frequency fields, or electric currents, or phototherapy fields, or pressure fields, or ultrasound, or a combination of them, which are similar in usability and functionality with the ones presented in the embodiments for energy combination systems embodiments from FIGS. 1A-27, can be incorporated in garments as caps or hats (for head treatment), or gloves (for hand treatments), or gloves with long sleeves (for hand and arm treatments), or vests and jackets (for chest and abdominal area treatments), or girdles (for abdominal area), or undergarments (for the groin area treatments), or elastic sleeves (for neck, shoulders, elbows, wrists, knees, or ankles treatments), or socks (for the legs, ankles, and foot area), etc. All these garments driven by energy combination systems similar to those presented in FIGS. 1A-27 should be operated by miniaturized batteries or special miniaturized rechargeable batteries that are using body or body parts or skin movements, which occur during usual day-to-day activities or even breathing, to produce small therapeutic electrical pulses and electric fields.

In another embodiment, electromagnetic fields can be created in specially designed shoes or soles that incorporate miniature electromagnetic-generators. Such shoes or soles with electromagnetic fields can be used for prevention of diabetic foot ulcers (DFU) or as secondary treatments in between main treatments performed with energy combination systems embodiments from FIGS. 1A-21E (incorporating two or more energy technologies, as presented in this invention) or by using independent energy devices (incorporating only one energy technology that are presented in other patents) that can produce shockwaves, ultrasound (contact or non-contact), phototherapy (LED/OLED or laser or ultraviolet light), topical negative pressure, electric stimulation, pneumatic compression, radio frequency, oxygen, etc.

The hydrotherapy treatment modality is most commonly applied using whirlpools, but more recently, other irrigation systems such as pulsed lavage with concurrent suction (PLWCS) have been designed for wound care. Instead of water different active substances can be used that provide broad antibacterial effect as protocatechuic acid, etc. It is usually used for debridement. Interesting to note that in the case of wound cleaning using hydrotherapy (involving the delivery of water or other fluids to the wound bed), shockwaves or ultrasound or combination energy systems, as the ones presented in energy combination systems embodiments from FIGS. 9A-9B, 21A-21E, can be used instead of whirlpool or pulsed lavage with concurrent suction. The advantage is that shockwaves or ultrasound or combination energy systems including both technologies can simultaneously deliver energy to the cellular materials/tissues/organs to help with healing via growth factors stimulation and the creation of new blood vessels to reduce ischemia in the targeted region and also generates the hydrotherapy with its benefic effects.

Many cellular processes involved in healing are known to be dependent on oxygen including collagen deposition, anti-microbial activity of leukocytes, growth factors, cytokine activity, and angiogenesis (formation of new blood vessels). Hyperbaric oxygen therapy (HBOT) is a technology defined as administration of oxygen at pressures >1 atmosphere absolute (ATA) to the acute or chronic wounds or through the lungs to the whole body. Administration of HBOT involves placing the patient in an air tight hyperbaric vessel and administration of 100% oxygen for respiration. HBOT sessions, or dives, are usually conducted for 45-120 minutes daily at pressures between 1.5 to 3.0 ATA. A typical course of treatment may involve 20 to 30 HBOT sessions or dives. Administration of HBOT requires specialized hyperbaric chambers under the supervision of medical doctors who have had specialized training. Based on the facts mentioned above, the delivery of oxygen or pressurized oxygen-reach active substances/medicines can be done locally to the wounds, lesions, tissues, or organs via specialized new devices that incorporates one or multiple energy delivery systems as presented in energy combination systems embodiments FIG. 1A-1B, 8A-8B, 13A-13B, 13E, 14A-14E, 15-20, 21A-21B, 23-24, 26 of this invention, which can enhance the healing and regeneration of different human or animal wounds, lesions, tissues, or organs.

Warming therapy is another example of energy-delivering technology, which taps into the mechanism of action that relies on the activation of heat shock proteins for healing. However, the heating therapy should be applied with gradually increase in temperature/heat or in pulsed manner to avoid burns, regardless of being used independently or in energy combination medical treatments. Combination energy systems that can be used for such thermal treatments are the ones incorporating high intensity contact ultrasound or specialized lasers for ablation or thermal radio frequency waves similar to those presented in energy combination systems embodiments from FIGS. 5A-5B, 6A-6B, 9A-9B, 10A-10B, 11, 12, 14A-14B, 18, and 20.

One of the common characteristics for any medical treatment across any possible technology is the treatment time/duration. This parameter can be calculated by combination of multiple parameters or can be an independent one. For example, in the case of shockwaves the treatment time is equal with the total number of shockwaves for a treatment session divided with the number of shockwaves per second, which is the frequency of shockwave pulses. For ultrasound, phototherapy, electromagnetic, electrical stimulation, hydrotherapy, negative pressure, thermal therapy, etc., to name a few, the treatment time is an independent parameter that is calculated based on the size/area/volume of wound, tissue, or skin condition, the extend of local infection and its severity (grade of infection—I to V, the type of pathogen(s), and the organ or type of tissue), the volume of tissues or organs that need treatment for bacterial infection or biofilm infection or fungal infection or parasite infection or harmful micro-organisms' infection, the volume of necrotic skin or necrotic tissue or necrotic bone or necrotic organ, the area of the burned tissue, the volume of hard (bone), semi-hard (ligaments or tendons) or soft tissue (skin, muscle, tissues, organs, etc.) that requires regeneration, the length of skin or ligament or tendon tears, the volume of muscle that exhibits contraction, the gap and location of bone non-unions, the volume of an organ affected by anormal functioning, the size of bone fissure or fracture, the location and size of backbone that requires fusion, the size of a bone spur, the size of heterotopic ossification or calcification, the size of dental plaque, the area affected by pain or inflammation or edema, the grade of obstruction and size of the plaque/calcification, vulnerable plaque, or restenosis of a blood vessel, the percentage/size of an occlusion of a blood vessel, the size of a blood clot (thrombus or embolus) that blocks a blood vessel, the diameter and length of a blood vessel aneurysm, the size of the tissue inflammation produced by blood vessels stenting (metal or drug eluting stents) or by angioplasty with balloons or drug eluting balloons, the length of in-stent restenosis of blood vessels, the length of a blood vessel that have spasms, the volume of muscle spasm and contraction, the area of tissue that requires capillaries' formation, the diameter and size of a lymphatic vessel that needs repair, the size of heart muscle ischemia or in general tissue ischemia, the size of area around the heart valves that requires their actuation muscles regrowth and revascularization, the size of heart muscle regeneration, the area of the heart sack that requires treatment for fluid accumulation, the area that requires treatments using any type of human or animal or artificial grafts and biologics from placenta or other human harvested tissues, the size of area affected by lymphedema, the grade/amount of organ or tissue or vessel hyperplasia, the size of chronic tissue or organ inflammation, the area of organ adhesions produced by surgeries, the length of a surgical incision, the extend and severity of capsular contraction around implants, the severity of implant infection (area of infection, grade of infection—I to V, and the type of pathogen(s)), the extend of area of cellulite, the volume or area of body sculpting, the volume or area that needs liposuction, area affected by spider veins, area of skin that requires rejuvenation, volume or area of the zone that needs collagen formation, the size/area of a scar or fibrotic tissue, the severity and size of tissue spasm/contraction, the area surrounding the painful peripheral nerves, the location and size of peripheral nerve that requires regeneration, the area of ischemic tissue that requires blood vessel growth and/or angiogenesis or vasculogenesis, the length and width (area) of veins that needs treatment, the area of tissue/skin affected by autoimmune diseases, the size/volume of cancer tumors or unwanted benign tissue, the grade and severity of bacterial or abacterial prostatitis (chronic pelvic syndrome), the area of the bladder affected by interstitial cystitis, the volume of tissue or organ and the quantity of stem cells' solution or DNA/RNA/mRNA fragments or gene cocktails or proliferation factors or growth factors or angiogenesis factors used for the treatment, the volume of tissue that requires stimulation of dormant stem cells, the area of tissue or organ that requires in vivo stem cell proliferation and differentiation, the volume of a blood vessel or of the tissue or of the organ that needs localized drug delivery, the location and size of a patch or implant or any biodegradable structure that delivers localized drugs or active substances or cellular materials or vaccines, the location and the volume of tissue or organ that is affected by an autoimmune disease, the size of pacemaker leads or any implant or prosthesis that requires loosening and extraction, the size of the implant that needs treatment for aseptic loosening, and the like. The treatment algorithms from FIGS. 31-35, 37A-37P and energy combination systems embodiments presented into this invention (see FIGS. 1A-30B) are using treatment time for one therapy session, as a general parameter across any medical technology applied for treatment of human or animal subjects for healing or alleviate a certain medical condition. For energy combination systems embodiments from FIGS. 1A-30B and the treatment algorithms from FIGS. 31-35, 37A-37P, the initial treatment time (determined by the criteria mentioned above) can be altered based on patient's existing comorbidities and medical conditions (as obesity, diabetic neuropathy, peripheral vascular disease, venous insufficiency/stasis, poor glycemic control, ischemia of small and large blood vessels, previous injuries, deep venous thrombosis, phlebitis, central and peripheral nervous system disease, organs or glands afflictions, respiratory or digestive or urinary tract conditions, genetic disease, autoimmune syndromes, etc.), hygiene style, sex, age, race, genetic make-up, biometrics (weight, height, body mass), lifestyle (cigarette smoking, excessive alcohol consumption, nutrition, etc.), and other intrinsic factors that dictate the acute or chronic manifestation of the symptoms for the respective disease/affliction, in a similar way to the algorithm presented in U.S. Pat. No. 10,888,715 related to shockwave technology. In this way, the energy combination systems embodiments from FIGS. 1A-30B and the treatment algorithms from FIGS. 31-35, 37A-37P can be used to perform a personalized medical treatment regimen, which is specifically tuned for the medical situation of each patient. The same principle of treatment time (as presented above for humans and animals) applies also for plant treatment using energy combination systems or devices or treatment energy protocols.

The energy combination systems presented in the embodiments from FIGS. 1A-21E can be used to decontaminate organs before implantation, without using any harmful substances that can decrease the viability of the harvested organs from humans or animals as pigs. If the implant organ is created through decellularization and repopulated with stem cells/cells from the recipient who needs the respective organ, the energy combination systems presented in the embodiments from FIGS. 1A-21E can be successfully used, since majority of energy technologies can stimulate blood vessels growth, cellular multiplication, stem cells differentiation, etc. Furthermore, the energy combination systems presented in the embodiments from FIGS. 1A-21E can be used during decellularization process for complete disinfection and then for repopulation of the scaffolding with cellular material to produce a completely functional organ. The advantage of the decellularization and the use of the patient's own cells to repopulate the organ scaffold is that the rejection of the organ after implantation is very unlikely. Thus, shockwaves, ultrasound (contact or non-contact), electromagnetic waves, LED/OLED phototherapy, laser phototherapy, photodynamic phototherapy, ultraviolet light, energy-based nano-technology, electrical currents at the level of bio-currents, radio frequency fields, pressurized fluid medicine, can be combined in energy combination systems as presented in the embodiments from FIGS. 1A-21E, which can be used in conjunction with stem cells and/or genetic material, etc., to repopulate with new cells the decellularized organ and create a viable organ for transplant, without the inconvenience of rejection.

The genetic material or genetic modified material or the base editing apparatus can be used in conjunction with energy combination systems presented in the embodiments from FIGS. 1A-21E for plants and animals to help with diseases or to increase crop resistance and yields. Of course, besides the genetic material, other components as DNA, RNA, mRNA, gRNA can be used for the same purpose of dealing with diseases in animals, or for modifying plants to increase yield and make them more resistant to diseases and parasites. This can create sustainable solutions to address some of the biggest issues facing our planet today, from public health crises to environmentally-friendly food production for a growing population. Also, such agricultural products will help farmers create greener, cleaner crops by precisely targeting a specific pest with non-toxic bio-controls, and without harming beneficial insects or leaving residues in the soil or water.

The energy combination systems presented in the embodiments from FIGS. 1A-21E can easily incorporate the magnetotherapy, which creates specific magnetic fields that are part of bioenergy therapy. The magnets can be added to any of the applicators described in the energy combination systems presented in the embodiments from FIGS. 1A-21E. The magnetic fields have been shown to affect cell permeability and improve oxygen delivery to the cells, which can lead to better absorption of nutrients, improved circulation and clearance of waste products. Magnets may also reduce inflammation and pain, and promote healing. All these actions are complementary to the other technologies incorporated in the energy combination systems presented in the embodiments from FIGS. 1A-21E. In this way a new energy mechanism of action can be used to target a certain disease with multiple ways to activate the body's healing process.

The energy combination systems presented in the embodiments from FIGS. 1A-21E can activate different energy technologies incorporated in them concomitantly or subsequently. On the other hand, different energy technologies can be used in individual and different devices that have only one of such technologies incorporated in them. These individual and different devices can be independently used in the same time or at different time points of the treatment with the energy combination systems presented in the embodiments from FIGS. 1A-21E. The important aspect of such combinations of devices/systems is the increase of benefic energy in the targeted treatment region, to keep upregulation/boost of benefic factors or inhibition of harmful factors, and thus to accelerate the healing. This approach opens the possibility to give custom treatments based on the particular aspects of each affliction and the associated existing comorbidities and medical conditions (as obesity, diabetic neuropathy, peripheral vascular disease, venous insufficiency/stasis, poor glycemic control, ischemia of small and large blood vessels, previous injuries, deep venous thrombosis, phlebitis, central and peripheral nervous system disease, organs or glands afflictions, respiratory or digestive or urinary tract conditions, genetic disease, autoimmune syndromes, etc.), hygiene style, sex, age, race, genetic make-up, biometrics (weight, height, body mass), lifestyle (cigarette smoking, excessive alcohol consumption, nutrition, etc.), and other intrinsic factors that dictate the acute or chronic manifestation of the symptoms for the respective disease/affliction (as mentioned before in this invention), to be able to find the optimal treatment package acting in a sustained way to accelerate the healing process. In the future a robotic system capable of providing different combinations and flavors of the possible treatments, which the physician is able to select from a myriad of options, based on predetermined possibilities that rely on his/her knowledge or with the help of artificial intelligence systems.

The IoT (Internet of Things) involves a system of devices, machines, or anything with the ability to transfer data without the need for a human to implement the communication. In general, the IoT describes devices with sensors, processing ability, software, and other technologies that connect and exchange data with other devices and systems over the internet or other communications networks, which encompasses electronics, communication, and computer science engineering. Based on the recent adaptation of a variety of enabling wireless technologies, such as radio frequency identification (RFID) tags, wearable sensor, and actuator nodes, the IoT has stepped out of its infancy and it is the next revolutionary technology in transforming the internet into a fully integrated future internet. IoT is important due to its machine-to-machine component, but also due to the potential of sensor-to-machine interactions. With the increasing development of health sensors, there is a growing opportunity to utilize the IoT for medical data collection and analysis, data that then can be used by artificial intelligence (AI) algorithms to develop new treatment options or more sophisticated medical devices that can be used for customized medical treatments. It is expected that an integration of these tools into the health-care model has the potential of significantly lowering the annual costs for chronic disease management. This is why IoT it is a natural system to be used and introduced into functionality and the structure of the energy combination systems presented in embodiments from FIGS. 1A-30B and the treatment algorithms from FIGS. 31-35, 37A-37P.

The energy combination systems embodiments from FIGS. 1A-30B can also activate through their energy output different gels that contain nanoparticles or active medical substances that are activated by different forms of energy (as the ones used in the construction of the energy combination systems embodiments from FIGS. 1A-30B). Thus, the energy combination systems embodiments from FIGS. 1A-30B can facilitate the transformation of external energy produced by them in other forms of energy inside the organisms (as mechanical/kinetic, bioelectrical, chemical, etc.) that easily interact with the cells, tissues, or organs or produce and/or stimulate different factors that makes the targeted cells, tissues, or organs (from humans or animals or from plants) to react favorable to the treatment.

Similar or equivalent device constructions, or algorithms and principles, as the ones presented in the energy combination systems embodiments from FIGS. 1A-30B and for the treatment algorithms from FIGS. 31-35, 37A-37P of this invention, can be used/applied to other medical treatments for soft, semi-hard, or hard tissues for healing, stimulation, and regeneration/repair of cells, tissue and organs, to reverse abnormal organ functioning, to treat infections, cartilage or muscle or skin or ligaments or tendons tears, erectile dysfunction (ED), cancers, bone fractures, bone fusions, bone spurs, heterotopic ossifications, calcifications, plaque formation on teeth, pain and inflammation management, tissue necrosis, autoimmune diseases, blood vessels plaques, cardiac and endovascular obstructions and/or occlusions, blood vessels restenosis after angioplasty or stenting, heart muscle ischemia, lymphedema, organs and tissue hyperplasia, adhesions between organs after surgeries in the abdominal or muscular or chest areas, capsular contracture around implants, organ blockages or illnesses or shut downs, implant infections, cellulite, body sculpting, spider veins, skin rejuvenation, scar tissue and fibrotic tissue, tissue spasm/contraction, peripheral nerves to reduce pain or promote nerve regeneration and repair, tissue growth and/or angiogenesis or vasculogenesis, auto-immune diseases as Systemic Lupus Erythematosus or Scleroderma or Crohn's Disease or Dermatomyositis, cancer tumors or unwanted benign tissue growths or organ hyperplasia, bacterial or abacterial prostatitis (chronic pelvic syndrome), interstitial cystitis, etc., to name a few. Also, the energy combination systems embodiments from FIGS. 1A-30B and the treatment algorithms from FIGS. 31-35, 37A-37P can be successfully applied to the afflictions that require treatments using any type of cadaver-harvested human or animal cells, tissues, grafts, patches, or organs and bioactive cells or substances or scaffolding used for biologic products from placenta, umbilical cord, bone marrow, etc. The same can be applied for artificial cells, or tissues, or grafts, or implants, or patches, or organs that are created to be used for medical purposes.

Important to note that the energy combination systems embodiments from FIGS. 1A-30B do not have moving parts, which increases exponentially their reliability and maintenance simplicity. This is why the combination energy medical treatment systems are the answer for stimulating cells, tissues, organs for humans or animals or any plant material (cells or tissues) in the most efficient and economic ways.

The energy combination systems embodiments from FIGS. 1A-27 of this invention can be used extracorporeally in direct contact with the skin 15 or wound/lesion/tissue condition 19 or human/animal body 20 via a gel, hydrogel, or any fluid substance (which were not specifically depicted in any of the figures of this invention, but they are well known in the scientific literature) as seen in the embodiments from FIGS. 1A-14E, 21E, 22-27, or can be used with non-contact with the skin the skin 15 or wound/lesion/tissue condition 19 or human/animal body 20 as presented in the FIGS. 15-20, and 21D. Also, the energy combination systems embodiments from FIGS. 1A-21E of this invention can be used with an indirect contact with the skin, where pads or similar devices (as presented in WO 2007/108854) that can be used on top of the applicator/coupling membrane 15 from the embodiments of this invention. The pads can contain or not gel-like bioactive substances. When bioactive substances are present inside the pads, such substances can be pushed out of the pad (via controlled porosity of its skin/outer layer) to the targeted zone for the treatment using different energy technologies presented in energy combination systems embodiments from FIGS. 1A-21E. However, these pads are not similar to the specially designed coupling bladders or spacers presented before in this invention, since the specially designed coupling bladders or spacers contain fluids capable of generated cavitation bubbles that collapse forming micro-jets and thus helping when the energy combination systems embodiments from FIGS. 1A-21E are used for the medical treatment. Due to their high viscosity, the gel-like substances from the pads inhibit the formation of cavitation bubbles under the stimulation/action of energy combination systems embodiments from FIGS. 1A-21E. The advantage of such pads is that the penetration inside the targeted zone can be varied based on the thickness of the pad. Thin pads will allow deeper penetration of the energies delivered by the energy combination systems embodiments from FIGS. 1A-21E inside the human or animal body and thicker pads will allow less penetration (a more superficial treatment). Different penetrations using a system of pads of different thicknesses can be used with only one energy combination applicator 11, as presented in the embodiments of this invention, which can significantly reduce the total number of energy combination applicators 11 that a company must use for different targeted medical treatments, which translates in a reduced inventory. Also, for the medical professionals it is much easier to change multiple pads that can be placed on the top of the applicator/coupling membrane 15 to vary the treatment penetration inside the human or animal body, instead of changing multiple energy combination applicators 11 that are capable of different fixed penetrations. That translates in more flexible and less time-consuming medical treatments using energy combination systems embodiments from FIGS. 1A-21E, which finally means increased efficiency for the medical professionals performing the treatment.

The energy combination systems or devices presented in this invention in FIGS. 1A-27 or any possible variations or combination of them were presented as an extracorporeal option for the treatment of different afflictions for soft, semi-hard, or hard tissues, or cellular material, or organs of humans or animals and even for plant cells or tissues (plant materials). However, the same energy combination systems or devices presented in this invention in FIGS. 1A-27 or any possible variations or combination of them can be miniaturized for intracorporeal use, where the energy combination applicators 11 or the smart bandages 220, 230, 240, 250, 260, and 270, presented in different embodiments of this invention, are replaced by intracorporeal catheter systems, similar to the ones presented in U.S. Pat. No. 10,238,405 or U.S. Pat. No. 10,639,051 patents, which incorporate at their distal end or along their length the appropriate elements/means to deliver multiple forms of energies concomitantly or subsequently for the medical treatments. Such energy combination intracorporeal catheters can deliver inside the body energy combination outputs (to tissues or cells or organs adjacent to the intracorporeal catheter) in the form of shockwaves and/or ultrasound and/or electromagnetic waves and/or LED/OLED light fields and/or laser fields and/or ultraviolet light fields and/or electric currents and/or radio frequency waves and/or energy-based nanoparticles and/or pressurized active fluid medicine, to name a few. To reach the targeted treatment area, the energy combination intracorporeal catheters can use the natural human and animal conduits or artificially created conduits. The use of multiple energy delivery systems/technologies on one energy combination intracorporeal catheter assures that the targeted tissues or organs are subject to multiple mechanisms of action that will be used to accomplish the goal of the treatment, as vessels plaque fragmentation, elimination of obstruction or occlusions or blood clots or embolus, triggering or stimulating the healing/revitalization of surrounding tissues, etc., which in the end assures a more efficient treatment and a faster healing.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim(s) with the subject matter of each other dependent claim(s) or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

Various embodiments of the invention have been described. While the invention has been described with reference to exemplary structures and methods in embodiments, the invention is not intended to be limited thereto, but to extend to modifications and improvements within the scope of equivalence of such claims to the invention. It will be evident that various modifications and changes may be made thereto, and additional embodiments may be implemented, without departing from the broader scope of the invention as set forth by the claims. This specification is to be regarded in an illustrative rather than a restrictive sense.

What is claimed is:

1. An apparatus for treating a target area of a living thing comprising:
a plurality of different energy generation sources configured to directly or indirectly couple to the living thing; and
a control console communicatively coupled to each of the different energy generation sources to provide a calculated dosing of respective energy from each energy source during a single treatment session of the living thing.

2. The apparatus of claim 1, wherein the control console includes an artificial intelligence central processing unit configured to calculate the doing of respective energy from each energy source.

3. The apparatus of claim 2, wherein each energy source of the plurality of different energy generation sources is selected from the group consisting of heat, pressure, mechanical vibration, electrical, electromagnetic, radio frequency, photonic and plasma.

4. The apparatus of claim 1, wherein each energy source of the plurality of different energy generation sources is selected from the group consisting of heat, pressure, mechanical vibration, electrical, electromagnetic, radio frequency, photonic and plasma.

5. The apparatus of claim 4, further comprising a display communicatively coupled to the control console and operable to show one or more regions of the living thing being treated during the single treatment session by each energy source and the selection of the plurality of different energy generation sources configured to directly or indirectly couple to the living thing;

6. The apparatus of claim 5, wherein the display is operable to show an amount of treatment from each of the energy sources remaining before, after or during the single treatment session.

7. The apparatus of claim 6, further comprising at least one wound, lesion or tissue applicator coupled to at least one energy source.

8. The apparatus of claim 5, further comprising at least one wound, lesion or tissue applicator coupled to at least one energy source.

9. The apparatus of claim 1, further comprising at least one wound, lesion or tissue applicator coupled to at least one energy source.

10. The apparatus of claim 4, wherein each energy source of the plurality of different energy generation sources produces a treatment effect selected from the group consisting of shockwaves, radial pressure waves, planar pressure waves, cylindrical pressure waves, non-contact ultrasound, contact ultrasound, electromagnetic waves, LED light therapy, OLED light therapy, phototherapy, laser phototherapy, ultraviolet light, cold plasma, radio-frequency, vacuum, electrical current, topical negative pressure, energy-activated nanotechnology, pneumatic compression, and hydrotherapy.

11. The apparatus of claim 1, wherein each energy source of the plurality of different energy generation sources produces a treatment effect selected from the group consisting of shockwaves, radial pressure waves, planar pressure waves, cylindrical pressure waves, non-contact ultrasound, contact ultrasound, electromagnetic waves, LED light therapy, OLED light therapy, phototherapy, laser phototherapy, ultraviolet light, cold plasma, radio-frequency, vacuum, electrical current, topical negative pressure, nanotechnology, pneumatic compression, and hydrotherapy.

12. The apparatus of claim 2, wherein each energy source of the plurality of different energy generation sources produces a treatment effect selected from the group consisting of shockwaves, radial pressure waves, planar pressure waves, cylindrical pressure waves, non-contact ultrasound, contact ultrasound, electromagnetic waves, LED light therapy, OLED light therapy, phototherapy, laser phototherapy, ultraviolet light, cold plasma, radio-frequency, vacuum, electrical current, topical negative pressure, nanotechnology, pneumatic compression, and hydrotherapy.

13. A method of treating a living thing comprising:
automatically determining a respective treatment protocol for delivery of energy treatment from each electronic energy-producing source of a plurality of different electronic energy sources based on a type of condition and one or more of the size, area and volume of a region of the condition to be treated; and
administering said each respective treatment protocol for delivery of energy treatment from each of the plurality of different electronic energy-producing sources from a single treatment application simultaneously controlling the plurality of different electronic energy-producing sources during a single treatment session.

14. The method of claim 13, wherein the respective treatment protocols are determined by an artificial intelligence processor.

15. The method of claim 14, wherein each energy source of the plurality of different energy generation sources is selected from the group consisting of heat, pressure, mechanical vibration, electrical, electromagnetic, radio frequency, photonic and plasma.

16. The method of claim 13, wherein each energy source of the plurality of different energy generation sources is selected from the group consisting of heat, pressure, mechanical vibration, electrical, electromagnetic, radio frequency, photonic and plasma.

17. The method of claim 16, wherein the living thing is a human or animal and the condition is a wound, lesion or tissue condition.

18. The method of claim 13, wherein each respective treatment protocol uses an energy treatment selected from the group consisting of shockwaves, radial pressure waves, planar pressure waves, cylindrical pressure waves, non-contact ultrasound, contact ultrasound, electromagnetic waves, LED light therapy, OLED light therapy, phototherapy, laser phototherapy, ultraviolet light, cold plasma, radio-frequency, vacuum, electrical current, topical negative pressure, energy-activated nanotechnology, pneumatic compression, and hydrotherapy.

19. The method of claim 14, wherein the living thing is a human or animal body and the condition is a wound, lesion or tissue condition, and further comprising using artificial intelligence to determine each respective treatment protocol based on or more of whether the condition is chronic or acute, whether the condition is internal or external to the body, whether the condition is ischemic or non-ischemic, whether the condition includes infection or is free of infection, whether the condition shows inflammation and whether the condition is causing pain.

20. The method of claim 18, wherein the living thing is a human or animal body and the condition is a wound, lesion or tissue condition, and further comprising using artificial intelligence to determine each respective treatment protocol based on or more of whether the condition is chronic or acute, whether the condition is internal or external to the body, whether the condition is ischemic or non-ischemic, whether the condition includes infection or is free of infection, whether the condition shows inflammation and whether the condition is causing pain.
